(19) United States
(12) Patent Application Publication
(10) Pub. No.: US 2023/0165825 A1
(43) Pub. Date: Jun. 1, 2023

(54) NOVEL ANHYDROUS COMPOSITIONS COMPRISED OF MARINE OILS

(71) Applicant: OMEZA HOLDINGS, INC., Sarasota, FL (US)

(72) Inventors: Griscom BETTLE, III, Sarasota, FL (US); Michael C. INMAN, Sarasota, FL (US); Sarah M. KITLOWSKI, Sarasota, FL (US)

(73) Assignee: OMEZA HOLDINGS, INC., Sarasota, FL (US)

(21) Appl. No.: 17/920,162

(22) PCT Filed: Apr. 20, 2021
(86) PCT No.: PCT/US2021/028056

§ 371 Date: Oct. 20, 2022

Related U.S. Application Data
(60) Provisional application No. 63/013,167, filed on Apr. 21, 2020.

(51) Int. Cl. A61K 31/202; A61K 9/00; A61K 35/60; A61K 36/185; A61K 36/889; A61K 31/23; A61K 38/39; A61K 31/167; A61K 9/06; A61K 8/04; A61K 8/92; A61Q 19/00; A61K 8/65; A61K 8/37; A61K 8/36; A61P 27/04; A61P 17/02; A61K 31/14; A61P 17/06; A61P 17/10; A61K 31/60

(52) U.S. Cl. CPC A61K 31/202; A61K 9/0014; A61K 35/60; A61K 36/185; A61K 36/889; A61K 31/23; A61K 38/39; A61K 31/167; A61K 9/06; A61K 8/042; A61K 8/922; A61K 8/925; A61Q 19/007; A61K 8/65; A61K 8/37; A61K 8/375; A61K 8/361; A61P 27/04; A61P 17/02; A61K 31/14; A61P 17/06; A61P 17/10; A61K 31/60; A61K 2800/5922

(57) ABSTRACT

The present disclosure describes a novel anhydrous topical pharmaceutical composition comprised of marine oil, such as fish oil or algae oil; vegetable oil having a an omega3 fat content greater than 9 wt %, such as hempseed oil, canola oil, flax seed oil, or cannabidiol; monolaurin, cetyl esters or wax; and medium chain triglycerides (“MCT”), and optionally fish collagen. The composition is useful for treating wounds, burns and skin conditions.

### FIELD OF THE DISCLOSURE

The present disclosure relates to an anhydrous topical composition comprised of marine oil, such as fish oil or algae oil, vegetable oil having an omega3 fatty acid content greater than 9 wt %, MCT (medium chain triglycerides), monoglyceride, such as monolaurin, and linear fatty acid esters, such as cetyl esters and/or waxes, and optionally collagen for treating wounds, burns and skin conditions.

### BACKGROUND OF THE DISCLOSURE

Marine oil, including cod liver oil, salmon oil and oil from algae, such as seaweed, contains, among other components, omega3 fatty acids, which have a variety of anti-inflammatory and immune-modulating effects that may be of relevance for treating diseases and conditions where inflammation is an underlying cause. Inflammation is the body's attempt at self-protection where the aim is to remove harmful stimuli and start the healing process. Inflammation may be divided into two types of inflammation: acute and chronic inflammation. Acute inflammation is short-term inflammation which starts rapidly and quickly in response to tissue damage. Examples of acute inflammation include acute bronchitis and acute appendicitis. Chronic inflammation is a slow and prolonged inflammation response which involves a progressive shift in the type of cells present at the site of inflammation characterized by the simultaneous destruction and repair of tissue during the inflammatory process. Examples of chronic inflammation include, but are not limited to, failure to eliminate the causing agent, an autoimmune response to a self-antigen and a chronic irritant of low intensity that persists. Chronic inflammation may, however, mature into severe diseases such as chronic obstructive pulmonary disease (COPD), cancer, atherosclerosis, Alzheimer's disease (AD), amyotrophic lateral sclerosis (ALS), and the like.

The omega3 fatty acids are essential to life at any stage, even before birth. They are essential building blocks of the membrane of every cell in the body, and their presence is a necessity for maintaining an adequate cell membrane. They also contribute in the regulation of most biological functions.

The richest dietary source of very long-chain omega3 polyunsaturated fatty acids (PUFA) comes from marine oil, such as fish oil and algae oil. Fatty acids are the building blocks of dietary fats and are stored substantially in the form of triglycerides. The body cannot, however, produce polyunsaturated fatty acids and must obtain them from food sources or from supplements. Four fatty acids that are present in the omega3 family include alpha-linolenic acid (ALA), stearidonic acid (SDA), eicosapentaenoic acid (EPA) and docosahexaenoic acid (DHA). ALA is found in, e.g., walnuts, some types of beans, hempseed oil and olive oils. EPA, SDA and DHA are found in fish, including fish oil and algae and supplements. These omega3 fatty acids are useful in treating wounds, burns and skin conditions.

A recent study at Brigham and Women's Hospital in Boston revealed that omega3 fatty acids actually convert into compounds that are 10,000 times more potent than the original fatty acids themselves. These compounds include resolvins, which help bring an inflammatory response in the body to an end. Resolvins and protectins are oxygenated metabolites derived from EPA and DHA, and a part of the molecular mechanisms contributing to removal of inflammatory cells and restoration of tissue once the need for inflammatory response is over. It has been shown that aspirin treatment enhances the conversion of EPA and DHA to resolvins which carry potent anti-inflammatory signals. The mechanisms by which their effects are exerted are still a matter of controversy, but it seems likely that these oxygenated metabolites play a significant role as they have potent anti-inflammatory and immunoregulatory actions even in concentrations in the nanomolar and picomolar range. As tissues return to normal, resolvins and protectins together with further oxygenated metabolites, such as lipoids and maresins, promote resolution of the inflammation through removal of leucocytes and cellular debris.

It is important to recognize that the healing of wounds, burns and skin conditions does not occur just at the situs of the burn or skin condition, but also requires assistance from the broadly-defined periwound and from increased blood flow, either from sharp debridement or blood-driven oxidative bursts (also known as “respiratory bursts”).

During the healing process, neutrophils are recruited to the injury site to destroy pathogenic organisms. Macrophages arrive at the injury site and engulf debris and dead cells. The area where the injury occurs usually becomes inflamed. This inflammatory phase ends when inflammatory cells are actively removed, likely via apoptosis. In the following phase, keratinocytes proliferate to reform the epidermis. Fibroblasts migrate to the injury site and are stimulated to proliferate by platelet-derived growth factor (PDGF) and other growth factors. During this phase, fibroblasts and their derived myofibroblasts break down the fibrin clots by secreting plasminogen activators that convert plasminogen to plasmin, which cleaves fibrin to dissolve the clot. Fibroblasts and myofibroblasts also secrete collagen and other extracellular matrix proteins that form granulation tissue. The formation of granulation tissue is accompanied by the development of new blood vessels, re-epithelialization at the site of injury, and contraction of the granulation tissue to bring the edges of the skin where the injury occurred closer together. In a final phase, fibroblasts, macrophages and endothelial cells secrete extracellular matrix proteins and matrix metalloproteinases that remodel the granulation tissue, replacing the collagen type III matrix with a stronger, more organized collagen type I matrix.

Reactive oxygen species (ROS) play a pivotal role in the orchestration of the normal healing response. They act as secondary messengers to many immunocytes and non-lymphoid cells, which are involved in the repair process, and appear to be important in coordinating the recruitment of lymphoid cells to the injury site and effective tissue repair. ROS also possess the ability to regulate the formation of blood vessels (angiogenesis) at the injury site and the optimal perfusion of blood into the injury-healing area. ROS act in the host's defense through phagocytes that induce a ROS burst onto the pathogens present at the injury site, leading to their destruction. Moreover, during this period, excess ROS leakage into the surrounding environment has further bacteriostatic effects. In light of these important roles of ROS in healing and the continued quest for therapeutic strategies to treat burns and skin conditions, the manipulation of ROS represents a promising avenue for improving these responses when they are stalled.

The treatment of wounds, burns and skin conditions requires oxygen to form Reactive Oxygen Species (ROS) that kill bacteria, fungi and viruses. But excess ROS can also damage cells. There must be a mechanism that allows sufficient ROS to sanitize the wound, burn or skin condition, but also quenches excess ROS free radicals to prevent tissue damage.

Non-inflammatory macrophages M2 clean up the debris from the ROS sanitation. EPA and DHA infiltrate the lipid bilayer shell of cells. As excess ROS attacks the cell wall, the antioxidant capability of polyunsaturated fatty acids (PUFA), such as EPA, SDA, ALA and DHA, quench the free radicals and provide a check and balance with respect to the ROS so to promote disinfection of the wound, burn or treated skin condition, improved signaling, the commencement of granulation and epithelization and healing.

Dermal macrophages for ROS sanitation are located in close proximity to hair follicles, in the surrounding connective tissue sheath.

As macrophages possess a high degree of phenotypic plasticity, they react not only to the treatments that are introduced, but also to the existing microenvironment, which may affect their ability to respond to treatment. This is an important consideration that therapies should address.

A number of recent studies have demonstrated that mitochondrial energy metabolism has a strong correlation with inflammatory responses, including macrophage polarization. Specifically, classically activated M1 macrophages depend on glycolysis and further lactate fermentation, even in the presence of excess oxygen in cytoplasm. In contrast, alternatively activated M2 macrophages seem to utilize oxidative phosphorylation (OXPHOS), for which fatty acids from dietary lipids might aid in the supply of substrate acetyl-CoA through β-oxidation.

In detail, energy metabolism of M1 macrophages is mainly dependent on glycolytic pathway releasing inflammatory lactate, whereas M2 macrophages rely on fatty acid oxidation (FAO) which provides non-inflammatory acetyl-CoA, a substrate for oxidative phosphorylation. Accordingly, M1 macrophages were shown to demand lower cellular oxygen than M0 macrophages, whereas M2 macrophages consume oxygen in an increased rate. That is, it takes more blood oxygen to generate anti-inflammatory M2 macrophages.

MCT is preferentially consumed (versus simple sugars) by cells for energy. MCT-fed cells produce anti-inflammatory macrophage M2 in intact skin adjacent to hair follicles.

MCT oil drives macrophages to exhibit anti-inflammatory functions by enhancing mitochondrial respiration. MCT oil exhibited no effect on oxygen consumption in a non-stimulated M0-like status. Interestingly, however, under M1-like inflammatory conditions following LPS (lipopolysaccharide, an endotoxin) stimulation, MCT oil significantly increased OCR (oxygen consumption rate) for basal respiration, maximal respiration, and ATP production indicating that MCT not only suppresses inflammatory responses in M1 status, but also actively upregulates the switch of macrophage function towards M2-like repolarization. MCT enhances anti-inflammatory responses of macrophages via up-regulating mitochondrial respirations in accordance with previous reports, in which increased energy expenditure by medium-chain fatty acids (MCFAs) drives macrophages to exhibit anti-obesity and anti-inflammatory phenotypes.

Overall, up-regulated β-oxidation by MCT contributes to the anti-inflammatory M2-like status of macrophages, which may aid in the prevention and/or amelioration of inflammation.

MCT also indirectly reduces pain. Not by actively reducing pain like lidocaine, novocaine and similar “-caines”, but by not producing inflammatory lactate. For example, it was found that a composition comprising the combination of camphor, lidocaine and MCT, present in total in the formulation at 60 wt %, reduces pain deep in the muscular tissue. Camphor cools surface skin as it evaporates; lidocaine temporarily reduces pain in the dermis; high levels of MCT reduce deep pain by NOT producing inflammatory lactate. Together, the patient does not feel pain for hours.

So MCT can stimulate anti-inflammatory macrophages to decrease inflammation and to help create ROS, but excess ROS has to be quenched. Free radical quenching is done by PUFA from vegetable and marine sources.

In particular, the lipophilic structure of PUFA and molecular space dimensions allow EPA fatty acids to insert efficiently into lipoprotein particles and cell lipid membranes where it scavenges free radicals. In contrast to EPA, DHA serves essential functions in nervous tissues where it is abundant and has pronounced effects on neuronal and retinal membrane organization.

Several lines of evidence show that EPA and DHA differ in their antioxidant properties as well as in their apparent effects on membrane lipid structure and dynamics. The antioxidant effects of EPA are attributed to its ability to quench reactive oxygen species associated with cellular membranes and lipoproteins. Following intercalation into the lipid particle or membrane, the multiple double bonds associated with EPA facilitate electron stabilization mechanisms that inhibit free radical propagation. The antioxidant effects of EPA could not be reproduced with vitamin E or other FDA-approved triglyceride-lowering agents under normal or hyperglycemic conditions in vitro. The antioxidant activity of EPA could not be reproduced over time with DHA in lipoprotein particles.

Many formulations for treating wounds, burns and skin disorders applied topically to the site of injury are oils, gels, salves, or paste and contain omega3 fatty acids. In order to effect treatment, the omega3 fats in the formulations must pass through the skin. However, these formulations are difficult to use topically because they partition into a retentate and a permeate fraction. The permeate passes through the stratum corneum into the dermis and then into the bloodstream (transdermal). The retentate often leaves a greasy-feeling residue on the skin surface. Skilled artisans overcome this defect by mixing oils into water-in-oil emulsions (or oil-in-water) which has the effect of reducing the mole fraction of oil residual on the skin surface.

Transdermal compositions are well known, where enhancers modify the barrier of the stratum corneum and allow low molecular weight, lipid-soluble active pharmaceutical ingredients (API), including omega3 fatty acids, to pass into the dermis and then into the bloodstream. In order for the API to enter the bloodstream, the API must pass through the skin. Skin is complex. First there is a thin outer barrier layer, the stratum corneum, part of the epidermis. The dermis is under the epidermis. The basement membrane, a wavy line, separates the epidermis from the dermis. The epidermis does not have blood flow; the dermis has strong blood flow. The dermis sits atop the hypodermis, a fatty layer. In addition, the hair follicle is rooted in the dermis and extends upwards through the epidermis.

There are two practical pathways for topical compositions to pass through the epidermis.

Across the stratum corneum (intercellular route)

Down the hair follicle (interfollicular route).

The intercellular route deposits permeate at the basement membrane separating the epidermis from the dermis (intradermal). The hair follicle route deposits unconsumed permeate at the cutaneous fat layer below the dermis (transdermal). The effect and ramifications of the API passing through the epidermis via the intercellular route or the hair follicle route are different.

There is very little research on the effect of topical omega3 fatty acids. Topical application of omega3 fatty acids has the enormous benefit of targeting the site with a dose commensurate with the need. On the other hand, when omega3 fatty acids are diluted, the dose thereof is not only diluted, but, in addition, the omega3-fatty acids are disseminated throughout the body. However, problems with a topical application of omega3 fatty acids isolated from fish oil is smell (fishy, rancid fat), greasy skin and the barrier associated with the stratum corneum, the outermost layer of the epidermis. Long chain fats do not migrate through this barrier easily and even if they do get across the outer barrier, they have to migrate through the rest of the epidermis and the “basement membrane” that separates the epidermis from the dermis. Only in the dermis can the omega3 fats enter the blood stream.

### SUMMARY OF THE DISCLOSURE

The present inventor has found novel pharmaceutical compositions comprised of omega3 fatty acids which heal wounds, burns and skin conditions and enter the bloodstream through the interfollicular pathway and/or the intercellular pathway. The present disclosure describes an anhydrous topical pharmaceutical composition comprised of fish oil or algae oil, whether grown in the salt water or non-salt water, such as fresh water, or are farm-raised or grown in flowerpots or in or any other growing surface for algae; vegetable oil having an omega3 fatty acid content greater than 9 wt %, such as hempseed oil, canola oil, flax seed oil, or cannabidiol; linear fatty acid ester having 12-50 carbon atoms, such as cetyl esters or wax; monoglyceride of the formula R—C(O)—OCH₂—CH(OH)—CH₂OH, such as monolaurin, where R is an alkyl group of 7-11 carbon atoms; and medium chain triglycerides (“MCT”), and optionally collagen; said vegetable oil having an omega3 fatty acid content greater than 9 wt % comprised of ALA, SDA, EPA and DHA, wherein the weight ratio of the sum of the weights of (ALA+SDA)/sum of the weights of (EPA and DHA) present in the composition ranges from about 0.5 to about 1.5, and in addition, in the absence of collagen, when the composition is an oil, the sum of the wt % of MCT, monoglyceride and linear fatty acid in said pharmaceutical composition is greater than or equal to 42 wt %, or in the presence of collagen, the sum of the wt % of MCT, monoglyceride and linear fatty acid esters in said pharmaceutical composition is greater than or equal to 20 wt %. An embodiment of the present composition contains no fish collagen or any collagen from any other source. An embodiment of the present disclosure contains collagen. The pharmaceutical compositions described herein, whether collagen free or containing collagen have permeate factors and retentate factors. In an embodiment of collagen-free composition, the permeate factor is greater than or equal to 3.0 and the retentate factor is less than or equal to 8.0. In another embodiment of the collagen free composition, the permeate factor is greater than or equal to 3.0 and the retentate factor is greater than 8.0. In still another embodiment of the collagen-free composition, the permeate factor is less than 3.0 and the retentate factor is less than or equal to 8.0. In a fourth embodiment of the collagen-free composition, the permeate factor is less than 3.0 and the retentate factor is greater than 8.0. The compositions herein are in the form of an oil, gel, salve, cream, paste or whip. With respect to the compositions comprised of collagen described herein, in an embodiment, the permeate factor is greater than or equal to 1.0. In an embodiment of the collagen containing composition, wherein the permeate factor is greater than or equal to 1.0, the retentate factor is greater than or equal to 8.0. In an embodiment of a pharmaceutical composition comprised of collagen, the permeate factor is greater than or equal to 1.0, and the retentate factor is greater than or equal to 8.0.

In an embodiment, the present disclosure also describes a topical pharmaceutical composition in the form of a gel which comprises cod liver oil, hempseed oil, monolaurin, cetyl esters, medium chain triglycerides (“MCT”), coconut oil and palm oil, wherein said composition has a C18:0 triglyceride concentration of 3 wt % or less, said composition being anhydrous and homogenous, and having a weight ratio of the sum of the weights of (ALA+SDA)/(sum of the weights of EPA and DHA) present in the composition ranging from about 0.5 to about 1.5; and the weight ratio of the sum of the weights of saturated (C8+C10 saturated triglycerides)/sum of the weights of unsaturated triglycerides larger than C10 ranging from about 1.0 to about 3.0, and the amount of monolaurin being greater than 6 wt % of the composition, and the amount of MCT present being greater than 30 wt %. Such gelled formulation exists either as a low viscosity gel or a higher viscosity gel. The gelling is formed by monolaurin and is independent of the liquid oil concentration. In an embodiment of the gel composition described herein, the cod liver oil is present in an amount ranging from about 5 wt % to about 30 wt %, hemp oil is present in an amount ranging from about 5 wt % to about 30 wt %, palm oil is present in an amount ranging from about 0 wt % to about 20 wt %, coconut oil is present in an amount ranging from about 0 wt % to 20 wt %, MCT is present in an amount ranging from about 30 wt % to about 65 wt %, cetyl esters are present in an amount ranging from about 0.5 to about 3.0 wt %, and monolaurin is present in an amount ranging from about 7 wt % to about 12 wt %, wherein the weight ratio of the sum of the weights of ALA+SDA/sum of the weights of EPA and DHA ranges from about 0.5 to about 1.5, and the composition has a C18:0 concentration of 3 wt % or less. In an embodiment, the weight ratio of palm oil to coconut oil ranges from about 4:1 to about 1:1.

The sum of the wt % of all of the above components in the formulations described herein ranges from about 80 wt % to 100 wt %. In an embodiment, the composition contains no collagen or sea salt. In another embodiment, no C8/C10 free fatty acids are added to the composition. In another aspect, the composition of the present disclosure is a drug carrier for oil soluble drugs including pain killers, such as lidocaine and aspirin. In an embodiment, the compositions of the present disclosure without medicament are carriers for drugs, such as pain killers, wherein the amount of drug present in the composition of the present disclosure as a carrier ranges from about 0.5% to about 4% of the composition.

In addition, an aspect of the present disclosure is directed to treating a wound, burn or skin condition on a subject by topically applying an effective amount of the pharmaceutical composition described herein. In addition, the compositions of the present invention are useful as cosmetic, as moisturizers and for treatment of dry skin. Further, the gelled compositions exist as high and low viscosity gels, and both viscosity gelled compositions are useful as cosmetics which moisturizes the skin, leaving a smooth silky finish.

Another aspect of the present disclosure is directed to a method of treating a subject who has dry eye syndrome which comprises applying an effective amount of the pharmaceutical composition described herein, topically to skin adjacent to the eye. In an embodiment, the composition for treating dry eye syndrome is a gelled composition. In another embodiment, the composition for treating dry eye syndrome is an oil. Gelled formulation and non-gelled formulations both are effective for treating dry eye syndrome, but gelled formulations are easier to apply to tender eye-surrounding skin. Collagen-containing formulations are not as useful for treating dry eye syndrome as collagen cakes on the skin after the compositions are absorbed. Without wishing to be bound, it is believed that omega3 fatty acid in combination with MCT drive the omega3 fatty acids into the skin adjacent to the dry eye. The gel has an advantage because of its viscosity and because they retard the composition from dripping directly onto the eyeball. Direct application to the eye irritates the eye. Transdermal compositions migrate to the blood flow feeding the eyes indirectly. The omega3 fatty acids thus get into the eye indirectly.

The oil versus gel decision is a personal decision of the user. Gels do not drip but are more difficult to apply uniformly. Many users use the “finger-tap” method of applying oil (i.e. apply oil to the finger and then tap the eye-surrounding skin to transfer oil to the skin without shear).

The compositions divide into three groups: gels, oils and collagen products. They are very different. Gels are made by increasing wax levels or gels are made by increasing monoglyceride levels, such as monolaurin levels, greater than or equal to 6.0 wt % of the compositions until a gel is formed. The gel can mechanically support a variety of additives to differentiate formulations. Gels are absorbed by the skin slowly, allowing time for the additives to be dragged through the epidermis.

Oils are used to help inhibit biofilm formation by leaving a greasy film on skin that does not allow bacteria to gain a foothold on skin, particularly open wounds. Oil-soluble additives, such as lidocaine, can be dissolved in the oils and dragged into the dermis.

Collagen products have inherently less oil because so much of the 100% formula is consumed by collagen. Collagen products are either leave-behind formulations where the collagen is eventually absorbed to help heal open wounds and treat skin conditions. In an embodiment, collagen products are used topically as a skin polish to exfoliate dead and dying skin by mechanically rubbing old skin off viable skin (when permeate factor is less than 1.0). In another embodiment, when the permeate factor is greater than or equal to 1.0, collagen products are used to treat minor wounds by absorbing exudates and inhibiting bacterial growth with antimicrobials, like benzethonium chloride.

### BRIEF DESCRIPTION OF THE DRAWINGS

The objects, features and advantages will become apparent to one of ordinary skill in the art, in view of the following detailed description taken in combination with the attached drawings.

FIG. 1 is a comparative graphical representation depicting the rise in palm temperature as a function of time when Oil BV and Oil D are each separately applied topically to the palm of a human subject.

FIG. 2 is a comparative graphical representation of the palm temperature change as a function of time after Oil D and Gel CS1C are separately applied topically to the palm of a human subject.

FIG. 3 is a comparative graphical representation of the palm temperature change when a low dose and a higher dose of Oil D are applied topically to the palm of a human subject.

FIG. 4 is a comparative graphical representation comparing the surface oxygen saturation of Lidocaine Lavage AA and Omeza Gel AF when applied topically to the leg of a human subject.

FIG. 5 is a comparative graphical representation comparing the average subsurface reticular dermis hemoglobin oxygen saturation of Lidocaine Lavage AA and Omeza Gel AF when applied topically to the leg of a human subject.

FIG. 6 are photographs that were taken prior to treatment with Collagen Matrix DF of a chemically burned large hole in the scalp of a woman and 20 days and 34 days after application thereto.

FIG. 7 are photographs depicting a severe acne problem on the upper back of a female prior to application of Omeza Acne F thereto and 4 days after topical application thereto.

FIG. 8 are photographs depicting a severe acne problem just below the neck of a female prior to application of Omeza Acne F thereto and 4 days after topical application thereto.

FIG. 9 shows photographs depicting a diabetic ulcer on the toe of a human male subject before and 1 and 14 days after topical treatment with the Omeza Bundle.

FIG. 10 shows photographs depicting a diabetic ulcer on the toe of a human subject before and 1 and 5 days after topical treatment with the Omeza Bundle.

FIGS. 11-19 tabulate and graphically depict the effectiveness of the Omeza Bundle in the treatment of wounds in elderly patients, illustrating the effects with photographs taken before and after treatment. The y-axis in each case is the surface area of the wound in cm², while the x-axis depicts time after application of Omega Bundle (days).

In FIG. 11, the wound was a stage 3 pressure wound on the left heel of a 98 year old patient. FIG. 11a tabulates the size of the wound before and after treatment with the Omeza Bundle, FIG. 11b graphically depicts the change in the size of the wound after treatment with the Omeza Bundle; FIG. 11c is a photograph of the wound prior to treatment with the Omeza Bundle, and FIG. 11d is a photograph of the wound after treatment was completed.

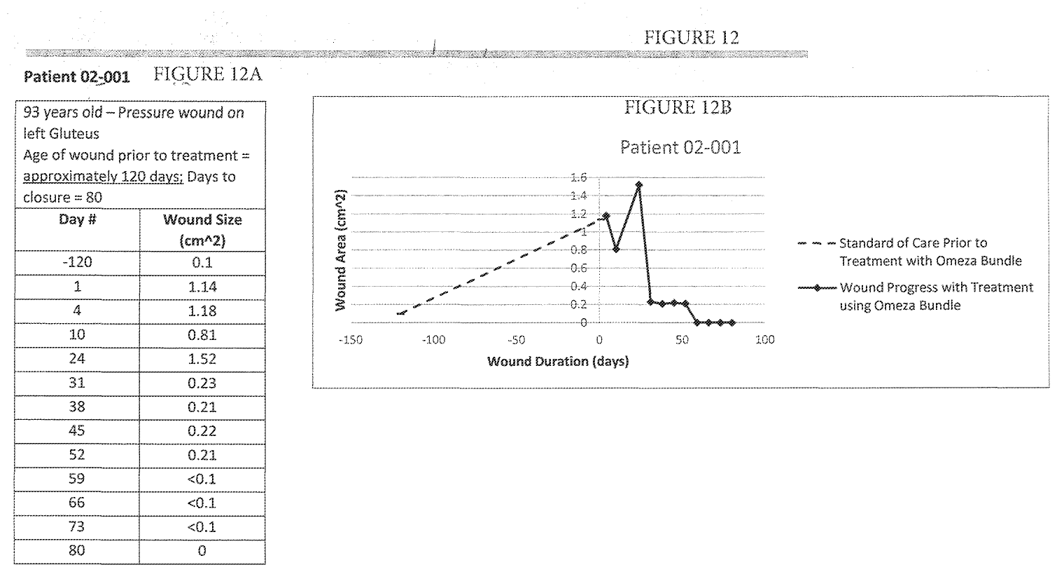

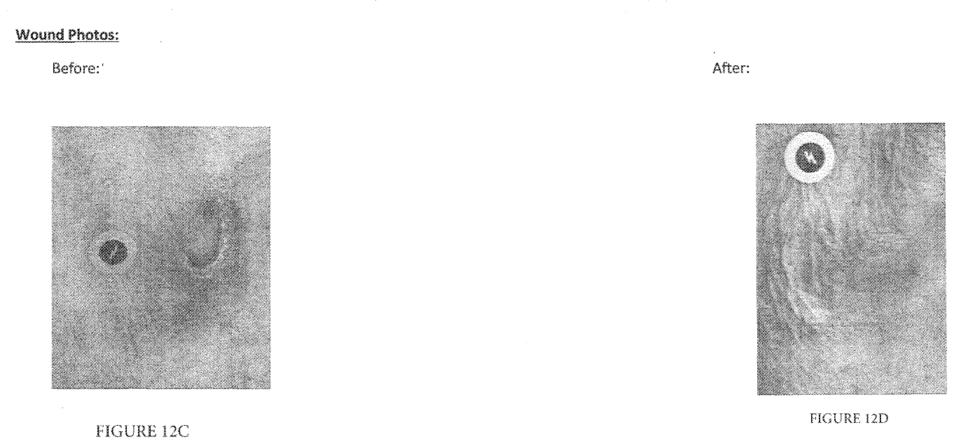

In FIG. 12, the wound was a stage 2 pressure wound on the left gluteus on a 93 year old patient. FIG. 12a tabulates the size of the wound before and after treatment with the Omeza Bundle, FIG. 12b graphically depicts the change in the size of the wound after treatment with the Omeza Bundle; FIG. 12c is a photograph of the wound prior to treatment with the Omeza Bundle, and FIG. 12d is a photograph of the wound after treatment was completed.

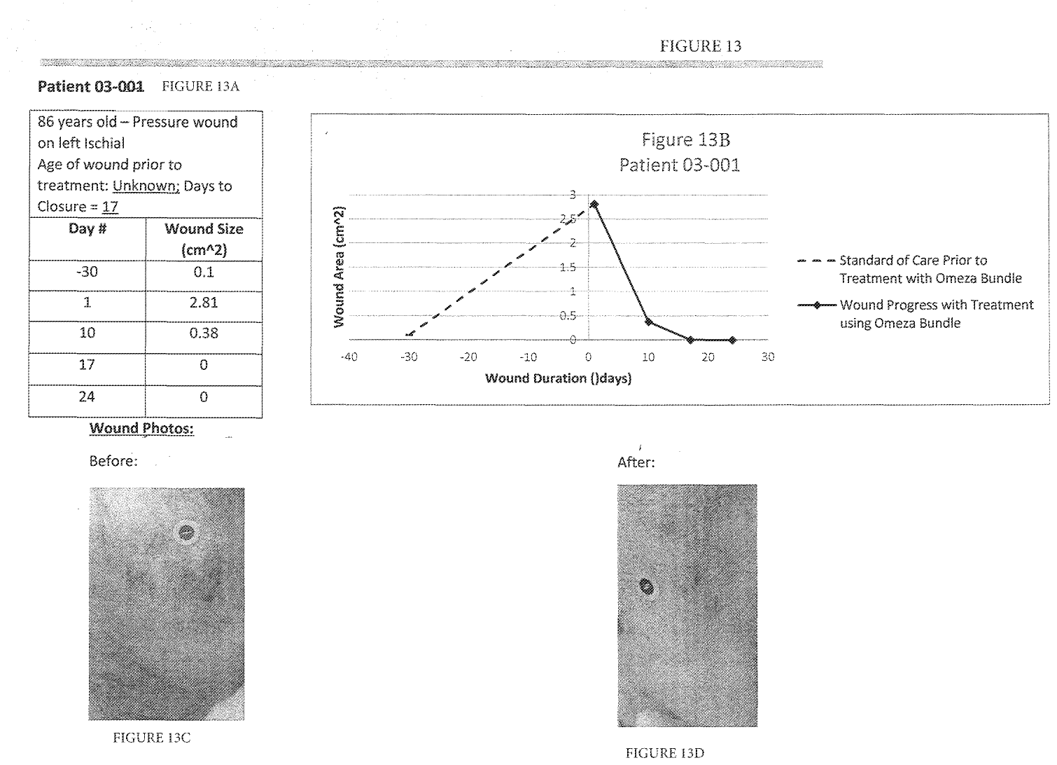

In FIG. 13, the wound was a stage 2 pressure wound on the left ischial of an 86 year old patient. FIG. 13a tabulates the size of the wound before and after treatment with the Omeza Bundle, FIG. 13b graphically depicts the change in the size of the wound after treatment with the Omeza Bundle; FIG. 13c is a photograph of the wound prior to treatment with the Omeza Bundle, and FIG. 13d is a photograph of the wound after treatment was completed.

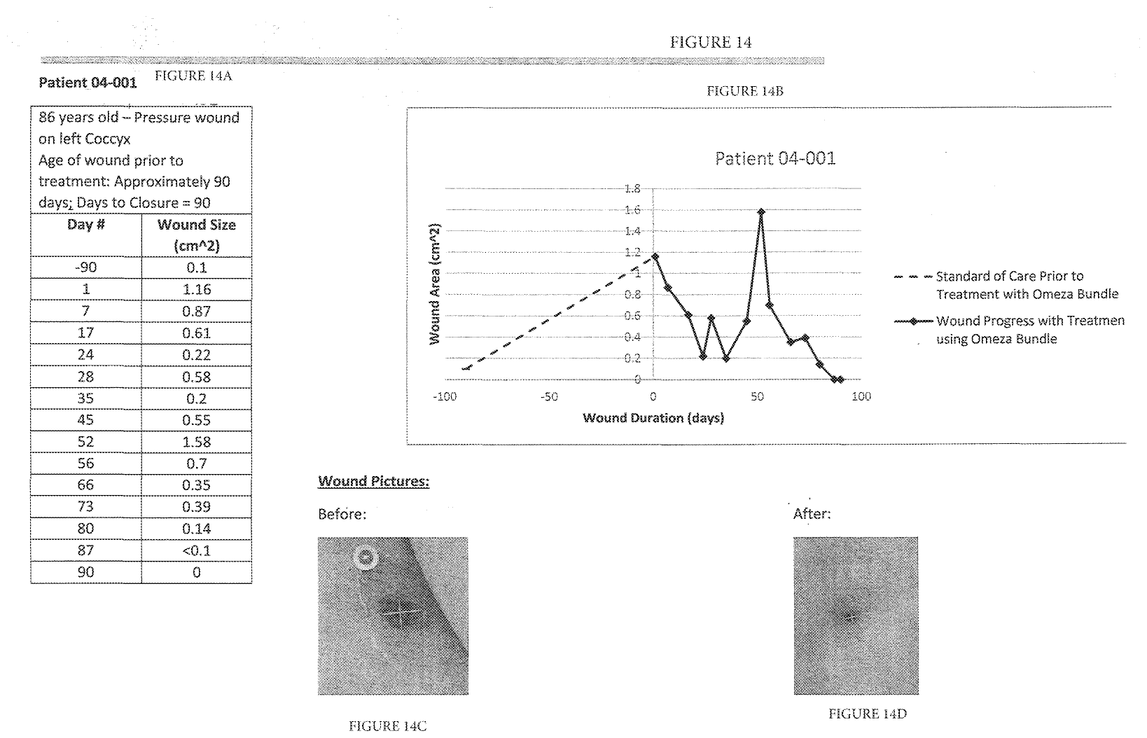

In FIG. 14, the wound is a left elbow laceration of a 97 year old patient. FIG. 14a tabulates the size of the wound before and after treatment with the Omeza Bundle, FIG. 14b graphically depicts the change in the size of the wound after treatment with the Omeza Bundle; FIG. 14c is a photograph of the wound prior to treatment with the Omeza Bundle, and FIG. 14d is a photograph of the wound after treatment was completed.

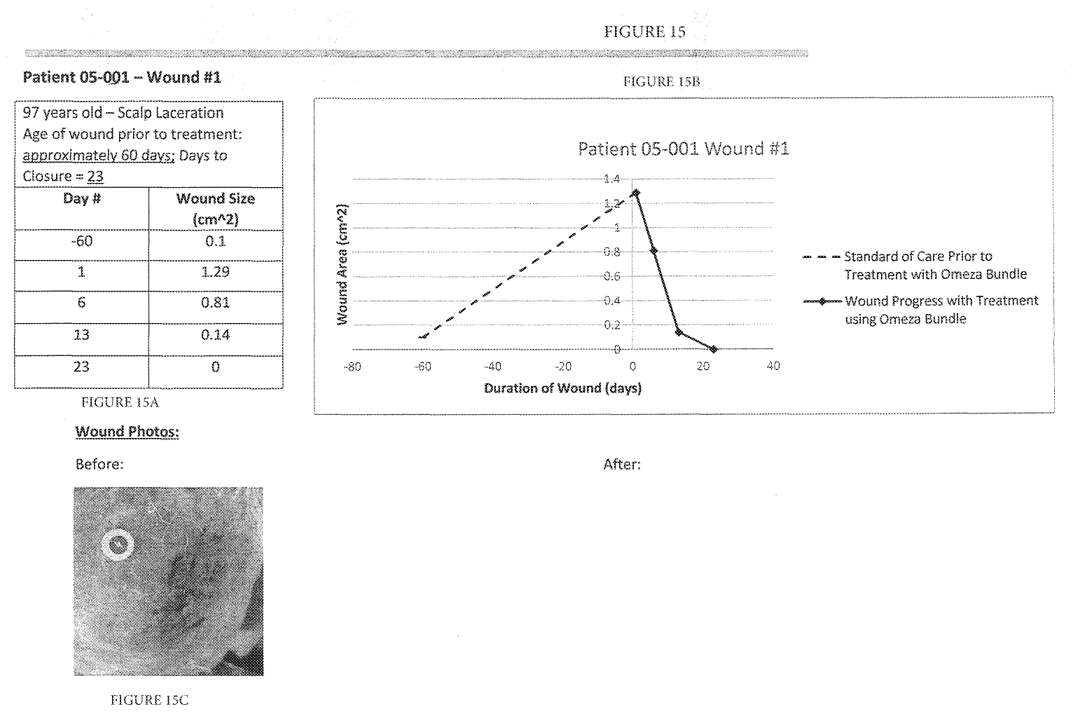

In FIG. 15, the wound is a scalp laceration in a 97 year old patient. FIG. 15a tabulates the size of the wound before and after treatment with the Omeza Bundle, FIG. 15b graphically depicts the change in the size of the wound after treatment with Omeza Bundle; FIG. 15c is a photograph of the wound prior to treatment with the Omeza Bundle.

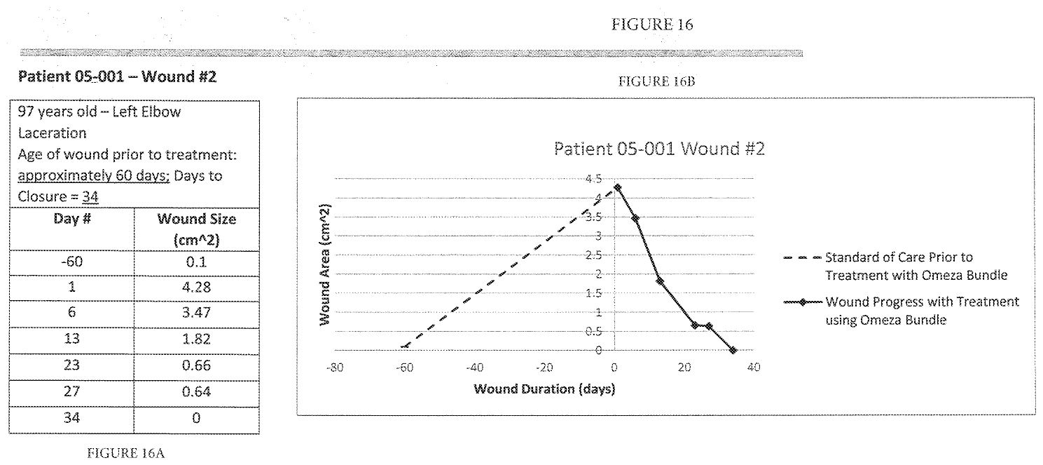

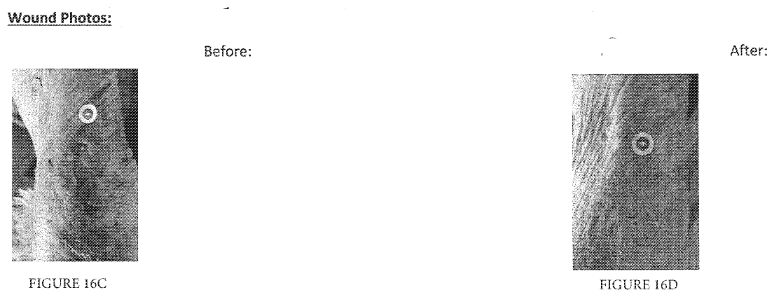

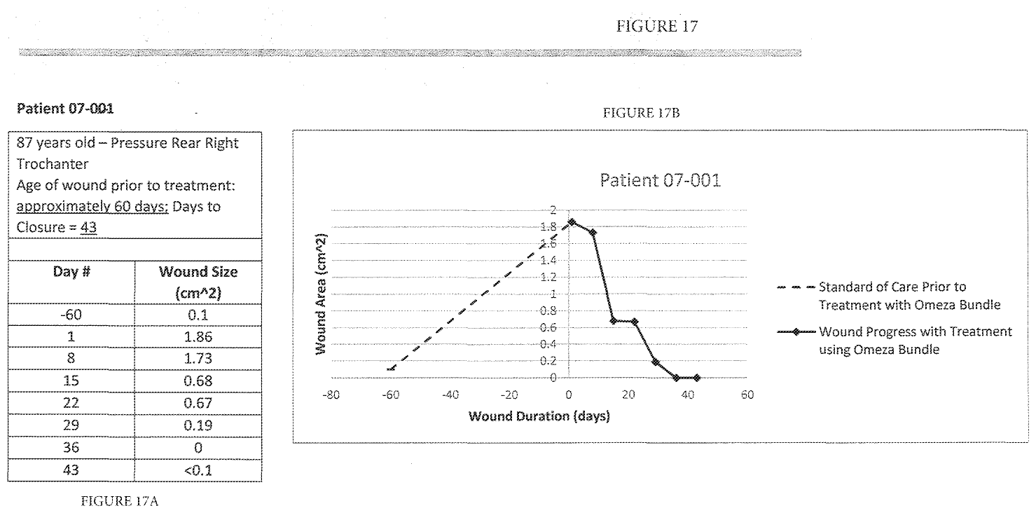

FIG. 16 tabulates and graphically depicts the effectiveness of the Omeza bundle on a wound on the left elbow of the 97 year old patient of FIG. 15, illustrating the effects with photographs taken before and after treatment. FIG. 16a tabulates the size of the wound before and after treatment with the Omeza Bundle, FIG. 16b graphically depicts the change in the size of the wound after treatment with the Omeza Bundle; FIG. 16c is a photograph of the wound prior to treatment with the Omeza Bundle, and FIG. 16d is a photograph of the wound after treatment was completed.

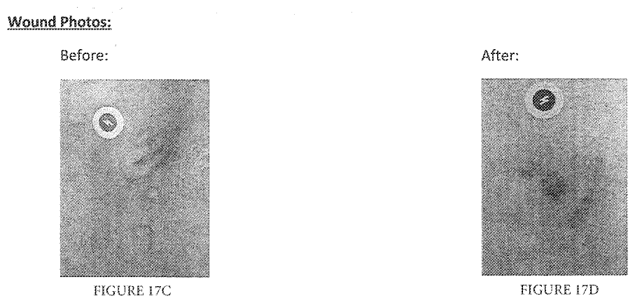

FIG. 17 tabulates and graphically depicts the effectiveness of the Omeza bundle on a wound on the hip of a 87 year old patient, illustrating the effects with photographs taken before and after treatment. FIG. 17a tabulates the size of the wound before and after treatment with the Omeza Bundle; FIG. 17b graphically depicts the change in the size of the wound after treatment with the Omeza Bundle; FIG. 17c is a photograph of the wound prior to treatment with the Omeza Bundle, and FIG. 17d is a photograph of the wound after treatment was completed.

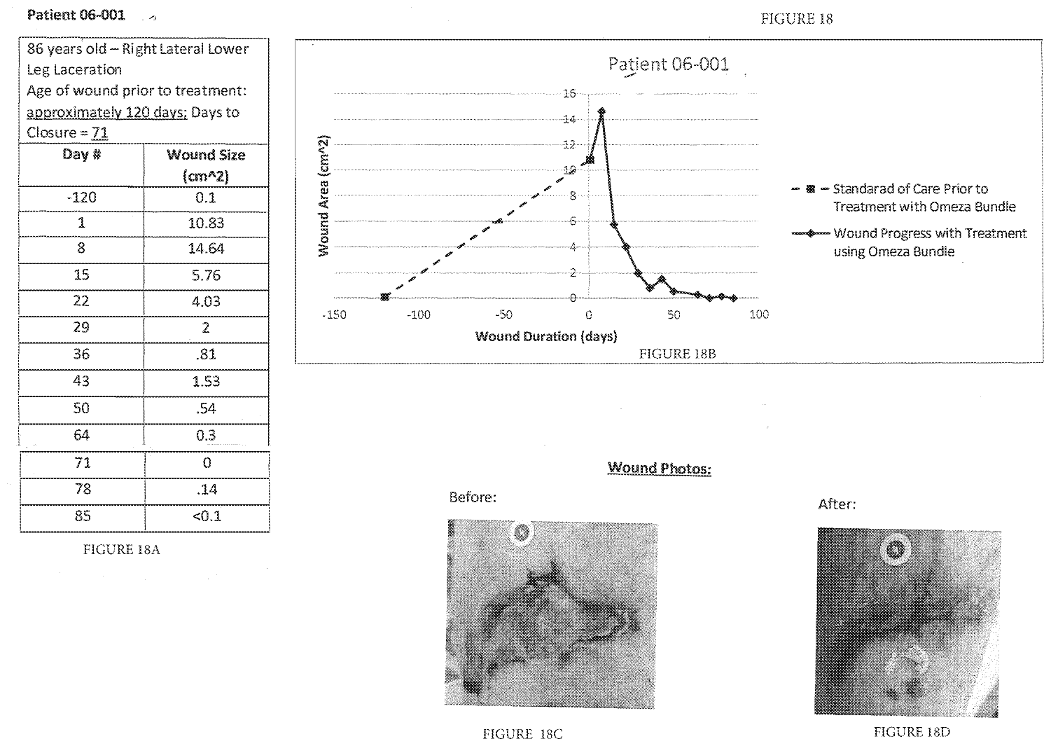

FIG. 18 tabulates and graphically depicts the effectiveness of the Omeza bundle on a right lateral lower leg laceration of an 86 year old patient, illustrating the effects with photographs taken before and after treatment. FIG. 18a tabulates the size of the wound before and after treatment with the Omeza Bundle; FIG. 18b graphically depicts the change in the size of the wound after treatment with the Omeza Bundle; FIG. 18c is a photograph of the wound prior to treatment with the Omeza Bundle, and FIG. 18d is a photograph of the wound after treatment was completed.

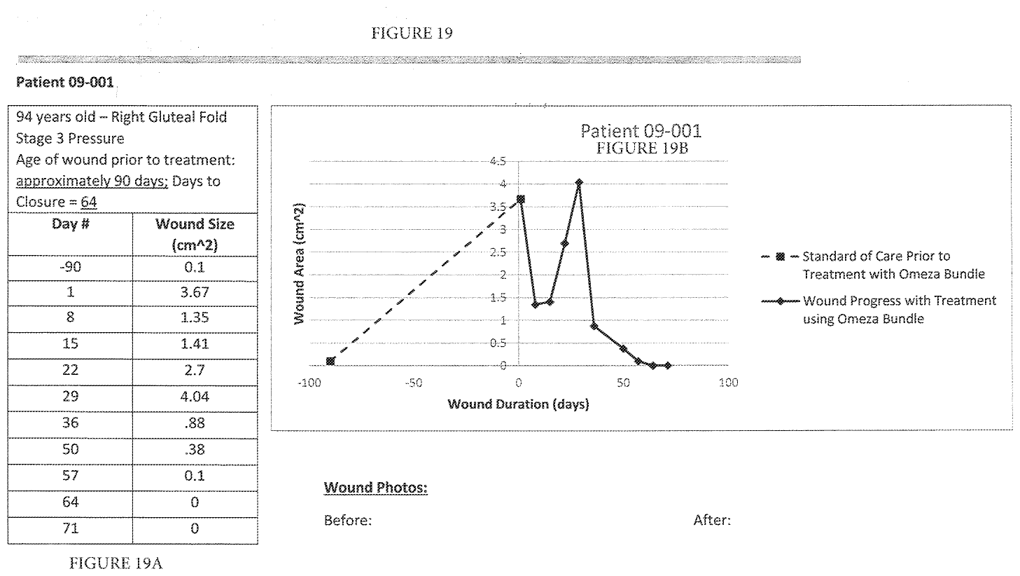

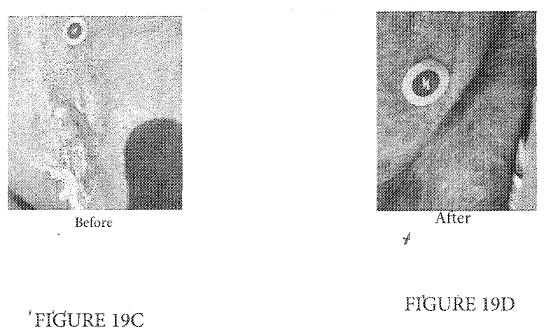

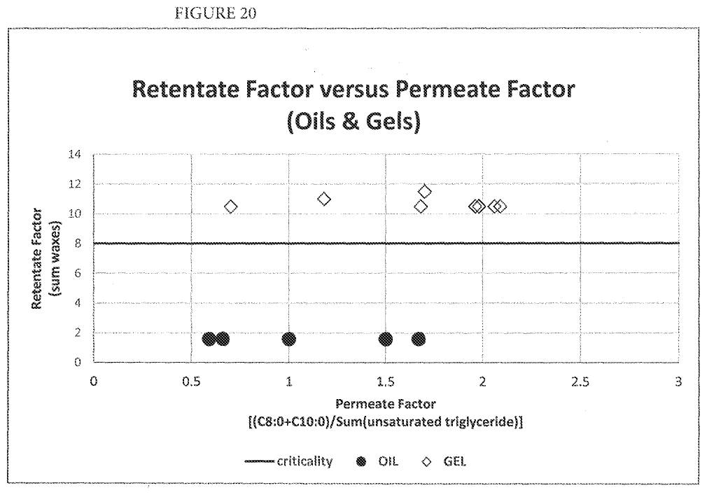

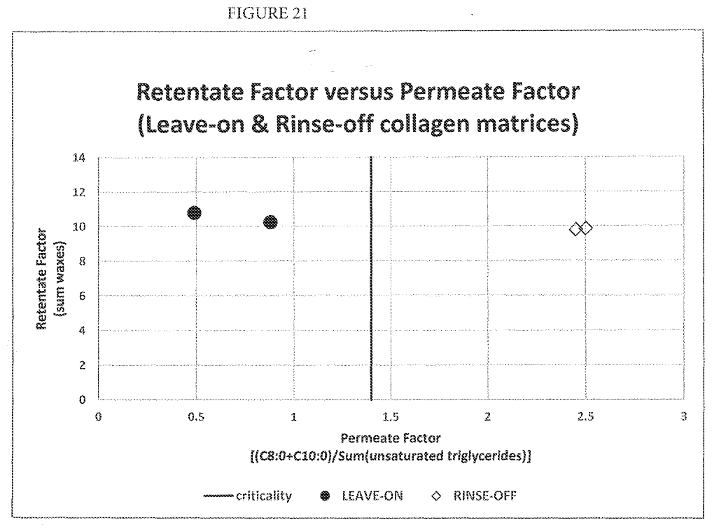

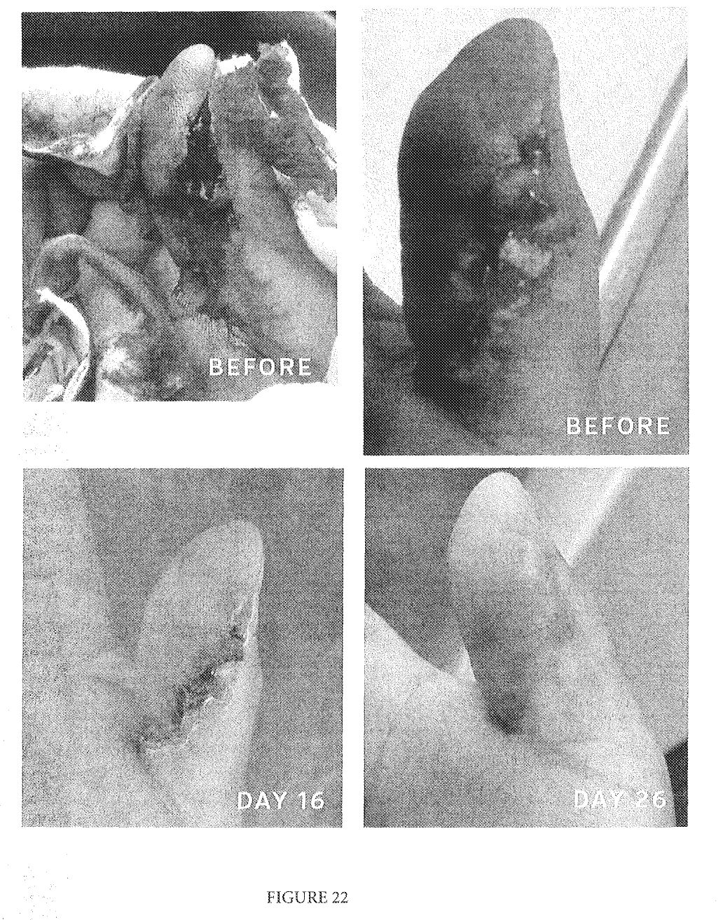

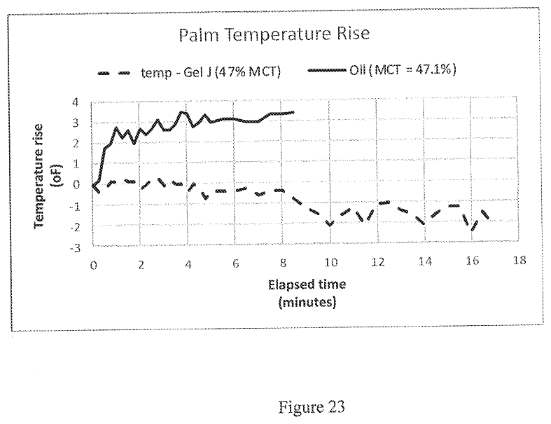

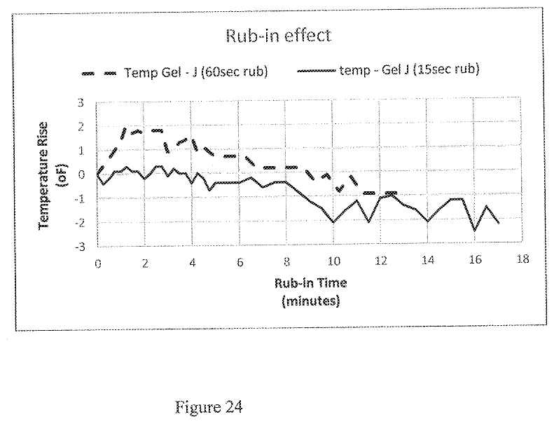

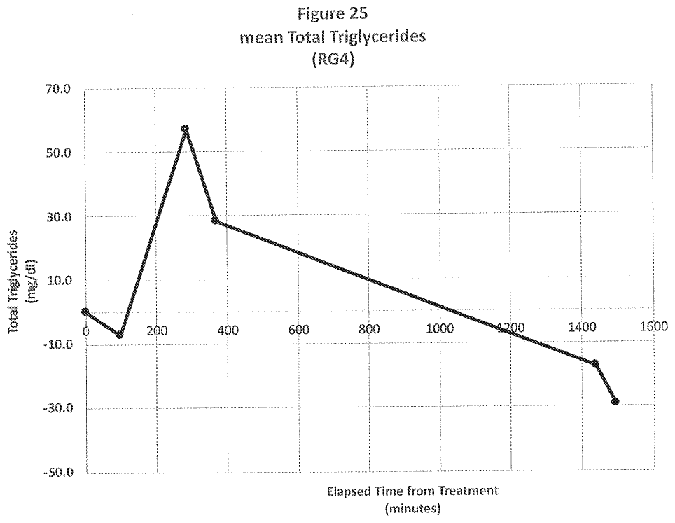

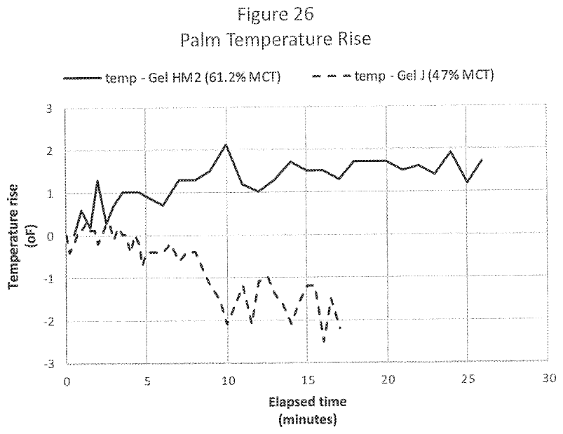

In FIG. 19, the wound was a stage 3 pressure wound on the right gluteal on a 94 year old patient. FIG. 19a tabulates the size of the wound before and after treatment with the Omeza Bundle; FIG. 19b graphically depicts the change in the size of the wound after treatment with the Omeza Bundle; FIG. 19c is a photograph of the wound prior to treatment with the Omeza Bundle; and FIG. 19d is a photograph of the wound after treatment was completed.

FIG. 20 is a graphical plot of the Retentate Factor versus Permeate Factor of various collagen-free oils and gels detailed in the examples.

FIG. 21 is a graphical plot of the Retentate Factor versus the Permeate Factor of various collagen containing embodiments.

FIG. 22 is a collage of photographs depicting healing over time of a badly injured thumb after treatment.

FIG. 23 is a comparative graphical representation depicting the rise in temperature as a function of time when the gelled composition of Example 6 and a prior art composition is applied to the skin of a subject.

FIG. 24 is a graphical representation of the temperature change when a gelled composition of the present disclosure is rubbed onto the heel of the palm of a subject for 15 seconds as compared to rubbing the gelled composition onto the heel of the palm of the hand for a minute.

FIG. 25 depicts graphically the mean total triglycerides present in arterial blood in three human subjects as a function of time after approximately 1 gram of RG4 was rubbed on their skin.

FIG. 26 depicts graphically the change in palm temperature over time after Gel HM2 and Gel J were rubbed on right hand palm of a human subject.

### DETAILED DESCRIPTION OF THE DISCLOSURE

As described hereinabove, in an embodiment, the present disclosure is directed to an anhydrous topical pharmaceutical composition comprised of marine oil, such as fish oil or algal oil; vegetable oil having an omega3 fatty acid content greater than 9 wt %, such as hempseed oil, canola oil, flax seed oil, or cannabidiol; linear fatty acid esters having 10-50 carbon atoms, such as cetyl esters or wax; monoglyceride of the formula R—C(O)—OCH₂—CH(OH)—CH₂OH, such as monolaurin, where R is an alkyl group of 7-11 carbon atoms; and medium chain triglycerides (“MCT”), wherein the weight ratio of the sum of the weights of (ALA+SDA)/sum of the weights of (EPA and DHA) present in the composition ranges from about 0.5 to about 1.5, and optionally collagen; said vegetable oil having an omega3 fatty acid content greater than 9 wt % comprised of ALA, SDA, EPA and DHA, wherein the weight ratio of the sum of the weights of (ALA+SDA)/sum of the weights of (EPA and DHA) present in the composition ranges from about 0.5 to about 1.5, and in addition, when the composition is an oil and is collagen free, the sum of the wt % of MCT, monoglyceride and linear fatty acid ester in said pharmaceutical composition is equal to or greater than 42 wt %, or when the composition contains collagen, the sum of the wt % of MCT, monoglyceride and linear fatty acid ester in said pharmaceutical composition is equal to or greater than 20 wt %.

As used herein, the descriptions described herein including descriptions of the embodiments of the present disclosure, refer to the novel compositions described herein.

The terms “omega3 fatty acids” and “omega3 fats”, “omega 3s”, and “omega3's”, whether in the plural or in the singular, are synonymous and are used interchangeably.

The compositions described herein are anhydrous and homogenous. Further, they do not exhibit the rancid, fish odor found in fish oil. Further when applied to the skin of a person, the person's skin is moisturized. Moreover, when applied to the skin in treating wounds, burns, or skin conditions, there is either no scarring, no visible discernment of scarring with the naked eye or an inappreciable amount of scarring.

Omega3 fatty acids can be isolated from various marine life. As defined herein, “fish oil” refers to the oil extracted from fish carcasses or crustaceans or any other marine animal life. Examples of fish containing omega3 fatty acids in its oil include salmon, tuna, swordfish, halibut, tilefish, cod fish, anchovies, green mussels and sardines. Cold-pressed fish oil and heat-treated fish oils are subsumed under the term “fish oil”. Examples of crustaceans from which the oil can be used include krill, a crustacean in the Antarctic (the source of krill oil) and the New Zealand green lipped mussel (also known as Perna canaliculus).

The terms “collagen-free” and “absence of collagen”, “non-collagen” or synonyms thereto are used interchangeably, signifying that the composition contains no measurable amount of collagen.

The terms “algal oil” and “algae oil” are synonymous and as used herein, these terms refer to oil made from certain marine or fresh water or farm-raised algae, such as algae grown in greenhouses or algae grown from any other source. Industrial sources of algae typically come from open recirculating ponds or from glass-enclosed growing containers (similar to greenhouses but are often vertical to better capture light). Open ponds are cheaper but are subject to contamination from wild algae. Glass-enclosed growing areas are used with specialty, high-efficiency algal strains. Useful oil is derived from marine vegetation, such as Marine algae, farm-raised algae, glass-raised algae and phytoplankton.

All of these fish oils and algae oil are sources of omega3 fatty acids. These oils are extracted from the livers of the fish or from algae using techniques known in the art.

For example, cod liver oil comes from cod fish, e.g., Atlantic cod (Gadus morhua) or from Pacific cod (Gadus microcephalus). Both Atlantic and Pacific cod liver oils are acceptable, but Pacific cod liver oil is preferred because the Bering Sea water is pristine with very little heavy metals. Pacific cod liver oil is obtained from wild, line-caught, adult cod. The immediately eviscerated liver, either from Pacific cod liver oil or Atlantic cod liver oil, is frozen on board to retain its nutrients. Frozen livers are transferred to a shore-side processing plant, rendered and pressed into commercial cod liver oil with almost no odor.

Nutritionally important omega3 fatty acids present in fish and algae oil include stearidonic acid (SDA, C18:4) (18 carbon, 4 polyunsaturated fatty acids), Eicosapentaenoic acid (EPA, C20:5) (20 carbon, 5 polyunsaturated fatty acids) and docosahexaenoic acid (DHA, C22:6) (22 carbon, 6 polyunsaturated fatty acids).

The term “vegetable oil having an omega3 fatty acid content greater than 9 wt %” refers to vegetable oils having more than 9 wt % omega3 fatty acids. The compositions of many vegetable oils are known. For example, canola oil has an alpha-linolenic acid content of 9.1 wt %. Flaxseed oil has an alpha-linolenic acid content of 53 wt %, and hempseed oil has an alpha-linolenic acid content of 22 wt %. Another vegetable oil that has a high omega3 fat content is cannabidiol, which is isolated from the Cannabis plant.

The terms “hempseed oil” and “hemp oil” as used herein, are synonymous. Hemp oil is prepared by pressing hemp seeds, especially hemp seeds that are cold-pressed. Unrefined hemp oil is dark to clear light green in color with a nutty flavor. However, in an embodiment, the hemp oil used herein is refined so that it is substantially free of tetrahydrocannabinol. It is manufactured from varieties of Cannabis sativa that are substantially free of tetrahydrocannabinol (THC). In the manufacturing process, THC is removed prior to pressing of the seed oil. In an embodiment, the hemp oil contains at most 1% by weight of THC and in another embodiment, less than or equal to 0.1% by weight THC, and in still further embodiment less than 0.01% by weight, if any, of THC. In another embodiment, the THC is not detectable, for example, less than 10 ppm. Hemp oil contains gamma-linolenic acid (GLA), which is an omega-6 fatty acid, as well as alpha-linolenic acid (ALA), which is an omega3 fatty acid. Hemp oil also contains about 14% oleic acid (C18:1), an omega-9 oil.

The term “medium-chain triglyceride(s) of C8/C10 saturated fatty acid”, “medium-chain triglyceride(s) of saturated C8/C10 fatty acids”, “medium-chain triglycerides of a mixture of saturated C8 and saturated C10 fatty acids”, “saturated C8/C10 triglycerides”, or “C8/C10 MCT” and “MCT” are interchangeable and refer to a triglyceride of saturated C8 fatty acids, a triglyceride of saturated C10 fatty acids or a triglyceride of a saturated C12 fatty acid or a mixture thereof. In addition, the term C8 triglyceride refers to a triglyceride of a saturated C8 fatty acid, and the term C10 triglyceride refers to a triglyceride of a saturated C10 fatty acid. In an embodiment, MCT is a mixture of triglycerides of saturated C8 and C10 fatty acids, wherein the amount by weight of triglycerides of C8 saturated fatty acids present is greater than the amount by weight of triglycerides of saturated C10 fatty acids present. It is to be understood that MCT comprises additional triglycerides besides saturated C8 and C10 triglycerides. MCT may comprise a triglyceride backbone having attached thereto three saturated fatty acid chains that are generally from about C6 to C12 in length, although shorter or longer chains may be included within the term in differing contexts, as understood by those having ordinary skill in the art; but these longer and shorter chains are present in negligible amounts, for example, usually less than 3% by weight of MCT. The three medium chain fatty acids that are attached to the triglyceride backbone of the MCT may be, but need not be, identical. Examples of medium chain fatty acids that comprise the medium chain triglycerides described herein include C6 (caproic fatty acid), C8 (caprylic fatty acid), C10 (capric fatty acid), and C12 (lauric fatty acid), as well as mixtures thereof. In an embodiment, MCTs comprise a mixture of from about 60 wt % saturated C8 triglycerides and about 40 wt % C10 saturated triglyceride to a mixture of about 70 wt % saturated C8 triglyceride and about 30 wt % saturated C10 triglyceride. In another embodiment, it comprises a mixture of about 51 wt % saturated C8 triglycerides and about 49 wt % saturated C10 triglycerides to about 70 wt % saturated C8 triglycerides to about 30 wt % saturated C10 triglycerides by weight; in another embodiment, it comprises about 55 wt % saturated C8 triglycerides and about 45 wt % saturated C10 triglycerides to about 65 wt % saturated C8 triglycerides to about 35 wt % C10 triglycerides by weight. Further, as indicated hereinabove, the MCTs of the present disclosure may include minor amounts of triglycerides of short or long chain fatty acids, such as C6 or C4 fatty acids or C12 or C14 or C16 fatty acids, but the short or long fatty acids are present in minor amounts, e.g., less than about 3 wt % by weight of MCT. In another embodiment, the MCT contains no triglyceride of a C12 fatty acid.

The medium-chain triglyceride(s) of C8/C10 fatty acids (MCT) are prepared by chemical techniques known in the art by esterifying the saturated fatty acid with glycerol. MCT is available commercially, such as from PG Chemicals, Inc.

The term “monoglyceride”, as used herein and unless indicated to the contrary, is a monoglyceride of the formula R—C(O)—OCH₂—CH(OH)—CH₂OH wherein R is an alkyl group containing 7-11 carbon atoms. The alkyl group may be linear or branched. The alkyl group does not contain any carbon-carbon multiple bonds, such as carbon-carbon double bonds or carbon-carbon triple bonds. In an embodiment, the alkyl group is linear. In another embodiment, the alkyl group is 7 carbons, 9 carbons or 11 carbons. An example of this monoglyceride is monolaurin.

Monolaurin, as used herein, is also known as glycerol monolaurate, glyceryl laurate or 1-Lauroyl-glycerol. It is a monoglyceride. It is the mono-ester formed from glycerol and lauric acid. Its chemical formula is C₁₅H₃₀O₄.

Compositions of the present disclosure all contain monoglycerides, such as monolaurin. However, the amount of monoglycerides varies. Some compositions contain less than 3 wt % of monoglyceride, such as monolaurin. These compositions are oils. Compositions containing greater than or equal to 6 wt % of monoglycerides, such as monolaurin, are gels. Other compositions of the present disclosure contain greater than 3 wt % monoglycerides, such as monolaurin but less than 6 wt % monoglyceride, such as monolaurin.

The term “linear fatty acid ester”, as used herein, has the formula R1COOR2, wherein R1 and R2 are independently straight-chained alkyl groups, wherein sum of the number of carbon atoms of R1 and R2 ranges from 9 to 49. In an embodiment, R1 and R2 independently contain 1-49 carbon atoms, as long as the sum of R1 and R2 ranges from 11-49. Examples are cetyl esters and waxes.

The terms “cetyl esters” and “cetyl ester” are used interchangeably. These terms, as defined herein, are unbranched esters formed from cetyl alcohol and a C14, C16 or C18 fatty acid or mixture thereof. The fatty acids may be saturated or unsaturated. As used herein, the term cetyl ester refers to esters of C14, C16, or C18 fatty acid or mixtures thereof and cetyl alcohol. Cetyl Esters is a synthetic wax that has similar composition and chemical properties to a natural wax which is found in the spermacetti of sperm whales. The esters that are found in Cetyl Esters include cetyl palmitate, cetyl stearate, myristyl myristate, myristyl stearate, cetyl myristate, and stearyl stearate. In an embodiment, cetyl ester is Cetyl Ester NF, CAS 540-10-3, 2598-99-4, EINECS 208-736, 220-000-6, typically sourced from Rita Corporation, Crystal Lake, Ill. Thus, as used herein, the term “cetyl ester” refers to one or more cetyl ester waxes.

Waxes are a diverse class of organic compounds that are lipophilic, malleable solids with a melting point above 40° C. (104° F.). They include higher alkanes and lipids containing from 12 to 50 carbon atoms. Waxes are insoluble in water but soluble in organic, nonpolar solvents. An example of a wax is beeswax, which is used by honeybees to make the honeycombs. Other waxes include spermaceti, which is found in the head cavities in sperm whales, and lanolin, which is a wax obtained from wool. Plant waxes included in the term waxes include carnauba wax, a hard wax obtained from the Brazilian palm Copernicia prumfera. Other waxes include jojoba oil, candelilla wax and ouricury wax, petroleum derived waxes, paraffin waxes and microcrystalline wax. Other examples of waxes within the term “wax’ include montan wax and polyethylene wax.

As defined herein, the terms “mixture of C8/C10 fatty acids”, “C8/C10 fatty acid” and “C8/C10 fatty acids” or “free fatty acids” or “FFA”, which are interchangeable, refer to a C8 fatty acid or C10 fatty acid or a mixture thereof. As used herein, the fatty acids are caprylic acid (C8 fatty acid), capric acid (C10 fatty acid) or a mixture thereof. In an embodiment, the terms refer to fatty acids that are a mixture of caprylic and capric acid. In an embodiment, in a mixture of C8 fatty acid and C10 fatty acid, the amount of C8 fatty acid present by weight is greater than the amount of C10 fatty acid present by weight. For example, in an embodiment, the weight ratio of C8 fatty acid to C10 fatty acid ranges from about 1.8 to about 1.1, while in another embodiment it ranges from about 1.6 to about 1.3. Capric/caprylic fatty acid is typically a natural product derived from coconut oil. Commercial product ranges from about 53% to about 63% C8 and about 47% to about 37% by weight C10. Thus, in an embodiment, the weight ratio of C8 fatty acid to C10 fatty acid ranges from about 53:47 to about 63:37, which is a ratio ranging from about 1.13 to about 1.71.

Free fatty acid refers to any added FFA, as defined herein as well as the linear carboxylic acid that is formed after the triglyceride is hydrolyzed into free fatty acid and glycerin or into free fatty acid and a monoglyceride or diglyceride. Industrial hydrolysis is complete (i.e. free fatty acid and glycerin). Enzymatic hydrolysis (e.g. in the body) is incomplete (free fatty acid and a glyceryl monoester, like monolaurin or a diglyceride).

Sea salt, as used herein, is the salt produced from the evaporation of seawater. The sea salt used herein may be refined or unrefined. The colors and variety of flavors in sea salt are due to local clays and algae found in the waters from which the salt is harvested. For example, some boutique salts from Korea and France are pinkish gray, some from India are black. The chemical composition of sea salt is typically the same as the ions dissolved in seawater. In an embodiment, the following ions are present by dry weight percent: Sodium, 30.8; Potassium, 1.1; Magnesium, 3.7; Calcium, 1.2; Chloride, 55.5; Sulfate, 7.7. Thus, sea salt, as the term is used herein, contains, as a minimum, those aforesaid ions. However, a study found that the amount of trace elements, such as titanium, silver, cobalt, and lead in synthetic sea salt are much higher than those in sea water. The magnitude of the difference can be as large as 10⁴ times. Unrefined sea salt contains small amounts of magnesium and calcium halides and sulphates, traces of algal products, salt-resistant bacteria and sediment particles. The calcium and magnesium salts confer a faintly bitter overtone, and they make unrefined sea salt hygroscopic (i.e., it gradually absorbs moisture from air if stored uncovered). Algal products contribute a mild “sea-air” smell, the latter from organobromine compounds. Sediments, the proportion of which varies with the source, give the salt a dull grey appearance. All of these different varieties of sea salts are contemplated by the term sea salt.

Collagen is a main protein component constituting connective tissue in animals and is characterized by having a collagen triple helical structure. A total of not less than 30 types of collagens have been reported which are respectively termed Type I, Type II, and so on. Type I collagen is the primary component of the derma, ligaments, tendons, bones and the like; and Type II collagen is the primary component of articular cartilage. Type III collagen is found in the skin, lungs, intestinal walls, and the walls of blood vessels. Further, Type IV collagen is mainly contained in a basal membrane, which is the undercoat of all epithelial tissues. Type I collagen is the most abundant collagen in the body. The term collagen includes the various types of collagens.

In the present disclosure, the fish collagen used is a collagen hydrolysate (hereinafter sometimes referred to as collagen peptide) and it refers to a low molecular weight (6-8 kDa) collagen obtained by hydrolyzing collagen from fish skins with an acid, alkali or enzyme. For example, a fish collagen hydrolysate can be obtained by immersing skins of fish in an acid or alkali solution to extract gelatin and treating the extracted gelatin with an enzyme or acid. The gelatin refers to the collagen pre-treated with an acid or alkali and then solubilized by heat hydrolysis. In an embodiment, the collagen is derived from marine life, such as cold-water fish. Cold water fish come from unpolluted water and do not have the diseases associated with land animals.

The collagen used in the present disclosure has a T_{g} (glass transition temperature) of less than 37° C. (normal body temperature). Practically this means that any marine collagen from a cold-water source is acceptable. An example is fish collagen. On the other hand, bovine, hog, horse collagens are not useful and are not included in the definition of collagen used herein.

The term “oils”, when used alone, refers to the all of the oils present in each of the compositions described herein.

The term “vegetable oil”, which is distinct from “vegetable oil having an omega3 fatty acid content greater than 9 wt %, as used herein, refers to vegetable oils containing no or less than or equal to 9.0 weight % of omega3 fatty acids in each vegetable oil used in the composition. Examples include coconut oil, corn oil, cottonseed oil, grapeseed oil, olive oil, palm oil, peanut oil, soybean oil, sunflower oil, cottonseed oil, and the like.

As used herein, the term “coconut oil” is a generic term that includes crude coconut oil and coconut oil that has been refined. Coconut oil is the raw minimally processed oil from coconuts, and as used herein, is referred to herein as “crude coconut oil” or sometimes “virgin coconut oil”. Refined coconut oil is coconut oil that has been refined, bleached and deodorized, and is referred to herein as refined or “RBD coconut oil.” Refined coconut oil has a higher melting point than crude coconut oil. Unless indicated to the contrary, the term “coconut oil” includes both crude coconut oil and refined coconut oil.

As used herein, the terms “palm olein” is synonymous with the term “palm oil” and both are used interchangeably. The term “palm oil” is the liquid portion which is separated from the semi-solid palm oil by fractionation. As used herein, the term includes red palm olein, and super red palm olein. The liquid portion is sold as cooking oils and the solid portion is known as “palm stearin”. When palm olein is fractionated again to get a more liquid fraction, such as by chilling and removing the solid fraction of C18:0 (saturated C18 fatty acids) and some C16:0 (saturated C16 fatty acids), it is known as “super palm olein” or “CP6” (Cloud Point 6° C., meaning the fractionation took place at 6° C.). Palm super olein is capable of withstanding colder temperature in comparison with palm olein after which they turn into solid. Palm olein is commonly used as cooking oil in the tropical countries. But the problem in temperate climate countries is that due to cold weather it tends to get cloudy and crystallize. To overcome this problem, palm olein is blended with more unsaturated vegetable oils. This blended form can be used in a wide range of climates and has a better cold stability. These blends are also cheaper than non-blended forms. The vegetable oils from rice bran, groundnuts and rapeseed are blended with palm olein to get a superior form in terms of quality and stability.

Palm oil, including red palm oil, is obtained from the endocarp of the palm fruit (the soft flesh); palm kernel oil is obtained from the seed (palm kernel oil). The oils are very much different. Red palm oil has the highest level of antioxidants of any seed crop. The carotenoids (the beta-carotene fraction is a Vitamin A precursors), tocopherols and tocotrienols (Vitamin E) present therein give red palm oil its distinctive color. The carotenoids are bright red; the tocopherols and tocotrienols are yellow; together they make red palm oil orange/red colored. These highly colored compounds are not readily absorbed by the skin and stain the skin surface and any clothing or bedding surface with which it comes in contact. When palm oil is refined, the carotenoids (primarily beta-carotene and lycopene) are removed and remaining palm oil which is RBD (refined, bleached, deodorized) palm oil is yellow colored because Vitamin E remains and the carotenoids are removed.

It is understood that the terms palm oil and palm olein includes the various types of palm oil, including, but not limited to, red palm oil or red palm olein, RBD palm oil, red palm concentrate, super palm olein, including super red palm olein, and the like.

In an embodiment, the palm oil, the red palm olein, and the super red palm olein and RBD palm oil contains a low amount of saturated C16 and C18 fatty acid. By low amount, it means less than 3% by weight.

The term “anhydrous”, as used herein refers to the water content of the composition of the present disclosure. As defined herein, the water content of the present composition refers to free water, that is, water not chemically bound to a substrate. As defined, the composition contains less than 5% by weight of free water or any range therein. A composition of the present invention may have a water content of less than 5, 4.5, 4, 3.5, 3, 2.5, 2, 1.5, 1, 0.5%, or any range therebetween, by weight of the composition. Water content may be measured by methods known to those of skill in the art, such as, but not limited to, Karl Fischer titration.

As defined herein the term “homogenous”, as it relates to a composition described herein, means that the components in the composition are substantially uniformly distributed throughout the composition.

The term “eutectic composition” is a mixture of chemical compounds or elements that has a single chemical composition that melts at a lower temperature than any other composition made up of the same ingredients. A composition comprising a eutectic is known as the eutectic composition and its melting temperature is known as the eutectic temperature.

The term “melting” point, as used herein, is used in three senses. It has the usual meaning, in one sense, i.e. the temperature at which a solid melts and forms a liquid. However, with many of the oily compositions described herein, it is difficult to determine or measure the specific melting point. In those cases, one of the following definitions is applicable, dependent upon the specific compositions. In some cases, such as, for example, for oily compositions comprised of hemp oil in the absence of fish collagen, the melting point is defined when the oily composition turns cloudy as the temperature is lowered. In other instances, such as compositions wherein the oily composition comprises hemp oil and collagen, it is defined when there is a visible change in viscosity when the temperature is raised. All three definitions are potentially applicable, but the term melting point with respect to the compositions discussed herein, for purposes of this application, is the lowest temperature in which the composition either melts, as in the traditional definition, or turns cloudy or where there is a visible change in viscosity.

As indicated hereinabove, in an embodiment, compositions of the present disclosure do not contain collagen. Embodiments of the collagen-free compositions of the present disclosure may exist as oils. In those collagen-free compositions, the amount of monoglyceride, such as monolaurin, present in the composition ranges from 0% (excluding 0%) to 3 wt %. For example, in an embodiment, the amount of monoglyceride, such as monolaurin ranges from about 0.1 wt to 3 wt %, and in another embodiment, from about 0.5 wt % to about 2.5 wt %. Other collagen-free embodiments of the present disclosure exist as gels. In these collagen-free embodiments, the wt % of monoglyceride, such as monolaurin, present in the composition, is greater than or equal to 6 wt %. In an embodiment, the amount of monoglyceride, such as monolaurin, that is present in the collagen-free compositions ranges from 6 wt % to about 14 wt %, and in another embodiment, from about 7 wt % to about 11 wt % and in another embodiment, from about 8 wt % to about 10 wt % and in another embodiment, about 9.5 wt %. In some collagen-free embodiments, the amount of monoglyceride present in the compositions of the present disclosure ranges from 3 wt % to 6 wt %. In other embodiments of the present disclosure, when the amount of monoglyceride, such as monolaurin, is present in these ranges, the composition contains collagen. In embodiments of the collagen containing compositions, the amount of monoglyceride, such as monolaurin, ranges from about 3.5 wt % to about 5.5 wt % and in another embodiment, from about 3.75 wt % to about 4.50 wt %. In other collagen containing embodiments of the present disclosure, the amount of monoglyceride, such as monolaurin, present is greater than or equal to 6 wt %, such as for example, from 6 wt % to about 15 wt %, and in other embodiments, from about 8 wt % to about 14 wt %, while in other embodiments, from about 9 wt % to about 12 wt %.

As described herein, an aspect of the present disclosure is that in the absence of collagen, when the composition is an oil, the sum of the wt % of MCT, monoglyceride and linear fatty acid ester in the pharmaceutical composition is greater than or equal to 42 wt %, or when the composition comprises collagen, the sum of the wt % of MCT, monoglyceride and linear fatty acid ester in said pharmaceutical composition is greater than or equal to 20 wt %. To determine this value, the sum of the percentages of each of the aforesaid ingredients in a formulation is added together. For example, if the formulation contains 40% wt % MCT, 10 wt % monolaurin and 1 wt % cetyl ester, then the sum of the three ingredients is 51%.

In collagen-free compositions, when the collagen-free composition is an oil, i.e., contains less than or equal to 3 wt % monoglyceride, such as monolaurin, the sum of the wt % of MCT, monoglyceride and linear fatty acid ester in the pharmaceutical composition is greater than or equal to 42 wt % of the composition. In an embodiment, collagen-free compositions of the present disclosure which is in the form of an oil, e.g., where the amount of monoglyceride, such as monolaurin, is 3 wt % or less, the sum of the wt % of MCT, monoglyceride, such as monolaurin, and linear fatty acid ester in the pharmaceutical composition ranges from 42 wt % to about 85 wt %, and in another embodiment, from 42 wt % to about 80 wt %. However, this limitation does not apply to collagen-free embodiments of the present disclosure in the form of a gel, e.g., contains more than or equal to 6 wt % monoglyceride, such as monolaurin. In these collagen-free gelled compositions, the sum of the wt % of MCT, monoglyceride and linear fatty acid ester in the pharmaceutical composition may be less than 42 wt %; for example, in such embodiments, the sum may be as low as 25 wt % or lower. For example, the sum of the wt % of MCT, monoglyceride and linear fatty acid ester in these gelled collagen free compositions may range from about 25 wt % to about 85 wt % and in another embodiment from about 35 wt % to about 85 wt %. In an embodiment, however, in the collagen-free gelled compositions, the sum ranges from 42 wt % to about 85 wt %, and in another embodiment, from 42 wt % to about 80 wt %. With respect to the collagen containing compositions of the present disclosure, the sum of the wt % of MCT, monoglyceride and linear fatty acid ester in the pharmaceutical composition is greater than or equal to 20 wt %. For example, in an embodiment, in the collagen containing compositions of the present disclosure, the sum of the wt % of MCT, monoglyceride and linear fatty acid ester ranges from about 22 wt % to about 40 wt %, and in another embodiment, from about 24 wt % to about 35 wt %.

The term “permeate factor”, as defined herein, refers to the sum by weight of the triglycerides of C8 and C10 saturated fatty acids (as triglycerides)/sum by weight of the triglycerides of saturated fatty acids larger than C10 present in a formulation of the present disclosure. Fatty acids are mainly composed of long chains of hydrocarbons ending in a carboxyl group. By definition, a fatty acid is a molecule of the formula R₄COOH, wherein R₄ is an organic group, such as a hydrocarbyl group, attached to the carboxyl group, COOH, containing an even number of carbon atoms and at least 6 carbon atoms and up to 30 carbon atoms. The carbon atoms may be a straight chain or branched or aromatic or a combination of aromatic and straight or branched chains. A fatty acid is saturated when the bonds between carbon atoms are all single bonds and is unsaturated when the bonds between the carbon atoms are unsaturated, e.g., carbon-carbon double or triple bonds or when the fatty acid contain an aromatic functionality. A triglyceride is by definition 3 fatty acids esterified to the three hydroxyl groups of a glycerol molecule (triacylglycerol). The fatty acids esterified to the glycerol molecule may be the same or different. Since the components used herein are natural products and/or are commercially available, the wt % of triglycerides of saturated fatty acids and triglycerides of unsaturated fatty acids in each composition can be easily determined by adding up the percentage by weight of the triglycerides of the saturated fatty acids and the unsaturated fatty acids in the various components present in each of the formulations. An example of this determination is shown in Example 6. As a shorthand notation, the permeate factor may be described as [(C8:0+C10:0 triglycerides)/(Sum unsaturated triglycerides)] or (C8+C10)/Sum Unsat; it is to be understood that these shorthand notations mean permeate factor.

In an embodiment, e.g., in the collagen free compositions, the weight ratio of the sum of the weights of saturated (C8+C10 triglycerides)/sum of the weights of unsaturated triglycerides larger than C10 ranges from about 0.3 to about 25.0, and in another embodiment, from about 0.4 to about 20.0, and in another embodiment, from about 0.45 to about 18.0. For example, the weight ratio of the sum of the weights of saturated (C8+C10)/sum of the weights of unsaturated fats larger than C10 may be 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8. 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0 or any value therebetween are contemplated within the scope of the topical compositions described herein. This value has been identified herein as the permeate value.

Since the components of the present formulation are known, one of ordinary skill in the art can manipulate the permeate factor to any value desired. For example, one of ordinary skill in the art can increase the value by increasing the amount of C8 and C10 saturated triglycerides present or by decreasing the amount of unsaturated triglycerides larger than C10. For example, the increase in permeate factor can easily be effected by increasing the amount of MCT. Alternatively, one of ordinary skill in the art can decrease the permeate factor by decreasing the amount of C8 and C10 saturated triglycerides present or by increasing the amount of unsaturated triglycerides larger than C10. For example, the decrease in permeate factor can easily be effected by decreasing the amount of MCT. The higher the value of the permeate factor exhibited by a composition, the greater the tendency for the composition to be absorbed through the skin.

The calculation of the permeate factor is straightforward, illustrated in the Example 6 Explanation. A spreadsheet is developed using art-recognized techniques. For example, each triglyceride ingredient is listed with its weight percent of each constituent fatty acid. The sum of all ingredients (triglycerides+other constituents) must equal 100%. From readily available literature sources, the fatty acid chain distribution for each triglyceride raw material is copied into the table of raw materials. Each raw material weight percent is multiplied by that particular ingredient's literature average chain length percent to determine the relative triglyceride contribution of a particular ingredient to the total formulation. Then the spreadsheet sums the relative contribution of all the ingredients to calculate the total weight percent of the particular chain length triglyceride. Skilled artisans immediately grasp that some ingredients, e.g. perfume, do not contribute to the weighted sum of triglycerides. The Example 6 explanation has 17 possible raw materials (listed vertically), 6 possible triglyceride-containing raw materials (listed horizontally) and 28 possible triglyceride chain lengths (saturated, monounsaturated and polyunsaturated). 56 possible sums and ratios are calculated to determine what, if any, sums and/or ratios are relevant to describe differences between formulations. With respect to the Example 6 explanation, skilled artisans recognize that the number of possible sums and ratios could be larger or smaller than 56 mentioned hereinabove. The permeate factor and retentate factor were used to separate products that demonstrated relevant clinical differences.

The Retentate Factor, as defined herein, represents the waxes that remain on the surface of the wound, burn, and/or skin condition. It is defined as the sum of saturated waxes present in the formulation of the present disclosure, and includes beeswax, cetyl ester, monolaurin and ascorbyl palmitate, and the like. The higher the value of the retentate factor, the greater the length of time for the composition to remain on the surface of the skin.

In an embodiment, the retentate factor can range from about 0.5 to about 48 and in another embodiment, from about 0.8 to about 45 and in another embodiment from about 1.0 to about 30. For example, the retentate factor can be 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5 or 48.0 or any value therebetween. For gelled non-collagen compositions described herein, in an embodiment, the retentate values range from about 6 to about 48, and in another embodiment, from about 8.0 to about 45, and in another embodiment, from about 10 to about 30.

The retentate value of a formulation can also be manipulated by one of ordinary skill in the art. As defined, the retentate value is defined as the sum of the % by weight of compounds of waxes and esters having C12:0 to C:38:0, monolaurin, cetyl esters and waxes. For example, one can increase this value by adding more monolaurin, or more cetyl esters or more waxes or a combination thereof, and one can decrease the value by removing esters having C12:0 to C:38:0, or removing monolaurin, or removing cetyl esters or removing waxes or a combination thereof.

As used herein, the term wax refers to an ester of a fatty acid, wherein the wax contains 13 to 40 carbon atoms, and the fatty acid is an organic acid of the formula R₅COOH containing 8 to 24 carbon atoms, i.e., R₅ is C7-C23. As used herein, a wax is saturated, i.e., except for the acyl group (C═O), the wax contains only single covalent bonds (carbon-carbon single bonds and carbon oxygen single bond). As used herein, there are no multiple carbon-carbon bonds present in the formulation. Since the components of the present formulations are natural products and/or commercially available, the amount by weight of the saturated waxes in the various components in the formulations can be determined by adding the percentage by weight of the saturated waxes present in each of the formulations described herein. An example of this determination is shown in Example 6.

Embodiments of the compositions of the present disclosure, the collagen free compositions and the collagen comprising compositions, exhibit “cosmetically elegant” non-sticky surface skin feel, while at the same time, are capable of transferring important ingredients into the targeted layers of the skin. By “cosmetically elegant”, it is meant that the composition

1. does not have that greasy feeling, but feels like a smooth lotion is applied to the skin;

2. is moisturizing in the absence of added moisture (via reducing TEWL);

3. has little or no off odors (like rancid fat (oxidized), like “fishy” smell (trimethylamine));

4. penetrates the epidermis during rub-in;

5. is “silky-smooth” post-rub-in skin feel;

6. has long-lasting skin feel; and

7. has little or no stickiness

As defined herein, the term “transdermal”, when used alone or in combination, refers to complete passage, such as absorption, through the three layers of the skin and into the circulatory system for systemic distribution.

As defined herein, the term “intradermal”, when used alone or in combination with other terms, refers to passage, such as absorption, from the surface of the skin into the epidermis and into the dermis, but not into the hypodermis. There are three layers of skin: the epidermis, the outermost layer of skin, which provides a waterproof barrier and creates skin tone; the dermis, beneath the epidermis, which contains the tough connective tissue, hair follicles, cutaneous fat cells (adipocytes) and sweat glands; and the deeper subcutaneous tissue (hypodernis) which is made of subcutaneous fat (also adipocytes) and connective tissue. Cutaneous fat cells migrate towards hair follicle bulbs to help activate hair follicles (telogen (resting) to anagen (growing)); subcutaneous fat cells do not migrate. Unlike “transdermal”, which refers to complete passage, such as absorption, through the three layers of the skin and into the circulatory system for systemic distribution, intradermal does not include passage into the hypodernis layer of the skin and thus does not extend as far as passage to the circulatory system for systemic distribution. Skilled artisans recognize that intradermal fluids migrate and are eventually distributed systemically. The intradermal fluids have a finite residence time in the intradermal zone; transdermal fluids are systemically distributed virtually immediately. It is believed, without wishing to be bound, that during the residency in the intradermal zone, omega3 fats are biologically converted into many biologically active compounds (e.g. prostaglandins, maresins, etc.)

As defined herein, there are various ranges of numbers or ratios provided herein. It is to be understood that the ranges include not only the endpoints (plus or minus 5% when modified by the term about), but also all of the integral numbers and fractions therebetween. Thus, for example, if the range is defined as ranging from 5 wt % to 10 wt %, it is understood that the values include the endpoints 5 wt % and 10 wt %, and also all the integers and fractional numbers and real numbers therebetween and each one of those values are within the scope of the present disclosure, for purposes of the teaching of the disclosure described herein. The only exception is when a component is listed as being present from 0% to a certain value. This signifies, for purposes of this disclosure, that the component is present and is present in some amount greater than 0%. Furthermore, unless indicated to the contrary, if the disclosure refers to a component being present in less than certain %, it is understood that the component is present in an amount ranging from 0%, but greater than 0%, (i.e., excludes 0%) to that certain %. Thus, for example, if a component is being described as being present in less than 3 wt %, it is understood that it is meant that the component is present in an amount ranging from 0 wt %, but greater than 0 wt % to 3 wt %, inclusive. If a component is not present, the application will so state using terms that signify that a particular component is absent, such as “absent of such component”, “free of such component” or any like term.

As used herein, the terms that list the number of carbon atoms followed by a colon and then a number, like C18:0, refer to the number of carbon atoms and the number of carbon-carbon double bonds in a compound, such as a triglyceride, in the topical composition. Triglycerides are esters formed from glycerol and three fatty acid groups. The number after the “C” refers to the number of carbon atoms in the fatty acid and the number after the colon refers to the number of carbon-carbon double bonds in the fatty acid. Thus, for example, C18:0 refers to the triglyceride wherein the fatty acid attached to the glycerol moiety contains 18 carbon atoms and has no carbon-carbon double bonds, while the term C18:1 refers to the triglyceride wherein the fatty acid attached to the glycerol contains 18 carbon atoms and has one carbon-carbon double bond, while the term C18:2 refers to the triglyceride wherein the fatty acid attached to the glycerol moiety contains 18 carbon atoms and has two carbon-carbon double bonds.

Unless indicated to the contrary, when it is indicated that a category of a component, such as linear fatty acid ester, is present in a certain range, it is understood that the total amount of that category is present in that range. For example, if the composition contains linear fatty acid esters ranging from about 10 to about 20 wt %, it is understood that the total amount of the weights of linear fatty acids esters present in the composition ranges from about 10 to about 20 wt %. For example, cetyl esters and/or other waxes, such as beeswax, are examples of linear fatty acids esters, as defined herein, it is understood that the term linear fatty acid esters ranging from about 10 to about 20 wt % means that the sum of the weights of cetyl esters and waxes, whichever is present, in an amount ranging from about 10 wt % to about 20 wt %. However, this does not signify that the composition necessarily only contains either one of cetyl esters or other waxes; it may contain cetyl esters or other waxes or both.

Unless indicated to the contrary, the terms “compositions” and “formulations” alone or in combination with other terms, such as for example, topical, are synonymous and can be used interchangeably.

Unless indicated to the contrary, the terms “drugs” and “medicament” are synonymous.

Treatment” or “treating,” as used herein refers to complete elimination as well as to any clinically or quantitatively measurable healing or alleviation of the symptoms of the wound or skin condition or burn.

A “therapeutically effective amount” means the amount of a composition that, when administered to a subject for treating a wound, burn or skin condition is sufficient to effect a desirable treatment for the wound, burn or skin condition, respectively. The “therapeutically effective amount” will vary depending on the particular composition, the condition and its type and severity, and the age, weight, etc., of the subject to be treated. The actual amount which comprises the “effective amount” will vary depending on a number of conditions including, but not limited to, the particular disorder being treated, the severity of the disorder, the size and health of the patient, and the route of administration. A skilled medical practitioner can readily determine the appropriate amount using methods known in the medical arts.

“Patient” or “subject” refers to animals, and can include any mammal, such as humans, rats, mice, cats, dogs, goats, sheep, horses, monkeys, apes, rabbits, cattle, bears, etc. The mammalian subject can be in any stage of development including adults, children, infants, and neonates.

Unless indicated to the contrary, percentages are by weight and ratios are weight ratios.

The term “about”, as used herein, when used before a number or numerical ranges, in an embodiment herein, refers to an amount±5% of that value of the number or range. For example, when referring to a range of about 9 to about 11, it is understood that this amount ranges from 8.65-11.45.

As used herein, the singular forms “a”, “an” and “the” include plural referents unless the context clearly dictates otherwise.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

Furthermore, as used herein, the terms “components” and “ingredients” are synonymous and are used interchangeably. In addition, the terms “thickener” and “thickening agent” are synonymous and are used interchangeably.

Further, as used herein, unless indicated to the contrary, the terms “composition” and “formulation” and “composition of the present disclosure” and “formulation of the present disclosure” and “compositions described herein” and “formulations described herein” and “topical compositions” and “topical formulations” and “topical formulations of the present disclosure” and “topical compositions of the present disclosure” and the like are being used interchangeably and synonymously.

The present disclosure refers to collagen-free compositions and collagen containing compositions. As described herein, the compositions either contain collagen or do not contain any collagen. The term “collagen containing compositions” are those compositions containing more than 5 wt % collagen, while the term “collagen free compositions” refers to compositions containing no collagen or less than or equal to 2.0 wt % collagen in the composition. As used herein, the term non-collagen containing composition or collagen-fee composition are synonymous and are used interchangeably. Hydrolyzed collagen is water-soluble, not oil soluble. However, small amounts of collagen can be incorporated into gels and can be mechanically stabilized to appear homogenous. Low amounts of suspended collagen can be advantageous, for example in very gentle facial scrubs or in first aid antiseptics.

In an embodiment, the present disclosure is directed to an anhydrous topical composition comprised of marine oil, such as fish oil or algal oil; vegetable oil having an omega3 fatty acid content greater than 9 wt %, such as hempseed oil, canola oil, flax seed oil, or cannabidiol; monoglyceride, such as monolaurin; linear fatty acid esters, such as cetyl esters, wax, and the like; and medium chain triglycerides (“MCT”), wherein the weight ratio of the sum of the weights of (ALA+SDA)/sum of the weights of (EPA and DHA) present in the composition ranges from about 0.5 to about 1.5 and in addition, in the absence of collagen, when the composition is an oil, the sum of the wt % of MCT, monoglyceride and linear fatty acid esters in said pharmaceutical composition is greater than or equal to 42 wt %, or in the presence of collagen, the sum of the wt % of MCT, monoglyceride and linear fatty acid esters in said pharmaceutical composition is greater than or equal to 20 wt %.

In an embodiment of the compositions described herein, for example, the gelled compositions, the weight ratio of the sum of the weights of (ALA+SDA)/sum of the weights of (EPA and DHA) present in the composition ranges from about 0.1 to about 1.5, and in another embodiment, from about 0.7 to about 1.3, and in another embodiment, from 0.9 to about 1.1, and in another embodiment, at about 1.0, and in another embodiment, at about 0.75 For example, a weight ratio of the sum of the weights of (ALA+SDA)/sum of the weights of (EPA and DHA) of 0.1, 0.2, 0.3, 0.4, 0.5, 0.6, 0.7, 0.8, 0.9, 1.0, 1.1, 1.2, 1.3, 1.4, or 1.5 or any value therebetween is contemplated within the scope of the topical compositions described herein.

From the various components described herein, the compositions of the present disclosure contain various organic molecules of different chain lengths, some of which are saturated, while others are unsaturated. For example, ALA (alpha-linolenic acid) and GLA (g-linolenic acid) are found in hempseed oil, while cod liver oil contains EPA (eicosapentaenoic acid), SDA (stearidonic acid) and DHA (docosahexaenoic acid). Further, hemp oil contains omega 6 fatty acids, such as GLA (Gamma-linolenic acid). As a result, the components present, may include, but are not limited to, saturated and unsaturated organic compounds of the following lengths: C8:0, C10:0, C12:0, C14:0, C16:0, C18:0, C20:0, C22:0, C24:0, C16:1, C18:1, C20:1, C22:1, C18:2, C20:2, C18:3, C18:4, C20:5, C22:6 and other chain lengths and isomers at trace amounts (<1 wt %). The listed chain lengths describe the three individual fatty acids attached to the glycerol backbone. The second number (after the colon) refers to the number of unsaturated double bonds on any particular fatty acid.

In an embodiment of the collagen free compositions, the permeate factor is greater than or equal to 3.0 and the retentate factor is less than or equal to 8.0. In this embodiment, which may be an oil, absorption is very rapid. In another embodiment of the collagen free compositions, the permeate factor is greater than or equal to 3.0 and the retentate factor is greater than 8.0. In this embodiment, which may be a gel, absorption is complete, but not rapid. In an alternative collagen-free embodiment, the permeate factor is less than 3.0 and the retentate factor is less than or equal to 8.0. In this embodiment, which may be an oil, absorption is slow and grease is intentionally left on the skin surface. In a still alternative collagen-free embodiment, the permeate factor is less than 3.0 and the retentate factor is greater than 8.0. In this embodiment, which may be a gel, absorption is slow and the grease/waxy retentate has a “lotion-applied” skin feel. With respect to the collagen containing composition, in some embodiments the permeate factor is less than 1.0, while in others, the permeate factor is greater than or equal to 1.0. Low permeate collagen embodiments release oils slowly over days, conducive to rapid wound healing. High permeate collagen embodiments are used for skin scrubs, where the oil penetrates rapidly leaving the collagen granules as a soft “sandpaper” to exfoliate dead and dying skin. A collagen containing composition containing a permeate factor of less than 1.0 is said to have a low permeate factor, while a collagen containing composition having a permeate factor of greater than or equal to 1.0 is said to have a high permeate factor. In an embodiment of the present disclosure, the collagen containing compositions of the present disclosure has retentate factor less than 8.0, while in another embodiment of the collagen comprising composition of the present disclosure, the retentate factor is greater than or equal to 8.0. In general, permeate levels are adjusted for rapid absorption (high permeate) or delayed release (low permeate). Low retentate collagen embodiments have a Retentate Factor less than 8; high retentate collagen embodiments have a Retentate Factor greater than or equal to 8. The difference is usually related to embodiment stability (higher retentates are needed to prevent oil weeping over time.)

When the permeate factor in the collagen-free composition is less than 3.0, it is to be referred to herein as a low permeate factor; when it is greater than or equal to 3.0, it will be referred to herein as a high permeate factor. It is to be understood, that the permeate factor in the formulations described herein is always greater than zero. With respect to the retentate factor in the collagen-free compositions, if it is less than or equal to 8.0, it will be referred to as a low retentate factor, but when it is greater than 8.0, it will be referred to as a high retentate factor. Again, it is to be understood that with the compositions described herein, the retentate value is always greater than zero.

In the collagen-free formulation, the low permeate factor ranges from about 0 (greater than 0), but up to and excluding 3.0. In an embodiment, the low permeate factor in the collagen-free compositions ranges from about 1.0 to 3.0, excluding 3.0. In another embodiment of the collagen-free formulation, the high permeate factor is less than 25.0, but greater than or equal to 3.0. In another collagen-free composition, the high permeate factor ranges from and including 3.0 and up to about 20.0.

With respect to the retentate factor in the collagen-free compositions, the low retentate factor ranges from about 0 (but greater than 0), but up to and including 8.0. In an embodiment of the collagen-free composition, the low retentate factor ranges from about 1.0 to and including 8.0, and in another embodiment, from about 1.4 to and including 8.0. Furthermore, in an embodiment, the high retentate factor in a collagen-free composition is less than or equal to 16. In an embodiment of the collagen-free composition, the high retentate factor ranges from 8.0 (excluding 8.0) to about 12.0.

In an embodiment, when the concentration of MCT is less than 60 wt % of the formulation, the sum of the % weight of marine oil and the vegetable oil having omega3 fatty acid content greater than 9 wt % in the formulation is greater than 20 wt % in collagen-free formulations. In an embodiment, when the concentration of MCT is less than 60 wt % of the formulation, the sum of the % weight of marine oil and the vegetable oil having omega3 fatty acid content greater than 9 wt % in the formulation is greater than 22 wt % in collagen-free formulations. In an embodiment, when the concentration of MCT is less than 60 wt % of the formulation, the sum of the % weight of marine oil and the vegetable oil having an omega3 fatty acid content greater than 9 wt % in the formulation is greater than 20 wt % but less than 55 wt % in collagen-free formulations.

In the formulations of the present disclosure, the function of the monoglyceride, such as monolaurin, when present, in the formulations described herein, is to help reduce the undesirable rancid smell of the marine oil, such as cod liver oil. Without wishing to be bound, it is believed that the monoglyceride, such as monolaurin, also helps the marine oil be absorbed by unbroken skin. It is believed that the faster the marine oil is absorbed, the less oxidation odor is generated.

Monoglycerides, such as monolaurin, in embodiments of the collagen-free composition, which is a gel, is also acting as a gelling agent with respect to the other components present in the compositions of the gelled formulations described herein. To form a stable gel in these embodiments, the monoglyceride, such as monolaurin, is present in amounts of 6 wt % or greater. In an embodiment, it is present from about 7 to about 14 wt % and in another embodiment, from about 8.5 to about 10.5 wt %, and in another embodiment from about 9 to about 10 wt %. In an embodiment, it is present in about 9.5 wt % of the composition. For example, monoglycerides, such as monolaurin, may be present in 6.0 wt %, 6.1 wt %, 6.2 wt %, 6.3 wt %, 6.4 wt %, 6.5 wt %, 6.6 wt %, 6.7 wt %, 6.8 wt %, 6.9 wt %, 7.0 wt %, 7.1 wt %, 7.2 wt %, 7.3 wt %, 7.4 wt %, 7.5 wt %, 7.6 wt %, 7.7 wt %, 7.8 wt %, 7.9 wt %, 8.0 wt %, 8.1 wt %, 8.2 wt %, 8.3 wt %, 8.4 wt %, 8.5 wt %, 8.6 wt %, 8.7 wt %, 8.8 wt %, 8.9 wt %, 9.0 wt %, 9.1 wt %, 9.2 wt %, 9.3 wt %, 9.4 wt %, 9.5 wt %, 9.6 wt %, 9.7 wt %, 9.8 wt %, 9.9 wt %, 10.0 wt %, 10.1 wt %, 10.2 wt %, 10.3 wt %, 10.4 wt %, 10.5 wt %, 10.6 wt %, 10.7 wt %, 10.8 wt %, 10.9 wt, 11.0 wt % or any value therebetween.

With respect to the collagen-free gel formulations described herein, without wishing to be bound, it is believed that monoglyceride, such as monolaurin, at concentrations of 6 wt % or greater, slow the rate of absorption significantly, for example, by as much as 75% (versus non-collagen cod liver oil compositions described in WO 2019/036578), but, in addition, they are actually permeation enhancers once through the stratum corneum and the remainder of the epidermis. In other words, these gelled formulations are absorbed slowly through the stratum corneum and in the remainder of the epidermis. That is, the gels are slow-to-release, but once in the epidermis, monoglycerides, such as monolaurin, help drag the cod liver oil and the hemp oil into the dermis. This was unexpected because monoglycerides, for example, monolaurin, between 2% and 3.5 wt % are sticky gels. Only when the concentration of monoglycerides, such as monolaurin, are greater than or equal to 6 wt % of the composition does the sticky gel transform into an elegant gel. Between 3.5 wt % and 6 wt % the gel increases in strength but is unstable to even moderate shear. Once above or equal to 6 wt %, the gel is metastable, that is, it is stable in a thick form, but with minor mixing can become a thin gel. With intense mixing, the gel is broken. If broken gel is reheated to clarity, the process begins again. In other words, the thick gel is reproducible in all of the gel that is melted.

In addition, another ingredient that helps remove the fishy odor in compositions described herein are the linear fatty acid esters, especially cetyl esters. In an embodiment, the cetyl esters are present in an amount ranging from about 0.5% to 3% by weight. In another embodiment, the cetyl esters are present in an amount ranging from about 0.8% to 2.2% by weight of the composition, and in another embodiment, from about 1.0 to about 2.0 wt %. In an embodiment, it is present in about 2 wt %. For example, cetyl esters may be present in 0.5 wt %, 0.6 wt %, 0.7 wt %, 0.8 wt %, 0.9 wt %, 1.0 wt %, 1.1 wt %, 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %, 3.0 wt % or any value therebetween. Further, it is believed that adding cetyl esters to the formulation creates a silky smooth skin surface (finish) that is very pleasant to the touch.

With respect to the formulations described herein, without wishing to be bound, it is believed that the monoglycerides, such as monolaurin, help drive the omega3 fatty acids through the stratum corneum, leaving only a small residual of potentially oxidizable fat on the skin surface. The Cetyl Esters without wishing to be bound, provides a late-drying odor-occlusive layer over any potentially oxidizable fat. The net result is no odor after about 30 seconds.

A further means of reducing the rancid fish oil in compositions described herein is to use less of the marine oil, such as cod liver oil. Vegetable oil having an omega3 fatty acid content greater than 9 wt %, such as hemp oil, is another source of omega3 fatty acids. Marine oil, such as cod liver oil, provides very long chain omega3 fatty acids (C>18); vegetable oil having an omega3 fatty acid content greater than 9 wt %, such as hemp oil, provides long chain omega3 fatty acids (C=18). The body enzymatically takes omega3 fatty acids and either shortens or lengthens the chain length to make, for example, prostaglandins. If the omega3 fatty acid is C18 or less, the elongation process produces both inflammatory and anti-inflammatory compounds. If the omega3 fatty acid is C>18 (i.e. greater than or equal to C20), then the produced products are all anti-inflammatory. Wounds need some inflammatory compounds for example to control bleeding, as well as anti-inflammatory compounds, for example, to increase blood flow by reducing resistance to blood drainage.

In compositions of the present disclosure, it is advantageous to use vegetable oil having an omega3 fatty acid content greater than 9 wt %, such as hemp oil, in addition to marine oil, such as cod liver oil. This combination provides the most effective ratios of C18+C>18 omega3-fatty acids. However, there is also the trade-off between inflammatory byproducts and anti-inflammatory products with odor control. Nevertheless, in compositions described herein, the combination of C18+C>18 omega3-fatty acids lowers the foul fish odor smell. Thus, by using a greater amount of vegetable oil having an omega3 fatty acid content greater than 9 wt %, such as hemp oil, less marine oil, such as cod fish liver oil, is necessary. Accordingly, as described hereinabove, by utilizing more vegetable oil having an omega3 fatty acid content greater than 9 wt %, such as hemp oil, and reducing the amount of marine oil, such as cod liver oil, the present formulations reduce the rancid smell of the marine oil, such as cod liver oil. However, with the amount of monoglyceride, such as monolaurin and linear fatty acid esters, such as cetyl esters present herein, any rancid fish smell of the marine oil, such as cod liver oil, is considerably reduced or eliminated. As described herein, the addition of marine oil, such as hemp oil, linear fatty acid esters, such as cetyl esters and MCT and monoglyceride such as monolaurin, to the fish oil composition in the amounts described herein reduces or eliminates completely the rancid fish odor, and when the composition is applied to the skin of a patient, the burn or skin condition or wound is reduced in size and the skin of the patient is moisturized.

Formulations of the present disclosure either contain fish collagen or are collagen-free, as defined herein. The collagen containing compositions are discussed subsequent to the collagen-free formulations.

In the formulations of the present disclosure which are collagen-free, and which are oils and not gels, the sum of the wt % of MCT, monoglyceride and linear fatty acid ester in said pharmaceutical composition is 42 wt % or greater. In another embodiment, in the formulations of the present disclosure which are collagen-free, and which are oils and not gels, the sum of the wt % of MCT, monoglyceride and linear fatty acid ester in said pharmaceutical composition is 42.3 wt % or greater, and in a further embodiment, in the formulations of the present disclosure which are collagen-free, and which are oils and not gels, the sum of the wt % of MCT, monoglyceride and linear fatty acid ester in said pharmaceutical composition is 42.6 wt % or greater. In the formulations of the present disclosure which are collagen-free, and which are oils and not gels, in an embodiment, the maximum sum of the wt % of MCT, monoglyceride and linear fatty acid ester in said pharmaceutical composition is less than or equal to 85.0 wt %.

In the formulations of the present disclosure which are collagen-free, and which are gels, the sum of the wt % of MCT, monoglyceride and linear fatty acid ester in the pharmaceutical composition may be less than 42 wt %; for example, in such embodiments, the sum may be as low as 25 wt % or lower. For example, the sum of the wt % of MCT, monoglyceride and linear fatty acid ester in these gelled collagen free compositions may range from about 25 wt % to about 85 wt % and in another embodiment from about 35 wt % to about 85 wt %. In an embodiment, however, in the collagen-free gelled compositions, the sum ranges from 42 wt % to about 85 wt %, and in another embodiment, from 42 wt % to about 80 wt %.

In an embodiment, with respect to the non-collagen containing composition of the present formulation, the permeate factor is greater than or equal to 3.0 and the retentate factor is greater than 8.0, i.e., high permeate factor and high retentate factor. In an embodiment of this composition, the marine oil content ranges from about 5 wt % to about 24 wt %, and in another embodiment, from about 9 to about 20 wt % and in a further embodiment, from about 10% wt to about 19 wt % of the formulation. In an embodiment, the vegetable oil having an omega3 fatty acid content greater than 9 wt % ranges from about 5 to about 20 wt %, and in another embodiment, from about 6 to about 15 wt % and in another embodiment, from about 7 to about 14 wt % of the formulation. In an embodiment of the non-collagen composition, monoglyceride, for example, monolaurin, is present in amounts ranging from about 4 wt % to about 15 wt %, and in another embodiment, from about 6 to about 14 wt % and in a further embodiment, from about 8 to about 13 wt % of the formulation. In an embodiment of the non-collagen composition, MCT is present in an amount ranging from about 25 to about 75 wt %, and in another embodiment, from about 28 to about 73 wt % and in another embodiment, from about 31 to about 68 wt % of the formulation. In an embodiment of the non-collagen composition, MCT is present in amounts from about 1 times to about five times the sum of the weight percents of the marine oil and the vegetable oil having an omega3 fatty acid content greater than 9 wt %, and in another embodiment, from about 1 times to about four times the sum of the weight percents of the marine oil and the vegetable oil having an omega3 fatty acid content greater than 9 wt % in the formulation. In an embodiment of the non-collagen composition, linear fatty acid esters range from about 0.5 wt % to about 3 wt % of the formulation and in another embodiment, from about 0.75 to about 2.5 wt % of the formulation, and in another embodiment from about 1 to about 2 wt % of the formulation.

In an embodiment of the non-collagen containing formulation having high permeate factor and a high retentate factor (i.e. absorption is substantially complete, but not rapid), the marine oil content ranges from about 7 wt % to about 30 wt %, the vegetable oil having an omega3 fatty acid content greater than 9 wt % ranges from about 5 wt % to about 20 wt %, monoglyceride, such as monolaurin, is present in amounts ranging from about 4 wt % to about 15 wt %, MCT is present in amounts ranging from about 25 wt % to about 75 wt %, and the linear fatty acid esters are present in amounts ranging from about 0.5 wt % to about 3 wt %. In another embodiment of this non-collagen composition, marine oil content ranges from about 9 wt % to about 20 wt %, the vegetable oil having an omega3 fatty acid content greater than 9 wt % ranges from about 6 wt % to about 15 wt %, monoglyceride, such as monolaurin, is present in amounts ranging from about 6 wt % to about to about 14 wt %, MCT is present in amounts ranging from about 28 wt % to about 73 wt %, and linear fatty acid esters are present in amounts ranging from about 0.75 wt % to about 2.5 wt %, and in a further embodiment, the marine oil ranges from about 10 wt % to about 19 wt %, the vegetable oil having an omega3 fatty acid content greater than 9 wt % ranges from about 7 wt % to about 14 wt %, monoglyceride, such as monolaurin, is present in amounts ranging from about 8 wt % to about 13 wt %, MCT is present in amounts ranging from about 31 wt % to about 68 wt %, and linear fatty acid esters are present in amounts ranging from about 1 wt % to about to about 2 wt %.

In an embodiment with respect to the non-collagen containing composition of the present formulation, the permeate factor is greater than or equal to 3.0 and the retentate factor is less than or equal to 8.0, i.e., high permeate factor and low retentate factor (rapid absorption). In an embodiment of this composition, the marine oil content ranges from about 8 wt % to about 35 wt %, and in another embodiment, from about 10 to about 33 wt % and in a further embodiment, from about 11.5% wt to about 30 wt % of the formulation. In an embodiment of this non-collagen composition, the vegetable oil having an omega3 fatty acid content greater than 9 wt % ranges from about 10 to about 33 wt %, and in another embodiment, from about 11 to about 30 wt % and in another embodiment, from about 11.5 to 28 wt % of the formulation. In an embodiment of the non-collagen composition, monoglyceride, such as monolaurin, is present in amounts ranging from about 0.1 wt % to about 1 wt %, and in another embodiment, from about 0.3 to about 0.8 wt % and in a further embodiment, from about 0.5 to about 0.7 wt % of the formulation. In an embodiment of the non-collagen composition, MCT is present in amounts ranging from about 30 to about 60 wt %, and in another embodiment, from about 35 to about 55 wt % and in another embodiment, from about 37 to about 53 wt % of the formulation. In an embodiment of the non-collagen composition, MCT is present in amounts from about 1 times to about two times the sum of the weight percents of the marine oil and the vegetable oil having an omega3 fatty acid content greater than 9 wt %, and in another embodiment, from about 0.8 times to about 2 times the sum of the weight percents of the marine oil and the vegetable oil having an omega3 fatty acid content greater than 9 wt % in the formulation. In an embodiment of the non-collagen composition, the linear fatty acid esters range from about 0.5 wt % to about 2 wt % of the formulation and in another embodiment, from about 0.8 to about 1.5 wt % of the formulation, and in another embodiment from about 0.9 to about 1.1 wt % of the formulation.

In the non-collagen containing formulation having high permeate factor and a low retentate factor (i.e. very rapidly absorbed), the marine oil ranges from about 8 wt % to about 35 wt %, the vegetable oil having an omega3 fatty acid content greater than 9 wt % ranges from about 10 wt % to about 33 wt %, monoglyceride, such as monolaurin, is present in amounts ranging from about 0.1 wt % to about to about 1 wt %, MCT is present in amounts ranging from about 30 wt % to about to about 60 wt %, and linear fatty acid esters are present in amounts ranging from about 0.5 wt % to about to about 2 wt %. In another embodiment of this non-collagen composition, the marine oil ranges from about 11 wt % to about 33 wt %, the vegetable oil having an omega3 fatty acid content greater than 9 wt % ranges from about 11 wt % to about 30 wt %, monoglyceride, such as monolaurin, is present in amounts ranging from about 0.3 wt % to about 0.8 wt %, MCT is present in amounts ranging from about 35 wt % to about 55 wt %, and the linear fatty acid esters are present in amounts ranging from about 0.8 wt % to about 1.5 wt %, and in a further embodiment, the marine oil is present from about 11.5 wt % to about 30 wt %, the vegetable oil having an omega3 fatty acid content greater than 9 wt % ranges from about 11.5 wt % to about 28 wt %, monoglyceride, such as monolaurin, is present in amounts ranging from about 0.5 wt % to about to about 0.7 wt %, MCT is present in amounts ranging from about 37 wt % to about to about 53 wt %, and linear fatty acid esters are present in amounts ranging from about 0.9 wt % to about to about 1.1 wt %.

In an embodiment, with respect to the non-collagen containing composition of the present formulation, the permeate factor is less than 3.0 and the retentate factor is less than or equal to 8.0, i.e., low permeate factor and low retentate factor (i.e. leaves a greasy, non-stick surface that makes it difficult for biofilms to attach to damaged skin). In this composition, the marine oil content ranges from about 20 wt % to about 30 wt %, and in another embodiment, from about 22 to about 28 wt % and in a further embodiment, from about 23% wt to about 27 wt % of the formulation. In an embodiment, the vegetable oil having an omega3 fatty acid content greater than 9 wt % ranges from about 20 to about 30 wt %, and in another embodiment, from about 22 to about 28 wt % and in another embodiment, from about 23 to 27 wt % of the formulation. In an embodiment of the non-collagen composition, monoglyceride, such as monolaurin, is present in amounts ranging from about 0.1 wt % to about to about 2 wt %, and in another embodiment, from about 0.3 to about 1 wt % and in a further embodiment, from about 0.5 to about 0.7 wt % of the formulation. In an embodiment of the non-collagen composition, MCT is present in an amounts ranging from about 25 to about 50 wt %, and in another embodiment, from about 28 to about 45 wt % and in another embodiment, from about 30 to 42 wt % of the formulation. In an embodiment of the non-collagen composition, the sum of the wt % of marine oil and vegetable oil having an omega3 fatty acid content greater than 9 wt % is about 1 times to about two times the % weight of MCT, and in another embodiment, from about 1.2 times to about 1.8 times the % weight of MCT. In an embodiment of the non-collagen composition, the linear fatty acid esters range from about 0.5 wt % to about 2 wt % of the formulation and in another embodiment, from about 0.7 to about 1.5 wt % of the formulation, and in another embodiment from about 0.8 to about 1.2 wt % of the formulation. In compositions of the non-collagen composition having a low permeate factor and low retentate factor, the weight ratio of the vegetable oil having an omega3 fatty acid content greater than 9 wt %, such as hemp oil, to marine oil, such as cod liver oil, ranges from 1:1 to about 1.5:1 and in another embodiment from 1:1 to about 1.3:1. In other words, in this embodiment, the weight of the vegetable oil having an omega3 fatty acid content greater than 9 wt % is greater than or equal to the weight of marine oil.

Low permeate/low retentate formulations of the non-collagen composition are used, for instance, when residual odor is not a concern, for example on treating wounded animals. In this situation, the objective is to saturate PUFA at the wound site, even if some PUFA does not soak into the skin (because the epidermis is saturated with PUFA). Any residual PUFA stays on the surface, but the treated skin has the maximum possible amount of absorbed PUFA. The surface residual can oxidize and develop oxidized odors that are not objectionable to the treated animal, but the wound heals more quickly. This is important, for example, with unbandaged surface wounds on horse's legs (“Summer Sores” (for example, a bandage will not stay on a horse's knee)).

In the non-collagen containing formulation having low permeate factor and a low retentate factor, the marine oil is present from about 20 wt % to about 30 wt %, the vegetable oil having an omega3 fatty acid content greater than 9 wt % ranges from about 20 wt % to about 30 wt %, monoglyceride, such as monolaurin, is present in amounts ranging from about 0.1 wt % to about 2 wt %, MCT is present in amounts ranging from about 25 wt % to about 50 wt %, and the linear fatty acid esters range from about 0.5 wt % to about 2 wt %. In another embodiment of this non-collagen composition, the marine oil ranges from about 22 wt % to about 28 wt %, the vegetable oil having an omega3 fatty acid content greater than 9 wt % ranges from about 22 wt % to about 28 wt %, monoglyceride, such as monolaurin, is present in amounts ranging from about 0.3 wt % to about 1 wt %, MCT is present in amounts ranging from about 28 wt % to about 45 wt %, and linear fatty acid esters are present in amounts ranging from about 0.7 wt % to about 1.5 wt %, and in a further embodiment, the marine oil is present from about 23 wt % to about 27 wt %, the vegetable oil having an omega3 fatty acid content greater than 9 wt % ranges from about 23 wt % to about 27 wt %, monoglyceride, such as monolaurin, is present in amounts ranging from about 0.5 wt % to about 0.7 wt %, MCT is present in amounts ranging from about 30 wt % to about 42 wt %, and linear fatty acid esters are present in amounts ranging from about 0.8 wt % to about 1.2 wt %.

In an embodiment, with respect to the non-collagen containing composition of the present formulation, the permeate factor is less than 3.0 and the retentate factor is greater than 8.0, i.e., low permeate factor and high retentate factor; in an embodiment, it is a gel that is absorbed slowly. Such an embodiment can be used as a night time sleeping aid. In an embodiment, it may also contain such volatile acids, like caprylic acid.

In a composition having a low permeate factor and a high retentate factor, the marine oil content ranges from about 20 wt % to about 30 wt %, and in another embodiment, from about 22 to about 28 wt % and in a further embodiment, from about 24 wt % to about 27 wt % of the formulation. In an embodiment of the non-collagen composition, the vegetable oil having an omega3 fat content greater than 9 wt % ranges from about 20 to about 30 wt %, and in another embodiment, from about 22 to about 28 wt % and in another embodiment, from about 24 to 27 wt % of the formulation. In an embodiment of the non-collagen composition, monoglyceride, such as monolaurin, is present in amounts ranging from about 7 wt % to about 14 wt %, and in another embodiment, from about 8 to about 12 wt % and in a further embodiment, from about 9 to about 10 wt % of the formulation. In an embodiment of the non-collagen composition, MCT is present in an amounts ranging from about 20 to about 40 wt %, and in another embodiment, from about 25 to about 35 wt % and in another embodiment, from about 28 to 33 wt % of the formulation. In an embodiment of the non-collagen composition, the sum of the linear fatty acid esters present, such as cetyl esters and/or wax are present in an amount ranging from about 8 wt % to about 17 wt % of the formulation and in another embodiment, from about 9 to about 13 wt % of the formulation, and in another embodiment from about 10 to about 12 wt % of the formulation. In an embodiment, a composition having a low permeate factor and a high retentate factor contains cetyl esters, but does not contain any other wax, such as beeswax. In an embodiment, the combination of monolaurin and cetyl esters gives the composition a silky-smooth finish.

In another embodiment the low permeate/high retentate factor contains high levels of caprylic acid (>1 wt %, <3 wt %) and/or camphor (about 4 wt % to 7 wt %). In this formulation, the product is designed not to penetrate the epidermis, but rather to stay on the skin for up to 2 hours. The camphor and the caprylic acid are heated by the body and evaporate. The vapors are breathed into the nose and clear the sinuses and lungs. Wheezing is eliminated for those with breathing issues such as asthma, viral infections and bronchial infections. There is considerable odor from the camphor, so the benefit is targeted at users with breathing issues or nasal infections. The caprylic acid upsets the fat layer on enveloped viruses like COVID 19 and nosocomial infections. In one embodiment (PM3, Example 10) the product is regulated as a sleep aid with camphor as the active ingredient.

In the non-collagen containing formulation having low permeate factor and a high retentate factor, the marine oil ranges from about 20 wt % to about 30 wt %, the vegetable oil having an omega3 fatty acid content greater than 9 wt % ranges from about 20 wt % to about 30 wt %, monoglyceride, such as monolaurin, is present in amounts ranging from about 7 wt % to about 14 wt %, MCT is present in amounts ranging from about 20 wt % to about 40 wt %, and the linear fatty acid ester present, e.g., cetyl esters and/or wax are present in amounts ranging from about 8 wt % to about to about 17 wt %. In another embodiment, the marine oil is present from about 22 wt % to about 28 wt %, the vegetable oil having an omega3 fatty acid content greater than 9 wt % ranges from about 22 wt % to about 28 wt %, monoglyceride, such as monolaurin, is present in amounts ranging from about 8 wt % to about 12 wt %, MCT is present in amounts ranging from about 25 wt % to about 35 wt %, and the linear fatty acid esters, e.g., cetyl esters and/or wax, are present in amounts ranging from about 9 wt % to about 13 wt %, and in a further embodiment, the marine oil is present from about 24 wt % to about 27 wt %, the vegetable oil having an omega3 fat content greater than 9 wt % ranges from about 24 wt % to about 27 wt %, monoglyceride, such as monolaurin, is present in amounts ranging from about 9 wt % to about 10 wt %, MCT is present in amounts ranging from about 28 wt % to about 33 wt %, and the linear fatty acid esters, e.g., the cetyl esters and/or wax are present in amounts ranging from about 10 wt % to about to about 12 wt %.

In each of the non-collagen formulations described herein, the weight ratio of marine oil, such as cod liver oil to vegetable oil having an omega3 fatty acid content greater than 9 wt %, such as hemp oil ranges from about 0.5:1 to about 1.5:1, and in another embodiment, from about 1:1.3 to about 1.3:1, and in another embodiment, from about 1:1 to about 1.3, and in a further embodiment, 1:1.3 to about 1:1. In another embodiment, vegetable oil having an omega3 fatty acid content greater than 9 wt %, such as hemp oil and the marine oil, such as cod liver oil are present in a weight ratio of about 1:1 to about 2:1, respectively; and in another embodiment, in a weight ratio of about 1:1 to about 1.5:1 and, in still another embodiment, they are both present in about equal weight amounts, i.e., about 1:1 by weight. For example, the weight ratio of vegetable oil having an omega3 fatty acid content greater than 9 wt %, e.g., hemp oil to marine oil, e.g., cod liver oil, may be 1.0, 1.1, 1.2, 1.3, 1.4, 1.5, 1.6, 1.7, 1.8, 1.9, 2.0 or any value therebetween.

In the compositions described herein the relative amount of vegetable oil having an omega3 fatty acid content greater than 9 wt %, such as hemp oil, in amounts greater than (versus less than) marine oil depends on the need for a composition that promotes more inflammation or less inflammation. For already inflamed skin, less inflammation is preferable. For normal skin, a balance of inflammation and anti-inflammation is preferred because the body picks and chooses (enzymatically) the fatty acid chain length that needs conversion into more biologically active compounds. This is a level of complexity impossible to predict, so a “buffet table” of fatty acid chain lengths is offered. The body chooses what it needs for the particular individual. More hemp oil (omega6-rich) increases inflammation; more fish oil (very long omega3-rich) reduces inflammation.

Within the non-collagen formulation, another factor that has shown to have an effect is the sum of the saturated fats having 14 or more carbon atoms. When the sum is greater than 10 wt %, the composition imparts a greasy feel to the skin and inhibits biofilm attachment, so that bacteria and viruses cannot adhere to the outer skin layer.

The compositions of the present formulation may be an oil in one embodiment, or a gel in another embodiment or foam, or salve (where salves have a specific gravity greater than 0.8 lbs/ft³), or lotion or whip (where whip formulations have a specific gravity less than or equal to 0.8 lbs/ft³) in other embodiments. As described hereinabove, to form a gel, the monoglyceride is present in at least 6 wt % of the composition in the non-collagen containing formulations of the present disclosure.

Another embodiment of the present formulation comprises an anhydrous topical pharmaceutical composition where collagen is present. In embodiments where collagen is present, the anhydrous topical composition is comprised of marine oil, such as fish oil or algal oil; vegetable oil having a an omega3 fatty acid content greater than 9 wt %, such as hempseed oil, canola oil, flax seed oil, or cannabidiol; monoglyceride, such as monolaurin; linear fatty acid esters, such as cetyl esters and wax; medium chain triglycerides (“MCT”), and fish collagen wherein the weight ratio of the sum of the weights of (ALA+SDA)/sum of the weights of (EPA and DHA) present in the composition ranging from about 0.5 to about 1.5, and the sum of the wt % of MCT, monoglyceride, such as monolaurin and linear fatty acid esters, such as cetyl ester in said pharmaceutical composition is 20 wt % or greater of the formulation of the present disclosure. For example, in an embodiment, in the collagen containing compositions of the present disclosure, the sum of the wt % of MCT, monoglyceride and linear fatty acid ester ranges from about 22 wt % to about 40 wt %, and in another embodiment, from about 24 wt % to about 35 wt %.

The compositions of the present disclosure comprised of collagen can be divided into four categories: high permeate factor/low retentate factor, high permeate factor/high retentate factor, low permeate factor/low retentate factor and low permeate factor and low retentate factor. However, generally, compositions with low permeate factors are unstable. Thus, in embodiments, the present compositions comprising collagen comprise high permeate factors, that is, the permeate factor in these compositions is greater than or equal to 1.0. In an embodiment comprised of collagen wherein the permeate factor is greater than or equal to 1.0, the retentate factor is less than or equal to 8. In another embodiment comprised of collagen wherein the permeate factor is greater than or equal to 1.0, the retentate factor is greater than or equal to 8.

The reason for such variation in permeate factor and retentate factor with collagen is because some applications are intended for breached skin (no epidermis) and some are intended for intact skin (with epidermis). With breached skin, the permeate factor is low and the retentate factor is high. This “dribbles” the triglycerides into the exposed dermis over several days as the high retentate “holds” the triglycerides and only releases them slowly. If the triglycerides went into the dermis fast, they would become transdermal and lost to the localized wound.

Conversely, on intact skin with collagen, the permeate factor is increased to drive the triglycerides through the epidermis. An example of this strategy is a skin polish designed to exfoliate dead and dying surface cells (e.g. corneocytes). The collagen acts as a “gentle sandpaper” (“gentle sandpaper” is a term meant to convey that the scrubbing action to remove dead and dying skin is gentle, but still effective); the triglycerides helps heal the skin after exfoliation.

Since collagen is one of the larger ingredients, the amount of each of the other ingredients also changes relative to compositions when collagen is not present. In compositions comprising collagen, in an embodiment, collagen is present in a wt % greater than any other ingredient present in the formulation. Where fish collagen is present, in an embodiment, sea salt may or may not be present. If sea salt is present, it is present in amounts ranging from about 0.7 to about 1.7 wt %, and in another embodiment, it is present in amounts ranging from about 0.8 to about 1.0 wt %, and in still another embodiment, about 0.9 wt %. The sea salt, when present, is a process aid in an anhydrous matrix. In the present formulation, when present, it is added as ground sea salt. The average size of the sea salt crystal is less than about 0.74 microns, and in another embodiment, the average size is less than about 37 microns. As described hereinbelow, the ground sea salt is added after the collagen is mixed with the oil/wax mixture and cooled to room temperature. Sea salt is added during a nitrogen-blanketed whipping. Sea salt acts to make the dry collagen/oil/nitrogen gas mix into freeze/thaw-stable extruded putty.

The sea salt is a surprising process aid in an anhydrous matrix. The sea salt must be ground and then post mixed after the collagen addition and cooling to <29° C. (i.e. below that temperature where the monolaurin first forms a gel). Sea salt acts to make the dry collagen/oil mix into extruded putty. This flow capability is important in treating non-healing wounds. Non-healing wounds have unpredictable crevices and fissures. An anhydrous mixture that can flow into all the wound surfaces is particularly advantageous for wound healing.

In an embodiment of the collagen containing compositions, the permeate factor is greater than or equal to 1.0 and the retentate factor is less than 8.0 (high permeate factor/low retentate factor), and the concentration of monoglyceride, such as monolaurin, is less than 6 wt %. In another embodiment of the collagen containing compositions, the permeate factor is greater than or equal to 1.0 and the retentate factor is greater than or equal to 8.0, and the concentration of monoglyceride, such as monolaurin, is greater than 6 wt %.

The collagen containing compositions may be in the form of an oil, gel, salve, paste, or whip.

Embodiments with and without collagen have different Permeate Factors and Retentate Factors because collagen uses up ˜40% of the 100% formulation room, leaving only 60% for oils, waxes and process aids.

An embodiment, with respect to the collagen containing composition of the present formulation, where the permeate factor is greater than or equal to 1.0 and the retentate factor is less than 8.0, i.e., high permeate factor and low retentate factor, is useful as a rinse-off product that does not remain on the skin for more than about 10 minutes. In this collagen containing composition, the marine oil ranges from about 4 wt % to about 16 wt %, and in another embodiment, from about 5 to about 14 wt % and in a further embodiment, from about 6% wt to about 12 wt % of the formulation. In an embodiment of the collagen containing composition, MCT is present in amounts ranging from about 15 wt % to about 30 wt %, and in another embodiment, from about 20 wt % to about 27 wt % and in another embodiment, from about 22 wt % to about 25 wt %. In an embodiment, the vegetable oil having an omega3 fatty acid content greater than 9 wt % ranges from about 4 to about 16 wt %, and in another embodiment, from about 5 to about 12 wt % and in another embodiment, from about 6 to 13 wt % of the formulation. In an embodiment of the collagen containing composition, monoglyceride, such as monolaurin, is present in amounts ranging from about 2 wt % to about 6 wt %, and in another embodiment, from about 3 to about 5 wt % and in a further embodiment, from about 4 to about 6 wt % of the formulation. In an embodiment of the collagen containing composition, linear fatty acid esters are present in an amount 2 wt % to about 8 wt %, and in another embodiment, from about 3 wt % to about 7 wt % and in another embodiment, from about 4 wt % to about 6 wt %. For example, beeswax is present from about 1 wt % to about 10 wt %, in another embodiment from about 2 wt % to about 6 wt % and in a third embodiment from about 2 wt % to about 5 wt %. In an embodiment, collagen is present in an amount ranging from about 35 wt % to about 45 wt %, and in another embodiment, from about 38 wt % to about 43 wt % and in still another embodiment, from about 40 wt % to about 42 wt %

In another embodiment of the collagen containing composition having a high permeate factor, the retentate factor is greater than or equal to 8, for example, between 9 and 10. Another embodiment of the collagen containing composition comprises the permeate factor being greater than or equal to 1.0 and the retentate factor being greater than or equal to 10.0.

In an embodiment of the collagen containing composition, MCT is present in amounts ranging from about 15 to about 30 wt %, and in another embodiment, from about 18 to about 28 wt % and in another embodiment, from about 20 to 25 wt % of the formulation. In an embodiment of the collagen containing composition having a high permeate factor, the linear fatty acid esters range from about 3 wt % to about 10 wt % of the formulation and in another embodiment, from about 4 to about 8 wt % of the formulation, and in another embodiment from about 4.5 to about 6 wt % of the formulation. In an embodiment, collagen is present in an amount ranging from about 38 wt % to about 45 wt % of the formulation and in another embodiment, from about 40 to about 43 wt % of the formulation, and in another embodiment from about 41 to about 42 wt % of the formulation.

In an embodiment, the marine oil ranges from about 8 wt % to about 16 wt %, the vegetable oil having an omega3 fatty acid content greater than 9 wt % ranges from about 2 to about 16 wt %, monoglyceride is present in amounts ranging from about 2 wt % to 18 wt %, MCT is present in an amount ranging from about 10 to about 25 wt %; linear fatty acid esters are present in amount ranging from about 0.5 to about 9 wt % and collagen is present in an amount ranging from about 30 to about 50 wt %. In another embodiment, marine oil is present in an amount ranging from about 9 to about 15 wt %, the vegetable oil having an omega3 fatty acid content greater than 9 wt % from about 3.5 to about 14 wt %, monoglyceride ranges from about 3 wt % to about 15 wt %, MCT is present in an amount ranging from about 12 to about 22 wt %; linear fatty acid esters are present in amount ranging from about 0.8 to about 7.0 wt % and collagen is present in an amount ranging from about 35 to about 45 wt %. In a further embodiment, marine oil ranges from about 10 wt % to about 13 wt %, vegetable oil having an omega3 fatty acid content greater than 9 wt % ranges from about 4.5 to 13 wt % of the formulation, monoglyceride ranges from about 4 to about 13 wt %, MCT is present in an amount ranging from about 14 to about 20 wt %; linear fatty acid esters are present in amount ranging from about 0.9 to about 6 wt % and collagen is present in an amount ranging from about 38 to about 43 wt %.

It has been discovered that compositions comprised of collagen with low permeate factors are generally unstable, because there is leakage. But, not all are unstable. The composition comprised of collagen having a high permeate factor and low retentate factor is particularly suited for skin tear repair. An example includes Collagen Matrix DF, described hereinafter. Skin tears occur in older skin when friction separates the epidermis from the dermis, leaving a painful, bloody open wound. Marine oil is reduced to allow needed inflammation to stem the bleeding. Collagen is used as a hemostat to absorb blood and exudate. Cetyl esters are not needed as improved skin feel is not needed in a skin tear repair. Lidocaine is typically added to temporarily reduce pain. MCT is increased. MCT is ingested by cells and produce anti-inflammatory M2 macrophages (after inflammation and exudate absorption have stopped the bleeding). M2 macrophages clean-up the wound and autolytically digest the stripped-off epidermis.

In the collagen containing formulation having high permeate factor and a high retentate factor (high permeate factor creates a waxy protective layer over the exposed skin-torn dermis (and has a higher concentration of lidocaine)), that is, the permeate factor is greater than or equal to 1.0 and the retentate factor is greater than or equal to 8.0, the marine oil is present from about 4 wt % to about 15 wt %, the vegetable oil having an omega3 fatty acid content greater than 9 wt % ranges from about 4 wt % to about 12 wt %, monoglyceride, such as monolaurin, is present in amounts ranging from 6 wt % (including 6 wt %) to about 15 wt %, MCT is present in amounts ranging from about 10 wt % to about 30 wt %, and the linear fatty acid esters are present in amounts ranging from about 0.3 wt % to about 10 wt %, and collagen is present in an amount ranging from about 35 wt % to about 45 wt %. In another embodiment, the marine oil is present from about 5 wt % to about 13 wt %, the vegetable oil having an omega3 fatty acid content greater than 9 wt % ranges from about 4 wt % to about 10 wt %, monoglyceride, such as monolaurin, is present in amounts ranging from about 7 wt % to about 12 wt %, MCT is present in amounts ranging from about 13 wt % to about 30 wt %, the linear fatty acid esters are present in amounts ranging from about 0.6 wt % to about 8 wt %, and collagen is present in an amount ranging from about 40 wt % to about 43 wt % and in a further embodiment, the marine oil is present from about 6 wt % to about 12 wt %, the vegetable oil having an omega3 fatty acid content greater than 9 wt % ranges from about 5 wt % to about 9 wt %, monoglyceride, such as monolaurin is present in amounts ranging from about 9 wt % to about 12 wt %, MCT is present in amounts ranging from about 15 wt % to about 25 wt %, the linear fatty acid esters are present in amounts ranging from about 0.9 wt % to about 6 wt %, and collagen is present in an amount ranging from about 38 wt % to about 43 wt % of the formulation. This embodiment is particularly suited for skin tears in a hospital setting (very weak patients, highly susceptible to large tears caused by bedding (e.g. as patients are rotated to avoid bed sores)). These skin tears are very painful to frail patients and require more lidocaine, with extended application (i.e. slow release from the waxy layer) for adequate pain relief.

Additional components may be present in the formulations described herein.

In another embodiment of the collagen containing composition, ascorbyl palmitate (“AP”) may be additionally present. It is a stabilizing, high melting point wax that acts like an adhesive. In the collagen containing compositions, if ascorbyl palmitate is present, it is present in about 0.3 to about 0.6 wt % of the composition, and in another embodiment, it is present at about 0.5 wt %. The ascorbyl palmitate has a much higher melting point relative to thickening agents such as beeswax. It has a large head group such that when the ascorbyl palmitate precipitates, steric hindrance prevents it from plugging the collagen pores. Instead, the ascorbyl palmitate, monoglyceride, such as monolaurin, linear fatty acid esters, such as cetyl esters and the other anhydrous components of the collagen containing compositions form a dense gel (because of monolaurin and the waxes). The gel does not penetrate the collagen pores. Without wishing to be bound, it is believed that the precipitated AP coats the outside of the collagen particles. The monolaurin/cetyl esters precipitate after cooling to a lower temperature; they form a gel-bridge between AP-coated collagen particles. Together, a firm multi-phase matrix is formed. The filled-pores are attached together to give the appearance of a uniform paste.

In addition, without wishing to be bound, it is believed that the ascorbyl palmitate stops or retards liquid leakage of the anhydrous components from the collagen matrix. The AP mixture clarifies @ 160° F. (71.1° C.). When fish collagen is added, the mixture is quenched below the AP freezing point. AP is shaped like a Tootsie Pop, so it is sterically hindered from entering the collagen pores. Instead, it is believed that the AP fatty esters coat (precipitate on) the outside of each collagen granule. Unlike AP, when beeswax or other thickening agents and the other linear esters precipitate, they are mobile greases and penetrate the interior pores of the collagen granule, displacing air. AP is one of the compounds affecting the Retentate Factor.

A further optional ingredient is the sea salt, which, if present, is present in the collagen containing formulations. Where fish collagen is present, in an embodiment, sea salt is present in amounts ranging from about 0 to about 1.7 wt %, and in another embodiment, it is present in amounts ranging from about 0.8 to about 1.0 wt %, and in still another embodiment, about 0.9 wt %. The sea salt is a process aid in an anhydrous matrix. In the present formulation, it is added as ground sea salt. The average size of the sea salt crystal is less than about 0.74 microns, and in another embodiment, the average size is less than about 37 microns. As described hereinbelow, the ground sea salt is added after the collagen is mixed with the oil/wax mixture and cooled to room temperature. Sea salt is added during a nitrogen-blanketed whipping after monolaurin has gelled. Sea salt acts to make the dry collagen/oil/nitrogen gas mix into freeze/thaw-stable extruded putty. This flow capability is important in treating non-healing wounds. Non-healing wounds have unpredictable crevices and fissures. An anhydrous mixture that can flow into and onto all the wound surfaces is particularly advantageous for wound healing.

Without wishing to be bound, it is believed that the sea salt plays a role in preventing liquid leakage of anhydrous components from the collagen pores. Without wishing to be bound, it is believed that adding sea salt in the amounts described herein mechanically helps prevent seepage of components from the collagen matrix, but only in combination with ascorbyl palmitate (AP). It is believed that finely ground salt fits into the interstices between adjacent collagen spheres and “plugs the hole”. The salt sticks in the interstices because of the AP sticking to the collagen surfaces, and also stops the seepage. Without the AP, the salt traverses down through the matrix to the bottom; as a result, seeping occurs. The presence of AP prevents this. However, too little salt (less than 0.3 wt %) allows the liquid anhydrous components of the collagen composition to seep through, while too much salt (greater than 1.8 wt %) also allows liquid anhydrous components to seep through. In an embodiment, the salt concentration is about 0.9±0.3 wt %. Without wishing to be bound, it is believed that if there is too little salt, the “holes” are not completely plugged. If there is too much salt, the holes are “stretched”, and new pathways are created between adjacent spheres. When sea salt is present in the collagen formulation at 0.9±0.3 wt %, the holes plug, and pathways are not stretched; the AP keeps the salt crystal glued in place.

Thus, AP and sea salt are mechanical process aids that indirectly make heat-stressed collagen matrix stable. Whipping small batches (@ 0.8 kg/3.5 quarts—about 25% of the mixing bowl volume) intimately mixes sea salt and precipitated fats and waxes, allowing time and added energy for interstice-plugging to occur.

In addition, the sea salt, when present, is ground. The average size of the salt crystal ranges from about 200 Tyler Mesh Screen (about 74 microns) to about 300 Tyler Mesh Screen (about 37 microns). Salt particles within this size range also plug the interstices in the collagen matrix. Larger salt particles migrate to the bottom of packaging containers during storage and distribution, and prevent extrusion emptying of the single use vial.

As another example, FFA may be present in any of the formulations described herein, regardless if the composition contains collagen or is collagen free. If present, it is present in less than 3 wt % of the formulation. In an embodiment, it is present in amounts ranging from about 0.1 wt % to about 2.5 wt % and in another embodiment, from about 0.2 wt % to about 2.2 wt %. However, in some embodiments, it is present in about 0.1 wt % to about 0.9 wt %.

Vegetable oils, as defined herein, may additionally be present in any of the formulations described. As defined for purposes of this disclosure, the term “vegetable oil having an omega3 fatty acid content greater than 9 wt %” is different from the term “vegetable oil”, as described hereinabove. Although any vegetable oils having an omega3 fatty acid content of 9 wt % or less may be present in any of the formulations described herein, in an embodiment, these vegetable oils are selected from the group consisting of palm oil (including, but not limited to red palm oil and RBD palm oil, and red palm concentrate), coconut oil, including refined coconut oil, and a combination thereof. The vegetable oil (in total if more than 1 vegetable oil is present in the formulation) may be present in an amount ranging from about 0.2 wt % to about 35 wt % and in another embodiment, from about 0.3 wt % to about 30 wt %. In an embodiment, coconut oil is present in combination with the red palm concentrate. This combination reduces the amount of oleic fatty acid (from the palm oil) and replaces it with lauric acid (C12:0) and myristic acid (C14:0) both from the crude coconut oil. Replacing palm oil with its inherent high concentration of Vitamin E with coconut oil which has no Vitamin E is counter intuitive because Vitamin E is widely regarded as an essential vitamin for robust skin health. Although palm oil, with its high concentration of Vitamin E, has skin moisturizing characteristics, what has been found is that the presence of coconut oil and red palm disrupts pathogen growth in wound healing products. In addition, C14:0 also prevents liquid anhydrous component product seepage, such as vegetable oil, monolaurin, fish oil, vegetable oil having more than 9 wt % of omega3 fatty acids and other liquid components from the collagen matrix during storage in collagen-containing products because C14:0 is solid at distribution temperatures.

Nevertheless, in embodiments of the present disclosure, regardless of whether the embodiment comprises collagen, if a formulation contains coconut oil, it is present in less than 10 wt % of the formulation. Coconut oil in anhydrous liquids can cause cloudiness during storage and distribution.

In another embodiment of the present formulation, whether collagen-free or comprised of collagen, the vegetable oil present in the composition may comprise RBD palm oil, which is present in the amounts indicated herein. In some embodiments, vegetable oil may only comprise RBD palm oil, and not red palm concentrate. Red palm concentrate contains carotenoids. Without wishing to be bound, it is believed that carotenoids can act as both antioxidants and pro-oxidants under different conditions. The antioxidant property of carotenoids is well-known. It is further believed that carotenoids can switch from antioxidant to pro-oxidant behavior as a function of oxygen concentration. Thus, carotenoids in a composition in an oxygen-barrier package will behave as antioxidants and protect the marine oil. Then when the composition is spread over a wound exposed to air, the carotenoids can become pro-oxidants. Some of the oxidized carotenoids become Reactive Oxygen Species (ROS). The ROS, free radical antimicrobial compounds, in turn help reduce the pathogen concentration in infected wounds. The carotenoids act as both an antioxidant and pro-oxidant when the composition is a mixture of fast absorbing compounds, like MCT, and slow absorbing compounds like marine oil, monoglycerides, such as monolaurin and linear free fatty acid esters, such as cetyl esters and waxes. As the MCT is absorbed, the carotenoid and retentate fraction of the marine oil concentrate on the skin surface, increasing the micro concentration of the carotenoids at the site of infection. What is observed is faster wound bed granulation and faster wound closure.

It is thought that the addition of different antioxidants, for example, Vitamin E and Ascorbyl Palmitate, increase the antioxidant capacity of the composition because each has its own mechanism of action. Thus, replacing Vitamin E-rich palm oil with nil Vitamin E coconut oil which does not contain significant concentration of Vitamin E and adding red palm concentrate increases the in vitro antioxidant capacity (in combination with oxygen barrier packaging) but decreases the in vivo antioxidant capacity and increases the pro-oxidation ROS sufficiently that infection is controlled without a negative effect on the healing rate. Adding MCT to the crude coconut oil and carotenoids accelerates the healing process by concentrating the ROS on the surface where there can be infection. It is counterintuitive to use the same composition to be an antioxidant during storage and distribution and a source of pro-oxidant, antimicrobial ROS when the composition is applied to the wound, as in the present formulation.

When palm oil is used with red palm concentrate, the palm oil Vitamin E and the carotenoids work together to keep the carotenoids in the antioxidant mode. The Vitamin E absorbs the ROS before any killing takes place and terminates free radical propagation. Thus, bacterial kill is not observed. But, when coconut oil replaces palm oil, there is no vitamin E in the mixture. The carotenoids are antioxidants in an oxygen barrier package and then are pro-oxidants when applied to skin and exposed to air. There is no vitamin E, so the carotenoid-produced ROS is not quenched and kills bacteria. Multi-log bacterial kill is observed.

In an embodiment, whether collagen is present or collagen-free, the red palm concentrate is present in less than 1% by weight. In an embodiment, the red palm concentrate is present in an amount ranging from about 0.05 wt % to about 0.75 wt %, and in another embodiment, about 0.1 to about 0.3 wt %.

In another embodiment, whether collagen is present or collagen-free, the vegetable oil is RBD palm oil, without the presence of red palm oil or red palm concentrate. In this embodiment, the RBD palm oil is present ranging from about 18 wt % to about 2 wt %, and in another embodiment, from about 16 wt % to about 3 wt %, and in another embodiment, from about 15 wt % to about 4 wt %. This embodiment is indicated for use on intact skin. The Vitamin E in the palm oil is advantageously used on intact skin; any red/orange residual color on the skin from the application of red palm concentrate on the skin is perceived by consumers as a defect. The previously described rapid absorption with an odor-occlusive silky-smooth film makes this a consumer-acceptable composition to bring topical omega3 fatty acids from the marine oil and vegetable oil having omega3 fatty acid content of more than 9.0 wt %, such as hemp oil, to intact skin.

Moisturizers may additionally be present in collagen-free compositions or compositions comprised of collagen. For example, lauric triglyceride and/or oleic triglycerides are moisturizers that may be added to any of the formulations described herein. However, lauric triglyceride is a better skin moisturizer than oleic triglyceride.

Colloidal oatmeal may additionally be present in any of the formulations described herein. Colloidal oatmeal is made by grinding oat grain or Avena sativa into a fine powder. It is commercially available. It is an emollient and has been categorized by the FDA in 2003 as a skin protectant if the concentration is greater than 0.07 wt % (there is no regulatory upper limit). It also has anti-inflammatory and antioxidant properties. In an embodiment, it is present in non-collagen formulations. Oatmeal is not included in oil compositions because of separation during storage. When present, it is present in gels @ amounts of less than 1 wt %, such as for example, from about 0.07 wt % to about 0.8 wt %, and in another embodiment, from about 0.4 to about 0.6 wt % and in another embodiment in about 0.5 wt %. In another embodiment, it is present at about 0.08%.

In embodiments of the present disclosure, compositions comprised of colloidal oatmeal do not contain cetyl esters because the oatmeal provides the same tactical skin feel smoothness as cetyl esters. In other embodiments of the present disclosure, compositions of the present disclosure do not comprise vegetable oil having an omega3 fatty acid content greater than 9 wt %, such as hemp oil. In some embodiments, compositions comprising colloidal oatmeal do not contain any cetyl esters or vegetable oil having an omega3 fatty acid content greater than 9 wt %, such as hemp oil.

For example, in an embodiment, a collagen-free composition comprises MCT in an amount ranging from about 30 wt % to about 52 wt %, marine oil ranging from about 40 wt % to about 55 wt %, monoglyceride, such as monolaurin, from about 8 wt % to about 14 wt %, linear fatty acid esters, such as beeswax (e.g., yellow beeswax) ranging from about 0 (but greater than 0) wt % to about 3 wt % and colloidal oatmeal ranging from about 0 (but greater than 0) wt % to about 0.3 wt %. In another embodiment, such collagen free composition comprises MCT in an amount ranging from about 35 wt % to about 45 wt %, marine oil ranging from about 45 wt % to about 50 wt %, monoglyceride, such as monolaurin, from about 8 wt % to about 12 wt %, linear fatty acid esters, such as beeswax (e.g., yellow beeswax) ranging from about 0.5 wt % to about 1.5 wt % and colloidal oatmeal ranging from about 0.1 wt % to about 0.2 wt %. In still another embodiment, such collagen free composition comprises MCT in an amount ranging from about 38 wt % to 42 wt %, marine oil ranging from about 46 wt % to about 48 wt %, monoglyceride, such as monolaurin, from about 9 wt % to about 11 wt %, linear fatty acid esters, such as beeswax (e.g., yellow beeswax) ranging from about 0.8 wt % to about 1.2 wt % and colloidal oatmeal ranging from about 0.11 wt % to about 0.13 wt %. This embodiment does not contain hemp oil.

In a collagen-free embodiment, the sum of (EPA+DHA) is greater than 6 wt %, such as about 10 wt %. Other collagen free compositions have Total (EPA+DHA) sums <6 wt %. In this latter embodiment, vegetable oil having an omega3 fatty acid content greater than 9 wt %, such as hemp oil, is not present. This latter collagen-free embodiment is a low permeate factor (1.18)/high retentate (11) composition (A8, Example 20) because oleic acid (C18:1) from hemp oil is largely replaced with EPA & DHA from marine oil. As such, the low permeate factor slowly delivers a large amount of very long chain omega3 fatty acids. “Excess” very long omega3 compounds with a high retentate factor (11) selects for passage through the follicular pathway. When these omega3 fatty acids enter the hair follicle (albeit at low absolute levels), follicle stem cells are stimulated, and their progeny exit the hair follicle bulb and migrate across the basement membrane separating the epidermis from the dermis as signaled for by inherent biological processes. As a result, excess stem cell progeny enters the wound to stimulate wound closure. What is different is the combination of high (>6 g/100 g) absolute amount of triglycerides with a Retentate Factor >10. This high retentate factor causes release of into the treated zone over 7 days. The ratio of (ALA+SDA)/(EPA+DHA)=0.11 is well outside the normal range of 0.5 to 1.5.

In an embodiment, a collagen-free composition which does not comprise vegetable oil having an omega3 fatty acid content greater than 9 wt %, such as hemp oil, the embodiment comprises MCT in an amount ranging from about 30 wt % to 52 wt %, marine oil ranging from about 40 wt % to about 55 wt %, monoglyceride, such as monolaurin, from about 8 wt % to about 14 wt %, linear fatty acid esters, such as beeswax (e.g., yellow beeswax) ranging from about 0 (but greater than 0) wt % to about 3 wt % and colloidal oatmeal ranging from about 0 wt % to about 0.3 wt %. In another embodiment, such collagen free composition comprises MCT in an amount ranging from about 35 wt % to 44 wt %, marine oil ranging from about 45 wt % to about 50 wt %, monoglyceride, such as monolaurin, from about 8 wt % to about 12 wt %, linear fatty acid esters, such as beeswax (e.g., yellow beeswax) ranging from about 0.5 wt % to about 1.5 wt % and colloidal oatmeal ranging from about 0.07 wt % to about 0.2 wt %.

In an embodiment, the present disclosure relates to a collagen-free topical composition in the form of a gel containing both coconut oil and palm oil. Such formulation comprises cod liver oil, hempseed oil, monolaurin, medium chain triglycerides (“MCT”), coconut oil and palm oil, wherein the monolaurin is present in an amount effective to form a gel and wherein said composition has a C18:0 triglyceride concentration of 3 wt % or less, said composition being anhydrous and homogenous, and having a weight ratio of the sum of the weights of (ALA+SDA)/sum of the weights of (EPA and DHA) present in the composition ranging from about 0.5 to about 1.5, and the weight ratio of the sum of the weights of saturated C8+C10 saturated triglycerides/to the sum of the weights of unsaturated triglycerides larger than C10, that is, all unsaturated triglycerides wherein the fatty acid components are >C10, ranging from about 1.0 to about 3.0, and the monolaurin present in an amount effective to form a gel and greater than 6 wt % but less than 12 wt %.

In a further embodiment of the collagen-free gel which comprises cod liver oil, hempseed oil, monolaurin, medium chain triglycerides (“MCT”), coconut oil and palm oil, the topical composition has a weight ratio of the sum of the weights of (ALA+SDA)/sum of the weights of (EPA and DHA) present in the composition ranging from about 0.5 to about 1.5 and in another embodiment, from about 0.7 to about 1.3, and in another embodiment, from 0.9 to about 1.1, and in another embodiment, at about 1.0. For example, a weight ratio of the sum of the weights of (ALA+SDA)/sum of the weights of (EPA and DHA) present in the composition of 0.5, 0.6, 0.7, 0.8, 0.9, 1.0, 1.1, 1.2, 1.3, 1.4, or 1.5 or any value therebetween is contemplated within the scope of the topical compositions described herein.

In a further embodiment of collagen-free topical composition in the form of a gel which comprises cod liver oil, hempseed oil, monolaurin, medium chain triglycerides (“MCT”), coconut oil and palm oil, the weight ratio of the sum of the weights of saturated (C8+C10 triglycerides)/sum of the weights of unsaturated triglycerides larger than C10 ranges, in an embodiment, from about 1.0 to about 3.0, and in another embodiment, from about 1.2 to about 2.1, and in another embodiment, from about 1.5 to about 2.1. For example, in this embodiment, the weight ratios of the sum of the weights of saturated (C8+C10)/sum of the weights of unsaturated fats larger than C10 of 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0 and any value therebetween are contemplated within the scope of the topical compositions described herein.

In a further embodiment of a topical composition in the form of a gel which comprises cod liver oil, hempseed oil, monolaurin, medium chain triglycerides (“MCT”), coconut oil and palm oil, the weight of fatty acids of C18:0 is present in less than 3 wt %, and in another embodiment, is less than 2 wt % and in another embodiment, less than 1 wt %. For example, in this embodiment, the weight of compounds of C18:0 is present in amounts greater than 0 wt %, such as 0.1 wt %, 0.2 wt %, 0.3 wt %, 0.4 wt %, 0.5 wt %, 0.6 wt %, 0.7 wt %, 0.8 wt %, 0.9 wt %, 1.0 wt %, 1.1 wt %, 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %, or 3 wt %, and any value therebetween.

In another embodiment of the topical composition in the form of a gel, saturated compounds of C14:0 or greater may be additionally present.

In a further embodiment of the topical composition in the form of a gel which comprises cod liver oil, hempseed oil, monolaurin, medium chain triglycerides (“MCT”), coconut oil and palm oil, the ratio of palm oil to coconut oil ranges from about 1:1 to about 4:1. and, in an embodiment, is about 3:1. Coconut oil is a permeation enhancer; palm oil is not. If the permeation is fast (more coconut oil), interstitial water is drained from the skin (e.g. bags under the eyes are reduced). If the permeation is slow (more coconut oil), skin moisture increases (e.g. more epidermal hydration).

In addition, when applied to the skin, the topical composition in the form of a gel which comprises cod liver oil, hempseed oil, monolaurin, medium chain triglycerides (“MCT”), coconut oil and palm oil, has a smooth texture. Without wishing to be bound it is believed that this latter characteristic is attributable to the combination of the presence of the hempseed oil, crude coconut oil and palm oil.

In a further embodiment of the topical composition in the form of a gel which comprises cod liver oil, hempseed oil, monolaurin, medium chain triglycerides (“MCT”), coconut oil and palm oil, the amount of C18:0 triglyceride in the composition ranges from 0 wt %, excluding 0 wt %, to 3 wt % C18:0. C18:0 refers to the carbon number of each individual fatty acid in the triglyceride. All 3 fatty acids in the triglyceride in an embodiment are C18:0. The C18:0 triglyceride can come from hemp oil, cod liver oil, coconut oil or palm oil, but in the present embodiment, it is the total concentration in which the triglyceride of C18:0≤3% w/w. For example, palm oil is winterized at 6° C. and then the precipitated solids are removed. This lowers the amount of C18:0 triglyceride in the palm oil, and by extension into the formulated oils. C18:0 triglyceride is greasy to the touch and is cosmetically inelegant. It also is a “permeation inhibitor”. Since one product objective is to get the omega3 fatty acids into the dermis (intradermal), but not into the bloodstream (transdermal), the objective is to minimize the permeation inhibitors so as to allow the composition to be absorbed into the skin but keep the omega3 fatty acids predominantly in the dermis. However, the components in the composition also contain permeation enhancers, which breach the epidermis (e.g. when reinforced without permeation inhibitors like C18:0 triglyceride). But then the extra permeation enhancers (like MCT), once through the epidermis, drag the omega3 fatty acid into the bloodstream (transdermal). The goal is to find a balance to achieve intradermal absorption without transdermal loss of omega3 fatty acid.

An embodiment of the present disclosure is directed to a topical composition that does not contain collagen or sea salt and is a gel. In another embodiment, it does not contain any red palm concentrate. In an embodiment of the collagen-free gel which comprises cod liver oil, hempseed oil, monolaurin, medium chain triglycerides (“MCT”), coconut oil and palm oil, it additionally comprises cetyl esters.

In a further embodiment of the collagen-free gel which comprises cod liver oil, hempseed oil, monolaurin, medium chain triglycerides (“MCT”), coconut oil and palm oil, the cod liver oil is present in an amount ranging from about 5 to about 30 wt %, MCT is present in an amount ranging from about 30 to about 60 wt %, hemp oil is present in an amount ranging from about 5 to about 30 wt %, monolaurin is present in an amount ranging from about 7 to about 11 wt %, palm oil being present in an amount ranging from about 5 to about 20 wt %, coconut oil being present in an amount ranging from about 3 to about 20 wt %, and cetyl esters are present in an amount ranging from about 0.5 to about 3 wt %.

In a further embodiment of the collagen-free gel which comprises cod liver oil, hempseed oil, monolaurin, medium chain triglycerides (“MCT”), coconut oil and palm oil, the cod liver oil is present in an amount ranging from about 5 wt % to about 30 wt %, and in another embodiment, from about 8 to about 20 wt %, and in another embodiment from about 10 to about 15 wt %. For example, in the composition described herein, cod liver oil can be present in 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt % or 30 wt % or any value therebetween.

In a further embodiment of the collagen-free gel which comprises cod liver oil, hempseed oil, monolaurin, medium chain triglycerides (“MCT”), coconut oil and palm oil, hemp oil is present in an amount from about 5 to about 30 wt % and in another embodiment, from about 8 to about 20 wt %, and in another embodiment, from about 10 to about 15 wt %. For example, in the compositions described herein, hemp oil can be present in 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt % or 30 wt % or any value therebetween.

In a further embodiment of the collagen-free gel which comprises cod liver oil, hempseed oil, monolaurin, medium chain triglycerides (“MCT”), coconut oil and palm oil, the weight ratio of hemp oil to cod liver oil ranges from about 0.5 to about 1.5, and in another embodiment, from about 0.7 to about 1.3 and in another embodiment, from about 0.9 to about 1.1 and in a further embodiment, about equal and in another embodiment, about 1:1. The ratio of hemp oil to cod liver oil in an embodiment of the collagen-free gel which comprises cod liver oil, hempseed oil, monolaurin, medium chain triglycerides (“MCT”), coconut oil and palm oil approximately equals that of the weight ratio of the sum of the weights of (ALA+SDA)/sum of the weights of (EPA and DHA).

In a further embodiment of the collagen-free gel which comprises cod liver oil, hempseed oil, monolaurin, medium chain triglycerides (“MCT”), coconut oil and palm oil, MCT is present in an amount greater than 30 wt %. In a further embodiment of the collagen-free gel which comprises cod liver oil, hempseed oil, monolaurin, medium chain triglycerides (“MCT”), coconut oil and palm oil, MCT is present in an amount less than 60 wt %. In another embodiment of the collagen-free gel which comprises cod liver oil, hempseed oil, monolaurin, medium chain triglycerides (“MCT”), coconut oil and palm oil, MCT is present in an amount ranging from about 30 wt % to about 60 wt % and in another embodiment, from about 35 to about 55 wt %, and in another embodiment, from about 40 to about 50 wt %. For example MCT may be present in an amount of 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt % or any value therebetween.

In a further embodiment of the collagen-free gel which comprises cod liver oil, hempseed oil, monolaurin, medium chain triglycerides (“MCT”), coconut oil and palm oil, palm oil is present, in an embodiment, ranging from about 5 wt % to about 20 wt % and in another embodiment, from about 8 to about 18 wt %, and in another embodiment, from about 10 to 15 wt %. For example, palm oil may be present in 5 wt %, 5.1 wt %, 5.2 wt %, 5.3 wt %, 5.4 wt %, 5.5 wt %, 5.6 wt %, 5.7 wt %, 5.8 wt %, 5.9 wt %, 6 wt %, 6.1 wt %, 6.2 wt %, 6.3 wt %, 6.4 wt %, 6.5 wt %, 6.6 wt %, 6.7 wt %, 6.8 wt %, 6.9 wt %, 7 wt %, 7.1 wt %, 7.2 wt %, 7.3 wt %, 7.4 wt %, 7.5 wt %, 7.6 wt %, 7.7 wt %, 7.8 wt %, 7.9 wt %, 8 wt %, 8.1 wt %, 8.2 wt %, 8.3 wt %, 8.4 wt %, 8.5 wt %, 8.6 wt %, 8.7 wt %, 8.8 wt %, 8.9 wt %, 9 wt %, 9.1 wt %, 9.2 wt %, 9.3 wt %, 9.4 wt %, 9.5 wt %, 9.6 wt %, 9.7 wt %, 9.8 wt %, 9.9 wt %, 10 wt %, 10.1 wt %, 10.2 wt %, 10.3 wt %, 10.4 wt %, 10.5 wt %, 10.6 wt %, 10.7 wt %, 10.8 wt %, 10.9 wt %, 11 wt %, 11.1 wt %, 11.2 wt %, 11.3 wt %, 11.4 wt %, 11.5 wt %, 11.6 wt %, 11.7 wt % 11.8 wt %, 11.9 wt %, 12 wt %, 12.1 wt %, 12.2 wt %, 12.3 wt %, 12.4 wt %, 12.5 wt %. 12.6 wt %, 12.7 wt %, 12.8 wt %, 12.9 wt %, 13 wt %, 13.1 wt %, 13.2 wt %, 13.3 wt %, 13.4 wt %, 13.5 wt %, 13.6 wt %, 13.7 wt %, 13.8 wt %, 13.9 wt %, 14 wt %, 14.1 wt %, 14.2 wt %, 14.3 wt %, 14.4 wt %, 14.5 wt %, 14.6 wt %, 14.7 wt %, 14.8 wt %, 14.9 wt %, 15 wt %, 15.1 wt %, 15.2 wt %, 15.3 wt %, 15.4 wt %, 15.5 wt %, 15.6 wt %, 15.7 wt %, 15.8 wt %, 15.9 wt %, 16 wt %, 16.1 wt %, 16.2 wt %, 16.3 wt %, 16.4 wt %, 16.5 wt %, 16.6 wt %, 16.7 wt %, 16.8 wt %, 16.9 wt %, 17 wt %, 17.1 wt %, 17.2 wt %, 17.3 wt %, 17.4 wt %, 17.5 wt %, 17.6 wt %, 17.7, wt %, 17.8 wt %, 17.9 wt %, 18 wt %, 18.1 wt %, 18.2 wt %, 18.3 wt %, 18.4 wt %, 18.5 wt %, 18.6 wt %, 18.7 wt %, 18.8 wt %, 18.9 wt %, 19 wt %, 19.1 wt %, 19.2 wt %, 19.3 wt %, 19.4 wt %, 19.5 wt %, 19.6 wt %, 19.7 wt %, 19.8 wt %, 19.9 wt %, 20 wt %, or any value therebetween.

In a further embodiment of the collagen-free gel which comprises cod liver oil, hempseed oil, monolaurin, medium chain triglycerides (“MCT”), coconut oil and palm oil, coconut oil is present in an amount ranging from about 3 wt % to about 20 wt % and in another embodiment, from about 5 to about 15 wt %, and in another embodiment, from about 10 to about 13 wt %. For example, coconut oil may be present in 3 wt %, 3.1 wt %, 3.2 wt %, 3.3 wt %, 3.4 wt %, 3.5 wt %, 3.6 wt %, 3.7 wt %, 3.8 wt %, 3.9 wt %, 4.0 wt %, 4.1 wt %, 4.2 wt %, 4.3 wt %, 4.4 wt %, 4.5 wt %, 4.6 wt % 4.7 wt %, 4.8 wt %, 4.9 wt %, 5 wt %, 5.1 wt %, 5.2 wt %, 5.3 wt %, 5.4 wt %, 5.5 wt %, 5.6 wt %, 5.7 wt %, 5.8 wt %, 5.9 wt %, 6 wt %, 6.1 wt %, 6.2 wt %, 6.3 wt %, 6.4 wt %, 6.5 wt %, 6.6 wt %, 6.7 wt %, 6.8 wt %, 6.9 wt %, 7 wt %, 7.1 wt %, 7.2 wt %, 7.3 wt %, 7.4 wt %, 7.5 wt %, 7.6 wt %, 7.7 wt %, 7.8 wt %, 7.9 wt %, 8 wt %, 8.1 wt %, 8.2 wt %, 8.3 wt %, 8.4 wt %, 8.5 wt %, 8.6 wt %, 8.7 wt %, 8.8 wt %, 8.9 wt %, 9 wt %, 9.1 wt %, 9.2 wt %, 9.3 wt %, 9.4 wt %, 9.5 wt %, 9.6 wt %, 9.7 wt %, 9.8 wt %, 9.9 wt %, 10 wt %, 10.1 wt %, 10.2 wt %, 10.3 wt %, 10.4 wt %, 10.5 wt %, 10.6 wt %, 10.7 wt %, 10.8 wt %, 10.9 wt %, 11 wt %, 11.1 wt %, 11.2 wt %, 11.3 wt %, 11.4 wt %, 11.5 wt %, 11.6 wt %, 11.7 wt % 11.8 wt %, 11.9 wt %, 12 wt %, 12.1 wt %, 12.2 wt %, 12.3 wt %, 12.4 wt %, 12.5 wt %. 12.6 wt %, 12.7 wt %, 12.8 wt %, 12.9 wt %, 13 wt %, or any value therebetween.

In an embodiment, to the composition of the present disclosures of the collagen-free gel which comprises cod liver oil, hempseed oil, monolaurin, medium chain triglycerides (“MCT”), coconut oil and palm oil, is added less than 5 wt % of C8 and C10 free fatty acids directly, as defined hereinabove, and in another embodiment, less than 3 wt % free fatty acids, and in another embodiment, less than 1 wt % free fatty acids, and in a further embodiment, no free fatty acids are added to the present compositions. However, it is understood that these fatty acids may be present in the components present that are described hereinabove.

All of the possible combinations and permutations of the components listed herein are contemplated to be within the scope of the present compositions. However, the sum of all of the components present in the compositions herein adds to 100 wt %. In an embodiment, sum of wt % of fish oil and algae oil, whichever is present; vegetable oil having an omega3 fatty acid content greater than 9 wt %; linear fatty acid ester having 12-50 carbon atoms; monoglyceride; medium chain triglycerides (“MCT”), and collagen, whenever present, in the compositions of the present disclosure are present in an amount ranging from about 80 wt % to about 100 wt % and in another embodiment, in another embodiment from about 90 wt % to 100 wt % and in another embodiment, from about 95 wt % to 100 wt % and in further embodiments from about 96 wt % to 100 wt %, or about 97 wt % to 100 wt %, about 98 wt % to 100 wt %, or about 99 wt % to 100 wt %, and any number therebetween, such as 80 wt %, 81 wt %, 82 wt %, 83 wt %, 84 wt %, 85 wt %, 86 wt %, 87 wt %, 88 wt %, 89 wt %, 90 wt %, 91 wt %, 92 wt %, 93 wt %, 94 wt %, 95 wt %, 96 wt %, 97 wt %, 98 wt %, 99 wt %, or 100 wt %. In another embodiment, the sum of wt % of fish oil and algae oil, whichever is present; vegetable oil having an omega3 fatty acid content greater than 9 wt %; linear fatty acid ester having 12-50 carbon atoms; monoglyceride; and medium chain triglycerides (“MCT”), in the collagen free-compositions of the present disclosure are present in an amount ranging from about 80 wt % to about 100 wt % and in another embodiment, in another embodiment from about 90 wt % to 100 wt % and in another embodiment, from about 95 wt % to 100 wt % and in further embodiments from about 96 wt % to 100 wt %, or about 97 wt % to 100 wt %, about 98 wt % to 100 wt %, or about 99 wt % to 100 wt %, and any number therebetween, such as 80 wt %, 81 wt %, 82 wt %, 83 wt %, 84 wt %, 85 wt %, 86 wt %, 87 wt %, 88 wt %, 89 wt %, 90 wt %, 91 wt %, 92 wt %, 93 wt %, 94 wt %, 95 wt %, 96 wt %, 97 wt %, 98 wt %, 99 wt %, or 100 wt %.

In an embodiment wherein the formulation is a non-collagen containing gel, when both coconut oil and palm oil are present in the formulation, the weight ratio of palm oil to coconut oil ranges from about 4:1 to about 0.5:1, and in another embodiment from about 2.5:1 to about 0.8:1, and in another embodiment, from about 2:1 to about 1:1, and in another embodiment, about 1:1, and in another embodiment, about 3:1. In an embodiment, there is more palm oil by weight present in the compositions described in the present disclosure than coconut oil by weight present in the composition. The weight ratio of palm oil to coconut oil may be 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 or any value therebetween.

In an embodiment, wherein the formulation is a non-collagen containing gel, the composition of the present disclosure contains less than 5 wt % red palm concentrate, and in another embodiment, less than 3 wt % red palm concentrate, and in another embodiment, less than 1 wt % red palm concentrate, and in a further embodiment, no red palm concentrate is present.

In embodiments of the present disclosure, regardless of whether the composition contains collagen or is collagen-free, the composition can comprise, in addition to the components described hereinabove, any one or more of therapeutically active compounds and pharmaceutically and/or cosmetically acceptable diluents, excipients or carriers. For example, the composition of the present disclosure can be used as a carrier for drugs useful for treating skin conditions or burns, as long as the drug is oil soluble or lipophilic. The term “lipophilic” as used in the invention implies that the drug is entirely being purely lipophilic or has both a hydrophilic and lipophilic character, but more lipophilic character than hydrophilic character. The term lipophilic therefore encompasses solubilities which range from exclusive solubility in non-polar, water-immiscible organic solvents, to complexes having solubility both in these solvents and non-aqueous water immiscible solvents. The gradation of lipophilicity of the compositions of the present invention can be established by reference to partition coefficients using n-octanol/water, or n-octanol/buffer, or n-octanol/saline. In general, those drugs having n-octanol/saline partition coefficients greater than about 10 and especially 100 are useful drugs for which the present compositions, whether containing fish collagen or not, can be carriers. Examples of drugs include anesthetics, such as lidocaine, in pain killing effective amounts. For example, the compositions of the present disclosure may contain a safe and effective amount of a topical anesthetic and/or analgesic which numbs tissue and reduces pain associated with the skin condition or wound. Examples of topical anesthetic drugs include benzocaine, lidocaine, bupivacaine, chlorprocaine, dibucaine, etidocaine, mepivacaine, tetracaine, dyclonine, hexylcaine, procaine, benzethonium chloride, methylbenzethonium chloride and benzalkonium chloride cocaine, ketamine, pramoxine, phenol, and pharmaceutically acceptable salts thereof. When present, the drug is present in a safe to be effective for and effective amount of a topical anesthetic. For example, in an embodiment, it may be present in an amount ranging from about 0.5% to about 1.5 wt %. In an embodiment, the anesthetic drug is lidocaine. However, the API (Active Pharmaceutical Agent) is lipophilic. Other non-inclusive examples include lipophilic salts such as Amphotericin B and itraconazole.

Besides the components listed above, the compositions of the present disclosure may contain other additional optional ingredients. For example, the composition may contain fragrances or perfumes to further conceal any remaining rancid fishy smell. If present, the perfumes or fragrances are present in less than 2 wt %, such as for example from about 0.01 wt % to about 1.5 wt %. Oil soluble fragrances include such components as vanilla, lemon oil, lavender, and the like. In addition, if necessary, the composition of the present disclosure may contain additional thickeners, such as beeswax, candeilla wax, carnauba wax, ceresine wax, microcrystalline wax, ozocerite wax, PEG 4000, PEG 600, paraffin wax, laurel wax, rice bran wax, vegerite wax and other high melting point waxes and/or gelling agents (e.g., hydroxypropyl cellulose, carboxymethyl cellulose (CMC)).

A wide variety of optional components/ingredients may be included in the compositions of the present invention. For example, the compositions may include absorbents, abrasives, anticaking agents, antifoaming agents, antimicrobial agents, binders, biological additives, buffering agents, bulking agents, chemical additives, biocides, denaturants, cosmetic astringents, drug astringents, external analgesics, film formers, humectants, opacifying agents, fragrances, pigments, colorings, essential oils, skin sensates, emollients, skin soothing agents, pH adjusters, plasticizers, preservatives, preservative enhancers, propellants, reducing agents, additional skin-conditioning agents, skin penetration enhancing agents, skin protectants, solvents, suspending agents, emulsifiers, thickening agents, solubilizing agents, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins and derivatives thereof, and natural extracts. Such other materials are known in the art. Nonexclusive examples of such materials are described in Harry's Cosmedcology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. ad., deNavarre (Van Nostrand 1962-1965); and in The Handbook of Cosmetic Science and Technology, 1st Ed. Knowlton & Pearce (Elsevier 1993).

The compositions described herein can be formulated as a gel, ointment, cream, balm, whip or lotion. It may be administered in any one of those forms or administered as an oil-impregnated wipe or spray, such as an aerosol spray, including a bag-on-valve aerosol spray. Topical administration can also be accomplished with a liquid spray, an aerosol, or via iontophoresis, or through the use of liposomes, microbubbles and/or microcapsules. Gels, ointments and creams may be formulated with additives, for example, with an aqueous or oily base with the addition of suitable thickening (e.g., wax, beeswax, PEG 4000, PEG 600, hard paraffin) and/or gelling agents (e.g., hydroxypropyl cellulose). Lotions may be formulated with additives, such as an aqueous or oily base and can also generally contain one or more emulsifying agents (e.g., wool wax alcohol, fatty acid glycol esters), stabilizing agents (e.g., polyoxyethylene sorbitan monolaurate, carboxy methyl cellulose), dispersing agents (e.g., sodium oleate, propylene glycol), suspending agents (e.g., methyl cellulose, chitosan, accacia, carboxymethyl cellulose, tragacanth, pectin), thickening agents, and/or coloring agents (e.g., dyes, lackes). In some embodiments, for example, the topical compositions can include pluronic gels, polaxamer gels, hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (carbopols). Embodiments may also include creams/ointments conventionally used for topical cosmetic or pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA. Oil soluble anti-oxidizers, such as Vitamin E, and astaxanthin. Oil soluble vitamins, such as Vitamin D can be added. Oil soluble fragrances such as vanilla, lemon oil and lavender and oil-soluble coloring agents may be added. In addition sebum can be added. Further, benzethonium chloride, methylbenzethonium chloride and benzalkonium chloride may be present.

Other additives that may be present in the formulation described herein. For example, antioxidants and preservatives may be added. Examples of antioxidants include, but are not limited to, tocopherols, ascorbic acid, Vitamin K, sodium pyrosulfite, butylhydroxytoluene, butylated hydroxyanisole, edetic acid, and edetate salts, and mixtures thereof. Examples of preservatives include, but are not limited to, citric acid, tartaric acid, lactic acid, malic acid, acetic acid, benzoic acid, and sorbic acid.

Statins are a family of useful active pharmaceutical ingredients. Statins are well known as topical wound healing compounds, but their use has been inhibited by the difficulty in getting them to cross the epidermal barrier. By mixing in high permeate factor gels, this deficiency has been overcome. Valsartan is an example of a statin; its molecular weight is less than 500 daltons. Other statins have a molecular weight greater than 500 daltons.

In an embodiment, valsartan at 1 wt % is mixed into a collagen-containing composition and placed into an open wound without an epidermal layer.

In embodiments of the present disclosure, the composition can comprise any one or more of therapeutically active compounds and pharmaceutically and/or cosmetically acceptable diluents, excipients or carriers. A composition that comprises the stabilized formulation can be formulated for topical application as described herein and sealed in an air-tight container suitable for a single use. Such a composition can be used to treat any of the skin conditions, burns and/or wounds described herein.

The formulations described herein do not have the rancid fishy smell.

Further, formulations of the present disclosure do not scar after application of the formulation described herein to the wound, burn or skin condition.

Wound healing is evolutionarily optimized for speed of healing under dirty conditions, where a multiplied redundant, compensating, rapid inflammatory response with overlapping cytokine and inflammatory cascades allows the wound to heal quickly to prevent infection and future wound breakdown.

Compositions of the present disclosure minimize the formation of scars in the process of treating wounds, burns, or skin conditions. They retard and/or inhibit the formation of scars in the process of treating wounds, burns, or skin conditions. Scars arise after almost every dermal injury. Scars are often considered trivial, but they can be disfiguring and aesthetically unpleasant and cause severe itching, tenderness, pain, sleep disturbance, anxiety, depression, and disruption of daily activities. Scars have heavy collagen with a little or no vascularity. Compositions which are collagen-free that have low Permeate Factors tend to accelerate angiogenesis and thus minimize scarring. The presence or absence of collagen in topical compositions does not appear to affect the scarring. The Permeate Factor (>3.0) and the ratio of (ALA+SDA)/(EPA+DHA) between 0.5 and 1.5 in non-collagen compositions are among the controlling factors for minimizing scarring. Gels and oils do not distinguish scarring.

Scar minimization is complex. To simplify, scarring can be split into wounds that heal by secondary intention and wounds that heal by approximation of the wound. Chronic wounds are examples of wounds that heal by secondary intention. In the prior art, external collagen is added topically to the open wound bed. Granulation occurs, gradually filling the wound bed with new, temporary tissue. With continued application, prior art collagen accelerates wound bed growth until hyper-granulation (i.e. skin above the height of the unbroken surrounding tissue) occurs. Ordinary practitioners stop excess granulation with silver nitrate (a controlled chemical burn) or by debriding the top skin. Skilled practitioners, in the prior art, stop adding topical collagen several weeks before the wound bed level matches the surrounding tissue level. Tissue “coasts” to a stop at the level of the surrounding tissue.

Compositions in the present disclosure stop the excess growth of collagen-boosted granulation by simultaneously encouraging epithelial growth using omega3 skin protectant oils and gels. The change is subtle because a monolayer of epithelial tissue covers the wound (invisible to the naked eye except for a visible gloss (caused by fat-rich light reflection) over the wound bed). The epithelial monolayer separates added collagen from the wound bed. It is important to note that the epithelial tissue has a negative charge and the wound bed has a positive charge that electrostatically “pulls” epithelial cells over the wound bed. Sea salt is added to omega3 collagen embodiments to increase exudate conductivity and thereby encourage electrical-attraction migration.

Collagen embodiments are formulated for stability and collagen efficacy and not for their effect on scarring. Collagen compositions of the present disclosure are formulated to minimize scarring by increasing the Permeate Factor >1 to encourage angiogenesis.

The present disclosure describes the bundled method of simultaneous collagen-stimulated granulation with stem-cell-progeny-accelerated epithelial cell growth. The glossy monolayer of epithelial tissue inhibits collagen transfer from the surface to the wound bed and thus prevents excess granulation (and associated scarring).

With gels of the present disclosure, without wishing to be bound, it is believed that transfer through the epidermis is deliberately slowed by a high retentate factor. This slowness allows time for some of the gel to cross the epithelial barrier via the hair follicle bulb. The follicle bulb has at least 3 “bulges” where stem cells rest until called for. The stimulated wound signals for stem cell help. The gels in the follicle provide necessary building blocks for growth of stem cell progeny. Progeny exit the follicle and migrate across the basement membrane onto the surface of the granulating bed. There are also sporadic stem cells in the intrafollicular epithelium in the basal cells above the basement membrane. The gels of the present disclosure stimulate epithelial stem cell progeny growth. Follicular progeny mix with basal cell progeny and jointly migrate to the periwound and onto the surface of the developing granulation bed. Electrical attraction helps distribute mixed progeny over the entire wound bed surface, preventing topical collagen from over-growing the wound bed.

Without wishing to be bound, it is believed that cells can absorb various compositions at a finite rate. Compositions that oversaturate the cells then passes by the satiated cell and into the blood stream. By using high Retentate Factor gels to slow the oil passage by an intradermal cell, more of the gelled composition is absorbed by intradermal cells and less passes by into the bloodstream. The net effect is to feed more gelled composition to intradermal cells.

By deliberately slowing the rate of absorption and increasing the absolute amount of topical omega3 and topical MCT with gels, follicular stem cells' production is accelerated. Accelerated progeny production then accelerates healing without scarring. The net effect is healing non-healing wounds without disfiguring scarring and healing “intentional” wounds (i.e. surgical, for example in breast reconstruction and augmentation) without visible scarring.

With collagen containing products, the no-scarring result is achieved by increasing the Permeate Factor to be greater than 1.0. This effect is not seen in wounds that heal by secondary intention (i.e. open sores) because open sores have to granulate and then epithelialize, a two-step process. Wounds that heal by primary intention (i.e. stitched or stapled together) “only” need a one-step process (platelet agglomeration). For primary intention, scar-free healing, a higher Permeate Factor is useful (in practice, CZ3 (1.50) & CZ4 (1.51) have a 3XPermeate Factor versus the Permeate Factor of CR (0.49)). CZ3 and CZ4 are designed for healing Primary Intention wounds.

The now-healing chronic wound bed becomes analogous to a wound healing by approximation because there is complete epithelial coverage of the wound. Continued use of topical skin protectants of the present disclosure is ethical and approved by regulators as the covered wound is now a “minor” wound. The epithelial tissue matures into the well-known skin layers separated by a basement membrane from the dermis.

With acute incision wounds and the like, the adjacent sides of the wound are approximated by sutures, staples, Steri-Strips and the like. In the first 3-7 days, the site goes through well-known steps of hemostasis, inflammation and propagation and the site is both scarred and “healed” (closed). When the mechanical closure devices are removed, oozing and “holes” are left in the skin. The collagen formulations of the present disclosure are used to absorb the exudate while adsorbed omega3 oils penetrate the skin to increase angiogenesis and minimize scarring.

Scars themselves are avascular accumulations of collagen. The body remodels scars over time to make the emergency collagen scaffold (weak) into a strong permanent collagen structure by first digesting the emergency scaffold and simultaneously replacing the scaffold with a higher density collagen structure. This requires blood flow (increased angiogenesis) and clean-up of remodeling debris (preferably anti-inflammatory M2 macrophages). Without wishing to be bound, it is believed that the oils and gels of the present disclosure provide omega3 fatty acids to increase angiogenesis. Omega3 fatty acids in oils, gels and collagen-containing compounds all contribute to the increase in angiogenesis (leads to less scarring). The actual dose of omega3 fatty acid is important. For example, 1.6 g of triglycerides and waxes (one vial) is almost all oil; 1.6 g (one vial) of Collagen Matrix's oil=1.6*(1−0.41.4)=0.93, a ˜42% reduction in delivered oil/vial. [This is just a hypothetical illustration as the actual amount of oil is formulation dependent, but illustrates the point]. When additional composition comes in from the skin protectant around the periwound, the angiogenesis-benefit of omega3 fatty acid is increased. The wound does not “care” if the omega3 is from a lidocaine numbing oil, a skin protectant periwound gel or from a collagen matrix. All contribute to angiogenesis and scar reduction. Excess MCT is consumed by cells and produce anti-inflammatory M2 macrophages when there is sufficient blood oxygen (from increased angiogenesis). Thus, there is the simultaneous need for more blood flow and debris scavenging M2 macrophages. High permeate factor (slows intradermal absorption rate), oils (MCT>40 wt %) and gels of the present disclosure are combined with sufficient omega3 (ALA+SDA+EPA+DHA)>5%) to promote simultaneous angiogenesis stimulation and M2 macrophage production. To reduce scarring, there must be high omega3 fatty acid to promote angiogenesis (to reduced inflammation) and high MCT—for example, MCT concentration greater than 40 wt % of the composition—to digest emergency collagen during remodeling.

Scarring in wounds is incompletely understood. Collagen is needed to heal a wound, but excess collagen can cause scars. Crossed linked, prior art collagen ECM (extracellular matrix) resists MMP (Matrix Metallo Proteins) remodeling and can exacerbate scarring. Likewise, extended inflammation increases scarring. More recently, research shows that hair follicle stimulation, a source of stem cells, can reduce scarring. Hair follicles were seen as a source of stem cell progeny supporting new skin.

Without wishing to be bound, it is believed that the collagen-free compositions described herein having high retentate stimulate hair follicle transition from telogen (resting) to anagen (growth). Most of omega3 fatty acid compositions transit the epidermis through the intercellular pathway, i.e. through the epidermal corneocytes to the basal cells sitting on the basement membrane. Only a small amount of the total omega3 fatty acid passes through the follicular pathway. However, the collagen-free compositions of the present disclosure are manipulated to increase interfollicular entry through the skin. The partition between follicle and intracellular can be controlled by the time it takes for the transit to occur. Gels are slower to transit the epidermis than oils, so gels select for the follicular pathway. In fact, the presence of omega3 compounds (as sum(ALA+SDA+EPA+DHA)≥5%) of collagen-free compositions affects the route that the compositions are absorbed into the skin of the mammal. Thus, higher sum omega3 fatty acid selects for the follicular pathway. The permeate factor is also important (>3.0) for non-collagen compositions because more MCT is needed to drive the omega3 compositions into the follicle.

Collagen wound-healing compounds are applied to open wounds; open wounds have no hair follicles as the epidermis is missing. Collagen-free compositions formulated as oils and gels are used to stimulate hair follicle activity just outside of the exposed wound surface. Without wishing to be bound, there are two possible pathways to follicle activation:

1. Gels (high retentate factor) slow the omega3 pathway through the interfollicular route; some passes into the top of the hair follicle. The follicle becomes activated.

2. Oils (low retentate factor) pass quickly through the interfollicular route (with the help of MCT). Oil is partially absorbed by cutaneous adipocytes (fat cells above the subcutaneous tissue). These cells migrate to the base of the hair follicle and surround the follicle bulb (well known). The hair follicle transitions from telogen (resting to anagen (growing); stem cells are activated by mechanisms currently poorly understood.

The mechanism of action is unknown, but it is hypothesized, without wishing to be bound, that high retentate collagen-free products leave a slightly grease-rich epidermis that aids stem cell progeny's physical migration from the hair follicle, across the basement membrane separating the epidermis from the dermis, over the immediate wound's periwound “ridge” and onto the exposed wound bed surface. The wound heals rapidly. The stem cell progeny migration to the exposed wound (after wound signaling from follicle across the basement membrane) is well known. The signaling-mechanism is poorly understood.

Without wishing to be bound, it is believed that an increase of the amount of omega3 fatty acids in collagen-free compositions to values greater than 5 wt % (e.g., greater than 10 wt %) will cause omega 3-fatty acids to enter into the follicles. These compositions have a permeate factor >3.0, as the retentate factor is low. Omega3-fatty acids will flow through to the dermis, but there will be excess omega3 and so some will get into the follicle. Collagen-free compositions having >1.0 permeate factor are used as skin protectant oils and gels where there are intact hair follicles.

An alternate is to use a collagen-free composition formulated as a gel having high retentate and a lower sum omega3 fatty acid. Gel will flow slowly through epidermis, so there is more time for sufficient omega3 fatty acid to partition into the follicle. High permeate/high retentate and low permeate/high retentate are typically used for gels.

Hair follicle activation requires only a very small amount of omega3 fatty acid in the hair follicle. The minimum mass has not been quantified, but if hair growth begins, then enough was transferred into the follicle.

Hair follicle activation releases stem cell progeny from the follicle bulb. Progeny travel up the infundibulum to the periwound and then into the wound bed itself. Wound healing begins. Simultaneously, omega3 triglycerides are anti-inflammatory and deposit a layer of fat on the wound surface. Hydrolyzed collagen peptides (i.e. not cross-linked collagen) are placed in the wound and used for wound repair. C8 FFA helps adjust the pH so that MMP (Matrix Metallo Proteins) are active, but not rampant.

Matrix metalloproteinases (MMPs), also known as matrix metallopeptidases or matrixins, are metalloproteinases that are calcium-dependent zinc-containing endopeptidases; other family members are adamalysins, serralysins, and astacins. The MMPs belong to a larger family of proteases known as the metzincin superfamily.

Collectively, these enzymes are capable of degrading all kinds of extracellular matrix proteins, but also can process a number of bioactive molecules. They are known to be involved in the cleavage of cell surface receptors, the release of apoptotic ligands (such as the FAS ligand), and chemokine/cytokine inactivation. MMPs are also thought to play a major role in cell behaviors such as cell proliferation, migration (adhesion/dispersion), differentiation, angiogenesis, apoptosis, and host defense.

MMPs are important for collagen remodeling, but excess MMPs inhibit wound healing. MMP activity is closely regulated by pH and other factors. Balance is required for rapid healing.

Clinically, it is observed that furry mammal wounds heal more rapidly than human wounds. It appears that hair follicle stimulation is the root cause. Furry animal follicle density/cm² of skin is much higher in animals than in humans (legs and body (not head)). Prior art oil sprayed on animal wounds heals without added collagen but does not heal human chronic wounds. When applied to animals, the anhydrous compositions described herein are absorbed rapidly through the skin and through the hair follicles, since animals have many more hair follicles as compared to humans. The macro mass of compositions passing through treated hair follicles (e.g. macro count of follicles/cm²*grams of composition/follicle is a hypothetical measure describing how little all-chain-length oil is needed to activate an individual hair follicle. The decreased “per follicle” efficiency is overcome by a massive increase in treated hair follicles, This concept can be visualized by envisioning spraying a cat's leg and a human leg. The cat has many more follicles than the human. Since only a small amount of oil is needed to activate a single follicle, the macro activation of the cat is larger than the human because the number of cat follicles per unit of skin area is very much greater than that of a human.)

The problem then is how to increase the amount of omega3 fatty acid compositions into the hair follicle (regardless of source). Gels of the present disclosure melt at body temperature (˜37° C.), so a gel will heat up and become a viscous liquid after application to human skin. Free triglycerides or triglycerides sequestered in gels can both be a source of omega3 fats once they are heated to body temperature. The specific chain length of each triglyceride molecule is not changed in oil or gel form. The physical form difference is overcome by heating to body temperature (both oils and gels are liquid at body temperature). Gels and oils are distinguished by the relative rate at which they partition through the epidermis. Once through the epidermis, free triglycerides are indistinguishable from the now-melted triglycerides in formerly triglycerides sequestered in gels. Triglycerides can come from homogeneous triglycerides applied at room temperature or it can come from a gel that was liquefied by body heat after application. This is done by converting the collagen-free compositions of the present disclosure formulated as oils into a gel. Gels are absorbed by the epidermis more slowly than oil formulations. There is first the heating up, then the melting and finally the higher melted-gel viscosity, all slowing the rate of transfer across the epidermis. An artifact of slower absorption is that unmelted gel stays on the follicle surface as part of the retentate longer. Eventually, gel retentate melts and is absorbed into the skin. Unexpectedly, the slower rate means that intradermal cells are exposed to gelled oil melt over a longer period of time. Each intradermal cell has a finite consumption rate for absorption. Any composition not consumed passes by the intradermal cell and moves towards downstream dermal cells. Thus, the intradermal cell consumption of MCT and omega3 fatty acid mass is the cross product of consumption rate multiplied by exposure time. Gels increase the exposure time and do not affect the rate-limited cell consumption rate. With more exposure time, there is more macro anti-inflammatory omega3 fatty acid incorporated into the cell lipid bilayer. With more exposure time there is less lactic acid production (from glycolysis) and more anti-inflammatory macrophage M2 production (from MCT OXPHOS fatty acid consumption).

It is important to understand that there is not exclusively one route through the epidermis. Without wishing to be bound, it is believed that Retentate Factor affects the partitioning coefficient between the intrafollicular route and the follicular route. Slowing the transfer rate through the intrafollicular route allows more time for the slower transfer into the top of the hair follicle sheath (follicular route). Thus, gels select for more follicular transfer; oils select for more intrafollicular transfer.

Analogously, hair follicles are even more sensitive to the rate of omega3 fatty acid migration. Using gels of the present disclosure to slow the rate of omega3 fatty acid migration, retentate above the hair follicle basement membrane is slowly released into the follicle bulb aperture at a rate the follicle cells can absorb. The net effect is a higher absolute mass of omega3 fatty acid into each follicle over the course of a 24 hour day. More stem cell progeny become available for scarless wound healing as progeny consume follicular oils.

It is unobvious to reduce the rate of omega3 fatty acid migration (via gelation) to increase the daily mass of omega3 fatty acid resident in the intradermal zone. It is also unobvious to use physical gels (versus liquid oils) to increase the number of stem cell progeny presented to follicle-free wound beds.

There is a partitioning between skin and follicle. In humans, the skin route is preferred because the surface area of skin is very much greater than the surface area of hair follicles. Skin absorbs oils in seconds; skin absorbs gel in minutes. The hair follicle is different. The hair follicle has basement membrane surface area above the bulb and surface area of the hair shaft itself. What is observed is that some of the gel attaches to the hair shaft as rubbed in, but then gel-on-shaft flows to the bulb opening and is absorbed by the follicle in about −30 minutes. The net effect is to meter gel into the hair follicle as body heat softens and melts the gel to make it flow. The absolute amount of gel into the hair follicle is directly proportional to the grams of gel applied to the site. To promote absorption through the hair follicular route, the gels of the present disclosure have a melting (softening) point at or below body temperature.

Thus, for example, the anhydrous topical pharmaceutical compositions of the present disclosure minimize scarring when applied to the wound, burn or skin condition when the composition is comprised of marine oil, vegetable oil having an omega3 fatty acid content greater than 9 wt %; monolaurin, cetyl esters or wax; and medium chain triglycerides (“MCT”), wherein the weight ratio of the sum of the weights of (ALA+SDA)/sum of the weights of (EPA and DHA) present in the composition ranging from about 0.5 to about 1.5, and in addition, either in collagen-free compositions which are oils, the sum of the wt % of MCT, monolaurin and cetyl ester in said pharmaceutical composition is greater than or equal to 42 wt % or in compositions where collagen is present, the sum of the wt % of MCT, monolaurin and cetyl ester in said pharmaceutical composition is greater than or equal to 20 wt %. Said another way, scarring is an artifact of healing via inflammation and over-production of collagen. The effective countermeasure is to heal by introduction of stem cell progeny simultaneously with anti-inflammatory compositions to create non-scarring epithelial tissue. Both strategies can seal the wound; one is inflexible and unsightly (scar), the other is flexible, grows appropriate hair follicles and resembles undamaged skin (no-scar).

Scarring is complex. All wounds will scar. The technical objective is to minimize visible scarring. With large open wounds that eventually heal, there are always visible scars. Since these are healed by collagen-containing compounds, collagen compounds correlate with scarring. But when the wounds are approximated (e.g. surgical wounds), collagen compounds minimize healing by reducing healing time (less chance for excess scar tissue to accumulate). When oils and gels are used, angiogenesis increases, and wounds heal faster (i.e. less time for excess scar tissue to accumulate). So periwound oil and gels correlate with less scarring.

The wound autolytically debrides (e.g. MCT encourages M2 macrophage production where increased angiogenesis brings needed oxygen to the treated site). The M2 macrophages consume necrotic tissue allowing for uniform healing (less scarring). There is no “silver bullet” to eliminate scarring, but rather a “buffet table” of compounds that contribute at various times during the healing process:

1. Increased angiogenesis brings more oxygen to the table

2. ROS kills pathogens

3. PUFA helps control ROS

4. M2 Macrophages clean-up necrotic tissue

5. Hydrolyzed collagen is available for new tissue synthesis

6. PUFA oils (from oil and/or gels) are converted into multiple downstream bioactive anti-inflammatory healing compounds.

7. Stem cell progeny migrate to the wound; their “stemness” causes rapid healing.

8. Unmacerated periwound is electrically pulled over the wound bed, closing the wound.

9. The closed wound remodels temporary collagen structures, reducing scar tissue.

Without wishing to be bound, it is believed that there are three ways to control the amount of omega3 fatty acid that enters the hair follicle:

1. Increase the total amount of product applied per cm².

a. This strategy increases the omega3 fatty acid available above what the skin can absorb. Excess omega3 fatty acid sitting on the surface, is cosmetically inelegant and is subject to oxidative malodor development.
b. Nevertheless, on animal applications where skin feel and odor are irrelevant and application can be problematic, this is a preferred route for applying compositions of the present disclosure to animals.
c. With humans this issue can be attenuated by applying the gross excess oil, gel or paste, waiting 15 minutes for absorption and then showering to remove excess oil, gel or paste.

2. Use gels to slow the rate of absorption

a. With more gel staged above each treated follicle, there is added time for gel to melt and enter the follicle

3. Apply oils with sum omega3>9 wt % and MCT≤30 wt %

a. There is an abundance of omega3 and a shortage of MCT. The net effect is to slow oil absorption, leaving oil available to transfer into each follicle bulb.

In practice, the gel approach is more consistent and more reliable. Gel is easier to apply. Oil tends to run off a curved lower leg and cause a slip/slide hazard at the application site. Gel does not run off. Gel also spreads the retentate waxes evenly over intact skin to form a semi-occlusive layer with lower TEWL (Trans Epidermal Water Loss). Thus, the gel activates hair growth, is less messy and improves skin hydration and helps encourage stem cell progeny to migrate to the wound site.

Scarring has an evolutionary bias in that it is in the best interest of the corpus to staunch bleeding and create a bacterial barrier immediately. The loss is skin flexibility. By encouraging stem cell progeny migration to the wound, the same rapid closure is achieved without unsightly inflexibility.

In conclusion, without wishing to be bound, it is believed that novel collagen-free compositions of the present disclosure having a low permeate factor and high concentration of MCT, for example, greater than 20 wt % of the composition, and having a sum of wt % of MCT plus monoglycerides, and linear fatty acid esters (e.g., cetyl esters and waxes) greater than 42 wt % will minimize scarring when used to treat wounds, burns or skin conditions. Moreover, it is believed that novel collagen free compositions of the present disclosure having high permeate factor and the ratio of (ALA+SDA)/(EPA+DHA) between 0.5 and 1.5 in non-collagen compositions will minimize scarring when used to treat wounds, burns or skin conditions. Moreover, that novel collagen-free compositions of the present disclosure with high retentate factor will minimize scarring when used to treat wounds, burns or skin conditions. Further, novel collagen-free compositions of the present disclosure comprising low permeate factor and high retentate factor and novel collagen free-compositions having high permeate factor and high retentate factor will also minimize scarring when used to treat wounds, burns or skin conditions. Finally, novel collagen compositions disclosed herein with high permeate factor will also minimize scarring when used to treat wounds, burns or skin conditions.

The formulations described herein are prepared by techniques known in the art, which techniques are described in U.S. Pat. No. 10,463,699 and WO 2019/200364, the contents of which are incorporated by reference. The non-collagen containing formulations are prepared by mixing the various components at slightly elevated temperatures, such as about 30° C. to about 60° C., and in another embodiment, at about 35° C. to about 50° C. and in another embodiment, at about 38° C. to about 43° C., until the resulting mixture is homogenous, and then allowing the mixture to cool to room temperature. For compositions comprised of collagen, all of the components except the collagen and sea salt and any fragrances are mixed together with stirring at a temperature sufficient to homogenously melt the components and form a homogenous paste. Typically, the composition is heated at temperatures ranging from about 130° F. to about 200° F., in an embodiment, and in another embodiment from about 150° F. to about 170° F. and in another embodiment at about 140° F. When the resulting mixture is homogenous, collagen is added at temperatures greater than 130° F., for, example at temperatures at about 150° F. to about 170° F. and the collagen is mixed in with the other components. When substantially homogenous, when the pores of the collagen are filled with oils, which takes about ten to about twenty minutes, the composition is cooled to room temperature. The ground sea salt, anti-microbial agents such as benzethonium chloride and any fragrances are then added at ambient temperatures. In an embodiment, they are added in an inert atmosphere, such as in the presence of an inert gas, such as nitrogen. The composition is again mixed until the composition is substantially homogenous, including incorporation of inert gas to form a collagen matrix with specific gravity <0.8.

The collagen-free gel compositions comprised of monoglyceride, such as monolaurin, in an amount of 6 wt % or greater are prepared by art recognized techniques. For example, they are prepared by thoroughly mixing in, for example, a mixing vessel, such as a mixing bowl, the components described hereinabove, with or without perfumes and fragrances with heating at temperatures and conditions sufficient to melt the oils in the composition, for example, at 145° F. The order of addition is not critical because the mixture is a single-phase fluid once the room temperature solids melt. Optionally inert gases may be present in the mixing vessel until all of the oils have melted. More specifically, the headspace in the mixing vessel, in an embodiment, may be an inert gas, such as argon, helium, and nitrogen and the like. In an embodiment, argon is used. Argon has the advantage of being heavier than air and helps prevent oxygen ingress into the mixing vessel headspace. In an embodiment, the mixture may be clear. The oil is cooled to 105° F. to 110° F. with gentle mixing. Perfumes and fragrances may be added after cooling or they may be added with the other components. If perfumes and fragrances are added at a later time, the additional gentle mixing may be, in an embodiment, for about an additional 2 minutes after addition of perfumes and/or fragrances. Regardless, the components are mixed with gentle stirring until thoroughly homogenous and/or until the composition becomes opaque as gelation begins. If in a mixing vessel, such as a mixing bowl, the contents of the mixing vessel are stirred gently, for example, using a dough hook stirrer at speeds of 1-5 rpm until the contents become opaque. In an embodiment, the temperature of the mixture when the mixture turns opaque is about 102.5° F.

The mixture is allowed to stand without any additional heating or stirring until the components of the mixture finish congealing and form a stable gel. A homogenous anhydrous, oil gel is formed at temperatures of less than or equal to about 102.5° F. This product that is formed is the high viscosity gel.

To form the gelled composition at lower viscosity, the viscosity of the thick gel can be manipulated by a very short (15 seconds) ambient temperature mix with a mixing tool (well known in the art), for example a “dough-hook” from a Kitchen Aid mixer. The thick gel is slightly agitated some more at, e.g., about 1 to about 5 rpm, such at 1.5 rpm, until the gel has the viscosity of the low viscosity gels described hereinabove. At this point, the gel should take on the consistency of applesauce. This product formed is the lower viscosity gel. Without wishing to be bound, it is believed that the gel is partially broken. 60 second mixing creates oil leakage; no mixing gives a thick gel that cannot be easily pumped. At 15 seconds, the gel will flow slowly; at 30 seconds, the gel will flow easily.

The two viscosity gels are packaged separately. The high viscosity gel is packaged when it is opaque as it is not as viscous when the temperature of the gel is above 102.5° F. The gel is held at that temperature above 102.5° F. during package filling. Preferably, the container is air tight, as the high viscosity gel of the present disclosure, in an embodiment, is sealed in an airless container for single use dispensing. The filled, sealed package cools to <97° F. and instantly gels. This phenomenon is not a slow gelation; it occurs virtually instantaneously. The gel is more robust if formed in the absence of shear (e.g. in the undisturbed, filled, seal package). If the gel is sheared, even once, it becomes a pumpable gel of low viscosity. If it is reheated to clarity (e.g. 145° F.), it reforms as a thick gel.

The low viscosity gel can be pumped and filled into packages.

The compositions of the present invention are useful for treating wounds, burns and skin conditions, particularly the periwound around the open wound. If the wound, burn or skin condition is approximated, then the gel can be used to treat the approximated wound directly. If the open wound or burn has closed by secondary intention, then the gel can be used directly on the previously open wound (or burn) during collagen remodeling. As used herein, a wound is defined as an injury to living tissue of the mammal in which the skin is cut or broken. It includes incisions, cuts, including paper cuts and shaving cuts and burns. However, as defined herein, the wound may or may not be accompanied by bleeding. Examples of wounds treatable by the composition described herein include, but are not limited to, incisions (including surgical incisions), lacerations, abrasions (such as in dermabrasion and microdermabrasion), ulcers, and the like. In some embodiments, the wound is a diabetic wound ulcer.

In an embodiment, the composition with fish collagen is used as a hemostat for minor wounds. For example, after a femoral artery access procedure, as described in U.S. Pat. No. 8,353,929, the contents of which are incorporated by reference, the apparatus therein described in FIG. 22 can be used for closure of the wound, and the fish collagen paste described herein replaces the powdered hemostat. In this embodiment, the paste configures to the irregularities caused by the mechanical skin compression by the closure device. The fish collagen absorbs blood and exudate and causes immediate hemostasis of the wounds described therein.

The wound may be the result of an accidental injury or be the consequence of a medical procedure. The wound may be a surgical incision. The wound may be an ischemic tissue flap, such as in the course of cosmetic surgery. The wound may be one caused in the course of other cosmetic surgery, such as dermabrasion, microdermabrasion, chemical peel, laser resurfacing, etc. The wound may be a chronic injury. The wound may be a skin tear.

As used herein the term chronic injury is a wound, burn or skin condition that does not heal in an expected way and in a predictable amount of time, unlike acute injuries. A chronic injury can be recognized by a number of symptoms including the loss of skin and/or tissue surrounding the wound, burn or skin condition or by the amount of time it takes to heal. Once a wound, burn or skin condition has become chronic, intensive medical intervention and treatments are normally required for healing. There are many types of chronic injuries, as well as many causes of chronic injuries, such as Diabetes, Vascular disease, Infection, Immobility, Trauma, Surgery, Burns, Radiation injury, electrical burns, chemical burns and the like. A chronic wound may never heal or may take years to do so. These wounds can cause severe emotional and physical stress; they can also create a significant financial burden on the patient and on the entire healthcare system.

Compositions described in the present disclosure are useful in treating chronic wounds. For instance, collagen-free compositions of high permeate factor and either low or high retentate factor that are described in the present disclosure are designed for healing chronic wounds. Chronic wounds are typically treated once per week. For example, oil compositions are designed to “trickle” into the wounds over 7 days (low permeate) and “hide” in the wax layer (high retentate) for up to one week. An open chronic wound has no epidermis, so there is no need for rapid permeation.

An acute wound is a wound that progresses through the phases of normal healing, resulting in the closure of the wound within a reasonable amount of time, for example, up to four weeks.

The compositions of the present invention are useful for treating chapped skin and other skin disorders. Examples of skin disorders include acne, psoriasis, eczema, dermatitis, alopecia, rosacea, burns, chapped skin, poison ivy, shingles and the like.

The term “acne” is meant to include any skin condition where a skin pore becomes blocked and/or thereby becomes inflamed. The term acne includes without limitation superficial acne, including comedones, inflamed papules, superficial cysts, and pustules; and deep acne, including deep inflamed modules and pus-filled cysts. Specific acne conditions can include, but are not limited to, acne vulgaris, acne comedo, papular acne, premenstrual acne, preadolescent acne, acne venenata, acne cosmetica, pomade acne, acne detergicans, acne excoriee, gram negative acne, acne rosacea, pseudofolliculitis barbae, folliculitis, perioral dermatitis, and hiddradenitis suppurativa. Acne is a common inflammatory pilosebaceous disease characterized by comedones, papules, pustules, inflamed nodules, superficial pus-filled cysts, and (in extreme cases) canalizing and deep, inflamed, sometimes purulent sacs. Acne involves an interaction between hormones, keratinization, sebum, and bacteria that somehow determines the course and severity of acne. It often begins at puberty, when the increase in androgens causes an increase in the size and activity of the pilosebaceous glands. The earliest microscopic change is thought to be intrafollicular hyperkeratosis, which leads to blockage of the pilosebaceous follicle with consequent formation of the comedo, composed of sebum, keratin, and microorganisms, particularly Propionibacterium acnes. Lipases from P. acnes break down triglycerides in the sebum to form free fatty acids (FFA), which irritate the follicular wall. Retention of sebaceous secretions and dilation of the follicle may lead to cyst formation.

Skin conditions also include, but are not limited to, dermatological conditions linked to disorders of keratinization involving differentiation and proliferation, in particular, acne vulgaris, comedonic or polymorphic acne, nodulocystic acne, acne conglobata, senile acne and secondary acnes such as solar, drug or occupational acne; for other types of keratinization disorders especially ichthyoses, ichthyosiform conditions, Darier's disease, palmoplantar keratoderma, leukoplakia and luecoplakiform conditions or lichen and lichen planus; dermatological disorders having an inflammatory or immunoallergic component, in particular, all forms of psoriases, either cutaneous, mucosal or ungual, and psoriatic rheumatism, and cutaneous atopy such as eczema or respiratory atopy, dry skin, inflammation of the skin, solar erythema, skin allergies or other skin disorders of the epidermis and dermis.

Psoriasis is a skin condition characterized by hyperplasia of keratinocytes resulting in thickening of the epidermis and the presence of red scaly plaques. The lesions in this chronic disease typically are subject to remissions and exacerbations. There are several patterns, of which plaque psoriasis is the most common. Guttate psoriasis, with raindrop shaped lesions scattered on the trunk and limbs, is the most frequent form in children, while pustular psoriasis is usually localized to the palms and soles. The classical inflammatory lesions vary from discrete erythematous papules and plaques covered with silvery scales, to scaly itching patches that bleed when the scales are removed. Psoriasis is a condition in which cell proliferation is increased up to 10 times the normal rate for an individual. The skin is the largest portion of the human body which is comprised of cells within three skin layers. Each of the skin layers is in a constant state of growth with the outer layer being formed of predominantly dead tissue which is naturally being discarded at a normal rate. Replacement of cells from underlying layers is accomplished by cell division and maturation where cells move upwardly and outwardly at a rate which varies dependent upon the age, sex, and/or health of an individual. Psoriasis causes an increased turnover of cells, which in turn increases the rate of cell growth and cell death. This increased rate of cell growth and cell death may result in injuries and/or disorders which accompany the increased synthesis of all tissue components and further elevate the strain placed upon skin or other tissue and the bio-synthetic capabilities of the cells within the affected area.

The terms eczema and dermatitis are generally used names for severe inflammation of the skin, usually with redness, swelling, oozing, rusting or scaling of lesions which are usually itchy. Eczema may take the form of contact dermatitis (due to skin contact with the cause) or atopic dermatitis in individuals who are “atopic” or allergic by nature. If the scalp is involved the disorder is known as seborrheic dermatitis. Dermatitis can be caused by chemicals, plants, shoes, clothing, metal compounds and even medicines used to treat dermatitis. In atopic dermatitis environmental temperature, humidity changes, bacterial skin infections, airborne allergens and garments, e.g., wool, may all bring about dermatitis.

Alopecia is a skin condition that results in the loss of hair on the scalp and elsewhere. It usually starts with one or more small, round, smooth patches and occurs in males and females of all ages. Loss of hair in one or several small spots is common, but it is possible to lose all scalp hair (alopecia totalis), or every hair on the body (alopecia universalis), which is rare.

The skin condition, rosacea is of an unknown origin. It usually affects the middle third of the face causing skin redness, prominent vascularization, papules, pustules and swelling, as well as a predisposition to flushing and blushing. However, rosacea can also occur on other parts of the body including the chest, neck, back, or scalp. The blood vessels near the skin dilate and become more visible there through, resulting in telangiectasia. The resulting papules and pustules resemble teenage acne, and are frequently mistaken for the same. Unlike acne, rosacea does not have blackheads or whiteheads. Rosacea, however, can occur in all age groups and in both sexes, where it tends to be more frequent in women but more severe in men. The flushing and blushing regions of the face are affected by rosacea. Emotional factors such as anxiety, embarrassment, or stress may evoke or aggravate rosacea. In addition, a flare-up may be caused by environmental or climate variances, and UV exposure is known to aggravate rosacea. Furthermore, diet is also known to aggravate rosacea. Spicy foods, alcoholic beverages, hot beverages, and smoking are known to cause flare-ups. Rosacea is not only an aesthetic complication. Rosacea is a chronic disease that has rarely been documented to reverse its progression. If untreated, the condition worsens and spreads. Untreated rosacea may cause a disfiguring nose condition called rhinophyma, which is characterized by a bulbous, red nose and inflamed cheeks. Severe rhinophyma may require surgery, an invasive procedure that may be avoided by timely treatment. Another problem of advanced rosacea is ocular. Persons afflicted with rosacea may experience conjunctivitis, a burning and grittiness of the eyes. If untreated, it may lead to serious complications such as rosacea keratitis, which damages the cornea and may impair vision.

Other skin conditions can include dry/chapped skin. In addition, vaginal dryness and erectile dysfunction are also treatable by the present compositions. Without wishing to be bound, these are partially a function of circulation impairment. When the composition is absorbed in these site-sensitive areas, circulation increases by reducing inflammation in the lymph and venous systems. Blood flow increases.

In another embodiment, the compositions are directed to treating Peripheral artery disease (PAD), which is a narrowing of the peripheral arteries to the legs, stomach, arms, and head. After vascular surgery of a subject having peripheral arterial disease (PAD), a topical composition of any of the compositions described herein, can be applied to the area where the surgery took place. For example, if PAD is in the legs, after vascular surgery in the legs, the composition described herein are topically applied to the legs' lower leg skin to increase blood circulation at the skin surface. For example, the composition may be applied as a spray

The compositions described herein are useful for treating skin conditions.

Burns involve a type of skin integrity rupture. Burns represent one of the most painful processes that can be established in this tissue, needing the establishment of a coordinated therapy to help its recovery and pain treatment. Burns can be caused by several factors, among which, exposure to high or low temperatures, exposure to chemical compounds, by electricity, by exposure to radiation and mechanical friction. Burn severity and its risk are evaluated according to the amount of affected tissue and depth reached. The amount of affected tissue is represented by the percentage of burned corporeal surface (BCS). In this type of evaluation, burns can be divided into small, moderate, large or massive burns, where regions inferior to 15% of BCS, from 15% up to 49% of BCS, from 50% up to 69% of BCS and over 70% of BCS, respectively. The extension of the affected area is determined through Lund-Browder scheme, which takes into consideration the burn proportion, in accordance with the age of the burned patient. Another rule that is most used for determining the extension of the affected area is that known as Wallace Rule or Rule of Nines, a technique less efficient than the foregoing, however, easy to memorize, being very much employed in emergency cases. This rule applies a value equaling nine or nine multiple to the affected parts, being 9% for each superior member, 9% for the head, 18% for each inferior member, 18% for each torso face and 1% for the genitalia.

The classification as first, second and third degree corresponds to burn depth. The first-degree injury corresponds to the burn that affects the skin most external layer (epidermis), not producing hemodynamic alterations, however the affected region is found hyperemic in absence of blisters or phlyctenae. This type of injury can be observed in erythemae resulting from sunrays or heated water. The second-degree injury affects either the epidermis as part of the dermis and is mainly characterized by the formation of blisters or phlyctenae, as those resulting from scalding or thermal injury resulting from overheated liquid. The third-degree injury endangers the totality of skin layers (epidermis and dermis) and, in many cases, can affect other tissues, as the subcutaneous cellular tissue, muscular tissue and bone tissue. Third-degree burns are considered as the most severe of all thermal injuries, producing deforming injuries. For being deeper, it eliminates the nerve endings responsible for shooting the painful message. These types of burns need transplanting for recomposing destroyed tissues, since the structures and organelles necessary for the natural recovery process, were eliminated. Since burns are wounds that involve the skin, they develop aforementioned complex process of regeneration and recomposition of injured tissue. The greater the area involved, the slower the speed or grade of re-epithelization of the affected region, considerably increasing the recovery time, when the injuries start to cover a body surface over 10% or 15%.

Immediately after the burn trauma, an inflammatory process develops wherein various agents are delivered, occurring deposition of fibrins and platelets activated on the wound surface. A matrix rich in organic material is yielded, able to enclosure bacteria and other strange substances, which frequently aggravates the case, due to sepsis that can follow trauma. During this inflammatory process a great quantity of exudates crop out of the burned region, leading the patient to an intense loss of liquids, which, depending on the burn extension and depth, can cause a severe dehydration case. The inflammatory process extends to adjacent tissues, factor that endangers the functions of these tissues initially intact.

Extensive and deep burns cause alterations that are extended far beyond the affected local, such as anatomic, metabolic, physiological, endocrinology and immune alterations, requiring special care. Significant fluid losses, delivery of inflammatory multi-mediators and contamination by bacteria, occur. When disseminated in central organs through circulation, bacteria and inflammatory mediators can cause cardiac endangerment, failure of gastrointestinal mucous integrity and in extreme cases, multi-organic failure.

Hemodynamic alterations that occur after severe thermal injuries include decrease of cardiac output and reduced volume of circulating plasma, contributing all to a hypovolemic shock. Inflammatory mediators (including cytokines, prostaglandin, nitric oxide and superoxide ions) have been implicated in causing further damage to tissues. It is believed that despite local benefit, such mediators induce undesirable effects when reaching significantly high levels. As an example, a greater damage to tissues can be caused by delivery of proteolitic enzymes and superoxide ions of macrophages and activated leucocytes.

Thus, burns are skin conditions that develop unbalance in a series of natural organic mechanisms, not limited to endangered tissues only, but involve numerous organs that can be affected. Additionally, large thermal injuries induce a sharp increase in basal metabolic rate. Large nitrogen corporeal losses, observed in burned patients, mainly occur due to protein exudation through burned skin and also by the fact that, under such catabolic stress situation, corporeal proteins can become the metabolic substrate used for production of 15 to 20% of total energy required by the organism. Further to these abnormalities, hormonal levels change with an increase in cathecolamines, cortisol and glucagons, in the presence of normal or slightly increased levels of insulin. These hormonal alterations promote increase of proteolysis and lipolysis. Thus, the entire complex process is characterized by imbalance. The quick recovery of the skin of a burned mammal is of utmost importance for recovery of his normal organic functions.

The novel formulations of the present disclosure are useful for treating burns.

Thus, the methods disclosed herein are useful for treating or ameliorating the skin against the effects of environmental conditions. According to an aspect of the present invention, either one or combination of formulations described herein, including compositions with or without collagen or is (are) applied topically to at least the part of the body of the patient containing the chapped skin or other skin condition, burn or wound.

For the treatment of wounds, burns and skin conditions, the composition of the present disclosure can be applied directly to the skin or wound as a gel, ointment, liquid, cream, or the like as described above. Alternatively, the stabilized formulation is administered in the form of a wound dressing, wherein the composition of the present disclosure is applied onto a wound dressing and the wound dressing with the composition of the present disclosure is then applied to cover the area of the skin at the location of the skin condition or wound. As used herein, the terms “wound dressing” and “dressing” refer broadly to any substrate when prepared for, and applied to, a wound for protection, absorbance, drainage, improvement of cell environment, etc., and may include any one of the numerous types of substrates and/or backings that are commercially available, including films (e.g., polyurethane films), hydrocolloids (e.g., hydrophilic colloidal particles bound to polyurethane foam), hydrogels (e.g., cross-linked polymers containing about at least 60% water), foams (hydrophilic or hydrophobic), calcium alginates (e.g. non-woven composites of fibers from calcium alginate), silicone, collagen, keratin, and cellophane (e.g. cellulose with a plasticizer). For example, the stabilized formulation can be applied to the surface of, or incorporated into, a solid contacting layer such as a dressing gauze or ECM matrix. Suitable gauze dressings may include, for example, dry woven or non-woven sponges, swabs, bandages and wraps with varying degrees of absorbency. Exemplary fabric composition may include, for example, cotton, polyester or rayon. In certain embodiments, gauzes and non-woven dressings may be available sterile or non-sterile in bulk and with or without an adhesive border. In certain embodiments the dressings also comprise one or more additional pharmaceutically active compound and/or carrier agent, including for example, saline, oil, zinc salts, cross-linked collagen, petrolatum, xeroform and scarlet red.

Various treatments may be employed. Skin surfaces of the most concern tend to be those not typically covered by clothing such as facial skin surfaces, hand and arm skin surfaces, foot and leg skin surfaces, and neck and chest skin surfaces. In particular, facial skin surfaces, including the forehead, perioral, chin, periorbital, nose, and/or cheek skin surfaces, may be treated with the compositions described herein.

The treatment method may include applying the composition(s) to a previously identified area of skin in need of treatment, or an area where one seeks to prevent, treat or reduce the appearance of chapped skin or other skin disorders. Many regimens exist for the application of the composition(s). The composition(s) may be applied at least once a day, twice a day, or on a more frequent daily basis, during a treatment period, as prescribed by the physician. When applied twice daily, the first and second applications are separated by at least 1 to 12 hours.

Typically, the composition(s) may be applied in the morning and/or in the evening before bed.

The treatment period is ideally of sufficient time to provide an improvement in the appearance of the skin. The treatment period may be at least 1 week, and in some embodiments the treatment period may last about 4 weeks, 8 weeks, or 12 weeks. In certain embodiments, the treatment period will extend over multiple months (i.e., 3-12 months) or multiple years. In one embodiment the composition is applied at least once a day during a treatment period of at least 4 weeks, 8 weeks, or 12 weeks. In one embodiment the composition is applied twice a day during a treatment period of at least 4 weeks, 8 weeks, or 12 weeks.

Effective amounts of each of the compositions are applied topically to the area to be treated. The dose varies with the individual and the skin condition. The ideal dose is that dose that provides as much of the compositions as the skin will absorb. An excess amount of the compositions on the surface of the skin can turn rancid. Patients who used the compositions described herein found that applying the compositions to the areas to be treated just prior to showering was the most effective way to administer these compositions. The skin absorbs all it can; the shower washes off the excess. A silky smooth, odorless finish remains on the skin for 24 hours.

The dosage regimen for treating skin conditions, burns and/or wounds is determined by a physician in the normal course of practice and is selected in accordance with a variety of factors, including the age, weight, sex, and medical condition of the patient, the severity of the condition, the route of administration, pharmacological considerations such as the activity, efficacy, pharmacokinetic and toxicology profiles of the particular composition used, whether a dressing or drug delivery system is used and whether the composition is administered as part of a drug combination.

The doses may be administered in single or divided applications. The doses may be administered once, or application may be repeated. Application may be repeated weekly until skin and/or wound healing is promoted, or a repeat application may be made in the event that healing slows or is stalled. Doses may be applied 1-7 days apart, or more. In the case of a chronic skin condition or wound, repeat applications may be made, for example, one or more times per day, weekly, or bi-weekly, or monthly or in any other frequency for example if and when healing slows or is stalled. For some indications more frequent dosing such as hourly application may be employed.

In an embodiment, depending upon the severity of the wound, burn or skin condition, a combination of one or more formulations described herein may be used to treat the wound, burn or skin condition. The physician will determine if this is necessary. However, if the wound, burn or skin condition is very painful to the patient, a first formulation comprising an analgesic, such as lidocaine, is applied to the locus of the wound, burn or skin condition in effective amounts to alleviate pain associated with the wound or skin condition. These compositions may or may not contain collagen. In an embodiment, when the composition contains an analgesic, the formulation does not contain collagen. In addition, such formulation helps dislodge necrotic tissue associated with the skin condition or wound. The solvent action of the MCT helps loosen necrotic tissue. The excess formulation and slough are wiped away. In an embodiment lidocaine is added to the oil mixture and heated during processing until there is a single-phase fluid. For example, a low concentration of lidocaine (for example, about 0.8 wt %) is added to the composition of the present disclosure containing collagen or not containing collagen to mitigate the pain and allow blood flow to resume to the damaged area. Burn victims have an immediate inflammatory response to stop further damage. When an anti-inflammatory oil is applied, blood flow increases and can cause sharp pain as vascular blood pressure expands damaged vesicles.

After the application of this composition, the doctor waits for sufficient time for the analgesic to numb the wound bed tissue, the burn tissue or the loci of the skin condition on the skin. Usually, this takes about 5 to about 30 minutes. The loose necrotic tissue and any unabsorbed composition is wiped out of the wound bed loci of the skin condition with a sterile gauze or wipe, Optionally, with a scalpel, additional necrotic tissue may be sharply debrided from the bed of the wound or skin condition or situs of the burn. As the wound begins to close and/or the skin condition begins to clear up and the situs of the burn begins to heal, the need for sharp debridement is reduced as viable new tissue replaces pre-existing necrotic tissue. The analgesic formulation of the present composition is then wiped off with sterile material, such as gauze. In an embodiment, especially if the wound or skin condition or burn is still painful, additional formulation of the present application containing the analgesic may be applied to the situs of the wound, burn or skin condition for another 5 to 60 minutes to further numb the pain, and then the analgesic formulation is wiped off.

In an embodiment, when using a combination of formulations of the present disclosure, the collagen containing formulation described herein is next applied to the loci of the wound, burn or skin condition in effective amounts to treat the wound, burn or skin condition and effect granulation. The collagen formulation encourages granulation. Since this composition is so viscous, the application thereof will be deposited as stripes, i.e., separate spots on the wound or skin condition. However, the body heat softens the collagen containing composition until it flows into the irregularities of the wound bed surface or burn surface or surface of the skin condition and conjoins with adjacent stripes to form a contiguous coating of the entire wound bed surface or surface of the loci where the skin condition or burn is located. The collagen formulation is then covered with a sterile cloth or gauze or band-aid. Periodically, for example, once a week or sooner, the situs of the wound, burn or skin condition is inspected to see if it is fully granulated. If the situs of the wound, burn or skin condition is not fully granulated, then the old collagen formulation is allowed to remain and new collagen with a fresh collagen formulation and covered again. At the beginning of the treatment, the wound, burn or skin condition is inspected more frequently; as the wound, burn or skin condition starts to heal and granulate, the inspection and replacement of the collagen composition with fresh collagen composition is less frequent. One of ordinary skill in the art, such as a physician can make that determination based upon various factors, such as the severity of the wound, burn or the skin condition, the age of the patient, the health of the patient and the size of the wound, burn or skin condition including the depth thereof and the like. The process of replacing the old collagen formulation with new collagen formulation of the present disclosure is repeated until the situs of wound or skin condition or burn is fully granulated. Once the wound or skin condition or burn is fully granulated and/or concurrent with the first collagen application, a skin protectant composition, which is a formulation described herein which does not contain collagen or analgesic is applied to the periwound surrounding the situs of the wound bed surface or surface of the skin condition or burn. The skin protectant composition is topically applied thereto. It covers completely the periwound around the situs of the wound, burn or skin condition and the intact skin adjacent to the area, such as the periwound, in an amount effective to protect the situs from infection and/or ROS species and/or to retard the growth of pathogens, such as bacteria. In an embodiment, the periwound is saturated with a skin protectant composition. Any excess of the compositions used herein is wiped away. This skin protectant is applied to the situs of the wound, particularly after the situs has closed, but not yet remodeled, the burn or skin condition, but also an area adjacent thereto, such as the periwound, for example, or in another embodiment, the area that borders or is adjacent to and in close proximity to the situs, for example, an area up to 6 inches in diameter around the situs. Periodically, the wound is inspected and, if the skilled medical practitioner so determines, the skin protectant composition is reapplied as before. Again, the frequency of removing and reapplying the skin protectant composition is determined by the physician, and is dependent upon many factors, including the health and age of the patient, the size of the wound or skin condition or burn and the drainage from the wound, skin condition or burn, the sex of the patient, and the like. The skin protectant can be used to stimulate hair growth. When this happens, stem cells are activated (if signaled for by the wound site). The stem cells do not migrate, but their progeny migrate across the basement layer separating the epidermis from the dermis and eventually onto the wound bed. The progeny form into a very thin film of epidermal material covering the granulating wound bed. Once formed, this monolayer of epithelial tissue reflects light (making it visible to the skilled practitioner) and separates the granulating bed from excess topical collagen. Topical collagen absorption is inhibited; the epithelial tissue thickens and forms an epidermis; scar formation is minimized, and the wound heals.

The process of replacing the absorbed skin protectant composition is repeated until the wound has healed and remodeling is complete. Typically, the skin protectant change occurs about once a day. And typically, it takes about two years for the wound to remodel, although the length of time for complete healing and remodeling will vary depending upon various factors, such as the size of the wound, the age and health of the patient, and the like.

In an embodiment, prior to the first step, i.e., prior to the application of the composition described herein comprised of analgesic, the skin protectant is applied to an area proximate to the skin condition or wound but not on the skin condition or wound. It is placed on a proximate area which is about 2 times the diameter of the size of the wound or skin condition up to about six inches from the skin condition or wound and then over the entire area approximate thereto. For example, if the wound or skin condition or burn is on a limb or finger or toe, then the skin protectant is applied topically over substantially the remainder of the limb, finger, or toe, respectfully. If it is on the shoulder, back or stomach then the skin protectant may be applied topically over substantially the rest of the shoulder, back, or stomach, respectively. The function thereof is to exfoliate old dead and dying skin in the proximate area of the wound or skin condition to bring new, viable skin condition to the surface. Without wishing to be bound, it is believed that as this composition is absorbed, the anti-inflammatory omega3 oils help reduce inflammation in the microvascular system. The vascular system, particularly the microcapillaries, relaxes to allow greater blood flow throughout the skin. The increased blood flow helps heal the wound more rapidly.

The skin-condition, burn, or wound may then be covered by gauze or band-aid or any other covering or dressing that is typically used to protect the skin-condition or wound from infection. In an embodiment, it is covered with a compression wrap. In another embodiment, especially when there is no bleeding or if the wound is a minor cut, such as a paper cut, after the collagen layer is applied, the wound or skin condition is not covered.

The patient periodically visits the doctor (in person or by telemedicine) for the doctor to see the progress of the healing of the wound. As determined by the doctor, in the next visit(s), the above protocol is repeated until the doctor indicates that treatment is no longer necessary.

In an embodiment, compositions in U.S. Pat. No. 10,463,699 or WO 2017/176753 or U.S. Ser. No. 63/013,167, the contents of which are incorporated by reference, may be utilized in conjunction with or replace one or two of the formulations described hereinabove with respect to the combination of formulations for the more severe wounds, burns or skin conditions. If the wound, burn or skin condition is chronic, then any of the formulations described in U.S. Pat. No. 10,463,699 or WO 2017/176753 or U.S. Ser. No. 63/013,167, the contents of which are incorporated by reference, may be used in combination with the formulations described herein. For example, an analgesic composition described in WO 2017/176753 or U.S. Ser. No. 63/013,167 may be utilized instead of the analgesic formulation described herein and the collagen-containing composition described in U.S. Pat. No. 10,463,699 or WO 2017/176753 or U.S. Ser. No. 63/013,167 may replace the collagen containing formulation described hereinabove in the treatment of chronic wounds or burns or skin conditions. For chronic wounds, burns or skin conditions, if the patient is in pain, an analgesic composition described in WO 2017/176753 or U.S. Ser. No. 63/013,167 may be utilized and applied to the situs of the wound, burn or skin condition. The non-analgesic and non-collagen formulations described herein may be applied to the periwound as described hereinabove. Treatment of the chronic condition (wounds, burns or skin condition) is used as described hereinabove, except the collagen containing formulation described in U.S. Pat. No. 10,463,699 or WO 2017/176753 or U.S. Ser. No. 63/013,167 is applied to the situs of the chronic wound, burn or skin condition until the wound, burn or skin condition begins to heal or until the physician determines that the wound, burn or skin condition is no longer chronic and is diagnosed as an acute condition, at which time, the physician will apply the appropriate formulation of the present disclosure, such as the collagen containing formulation of the present disclosure or the non-analgesic non-collagen containing formulation of the present disclosure in amounts and time effective to heal the acute condition, as determined by the physician based upon many factors, including the health and age of the patient, the size of the wound or skin condition or burn and the drainage from the wound, skin condition or burn, the sex of the patient, and the like.

In summary, the various compositions described in this disclosure can be used individually or sequentially as part of a healing process. For example, if used sequentially, the compositions described herein applied to intact skin need to permeate the skin leaving little greasy retentate. Compositions described herein applied to broken skin (i.e. no epidermis) are designed to leave more retentate on the surface. This strategy coats the broken skin with fat, inhibiting moisture loss and leaving behind a greasy surface that inhibits biofilm attachment and consequent infection. When collagen products are used, excess low retentate/low permeate oil is applied first and then the collagen matrix is applied. The total permeate mass saturates the wound bed with oil and the excess is not absorbed but wiped out with sterile gauze or any other sterile material utilized by physicians; the collagen matrix is added second and provides a slow release, low permeate/high retentate oil. Together, there is an initial skin-saturating spike flow followed by continuous low flow of oil to the wound bed over days. In one preferred embodiment, a third product is applied to intact periwound skin in addition to the low permeate/slow release collagen matrix. The third product is not added to the site where the low permeate/high retentate products are used, but rather it is added to the intact periwound skin surrounding the wound bed. The third product is a high retentate/high permeate product designed to bring oils to the periwound intradermal zone on either side of the basement membrane, including the hair follicles. By using three different products as a system, infection is eliminated, a structure is formed in the wound bed that attracts healing compounds, moisture is controlled and healing compounds from the hair follicles are nurtured and made available when signaled for. The combination accelerates healing in unexpected ways.

Once the wound has closed, healing is not complete. Temporary Collagen III needs to be remodeled into permanent and stronger Collagen I, a multi-year process. Using the high retentate/high permeate third product prophylactically during long-term remodeling meters essential fatty acids and non-inflammatory macrophages to the body for continuous use to strengthen the repair.

Without wishing to be bound, it is believed that the mechanism of action of the present disclosure is as follows: Linear fatty acid esters, such as Cetyl esters and monoglycerides, such as monolaurin and MCT form a semi-occlusive film over skin, which film reduces moisture loss, i.e., reduced transepidermal water loss or TEWL. Excess MCT (i.e. the excess MCT greater than that needed to drive oils through the skin) helps monoglycerides, such as monolaurin and linear fatty acid esters, such as cetyl esters, flow laterally across the skin surface during active rub-in. This makes the TEWL-reduction film thickness more uniform, the excess MCT is eventually absorbed through the follicular and intercellular pathways, carrying with it, the omega3 fatty acids. When the film forming factor in the formulation of the present disclosure is greater than equal to 42 wt % in non-collagen containing formulations or about 20% or greater in collagen containing formulation and when applied to the wound, burn or skin condition and to the surrounding tissue, the formulation promotes the film forming process on the wound, burn and skin conditions and surrounding tissue The monolaurin, MCT and cetyl esters, if any, are present in sufficient amount to reduce water loss, and keep pollutants, bacteria and harmful viruses out. In addition, it causes the lateral distribution of the components in the formulation, including the omega3 fatty acids therein. When also applied to the surrounding tissue, it promotes the absorption of the composition, including the omega3 fatty acids through the interfollicular pathway and promote the healing mechanism in humans. The oils described herein have higher FFF (film forming factor) values so that they have more uniform semi-occlusive films (with a silky-smooth finish), and the gels (with much higher monolaurin levels) require a more uniform film thickness to prevent spotty, non-uniform greasiness (“hot spots”). Further, the collagen containing compositions herein, on intact skin, has a finite follicular and intercellular transfer rate, so higher Film Forming Factor (FFF) greater than 20 wt % of the composition generates excess MCT and form a uniform TEWL-reducing film on the skin surface.

The Permeate Factor is a measure of driving oils through the epidermis into the dermis. The Retentate Factor describes the amount of wax needed to coat the skin surface. The FFF (Film Forming Factor) describes the excess solvent needed to spread the wax and form a uniform semi-occlusive film over epidermal skin.

Without wishing to be bound, it is further believed that unsaturated fats and medium chain triglycerides (C8:0 & C10:0) work together (analogous to a solvent) to move anhydrous molecules via the intercellular route. Saturated fats (C12:0 to C24:0), waxes and saturated fatty esters tend to coat the skin surface and impede transfer via the intercellular route. As the intercellular route velocity is reduced, anhydrous molecules partition into the hair follicle route, thereby, increasing the amount of surface resistance, selects for the interfollicular route. The sum of the wt % MCT, monolaurin and cetyl esters, takes into account both the Permeate Factor and the Retentate Factor. Both the Permeate Factor and the Retentate Factor can be separately manipulated to drive oils into the dermis via the intradermal, transdermal, interfollicular or intercellular route. They can also be independently changed to select for a higher percent following the transdermal hair follicle route. Caprylic and capric triglycerides (MCT) are solvents that drag longer unsaturated fats through the epidermis (high Permeate Factor). The higher the MCT level, the more unsaturated lipids permeate into the dermis. Monolaurin, a wax ester, creates an additional barrier to the inherent epidermal barrier (high Retentate Factor). At higher wax levels, more lipids are retained on the skin surface. Surprisingly, high MCT and high monoglyceride, such as monolaurin, together create a sustained, slow-release, long-lipid permeate that transfers some PUFA into the dermis over extended time with very little lipid retentate on the skin surface. Some of the PUFA is transmitted through the hair follicle.

Without wishing to be bound, it is believed that permeate passes through both the intercellular and interfollicular routes simultaneously. The intercellular route is preferred because the surface area of intercellular skin far exceeds the surface area of the sum of individual follicles. When permeate passage is accelerated, for example with low Retentate Factor oil, then most of the permeate bypasses the hair follicles. However, when the permeate is gelled (e.g. with monoglyceride, such as monolaurin, >6%), even though it has a high Permeate Factor, its permeation rate slows considerably as the high Retentate Factor impedes permeation. In gelled high Permeation Factor formulations, more time for complete permeation means there is more time for permeation into the hair follicle. Both the skin and the hair follicle have a basement membrane that restricts transfer to the dermis. The hair follicle is built like a flower vase filled with “marbles”. Staged permeate fills the voids between “marbles” and is stored until passage through the follicular basement membrane. Thus, slowing the overall rate of permeation means more of the temporary retentate is staged in the follicle “marble” voids. Then slowly, staged follicular retentate becomes follicular permeate. Gelled high Permeate Factor/high Retentate Factor oils selects for more interfollicular mass passing through the follicular basement membrane. It is believed that this increase in interfollicular passageway is not large in the absolute. Transfer into the dermis is not the technical objective. The objective is to bring sufficient MCT and omega3 fatty acid into the follicular bowl to cause the hair follicle to change from telogen (resting) to anagen (growth). This clinically observed transition has the well-known effect of causing the dermal papilla (base of the follicular bulb) to grow and extend down to the subcutaneous layer (third layer) of the skin. This follicle bulb growth (towards the subcutaneous layer) also increases the macro interstitial space between the follicular “marbles”, i.e. the available volume for holding temporary retentate via an increase in hair follicle bulb length.

The Retentate Factor indirectly measures the rate of the compositions remaining above the basement membrane surface during permeation. The rate is primarily controlled by gelation. Within the retentate there is a mixture of saturated fats and wax esters that determine skin feel (greasy to silky smooth). The mass of retentate also affects TEWL (transepidermal water loss), as layered wax is a barrier to moisture loss.

The gel is specially formulated to melt at body temperature. Thus, once heated and melted, the gel becomes a higher viscosity oil. Further, the Permeate Factor and Retentate Factor can also be adjusted so that some oil compositions of the present disclosure (or melted gel) is simultaneously transmitted intradermally and some transmitted through transdermal pathways. The Permeate Factor >3.0 embodiments can drive API molecules through the intercellular epidermis. But the API molecules are sterically hindered so transfer is slow. Gels are used to slow the transfer process, so the “solvents” do not leave the API behind. This is analogous to a sand filter where large “dirt” stays on the filter surface (retentate) and dissolved and small entrained solids pass through the sand filter (permeate). In this case the API are the small entrained solids that pass through the epidermis filter. This is particularly important when transporting OTC (over the counter) API molecules, such as lidocaine, camphor, salicylic acid, colloidal oatmeal (colloidal oatmeal is a retentate that stays on the skin surface) and other OTC active compounds. It is believed that longer chain saturated fats and waxy esters add resistance to the intercellular pathway at higher concentrations (or not, at lower concentrations). Increasing MCT increases the solvent transport of unsaturated oils through the intercellular pathway. Without wishing to be bound, it is believed that when the intercellular pathway is impeded, oils (or melted gels) partition into the hair follicle pathway. Thus, for example, when the retentate factor of the collagen-free compositions of the present disclosure is greater than 8.0, enough of the compositions are forced into the hair follicle pathway to induce telogen-to-anagen activation (observed clinically). When the formulations enter the hair follicle pathway, MCT, along with the other components of the composition feeds the stem cells and help them produce progeny. Very long chain omega3 fatty acid are inherently anti-inflammatory and help reduce scarring. Omega3 fatty acids are also biologically converted into other signaling compounds that help draw stem cells to the wound site.

In other words, without wishing to be bound, it is believed that the “Permeate Factor” is a measure that indirectly categorizes mixtures of lipids into faster permeation (Permeate Factor ≥3.0) and slower permeation (Permeate Factor <3.0) mixtures. Further, it is believed that the Retentate Factor being greater than 8.0 of the collagen-free compositions ensures a thick layer of fat on the breached skin surface, so that biofilm cannot attach to the greasy layer.

It is important to consider that the follicular route can only happen where there are follicles (i.e. not in breached skin). It is also important to recognize that follicular/intercellular migration is not a yes/no response. The correct response is one of more partition one way or the other depending on permeation and retentate values. The easiest way to determine whether or not the follicular pathway has achieved a critical mass of transfer is whether or not hair growth is perceived. If less than critical mass, there is no quantitative hair growth; if ≥critical mass, then there is hair growth.

Without wishing to be bound, it is believed that control of the oil compositions, collagen composition and melted gels into the follicular pathway occurs three ways:

1. A gross excess of the compositions of the present disclosure will exceed the absorptive capacity of the treated skin, leaving excess oil on the surface that will follow the path of least resistance into the hair follicles. This is the prior art strategy but leaves excess oil on the interfollicular surface that is cosmetically inelegant and can turn rancid when exposed to air.

2. Gels with high permeate and high retentate will slow the transfer of melted gel into the skin. A corollary is that melted gel is adjacent to the follicle bulb surface opening longer and more melted gel will enter the follicle. Hair will transition from telogen (resting) to anagen (growth). Stem cell progeny growth will occur; progeny will migrate out of the follicle to wherever they are called. This is the preferred approach because there is no spillage, there is a cosmetically elegant skin finish and there is no malodor.

3. Non-collagen compositions with sum of the weights of omega3 greater than 8 wt %, permeate factor <3.0 and low retentate factor will have excess omega3, some of which will transfer via the follicular pathway. The problem with this approach is transfer via the follicle is uncontrolled and anagen activation may or may not happen.

A high retentate factor and low permeate factor maximizes the oxidative burst but intentionally leaves excess lipid retentate on open or breached skin as a protective layer. The inherent anti-inflammatory capability of PUFA permeate creates an oxidative burst to sanitize the wound surface (because it does not penetrate deeply) and quenches excess free radicals using the inherent antioxidant capability of PUFA to help control the burst. Thus, compositions with a high permeate factor and low retentate factor promotes the passage of the PUFA into the dermis and leaves a reduced amount of free lipids (per gram of oil) on the surface of the wound (low Retentate Factor). In an embodiment, excess compositions with high retentate is added to overcome the low retentate factor (per gram) to ensure that there is excess retentate on the surface. This is true of any formulation. But applying the compositions in an oil formulation in excess of what the skin can absorb ends up with a greasy, smelly surface and slippery run-off from the target to the floor or field. If oil formulations are used, it should be washed off in about 15-20 minutes after application, unless covered by a bandage or other covering used in the art Surface lipids protect breached skin, i.e. skin without a functional stratum corneum barrier, from excess moisture loss to keep the wound moist. Moist wounds heal faster.

There are two independent factors affecting retentate: wax and saturated fat (C≥12). The mass of long chain saturated fat comes from the selection of excipient fats chosen (e.g. palm oil and coconut oil). The wax comes from gel/no-gel formulations (i.e., monoglyceride, such as monolaurin, greater than or equal to 6 wt % versus <1 wt %). In practice, a fixed level of monolaurin is chosen for oils (0.6 wt %) and a second usually-fixed level (9.5%) for gels. Cetyl Esters is generally 1 wt % in oil and 1 wt % in gels. The Cetyl Ester level is selected for its silky-smooth skin feel. The monoglyceride, such as monolaurin, level is chosen for gelation reasons. In an anhydrous composition, an increase in monoglyceride, such as monolaurin, to make a gel is combined with a corresponding reduction in oily components (to maintain 100% material balance). The reduction is generally made in oily components with significant saturated fat (i.e. coconut or palm). So, in general, going from a gel to an oil reduces wax and raises saturated fats, increasing the saturated fat retentate contribution, but reducing the wax retentate factor. Oils are generally not miscible in body temperature wax and easily pass through wax layers (when the waxes are liquid @<37° C.); the monoglycerides, such as monolaurin, are liquid waxes in the gels disclosed herein at body temperature).

In products with caprylic acid, the skin feel is different. Once caprylic acid interacts with skin moisture, some of the acid remains protonated and some of the acid is unprotonated. Unprotonated caprylic acid is water soluble and rinses off the hands when first washed. The hand feel is extraordinary elegant with a saturated coating of protonated free fatty acid mixed with wax and unabsorbed long chain saturated fatty acids.

From FIG. 20, gels are usually high permeation/high retentate products. Oils are usually low permeate/low retentate formulations. But gels are more versatile than oils. They can be high permeation/high retentate products or, alternately, low permeation/high retentate. In an embodiment, a gel is a low permeate/high retentate formula with the volatile active ingredient camphor. In one embodiment (PM3, Example 10), high caprylic FFA (2%) gel is rubbed on the chest. Both the FFA and the camphor become vapor when heated by the body. They are inhaled through the nose into the lungs. Sinuses clear and lung wheezing stops. The C8 FFA vapors are anti-inflammatory and are also reported to inhibit bacterial infection. This particular embodiment is unique in that the primary benefit is to volatilize caprylic acid, so it does not stay on the skin surface, but rather is heated by the chest, evaporates and then enters the blood stream by breathing through the nose and into the lungs. Chest surface temperature declines for about 15 minutes (during evaporation); chest surface temperature returns to normal after 15 minutes. Clinically, PM3 clears clogged sinuses and eliminates chronic wheezing. [Caprylic acid is well known as an antibacterial compound.]

A second gel embodiment (A8, Example 20) has a permeate factor of 5.05 (>3.0) and a high retentate factor (11.0). A8 replaces cetyl esters with yellow beeswax. This change increases the melting point of the gel above body temperature. This apparent small difference has the surprising benefit of making commercially woven collagens (e.g. cross-linked collagen (not hydrolyzed collagen) such as J&J's Promogran, the industry standard for wound healing) more useful by “caulking” the edges of the woven collagen to the periwound. The above-body-temperature-melting gel coats the periwound and prevents periwound maceration. This embodiment is used on wounds larger than 18 cm². In this case, unsaturated fats are additionally retained as retentate because the A8 gel does not soften at body temperature. In an embodiment, a dry woven, cross-linked collagen sheet (e.g. Prisma) is sprayed with a lidocaine oil (e.g. AA, Example 22) to make the sheet flexible (an anhydrous treatment analogous to spraying said sheet with aqueous saline). The now-flexible sheet is cut and pasted over the large wound and then the sheet edges are caulked in place with A8 “high-temperature” gel. The A8 caulk prevents the sheet from drying out and lifting off the edge of the wound bed. The A8 gel also provides a semi-occlusive protective layer over the immediate periwound. The immediate periwound (i.e. the periwound in intimate contact with the breached surface) does not macerate and swell so stem cell progeny can migrate easily over the periwound “dam” and migrate onto the wound surface to begin making epithelial tissue. The net effect is to begin healing very large wounds that have typically heretofore been expanding in size. The low-cost combination of an anhydrous oil spray, a woven cross-linked collagen-rich sheet and a non-melting perimeter gel is used in lieu of very high cost barometric chambers and negative pressure treatments to heal large wounds.

Without wishing to be bound, it is believed that a high permeate factor and high retentate factor of the collagen free compositions promotes the migration of mono and polyunsaturated lipids through the epidermis with the help of medium chain lipids (e.g. capric and caprylic fats). The longer chain saturated fats primarily partition on the surface of the epidermis and with retentate wax, deliberately slow the instantaneous rate of permeation into the dermis (high Retentate Factor) to reduce the oxidative burst but extend the duration of lipid delivery to the hair follicle bulb. Dermal oxygen perfusion (via the intercellular pathway) is decreased (i.e. decrease blood flow) modestly, to minimize the oxidative burst. In other words, the transmission of lipids is accelerated by bypassing the epidermal barrier while simultaneously increasing the resistance to said transmission.

Moreover, it is believed that when monoglycerides, such as monolaurin, are present in anhydrous oils @ greater than or equal to 6 wt %, the monoglycerides, such as monolaurin, form a weak gel that melts at body temperature. The weak gel can be used to support anhydrous additions of lipid-insoluble materials such that the gel appears to be homogeneous, even though it is not. It is further believed that the actual structure of the gel is such that “shelves-of-gel” support lipid-insoluble precipitate. For example, lidocaine is soluble in these lipid systems at about 1 wt %. A 4 wt % level is needed for deep pain relief. In the gel with 4 wt % lidocaine, 1 wt % of lidocaine is dissolved and 3 wt % is mechanically supported by the gel. When rubbed in, the undissolved lidocaine is transported deep into the tissue where pain relief can occur, for example at the sciatic nerve.

Moreover, colloidal oatmeal, that is water soluble and not lipid soluble, can be added. The colloidal oatmeal is evenly distributed by the gel without being dissolved in it. Oatmeal is continuously mixed in hot oil to keep it suspended. Once filled into consumer packages, gel forms as hot oil cools (inside the sealed package), trapping otherwise-precipitating oatmeal uniformly in the gel. A variety of OTC API actives can be delivered to the blood stream (transdermal) in the same fashion.

When the Permeate Factor ≥1.0 and monoglyceride, e.g., monolaurin greater than or equal to 6 wt % (e.g. Retentate Factor >8.0), there is a slow permeate release best for intact skin.

The monoglyceride, such as monolaurin, gel has an additional attribute, heretofore unknown. What is observed clinically is that inflammation is reduced only in the treated area. This unexpected event has strong clinical ramifications. For example, in a leg wound, a gel product has to be applied to the entire lower leg to transport inflammatory compounds out of the leg. As another example, in a lidocaine product, lidocaine is contained, not dissipated, to the treated area and penetrates deep into the tissue (because of the hair follicle transport selection), for example, towards the sciatic nerve instead of laterally away.

Further, it is believed that collagen skin polishes (e.g. rinse-off collagen products) are products with high MCT (high Permeate Factor) and saturated fats and monoglyceride, such as monolaurin, <6 wt %, and various waxes (high Retentate Factor) (FIG. 18). The intradermal penetration process loosens dead and dying cells in the epidermis and allows them to be easily scrubbed off by undissolved collagen particles and sea salt. In one embodiment, salicylic acid is added to make a specialty acne skin polish. The residual retentate is rinsed off with water after polishing, leaving glowing skin, for example on the face.

Further, it is believed that a low Retentate Factor; low Permeate Factor collagen free composition formulated as an oil, such as Oil D (Example 1) instantly penetrates the epidermis and leaves no fatty trace on the skin. This means that such composition does not require a high driving force to enter the dermis via the intercellular route because there is very little resistance from retentate wax. The benefit of this fast-penetrating oil is to provide dermal omega3 without odor and without greasiness. It makes a wonderful face and eye oil that allows cosmetic application on un-inflamed skin with very small pores. Oil D does not partition into the hair follicle as much as high retentate embodiments. Oil D can be used as a stand-alone cosmetic, but Oil D can also be used as a base for applying a user's personal portfolio of cosmetics because the pores are smaller and there is no greasy base on which cosmetics are overlaid. As a result, for example, the cosmetics will be applied to the face more smoothly.

The following table summarizes the differences and benefits of collagen and non-collagen oil and gel products vis-à-vis their retentate factor, permeate factor, the sum of omega3 fats and the amount of saturated fat ≥C14:0. The column on the right describes the primary use of that family of formulas.

TABLE 1

| | Low | High | Low | High |
|---|---|---|---|---|
| Situation | Permeate | Permeate | Retentate | Retentate |
| Cosmetic oil | | Yes | Yes | |
| Collagen - open wound | Yes | | | Yes |
| Collagen - approximated | | Yes | | Yes |
| wound | | | | |
| Collagen - cosmetic | | Yes | | Yes |
| Skin Protectant Oil | | Yes | Yes | |
| Skin Protectant Gel | | Yes | | Yes |
| Maximum omega3 oil | Yes | | Yes | |
| Maximum Pain Relief | | Yes | | Yes |
| Acne Gel | | Yes | | Yes |

Collagen-free composition products with a high retentate factor are gels. When they have a low amount of saturated fat (<8%), then additives can be included to make specialized products like skin protectants, topical analgesics, active pharmaceutical ingredients and the like.

If the collagen-free composition product has a high permeate factor (>3.0), a high retentate factor (>8) and high saturated fat (≥10%), then the product does not accept added active compounds easily.

If the collagen-free composition has a low retentate factor (8), it is an oil. If it also has a high saturated fat content (>10), it is designed to leave a layer of grease on the skin that inhibits bacterial adherence to the surface and thus indirectly inhibits biofilm development. Formulations with even higher saturated fats are part of the prior art and they are generally just too greasy to use on the skin.

Products with low permeate, high retentate and low saturated fats are specialty products. For example, A8 has no hemp oil, so it is a product designed to deliver very high level of EPA & DHA.

Collagen products with high permeate (≥1.0) are designed as intact skin exfoliant skin polishes or first aid antiseptics for minor wounds. The small amount of oil enters viable skin and loosens dead and dying the skin such that the leave behind collagen is a mild abrasive that removes the dead and dying residue.

Other collagen products with high permeate and low saturated fats (<8%) can be loaded with additives (e.g. lidocaine) to make a topical analgesic and a first aid antiseptic.

The following non-limiting examples further illustrate the present invention. In the examples, the monoglyceride is monolaurin, and the linear fatty esters utilized are cetyl esters and beeswax. Ascorbyl palmitate is a non-linear, fatty ester. Each of the examples provides the Permeate Factor (the sum of (C8:0+C10:0) saturated triglycerides/Sum of longer, saturated triglycerides (>C10:0)), the Retentate Factor (the sum of waxes, e.g., the sum of cetyl esters, monolaurin, beeswax and ascorbyl palmitate, when present) omega3. In each example, the Permeate Factor in the Table is congruent with the “Sat Fat Permeate Factor” listed in each example. The compositions described in the examples are prepared in accordance with the procedures described herein.

### EXAMPLES

The following examples provide the following information:
1. Sat Fat Permeate Factor=(C8+C10)/(sum saturated fat >C10:0)
   a. Sometimes shortened to “Permeation Factor”
2. Retentate Factor=(sum wax=monolaurin+cetyl ester+beeswax+ascorbyl palmitate)
3. Sum (MCT+monolaurin+cetyl esters+beeswax) . . . N.B. ascorbyl palmitate is not in this sum
   At the end of the Example section, a table summarizes all example formulae and the three ratios.

### Example 1

These compositions were prepared in accordance with the procedures described herein. The various components are gently mixed at about 40° C. until homogenous. Once a homogenous product is obtained, the resulting product is allowed to cool to 95° F. with gentle stirring and then further cooled to ambient in the absence of stirring.

Oil and gel products indicated below were rubbed into the right hand of an adult male for 30 seconds (left hand was gloved). The temperature of the base of the palm (hypothenar muscle) was recorded at the start and each minute thereafter for 5 minutes to establish a mean starting temperature. For 13 minutes, the base of the palm temperature was recorded every 30 seconds; thereafter the base of the palm temperature was recorded each 60 seconds, for 25 minutes total time.

Omega Oil BV

| | Weight % |
|---|---|
| MCT Oil | 30.0% |
| C8 FFA | 0.3% |
| RBD Palm Oil | 13.8% |
| Hemp Oil | 27.0% |
| Cod Liver Oil | 27.0% |
| Monolaurin | 0.6% |
| Cetyl esters NF | 1.0% |
| Lavender Oil | 0.1% |
| Lemon oil | 0.2% |
| Total | 100.0% |
| C8 + 10/sum unsat Unsat Fat Permeate Factor) | 0.59 |
| sum wax (Retentate Factor) | 1.6 |
| (ALA + SDA)/(EPA + DHA) | 0.97 |
| ALA + SDA + EPA + DHA (sum omega3) | 11.29 |
| Sat'd Fat > C10:0 | 14.25 |
| (C8 + 10)/sum fat > C10:0 (Sat Fat Permeate Factor) | 2.10 |
| C18:0 | 2.01 |

Omeza Topical Supplement - D Spray

| | |
|---|---|
| MCT Oil | 41.30% |
| Hemp Oil | 24.97% |
| Cod Liver Oil | 24.000% |
| Monolaurin | 0.60% |
| Cetyl esters NF | 1.00% |
| virgin coconut oil | 4.00% |
| RBD palm oil | 4.00% |
| perfume-B | 0.13% |
| Total | 100.00% |
| C8 + 10/sum unsat (Unsat Fat Permeate Factor) | 1.00 |
| sum wax (Retentate Factor) | 1.6 |
| (ALA + SDA)/(EPA + DHA) [PUFA Ratio] | 1.00 |
| ALA + SDA + EPA + DHA (sum omega3) | 10.17 |
| Sat'd Fat > C10:0 | 11.73 |
| (C8 + 10)/sum fat > C10:0 (Sat Fat Permeate Factor) | 3.56 |
| C18:0 | 1.53 |

Omeza Gel CS1 Skin Protectant

| | |
|---|---|
| MCT Oil | 52.09% |
| Hemp Oil | 12.00% |
| Cod Liver Oil | 16.21% |
| Monolaurin | 9.50% |
| Cetyl esters NF | 1.00% |
| virgin coconut oil | 0.33% |
| Colloidal oatmeal | 0.50% |
| RBD palm oil | 8.25% |
| perfume B | 0.12% |
| Total | 100.00% |
| C8 + 10/sum unsat (Unsat Fat Permeate Factor) | 1.96 |
| sum wax (Retentate Factor) | 10.5 |
| (ALA + SDA)/(EPA + DHA) [PUFA Ratio] | 0.75 |
| ALA + SDA + EPA + DHA (sum omega3) | 6.02 |
| Sat'd Fat > C10:0 | 8.35 |
| (C8 + 10)/sum fat > C10:0 (Sat Fat Permeate Factor) | 6.23 |
| C18:0 | 1.10 |

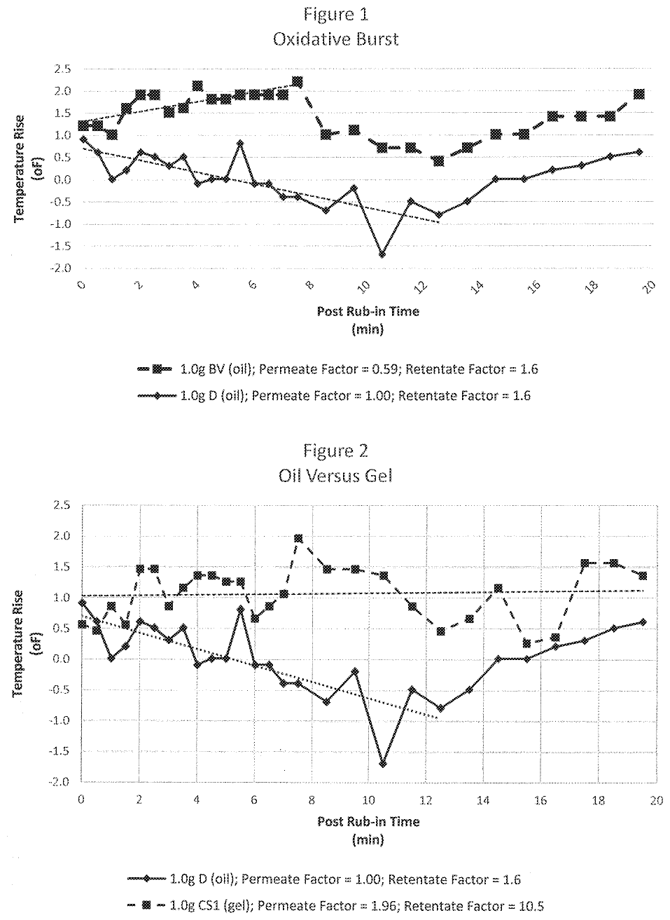

The change in base of the palm temperature versus the pre-application mean temperature was plotted for two oils and one gel. The results are provided in FIGS. 1-3.

### Explanation of Example 1

Oil BV (Sat Fat Permeate Factor=2.10; Retentate Factor=1.6) increases palm temperature during the first 8 minutes. Oil D (Sat Fat Permeate Factor=3.56; Retentate Factor=1.6) reduces palm temperature during the first 13 minutes.

Lower Permeate Factors keep saturated C>10:0 oils adjacent to the epithelial/dermal interface on the dermal side of the basement membrane (“intradermal”). Unsaturated fats, including PUFA are dragged through the epidermis by MCT, independent of the ratio of MCT/unsaturated fats (“Unsat Fat Permeate Factor”). PUFA anti-inflammatory action temporarily increases blood flow at the basement membrane interface, raising palm surface temperature. Higher Permeate Factors transfer long saturated fats deeper in the dermis and away from the skin surface. Surface temperature drops as blood flow is directed deeper in the dermis.

FIG. 1 shows increased palm temperature using oil BV, an indirect indicator of increased blood flow (oxidative burst) near the skin surface. With breached skin, e.g. chronic wound surfaces, the temporary oxidative burst brings ROS (Reactive Oxygen Species) to the surface. ROS kills bacteria and viruses that may be present on the surface. In controlled doses, ROS is beneficial for infection control.

FIG. 1 also shows the temperature response for Oil D (Sat Fat Permeate Factor=3.56; Retentate Factor=1.6 and BV oil. The initial palm temperature dropped during the first 13 minutes. Oils BV and D have similar PUFA (sum omega3) concentrations, but Oil D has a larger Permeate Factor (3.56 versus BV @ 2.10) because of higher MCT. Oil D is driven into the dermis and away from the surface. Intradermal blood flow is diverted away from the epithelial/dermal interface and towards the deep dermis (decreased surface perfusion).

Prior art oils (e.g. BV) have Permeate Factors <3; oils of the present disclosure have Permeate Factors ≥3. Prior art oils are prone to cloudiness when passing through freeze/thaw cycles during distribution. Cloudy oils do not recover unless heated above 40° C. Cloudy oils can also clog spray nozzles in multi-use packages. Cloudy oils do not have an impact when packed in single use vials because all the oil is dispensed, and cloudy particles are rubbed in. Permeate Factors are increased by using more MCT or by changing the relative amount of the vegetable oils. For example, Oil D has more MCT than BV, less palm oil (half saturated/half unsaturated) and some coconut oil added. Adding coconut oil is counterintuitive because coconut oil is all saturated fat, reducing the Sat Fat Permeate Factor, not increasing it to >3. It was found that when the permeate factor of an oil formulation is below 3, nozzles clog and the oils become cloudy; at >3, the oil is clear and dispenses uniformly.

Most gels, such as CS1, have Sat Fat Permeate Factors well above 3 because the waxes added to cause gelation replace vegetable oils and their inherent long chain fats.

In FIG. 2, the temperature responses in the palm with respect to Oil D and Gel CS1 (Example 6) were compared. In Gel CS1, the Retentate Factor was increased by increasing monolaurin gel (>6%) combined with a reduction in [sum (unsaturated triglycerides)], and an increase in MCT.

As shown in FIG. 2, there is a step increase in Gel CS1 palm temperature (to ˜+1° F.) and then there is no further temperature rise for the duration of the experiment. Gel CS1 has a higher Sat Fat Permeate Factor (6.23), but the monolaurin gel slows down the oil transfer into the skin (high Retentate Factor (10.5), <8). This provides time for the natural blood flow control strategies to adjust blood flow to keep the palm temperature constant.

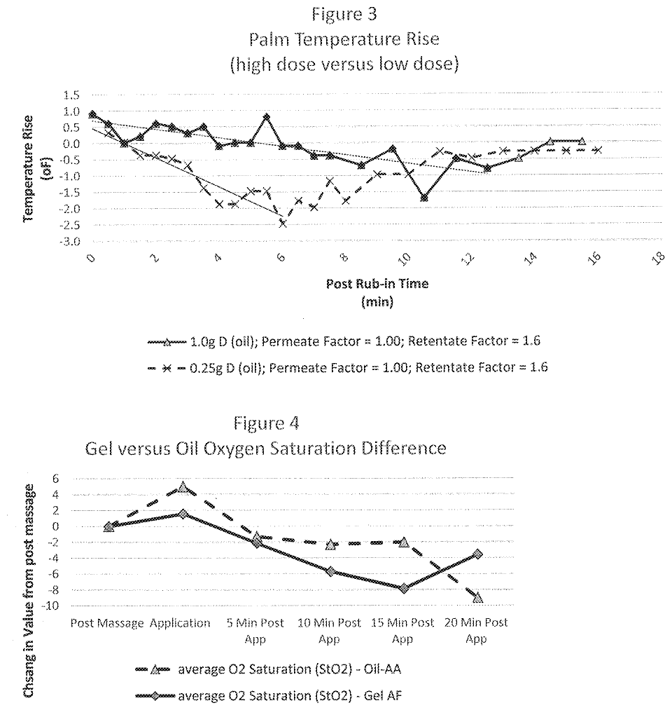

FIG. 3 compares the palm temperature response of a low dose (0.25 g) of Oil D versus a normal dose (1.0 g) of Oil D. The lower dose temperature drop is over in 6 minutes; the higher dose temperature drop is over in 13 minutes. Further, the higher dose has a longer effect as there is more mass to transfer.

### Example 2

Example 1 is a small-scale in-vivo test on the palm of one hand. Example 2 is also an in-vivo test, but on a population of 7 people. One leg was tested with an oil (Lidocaine Lavage—AA; Sat Fat Permeate Factor=2.25 (low); Retentate Factor=1.6 (low); the other was tested with Gel AF (Sat Fat Permeate Factor=1.32 (low); Retentate Factor=11.5 (high)).

Omeza Lidocaine Lavage - AA

| | Weight % |
|---|---|
| MCT Oil | 32.0% |
| C8 FFA | 0.3% |
| Monolaurin | 0.6% |
| Cetyl Esters NF | 1.0% |
| Lidocaine | 0.8% |
| RBD Palm Oil - CP6 | 14.64% |
| Red Palm concentrate | 0.26% |
| Hemp Oil | 25.2% |
| Cod Liver Oil | 25.2% |
| Total | 100.00% |
| C8 + 10/sum unsat (Unsat Fat Permeate Factor) | 0.66 |
| sum wax (Retentate Factor) | 1.6 |
| (ALA + SDA)/(EPA + DHA) [PUFA Ratio] | 0.97 |
| ALA + SDA + EPA + DHA (sum omega3) | 10.54 |
| Sat'd Fat > C10:0 | 14.20 |
| (C8 + 10)/sum fat > C10:0 (Sat Fat Permeate Factor) | 2.25 |
| C18:0 | 1.95 |

Omeza Gel AF
Skin Protectant
Omeza Skin Protectant

| | |
|---|---|
| MCT Oil | 32.14% |
| C8 FFA | 0.79% |
| Monolaurin | 9.50% |
| Cetyl esters NF | 2.00% |
| Coconut oil | 29.70% |
| Hemp Oil | 13.20% |
| Cod Liver Oil | 12.64% |
| perfume | 0.03% |
| Total | 100.00% |
| C8 + 10/sum unsat (Unsat Fat Permeate Factor) | 1.70 |
| sum wax (Retentate Factor) | 11.5 |
| (ALA + SDA)/(EPA + DHA) [PUFA Ratio] | 1.000 |
| ALA + SDA + EPA + DHA (sum omega3) | 5.36 |
| (C8 + 10)/sum fat > C10:0 (Sat Fat Permeate Factor) | 1.32 |
| C18:0 | 1.43 |

These compositions were prepared in accordance with the procedures described herein. The various components listed above are mixed together at 140° F. until a homogenous product is prepared. The resulting product is allowed to cool to 95° F. with gentle stirring and then no further stirring.

The Modulim Clarifi® system was used to quantify surface oxygen saturation and subsurface dermal perfusion. Modulim's Clarifi® Imaging System is the first non-contact, noninvasive microvascular assessment tool that uses a proprietary optical imaging technique spatial frequency domain imaging (SFDI) to provide insight into the delivery and extraction of oxygen to tissue. This information can help clinicians identify patients who are at risk for vascular complications and pinpoint specific areas of potential compromised circulation before the presence of visible symptoms (e.g., ulceration).

Noninvasive microvascular assessment powered by SFDI, the Clarifi® Imaging System provides a more complete picture of tissue health by quantifying and mapping key microvascular biomarkers. Tissue oxygenation and perfusion data are displayed in the form of color-coded maps, which helps clinicians to quickly identify specific regions of interest. Five key hemoglobin biomarkers are displayed on the Clarifi® user interface that provide insight into the oxygen delivery and extraction: average oxygen saturation; average oxygen saturation on hemoglobin; hemoglobin oxysaturated oxygen, average hemoglobin concentration, and two unique perfusion biomarkers—superficial (papillary dermis) hemoglobin (HbT₁) and subsurface (reticular dermis) hemoglobin (HbT₂):

The ability of Clarifi® to measure hemoglobin concentration and distribution in the papillary and reticular dermis reveals otherwise unavailable insight into the delivery of oxygen-carrying hemoglobin to the capillary network. Equipped with this new information, Clarifi® helps clinicians identify at-risk patients earlier, quantify the nature and level of their risk, and target care to prevent complications.

### Explanation of Example 2

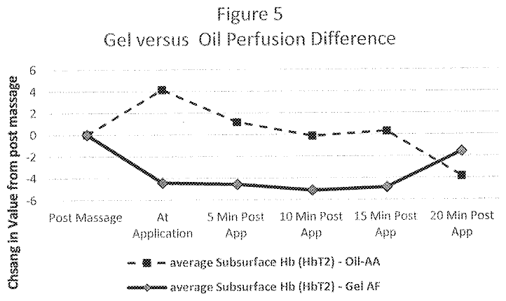

The Modulim system optically measures oxygen saturation in the top 0-1 mm of the dermis and then perfusion at 1-3 mm into the dermis. The results are indicated in FIGS. 4 and 5. FIG. 4 compares the average oxygen saturation of the gel and the oil, while FIG. 5 compares the average subsurface reticular dermis hemoglobin. In FIGS. 4 and 5, the y-axis is a unit-less number that compares a time point with a baseline. FIG. 4 shows surface oxygen saturation of an oil is about double the oxygen saturation of a gel as each is applied to the skin (1.6 to 5.0: +212%). FIG. 5 shows that oil perfusion (+4.1) is much greater at application than the gel (−4.4) because the Retentate Factor of the gel is much higher than the oil.

These differences are consistent with the impact of monolaurin levels of greater than or equal to 6% where a gel is formed. The gel reduces the oxygen burst and reduces perfusion to a constant rate for 15 minutes.

### Example 3

In Example 3, the skin penetration rate of Oil CB (Sat Fat Permeate Factor=1.59; Retentate Factor=1.6) was compared to Example 2 Gel AF (Sat Fat Permeate Factor=1.32; Retentate Factor=11.50). The composition of the Oil CB and the Omeza Gel AF are provided hereinbelow:

Omeza Oil CB
Skin Protectant

| | |
|---|---|
| MCT Oil | 38.70% |
| Crude coconut oil | 18.30% |
| Red Palm concentrate | 0.10% |
| Hemp Oil | 12.00% |
| Cod Liver Oil | 11.80% |
| RBD Palm Oil | 16.00% |
| caprylic acid | 1.50% |
| Monolaurin | 0.60% |
| Cetyl esters NF | 1.00% |
| Total | 100.00% |
| C8 + 10/sum unsat (Unsat Fat Permeate Factor) | 1.50 |
| sum wax (Retentate Factor) | 1.60 |
| (ALA + SDA)/(EPA + DHA) | 1.00 |
| ALA + SDA + EPA + DHA (sum omega3) | 5.01 |
| Sat'd Fat > C10:0 | 25.88 |
| (C8 + 10)/sum fat > C10:0 (Sat Fat Permeate Factor) | 1.59 |
| C18:0 | 1.80 |

Omeza Gel AF
Skin Protectant
Omeza Skin Protectant

| | |
|---|---|
| MCT Oil | 32.14% |
| C8 FFA | 0.79% |
| Monolaurin | 9.50% |
| Cetyl esters NF | 2.00% |
| Coconut oil | 29.70% |
| Hemp Oil | 13.20% |
| Cod Liver Oil | 12.64% |
| perfume | 0.03% |
| Total | 100.00% |
| C8 + 10/sum unsat (Unsat Fat Permeate Factor) | 1.70 |
| sum wax (Retentate Factor) | 11.5 |
| (ALA + SDA)/(EPA + DHA) [PUFA Ratio] | 1.000 |
| ALA + SDA + EPA + DHA (sum omega3) | 5.36 |
| (C8 + 10)/sum fat > C10:0 (Sat Fat Permeate Factor) | 1.32 |
| C18:0 | 1.43 |

These formulations are prepared in accordance with the procedures described herein.

The contact angle of a 1 microliter drop is monitored continuously @ 37° C. on 4 human forearms. Remaining-drop volume is calculated at each time interval. Comparison of absorption volume is made when 50% of the starting volume is consumed. The results are tabulated hereinbelow.

| | Omeza Skin | |
|---|---|---|
| | Protectant | Gel AF |
| | Average Time | Average Time |
| | for 50% | for 50% |
| | Drop Sorption | Drop Sorption |
| | into Skin | into Skin |
| Skin | (seconds) | (seconds) |
| 19 yr old White female Forearm | 39.5 | 70.5 |
| 35 yr old White Female Abdomen | 44.0 | 74.0 |
| 51 yr old White Male Forearm | 50.0 | 85.5 |
| 28 yr old Biack Female Forearm | 51.0 | 92.5 |
| Average of Four Skin Types | 46.0 | 80.5 |

### Explanation of Example 3

Example 3 shows that the oil absorbs 1.75× faster than the gel (80.5/46.0=1.75). The Sat Fat Permeate Factors are similar (CB=1.59; AF=1.32 (both low)); but there is a large increase in Retentate Factor (CB=1.6 (low); AF=11.5 (high)). The increased retentate slows the rate of absorption into the skin.

From Experiments 1-3, it was shown that:

Gels (high Retentate Factor) slow the absorption rate into the skin.

Oils (low Retentate Factor) increase the surface oxygen burst (oxygen saturation).

Gels reduce oxygen perfusion deeper in the dermis

The Permeate Factor is used to separate anhydrous oil/wax formulations into rapid penetration or slow penetration.

There is product differentiation @ Sat Fat Permeate Factor >3.0 (fast) and ≤3.0 (slow).

The Retentate Factor also has product differentiation (<8.0 (little is retained); ≥10.0 (much is retained).

Together, these variables describe a way to increase skin penetration (higher Permeate Factor) and/or to reduce skin penetration (higher Retentate Factor) or a little of both.

The Sat Fat Permeate Factor focuses on the penetration of longer saturated fats (C>10:0) because it was found that unsaturated fats are dragged into the dermis by MCT independent of MCT concentration and that longer saturated fats required more MCT (Permeate Factor >3) to transit the epidermis. The Retentate Factor is a measure of the residual wax on the skin.

Skilled artisans will quickly note that different products can be formulated by mixing and matching these two factors (high permeate:high retentate; low permeate:low retentate; high permeate:low retentate; low permeate:high retentate). For example, coconut, MCT and palm oils affect the ratios differently:

Coconut oil (86.9%≥C12:0) reduces the Permeate Factor by increasing the denominator.

RBD palm oil (˜50%≥C12:0; ˜50% unsaturated) increases permeate, but at ˜half the rate that coconut oil does.

MCT (100%<C12:0) affects the Permeate Factor directly (increases the numerator).

It is important to point out that the Retentate Factor only measures the wax and fatty ester subset of the total retentate and is a relative rate (g/sec) indicator. The actual total mass of retentate is the sum of (wax+fatty esters+not-yet-transferred saturated fats+not-yet-transferred unsaturated fats) times total mass of oil/gel. Thus, a low Retentate Factor does not necessarily mean low retained mass if there is a large amount (e.g. excess applied oil (i.e. saturated and unsaturated oil that did not penetrate in 15 minutes)) added to the skin. The wax portion is permanently in the retentate. The saturated oils are temporarily in the retentate. That is why the Retentate Factor is only waxes. C12:0 is not in the Permeate Factor even though it is the transition chain length between permeate and saturated fat retentate. C12:0 is a comedogenic fat (blocks the pores of the skin). C12:0 partially penetrates the epidermis but does not pass through. Shorter saturated fats (C8:0 & C10:0) pass through; longer saturated fats (C≥14:0) do not penetrate.

### Comparative Example 1

The following examples are described in WO 2019/200364

Example 29

| | Weight % |
|---|---|
| MCT Oil | 40.00% |
| C8 FFA | 0.30% |
| Monolaurin | 0.60% |
| Cetyl Esters NF | 1.00% |
| RBD Palm Oil - CP6 | 18.00% |
| red palm concentrate | 0.10% |
| Coconut oil | 20.00% |
| Hemp Oil | 10.00% |
| Cod Liver Oil | 10.00% |
| Total | 100.000% |
| C8 + 10/sum unsat (Unsat Fat Permeate Factor) | 1.68 |
| sum wax (Retentate Factor) | 1.60 |
| (ALA + SDA)/(EPA + DHA) [PUFA Ratio] | 0.24 |
| (DHA + EPA + SDA + ALA; sum omega3) | 4.23 |
| Sat'd Fat > C10:0 | 27.70 |
| (C8 + 10)/sum fat > C10:0 (Sat Fat Permeate Factor) | 1.54 |
| C18:0 | 1.84 |

Example 30

| | Weight % |
|---|---|
| MCT Oil | 40.00% |
| C8 FFA | 0.30% |
| Monolaurin | 0.60% |
| Cetyl Esters NF | 1.00% |
| RBD Palm Oil - CP6 | 20.00% |
| red palm concentrate | 0.10% |
| Coconut oil | 18.00% |
| Hemp Oil | 10.00% |
| Cod Liver Oil | 10.00% |
| Total | 100.00% |
| C8 + 10/sum unsat (Unsat Fat Permeate Factor) | 1.61 |
| sum wax (Retentate Factor) | 1.60 |
| (ALA + SDA)/(EPA + DHA) [PUFA Ratio] | 1.00 |
| ALA + SDA + EPA + DHA (sum omega3) | 4.24 |
| Sat'd Fat > C10:0 | 27.15 |
| (C8 + 10)/sum fat > C10:0 (Sat Fat Permeate Factor) | 1.56 |
| C18:0 | 1.88 |

Examples 29 and 30 have a Sat Fat Permeate Factor <3 (low). On the other hand, oils (like Oil D) have a Sat Fat Permeate Factor ≥3 (high) and consequently do not form cloudiness after freeze/thaw.

### Example 4

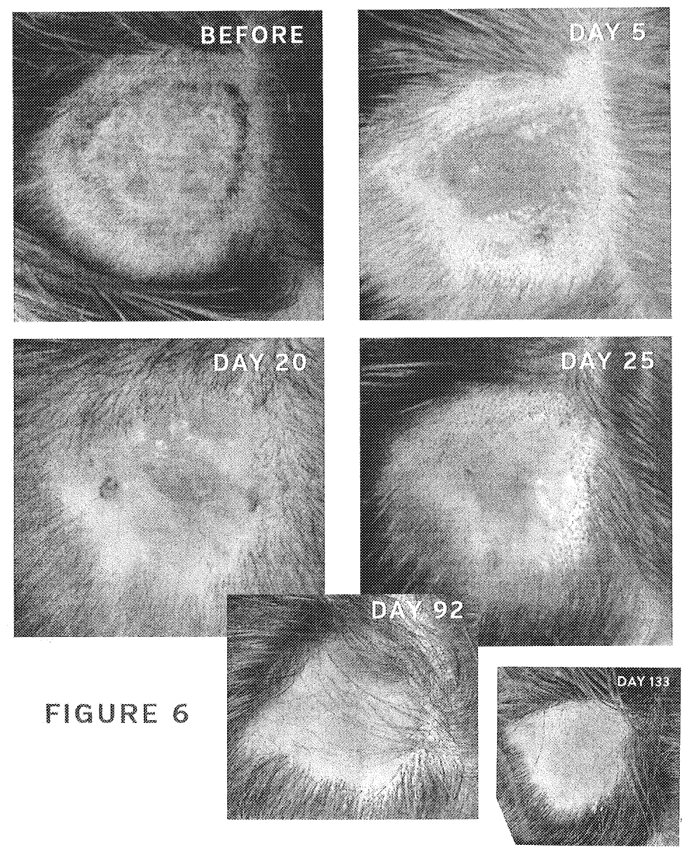

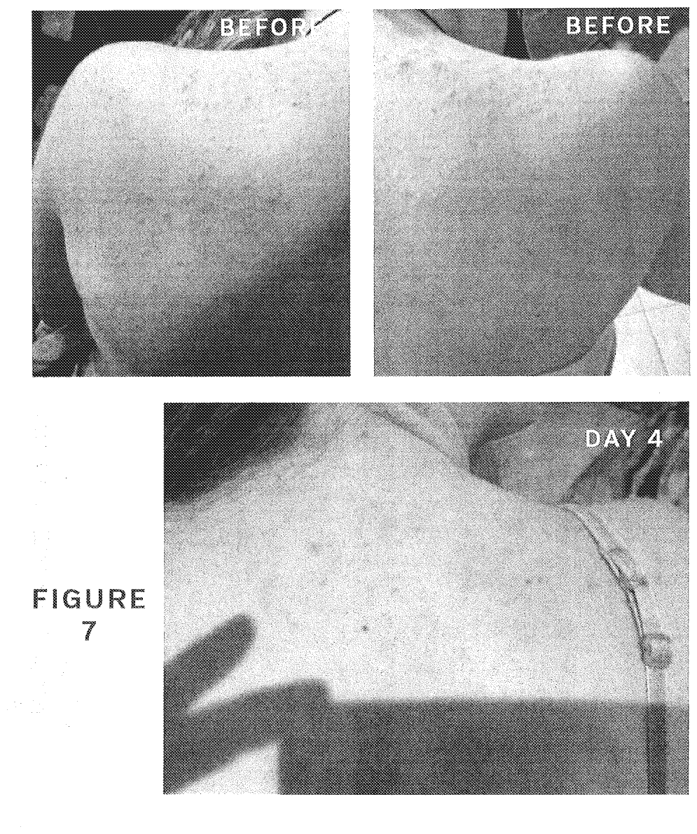

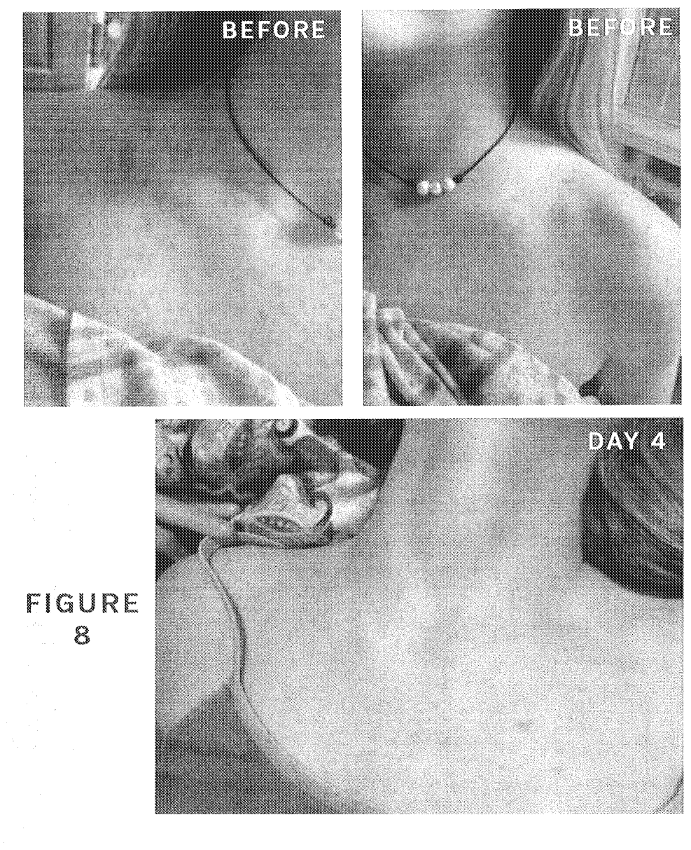

A young woman had a hair treatment that chemically burned a large hole in her scalp. The picture is on the left is the as-initially-treated at the hospital burn center photo in FIG. 6. The pictures on the right in FIG. 6 are ˜1-19 weeks later.

After discharge from the burn center, this woman was treated with First Aid DF (collagen; Sat Fat Permeate Factor=2.88 (high); Retentate Factor=10.25 (high; ≥8); the retentate factor=(monolaurin+cetyl esters+yellow beeswax+ascorbyl palmitate, all different waxes) every third day for 3 weeks and then with LL-AA (Permeate Factor=2.25 (low); Retentate Factor=1.6 (low)) daily (after the Day 25 photo on the right was taken). The compostions of these two formulations are provided hereinbelow:

Collagen Matrix DF
First Aid Antiseptic; whip

| | |
|---|---|
| MCT Oil | 15.36% |
| C8 FFA | 0.74% |
| Monolaurin | 4.09% |
| Cetyl Esters | 1.89% |
| Yellow Beeswax | 3.80% |
| Virgin Coconut Oil | 10.59% |
| Ascorbyl Palmitate | 0.47% |
| Hemp oil | 11.90% |
| Cod Liver Oil | 11.40% |
| Fish Collagen | 39.59% |
| benzethonium chloride | 0.17% |
| Total | 100.00% |
| C8 + 10/sum unsat (Unsat Fat Permeate Factor) | 0.88 |
| sum wax (Retentate Factor) | 10.25 |
| (ALA + SDA)/(EPA + DHA) [PUFA Ratio] | 1.00 |
| ALA + SDA + EPA + DHA (sum omega3) | 4.83 |
| Sat'd Fat > C10:0 | 11.55 |
| (C8 + 10)/sum fat > C10:0 (Sat Fat Permeate Factor) | 1.45 |
| C18:0 | 0.87 |

Omeza Lidocaine Lavage - AA

| | Weight % |
|---|---|
| MCT Oil | 32.0% |
| C8 FFA | 0.3% |
| Monolaurin | 0.6% |
| Cetyl Esters NF | 1.0% |
| Lidocaine | 0.8% |
| RBD Palm Oil - CP6 | 14.64% |
| Red Palm concentrate | 0.26% |
| Hemp Oil | 25.2% |
| Cod Liver Oil | 25.2% |
| Total | 100.00% |
| C8 + 10/sum unsat (Unsat Fat Permeate Factor) | 0.66 |
| sum wax (Retentate Factor) | 1.6 |
| (ALA + SDA)/(EPA + DHA) [PUFA Ratio] | 0.97 |
| ALA + SDA + EPA + DHA (sum omega3) | 10.54 |
| Sat'd Fat > C10:0 | 14.20 |
| (C8 + 10)/sum fat > C10:0 (Sat Fat Permeate Factor) | 2.25 |
| C18:0 | 1.95 |

The formulation for DF was prepared in accordance with the procedures described herein. All of the components except for collagen and benzethonium chloride are mixed together at about 160° F. (to melt the ascorbyl palmitate) until the mixture is a homogenous paste. When the resulting mixture is homogenous, collagen is added at temperatures at about 160° F. and then mixed with the other components until a substantially homogenous product is prepared. When substantially homogenous, which takes about ten to about twenty minutes, the composition is cooled to room temperature with gentle stirring. Benzethonium chloride is whipped under nitrogen at room temperature until the composition is substantially homogenous and the specific gravity <0.8.

AA is prepared as described herein in Example 2.

### Explanation of Example 4

Collagen Matrix DF has benzethonium chloride as a first aid antiseptic, and no sea salt is present in the composition. The MCT oil is increased to increase the Sat Fat Permeate Factor to 1.45 (i.e. high (≥1); versus Matrix CR @ 0.83). The Retentate Factor (10.25 (high; ≥10); Permeate Factor=1.45, (≥1; high)). The Retentate Factor is the sum of monolaurin, beeswax, cetyl esters and ascorbyl palmitate.

In FIG. 6, the as-initially-treated photo (left—“before”) shows burned necrotic tissue without vascularization. The Day 20 picture in the center shows a healthy pink wound bed that has robust, but incomplete, granulation. Islands of new white epithelial tissue are evident, along with an advancing periwound of new, white epithelial tissue. New, brown hair follicles are beginning to populate the new epithelial tissue in the Day 25 picture. The final picture (Day 133) shows the wound slowly contracting. The attending plastic surgeon originally recommended major surgery to approximate the wound with a skin transplant from her leg. The simple topical use of Collagen Matrix DF eliminated the need for surgery.

Collagen Matrix DF use was discontinued after 26 days because the need for collagen had passed. Collagen Matrix DF was replaced with LL-AA at about Day 60 (no treatment between Days 26 and 60) to alleviate headaches (contains lidocaine) and to provide a glossy, fatty protective coating (Sat'd Fat=14.14%) over the wound bed as epithelial tissue matures.

When a burn wound is first treated, there is little vascularity. Rapid blood flow is not indicated as it will cause pain in the renewing arterial system. Once angiogenesis has matured, rapid blood flow is indicated. Practically, this means that a high Retentate Factor/low Permeate Factor collagen matrix should be used after healing begins, followed by a low Retentate Factor oil once the inherent healing cascade is past the inflammation stage. To manage the blood flow, the oxidative burst and/or surface perfusion, depending on whether the wound is chronically in the inflammation phase or has transitioned into choreographed healing, are among the considerations in treating the wound.

Collagen Matrix DF (Example 4) is similar but different than Collagen Matrix CR (Example 6). Both have collagen and triglycerides. But the Permeate Factors are different (CR=0.83, very low; DF=1.45; low). Collagen Matrix DF Permeate Factor is ˜75% higher than Collagen Matrix CR. However, the Retentate Factors are about the same (CR=10.8; DF=10.25).

Collagen Matrix DF replaces Collagen Matrix CR process aids triethyl citrate and ethyl linoleate with additional MCT. These changes make DF collagen dissolve more rapidly than CR collagen (though the collagens are the same).

It has been determined that in an acute wound, the wound is not stuck in the inflammation stage and more early collagen availability is beneficial. However, in a chronic wound, collagen metered out over time is beneficial to allow time for the chronic wound to transition out of the inflammation stage.

### Example 5

This example compares Collagen Matrix CR with Collagen Matrix DF (Aka First Aid Antiseptic DF).

Collagen Matrix DF
First Aid Antiseptic; whip

| | |
|---|---|
| MCT Oil | 15.36% |
| C8 FFA | 0.74% |
| Monolaurin | 4.09% |
| Cetyl Esters | 1.89% |
| Yellow Beeswax | 3.80% |
| Virgin Coconut Oil | 10.59% |
| Ascorbyl Palmitate | 0.47% |
| Hemp oil | 11.90% |
| Cod Liver Oil | 11.40% |
| Fish Collagen | 39.59% |
| benzethonium chloride | 0.17% |
| Total | 100.00% |
| C8 + 10/sum unsat (Unsat Fat Permeate Factor) | 0.88 |
| sum wax (Retentate Factor) | 10.25 |
| (ALA + SDA)/(EPA + DHA) [PUFA Ratio] | 1.00 |
| ALA + SDA + EPA + DHA (sum omega3) | 4.83 |
| Sat'd Fat > C10:0 | 11.55 |
| (C8 + 10)/sum fat > C10:0 (Sat Fat Permeate Factor) | 1.45 |
| C18:0 | 0.87 |

Collagen Matrix
Collagen Matrix CR

| | Weight % |
|---|---|
| MCT Oil | 8.00% |
| C8 FFA | 1.30% |
| Monolaurin | 4.30% |
| Cetyl Esters | 2.00% |
| Yellow Beeswax | 4.00% |
| Triethyl citrate | 2.00% |
| Ethyl linoleate | 2.00% |
| Virgin Coconut Oil | 10.00% |
| Ascorbyl Palmitate | 0.50% |
| Red palm concentrate | 0.20% |
| Hemp oil | 12.00% |
| Cod Liver Oil | 11.40% |
| Fish Collagen | 41.40% |
| Ground Sea Salt | 0.90% |
| Total | 100.00% |
| C8 + 10/sum unsat (Unsat Fat Permeate Factor) | 0.49 |
| sum wax (Retentate Factor) | 10.8 |
| (ALA + SDA)/(EPA + DHA) [PUFA Ratio] | 1.01 |
| ALA + SDA + DHA + EPA (sum omega3) | 4.68 |
| Sat'd Fat > C10:0 | 11.17 |
| (C8 + 10)/sum fat > C10:0 (Sat Fat Permeate Factor) | 0.83 |
| C18:0 | 0.86 |

Collagen Matrix CR is an excellent product, designed to heal chronic, breached-surface, no-epithelium non-healing wounds. Collagen Matrix DF is an analogous product designed to heal minor acute wounds such as scrapes and approximated surgical wounds. There are important differences between Collagen Matrix CR and Collagen Matrix DF:

Sea salt versus benzethonium chloride

˜Double the MCT

Red palm concentrate (antioxidant) versus none

Triethyl citrate & ethyl linoleate versus none

Clinically, Collagen Matrix CR is a slow-collagen-release paste, while Collagen Matrix DF is a slightly more rapid collagen release paste. Collagen Matrix CR's Sat Fat Permeate Factor (0.83) is <1; Collagen Matrix DF's Permeate Factor (1.45) is ≥1. The Collagen Matrix DF Permeate Factor is ˜75% greater than the CR Permeate Factor. The Retentate Factors, sum omega3 and Sat'd Fat are almost identical.

### Explanation of Example 5

Reducing process aids (sea salt, triethyl citrate, ethyl linoleate) and replacing them with MCT has the effect of increasing the rate of hydrolyzed collagen dissolving (by driving the encapsulating oil into the wound bed faster). The inherent collagen solubility is unchanged; exudate interaction with the same collagen is increased by disrupting the wax that coats the collagen particles. This leads to faster dissolving (more uncoated collagen surface area).

The 75% increase of Permeate Factor changes how the product flows. Without wishing to be bound, it is believed that the wax and collagen set the viscosity of the anhydrous paste. The ascorbyl palmitate controls the amount of gas the matrix can hold (Pickering effect). MCT does not really dissolve the C>10:0 saturated fats in a collagen matrix. Rather, more MCT “softens” the saturated fat turning a “hard” fat into a soft grease surrounding particles of ascorbyl palmitate. The soft grease “balls” fill the interstitial volume between wax-coated collagen particles to help prevent weeping and to trap whipped-gas.

Replacing red palm concentrate with benzethonium chloride replaces a ROS quenching antioxidant with an antimicrobial active ingredient. Chronic wounds are more likely to be infected than acute wounds, so the trade off from free radical attack to chemical inhibition of pathogens is an attractive trade-off. Emerging tissue is less damaged. This change is clinically important for minor wounds and acute, approximated wounds because these wounds are not stalled and need dissolved collagen immediately to heal without scarring.

Chronic wounds that are stuck in the inflammation stage need to progress out of the inflammatory stage into the granulation stage before they need soluble collagen. This is a slower process.

The difference between Collagen Matrix CR and Collagen Matrix DF is the modulation of the rate of collagen dissolution by soft grease (around ascorbyl palmitate crystals). The goal is to match collagen availability to collagen need. When there are mismatches, excess collagen is destroyed by MMP (Matrix Metalloproteases) and is not available for tissue granulation.

Collagen Matrix DF is a vegetable/cod liver oil/hydrolyzed collagen mixture with a Sat Fat Permeate Factor ≥1, benzethonium chloride between 0.1 and 0.2 mg/l and no sea salt.

### Example 6

The following formulation is prepared in accordance with the procedure described herein, except it is heated to 165° F. (to melt the ascorbyl palmitate).

Omeza Gel CS1c
Skin Protectant

| | |
|---|---|
| MCT Oil | 52.09% |
| Hemp Oil | 12.00% |
| Cod Liver Oil | 16.21% |
| Monolaurin | 9.50% |
| Cetyl esters NF | 2.00% |
| virgin coconut oil | 0.33% |
| Colloidal oatmeal | 0.50% |
| RBD palm oil | 7.12% |
| ascorbyl palmitate | 0.25% |
| Total | 100.00% |
| C8 + 10/sum unsat (Unsat Fat Permeate Factor) | 2.00 |
| sum wax (Retentate Factor) | 11.75 |
| (ALA + SDA)/(EPA + DHA) [PUFA Ratio] | 0.75 |
| ALA + SDA + EPA + DHA (sum omega3) | 6.01 |
| Sat'd Fat > C10:0 | 7.78 |
| (C8 + 10)/sum fat > C10:0 (Sat Fat Permeate Factor) | 6.69 |
| C18:0 | 1.05 |

It was found that that the Example 6 formula (CS1c) had one optimum for transdermal delivery of unsaturated triglycerides (permeate) and a second optimum for residual surface waxes (retentate). The permeate affects healing; the retentate affects the cosmetic feel of the skin surface.

Omeza Gel CS1c has 0.5% colloidal oatmeal, a skin protectant active ingredient and 0.25% ascorbyl palmitate. The ascorbyl palmitate is a process aid to stabilize the gel @ 40° C. (potential distribution temperature abuse).

As will be shown in subsequent examples, over 90% of the mass of CS1c is kept unchanged, and up to 10% of the mass of CS1c can be changed to make different products

Colloidal oatmeal is water soluble, not lipid soluble. To make a homogeneous product, CS1c is heated to 165° F. (to melt the ascorbyl palmitate) and continuously mixed to keep the oatmeal suspended during hot melt filling. The gel forms in the finished package, suspending the oatmeal on “gel shelves”, not unlike pectin gels support fruit preserves in jam. The oatmeal also absorbs any fish odors.

### Explanation of Example 6

High Sat Fat Permeate Factor in oils and gels mixtures (i.e. ≥3.0) transport “almost all” unsaturated and some saturated triglycerides through the epidermis and then through the dermis (as permeate). Residual saturated fats, wax and a few unsaturated fats are left on the skin surface (retentate). It was found that the C12:0 and C14:0 fractions of virgin coconut oil are the primary fats affecting skin feel sensation. Thus only 0.33% of the base formula is virgin coconut oil, a balance between “lotion-feel” and “greasiness”. Hemp oil, cod liver oil and palm oil are the sources of unsaturated fats. MCT is 52.09% of the composition.

Omeza Gel CS1c is a mixture of vegetable oil, and cod liver oil such that the Permeate Factor ≥3.0. The monolaurin concentration (9.5%) is >6% to increase the Retentate Factor (11.75; (≥8)). In CS1c, optional ingredient colloidal oatmeal, is added. Using esters to increase retentate rather than fat creates a silky-smooth finish rather than a greasy finish.

Palm oil is a “universal donor” and can be adjusted up or down to make formula room for other added ingredients because the Sat Fat Permeate Factor (6.69) is >>than the Sat Fat Permeate Factor break point of 3.

This feature means that oil-soluble OTC active ingredients, e.g. lidocaine, can be driven deep into tissue (transdermal) for deep tissue pain relief. It also means that non-oil-soluble OTC active ingredients (e.g. colloidal oatmeal) stay on the skin surface for skin protection.

By mixing and matching different Permeation Factors with different Retentate Factors, intermediate OTC actives (e.g. salicylic acid) can be kept in the dermis (or the top of the hair follicle) to help prevent, for example, acne.

The following Omeza Gel CS1c table is used for all formulation calculations. Skilled artisans will recognize that the first two columns are the formula amounts for CS1c (or any other formulation containing the listed ingredients). The third column lists the various possible average triglyceride chain lengths (from public literature sources). Columns 4-9 represent the fixed chain length percent for each raw material. The right-hand column is the calculated weighted average of each chain length as: sum((% in formula*average % in raw material)=weighted sum in formula. The spreadsheet is used to calculate the Permeation Factor, Retentate Factor, sum omega3 and Sat'd Fats as a function of the relative weight of different ingredients for all formulations in the examples (all in “Bold” in the spreadsheet). At the bottom of the spreadsheet (“Analysis Result”), various ratios and sums are calculated. Relevant ratios and sums are appended to each formula in all the examples. Skilled artisans will recognize that the actual analytical percent of each chain length in each raw material may vary slightly from the literature-based average used in the calculation spreadsheet.

Omeza Gel CS1c

| Omeza Gel CS1c | | | cod | red | | | palm | palm | | weighted |
|---|---|---|---|---|---|---|---|---|---|---|
| 0 | weight % | | liver | Conc | C8/10 | coconut | stearin | oil | hemp | sum |
| palm oil | 7.1200% | C8:0 | | 0.034 | 59.7 | 7.3 | 0.06 | | | 31.12 |
| C8/10 fat | 52.0900% | C10:0 | | 0 | 40.1 | 5.8 | 0.06 | | | 20.91 |
| coconut oil | 0.3300% | C12:0 | | 0.173 | 0.1 | 49.1 | 0.87 | 0.2 | | 0.23 |
| C8/10 FFA | 0.000% | C14:0 | 3.60 | 0.961 | | 19.4 | 1.22 | 1.1 | 0.04 | 0.73 |
| Cetyl esters NF | 2.000% | C15:0 | | | | | | | | 0.00 |
| monolaurin | 9.500% | C16:0 | 10.40 | 42.465 | | 8.7 | 46.44 | 44 | 6.5 | 5.63 |
| hemp oil | 12.000% | C17:0 | | | | | | | | 0.00 |
| cod liver oil | 16.210% | C18:0 | 2.60 | 0.395 | | 2.6 | 5.07 | 4.5 | 2.5 | 1.05 |
| red palm concentrate | 0.000% | C20:0 | | | | | 0.38 | 0.4 | 0.65 | 0.11 |
| fish collagen | 0.000% | C22:0 | | 0.059 | | | 0.09 | | 0.23 | 0.03 |
| lidocaine | | C23:0 | | 0.022 | | | | | | 0.00 |
| Cetyl alcohol | 0.000% | C24:0 | | 0.067 | | | 0.09 | | 0.1 | 0.01 |
| perfume B | | C14.1 | | | | | | | | 0.00 |
| beeswax | | C16.1 | 6.50 | | | | 0.13 | | 0.13 | 1.07 |
| triethyl citrate | | C17.1 | | | | | | | | 0.00 |
| ethyl linoleate | | C18:1 | 20.60 | 44.616 | | | 36.03 | 39.2 | 14 | 7.81 |
| sea salt | 0.000% | C20:1 | 11.00 | | | | 0.12 | | 0.37 | 1.83 |
| ascorbyl palmitate | 0.250% | C22:1 | 8.40 | | | | | | 0.15 | 1.38 |
| benzethonium chloride | 0.000% | C24:1 | | | | | | | | 0.00 |
| salicylic acid | | C18:2 | 1.50 | 10.372 | | 0.7 | | 10.1 | 56.5 | 7.74 |
| camphor | 0.00% | C20:2 | | | | | | | 0.05 | 0.01 |
| colloidal oatmeal | 0.50% | g-18:3 | | | | | | | 0.85 | 0.10 |
| total | 100.0000% | a-C18:3 | | 0.257 | | | | 0.4 | 18 | 2.19 |
| | | C20:3 | | | | | | | | 0.00 |
| sum oil | 87.750% | C18:4 | 2.40 | | | | | | | 0.39 |
| | | C20:4 | | | | | | | | 0.00 |
| | | C20:5 | 9.30 | | | | | | | 1.51 |
| | | C22:5 | | | | | | | | 0.00 |
| | | C22.6 | 11.90 | | | | | | | 1.93 |
| | | \hat{ }3 | | | | | | | | |

| Analysis | Result |
|---|---|
| C8/10 | 52.03 |
| Sum unsat | 25.95 |
| C8 + 10/sum unsat (Unsat Fat | 2.00 |
| Permeate Factor) | |
| sum unsat >18:1 | 17.07 |
| (C8 + 10)/(sum unsat + sum | 1.54 |
| other sat) | |
| sum omega 3 | 5.63 |
| C16:0/C18:1 | 0.72 |
| (C8 + C10)/(C16:0 + C18:1) | 3.87 |
| (C8 + C10)/(sum(C12 to C24)) | 1.54 |
| PUFA | 13.87 |
| mono/PUFA | 0.87 |
| 8 + 10/PUFA | 3.75 |
| omega6 | 7.74 |
| omega6 + 3 | 13.37 |
| 8 + 10/omega6 + 3 | 3.89 |
| 18:2/sum(DHA + EPA) | 2.25 |
| n3 | 6.01 |
| n6 | 7.9 |
| n3/n6 | 0.77 |
| (C14:0-C24:0)/(C14:1-C24:1) | 0.63 |
| (DHA + E PAJ/ALA | 1.31 |
| ALA + SDA + EPA + DHA | 6.01 |
| (sum omega3) | |
| (ALA + SDA)/(EPA + DHA) | 0.75 |
| [PUFA Ratio] | |
| sum (C12:0 to C24:0) | 7.78 |
| C8&10/C16:0 | 9.25 |
| C8&10/C18:1 | 6.66 |
| C8:0 to C12:0/monolaurin | 5.50 |
| C12:0 to C24:0/monolaurin | 6.30 |
| C8:0 to C10:0/(monolaurin + | 4.52 |
| cetyl esters) | |
| (C8 to C12:0)/C > 12:0 | 1.56 |
| C > 12:0/C8:0 to C12:0 | 0.64 |
| (C8 + 10 + C12)/(ALA + SDA + | 8.69 |
| EPA + DHA) | |
| (C8 + 10 + C12)/(ALA + SDA + | 2.69 |
| EPA + DHA + C16:0 + C18:1) | |
| Sat'd Fat >C10:0 | 7.78 |
| sum(C8 + 10)/(PUFA + mono + | 2.05 |
| cetyl) | |
| sum(C8 + 10 + C12)/(PUFA + | 2.06 |
| mono + cetyl) | |
| (C8 + C10)/C > 10 | 1.54 |
| (C8 + C10)/(C18:1 + monolaurin) | 3.01 |
| C18:0 | 1.05 |
| (C ≤ 12 + FFA)/C > 12 | 1.560 |
| MCT + Mono + cetyl ester | 63.59 |
| (8 + 10)/(omega3 + sum sat ≥ | 3.11 |
| C18:0 + monolaurin) | |
| (C12:0 to C24:0 + wax) | 19.28 |
| (8 + 10) + sum(unsat) | 77.98 |
| (8 to 12)/(other glycerides) | 1.22 |
| (C8 + C10)/PUFA | 3.75 |
| (C8 + C10)/(PUFA + monolaurin) | 2.23 |
| collagen/(C8 + C10) | 0.00 |
| EPA + DHA | 3.44 |
| (C8 + 10)/sum(sat fat >10 + | 2.66 |
| esters + wax + AP) | |
| sum(sat fat C ≥ 12 + wax) | 19.53 |
| sum wax (Retentate Factor) | 11.75 |
| (C8 + 10)/sum fat > C10:0 | 6.69 |
| (Sat Fat Permeate Factor) | |

### Example 7

The following Formulation is prepared as described herein, heated to 140° F. (as there is no ascorbyl palmitate).

Psoriasis Relief Gel CS3a

| | |
|---|---|
| MCT Oil | 52.09% |
| virgin coconut oil | 0.33% |
| Hemp Oil | 12.00% |
| Cod Liver Oil | 16.21% |
| RBD palm oil | 5.77% |
| Monolaurin | 9.50% |
| Cetyl esters NF | 1.00% |
| colloidal oatmeal | 0.50% |
| lidocaine | 0.50% |
| camphor | 0.10% |
| salicylic acid | 2.00% |
| Total | 100.00% |
| C8 + 10/sum unsat (Unsat Fat Permeate Factor) | 2.06 |
| sum wax (Retentate Factor) | 10.50 |
| (ALA + SDA)/(EPA + DHA) [PUFA Ratio] | 0.75 |
| ALA + SDA + EPA + DHA (sum omega3) | 6.01 |
| Sat'd Fat > C10:0 | 7.11 |
| (C8 + 10)/sum fat > C10:0 (Sat Fat Permeate Factor) | 7.32 |
| C18:0 | 0.99 |

An unexpected synergy is found with lidocaine plus camphor in the present formulation. Pain relief happens at the skin surface and deep in the dermis. Salicylic acid is a well-known OTC psoriasis-relief active ingredient.

Psoriasis sufferers become asymptomatic (i.e. not cured) using Psoriasis Gel-CS3a.

### Explanation of Example 7

Psoriasis Gel-CS3a provides simultaneous shallow and deep pain relief because the camphor is in the retentate and lidocaine is in the permeate. Thus, temporary pain relief occurs on the itchy surface and the deep dermis. The salicylic acid helps remove the psoriasis plaque.

Psoriasis Gel-CS3a is a mixture of vegetable oils and cod liver oil with a high Sat Fat Permeate Factor (≥3 (@ 7.32)). Monolaurin @ >6%, combined with lidocaine, camphor, colloidal oatmeal and salicylic acid has a high Retentate Factor of 10.5 (≥8).

### Example 8

The following formulation is prepared in accordance with the procedure described herein in Example 7:

Eczema Relief

Eczema Analgesic Gel CS4

| | |
|---|---|
| MCT Oil | 52.09% |
| virgin coconut oil | 0.33% |
| Hemp Oil | 12.00% |
| Cod Liver Oil | 16.21% |
| RBD palm oil | 7.77% |
| Monolaurin | 9.50% |
| Cetyl esters NF | 1.00% |
| colloidal oatmeal | 0.50% |
| lidocaine | 0.50% |
| camphor | 0.10% |
| Total | 100.00% |
| C8 + 10/sum unsat (Unsat Fat Permeate Factor) | 1.98 |
| sum wax (Retentate Factor) | 10.50 |
| (ALA + SDA)/(EPA + DHA) [PUFA Ratio] | 0.75 |
| ALA + SDA + EPA + DHA (sum omega3) | 6.02 |
| Sat'd Fat > C10:0 | 8.11 |
| (C8 + 10)/sum fat > C10:0 (Sat Fat Permeate Factor) | 6.42 |
| C18:0 | 1.08 |

Eczema Pain Relief Gel—CS4 is a mixture of vegetable oils and cod liver oil with a high Sat Fat Permeate Factor (≥3 (6.42)). This formulation in which monolaurin @ >6% combined with lidocaine, camphor and colloidal oatmeal has a high Retentate Factor (10.5; ≥8). Colloidal oatmeal is an OTC active ingredient for eczema relief.

### Explanation of Example 8

Example 8 is yet another variation of the Omega Gel CS1c formula with three different OTC active ingredients. Example 8 illustrates the breadth of products that can be formulated using the CS1c base.

### Example 9

The following formulation is prepared in accordance with the procedure described herein:

Omeza Deep Pain Relief - CN
4% + camphor

| | |
|---|---|
| MCT Oil | 52.09% |
| Hemp Oil | 12.00% |
| Cod Liver Oil | 16.21% |
| Monolaurin | 9.50% |
| Cetyl esters NF | 1.00% |
| RBD palm oil | 4.90% |
| Lidocaine | 4.00% |
| Camphor | 0.30% |
| Total | 100.00% |
| C8 + 10/sum unsat (Unsat Fat Permeate Factor) | 2.09 |
| sum wax (Retentate Factor) | 10.5 |
| (ALA + SDA)/(EPA + DHA) [ PUFA Ratio] | 0.75 |
| ALA + SDA + EPA + DHA (sum omega3) | 6.01 |
| Sat'd Fat > C10:0 | 6.41 |
| (C8 + 10)/sum fat > C10:0 (Sat Fat Permeate Factor) | 8.12 |
| C18:0 | 0.94 |

Omeza Deep Pain Relief—CN is based on the Omeza Gel CS1c formula except 4% lidocaine and 0.3% camphor are added (palm oil is adjusted to balance the formula to 100%). The lidocaine is soluble up to ˜1% in the Omeza Gel CS1c base, so 3% of lidocaine is insoluble and would otherwise precipitate in an oil. Ascorbyl palmitate is not in CN, but optionally could be added for more temperature-abuse stability in CN and all CS1c derivative gels. If it were added, palm oil would be adjusted to balance.

It was found that CN alleviates sciatic nerve pain deep in the lower back and legs. This is a surprise because topical lidocaine is used typically for surface pain relief.

### Explanation of Example 9

Omeza Deep Pain Relief-CN is a mixture of vegetable oils and cod liver oil with a high Sat Fat Permeate Factor (8.12; ≥3). The high Retentate Factor is 10.5 (≥8). Monolaurin @ >6%, to increase the Retentate Factor, is combined with lidocaine and camphor.

Undissolved lidocaine is gently mixed in melted CN until hot melt filling. The filled package cools and the gel forms in the package, trapping undissolved lidocaine in the gel interstices.

When the gel is rubbed on the skin, the gel melts and the undissolved lidocaine is released to the skin surface. Without wishing to be bound, it is believed that high levels of MCT would normally drag the lidocaine through the epidermis into the dermis, but the high Retentate Factor diverts lidocaine to the hair follicle pathway. The outlet of the hair follicle is in the subcutaneous tissue adjacent to the sciatic nerve. Even as some of the MCT is metabolized in the hair follicle, there is still enough remaining MCT to transport the lidocaine deep in the tissue below the treatment area.

The camphor stays on the surface as retentate, cooling and providing superficial pain relief. The combination of surface pain relief (via camphor) and deep pain relief (via lidocaine) “confuses” the pain sensation and patients report pain relief for hours.

### Example 10

The following formulation is prepared in accordance with the procedure described herein:

Omeza Sleep Aid - PM3

| | |
|---|---|
| MCT Oil | 30.00% |
| Hemp Oil | 26.90% |
| Cod Liver Oil | 25.90% |
| Monolaurin | 9.50% |
| Cetyl esters NF | 1.00% |
| C8 FFA | 2.00% |
| camphor | 4.70% |
| Total | 100.00% |
| C8 + 10/sum unsat (Unsat Fat Permeate Factor) | 0.70 |
| sum wax (Retentate Factor) | 10.50 |
| (ALA + SDA)/(EPA + DHA) [PUFA Ratio] | 1.00 |
| ALA + SDA + EPA + DHA (sum omega3) | 10.95 |
| Sat'd Fat > C10:0 | 7.02 |
| (C8 + 10)/sum fat > C10:0 (Sat Fat Permeate Factor) | 4.26 |
| C18:0 | 1.35 |

Sleep Aid PM3 combines hemp oil and cod liver oil (high Sat Fat Permeate Factor=4.26, ≥3) with higher levels of caprylic free fatty acid (C8 FFA @ 2%) and camphor (4.7%). The FFA and camphor evaporate when rubbed onto the warm chest, escape, and are inhaled through the nose and then into the lungs.

Sinuses become clear; lung wheezing stops and the user gets a full night's sleep. The camphor is an OTC active ingredient for antitussives (treats coughs and congestion) and aids in getting to sleep.

### Explanation of Example 10

Omeza Sleep Aid-PM3 uses a high Permeate Factor combined with a high Retentate Factor to keep the FFA/camphor in the retentate (i.e. not drag it through the stratum corneum) while simultaneously increasing sum omega3 (10.95%) and MCT to drag permeate through the stratum corneum. Increased PUFA (polyunsaturated fatty acid) permeate increases superficial blood flow to raise treated-skin temperature. The increased heat is used to volatilize FFA and camphor over a ˜15 minute post application period.

Caprylic acid is well known as a bactericidal compound. When slowly inhaled with camphor, the sinuses and lungs clear in ways poorly understood.

Palm and coconut oil are intentionally absent to keep them out of the retentate. Solid fat on the skin surface would bind the FFA and prevent it from vaporizing.

The positive breathing effect is most pronounced on patients with restricted breathing. After one week, these patients have clear lungs. Omeza Sleep Aid-PM3 use is continued thereafter as a prophylactic maintenance measure.

### Example 11

The following formulation is prepared in accordance with the procedure described herein:

Omeza Topical Supplement - D
Spray

| | |
|---|---|
| MCT Oil | 41.30% |
| Hemp Oil | 24.97% |
| Cod Liver Oil | 24.000% |
| Monolaurin | 0.60% |
| Cetyl esters NF | 1.00% |
| virgin coconut oil | 4.00% |
| RBD palm oil | 4.00% |
| perfume-B | 0.13% |
| Total | 100.00% |
| C8 + 10/sum unsat (Unsat Fat Permeate Factor) | 1.00 |
| sum wax (Retentate Factor) | 1.6 |
| (ALA + SDA)/(EPA + DHA) [PUFA Ratio] | 1.00 |
| ALA + SDA + EPA + DHA (sum omega3) | 10.17 |
| Sat'd Fat > C10:0 | 11.73 |
| (C8 + 10)/sum fat > C10:0 (Sat Fat Permeate Factor) | 3.56 |
| C18:0 | 1.53 |

In Topical Supplement-D, the Sat Fat Permeate Factor is high (D=3.56, ≥3), The Retentate Factor is also low (D=1.6; <8). The sum omega3 is high, 10.17%.

Topical Supplement-D is designed for permeation with almost no retentate. It is used on the face and eye. Cosmetic users receive the benefit of reduced inflammation (from PUFA deep in the dermis) plus a non-greasy skin surface for applying make-up easily. The non-greasy surface is achieved by minimizing palm and coconut oil concentration. The high Permeate Factor prevents precipitation (cloudiness) in distribution, so all triglycerides and waxes are in solution upon application.

Topical Supplement-D can also be used as a transdermal platform for transporting lipid soluble API into the blood stream topically. When used as an API transport (Example 15; RG4), palm oil is reduced proportionately, and perfume is eliminated.

### Explanation of Example 11

Topical Supplement-D is designed to bring high levels of omega3 triglycerides into the dermis while leaving only a small amount of retentate on the skin surface. Some APIs are completely lipid soluble at therapeutic doses; some APIs are partially lipid soluble at therapeutic doses. Some are lipophobic. Topical Supplement—D is used for APIs that are completely lipid soluble. Supplement D is used on the face and eyes, but very little mass is used at any one application. This means there is little oil or wax-residue on the skin surface (de minimis). That is, the small amount used is almost completely absorbed. This is the opposite result-pattern compared to AA (excess usage to saturate exposed dermis) when it is applied to an open wound; it leaves behind a topical layer of grease.

Omega Gel CS1c is used for APIs that are partially lipid soluble at therapeutic doses.

### Example 12

The following formulation is prepared in accordance with the procedure described herein:

Collagen Matrix DD
Skin Polish

| | Weight % |
|---|---|
| MCT Oil | 23.60% |
| C8 FFA | 1.00% |
| Monolaurin | 4.30% |
| Cetyl Esters | 1.00% |
| Yellow Beeswax | 4.00% |
| Virgin Coconut Oil | 10.23% |
| Ascorbyl Palmitate | 0.50% |
| Hemp oil | 6.37% |
| Cod Liver Oil | 6.10% |
| Fish Collagen | 41.40% |
| sea salt | 1.50% |
| Total | 100.00% |
| C8 + 10/sum unsat (Unsat Fat Permeate Factor) | 2.45 |
| sum wax (Retentate Factor) | 9.80 |
| (ALA + SDA)/(EPA + DHA) [PUFA Ratio] | 1.00 |
| ALA + SDA + EPA + DHA (sum omega3) | 2.59 |
| Sat'd Fat > C10:0 | 9.84 |
| (C8 + 10)/sum fat > C10:0 (Sat Fat Permeate Factor) | 2.53 |
| C18:0 | 0.58 |

Skin Polish DD is a soft scrub skin polish to exfoliate dead and dying keratinocytes from the intact skin of the face. Skin Polish DD has a high Retentate Factor (9.8; (<8.0)). Skin Polish DD has a high Sat Fat Permeate Factor (2.53; ≥1), as a result of the MCT oil (larger numerator) and low PUFA (smaller denominator). Undissolved collagen is a gentle scrubbing abrasive; sea salt is the more aggressive scrubbing abrasive. They work together to remove dead skin without irritation.

The high Permeate Factor ensures that omega3 oils penetrate the dermis, but the low absolute concentration of omega3 oils (2.59 wt %) and the high dose of collagen means most of the product stays on the skin until rinsed off with water. Leave-behind products (e.g. CR, Example 4) have a low permeate factor (slow oil release); rinse-off products (e.g. DD) have a high permeate factor (release and absorb low-dose oil quickly, before aqueous rinse-off).

Users report that DD makes their skin “shine” like a baby's skin. Sea salt is increased to 1.5% to match consumer's perception for ideal skin polishing.

### Explanation of Example 12

Exfoliation is a weekly face treatment for many cosmetic users. These users want to remove dead and dying skin to create a vibrant platform for their cosmetic protocol. These consumers are also sensitive to odors, like oxidized omega3 fatty acids.

The polish is applied and then immediately rinsed off. Dead skin and any residual odors are rinsed away.

### Example 13

The following formulation is prepared in accordance with the procedure described herein:

Collagen Acne Matrix DE
Skin Polish

| | |
|---|---|
| MCT Oil | 24.17% |
| Hemp oil | 6.37% |
| Cod Liver Oil | 6.10% |
| Monolaurin | 4.36% |
| Cetyl Esters | 1.00% |
| Virgin Coconut Oil | 10.00% |
| Yellow Beeswax | 4.00% |
| Ascorbyl Palmitate | 0.50% |
| Fish Collagen | 41.40% |
| salicylic acid | 0.60% |
| sea salt | 1.50% |
| Total | 100.00% |
| C8 + 10/sum unsat (Unsat Fat Permeate Factor) | 2.50 |
| sum wax (Retentate Factor) | 9.86 |
| (ALA + SDA)/(EPA + DHA) [PUFA Ratio] | 1.00 |
| ALA + SDA + EPA + DHA (sum omega3) | 2.59 |
| Sat'd Fat > C10:0 | 9.66 |
| (C8 + 10)/sum fat > C10:0 (Sat Fat Permeate Factor) | 2.63 |
| C18:0 | 0.58 |

Acne Matrix DE Skin Polish has the same basic formula as Collagen Matrix DD except that 0.6 wt % salicylic acid is added, an OTC acne active ingredient. The Collagen Matrix DD formula is adjusted to balance (via MCT & coconut oil adjustment).

Acne sufferers polish their skin with Collagen Matrix DE.

### Example 14

The following formulation is prepared in accordance with the procedure described herein:

Omeza Acne - F

| | Weight % |
|---|---|
| MCT Oil | 67.39% |
| Hemp Oil | 7.41% |
| Cod Liver Oil | 10.00% |
| Monolaurin | 9.50% |
| Cetyl esters NF | 1.00% |
| RBD palm oil | 4.00% |
| lavender | 0.10% |
| Salicylic Acid | 0.60% |
| Total | 100.000% |
| C8 + 10/sum unsat (Unsat Fat Permeate Factor) | 4.25 |
| sum wax (Retentate Factor) | 10.50 |
| (ALA + SDA)/(EPA + DHA) [PUFA Ratio] | 0.75 |
| ALA + SDA + EPA + DHA (sum omega3) | 3.71 |
| Sat'd Fat > C10:0 | 4.48 |
| (C8 + 10)/sum fat > C10:0 (Sat Fat Permeate Factor) | 15.02 |
| C18:0 | 0.63 |

Omeza Acne F is a gel with a very high Sat Fat Permeate Factor (15.02; (≥3)) and no comedogenic coconut oil (can block skin pores). The Retentate Factor is high (10.5); ≥8.0). Omeza Acne F has 0.6% salicylic acid, a lipid-soluble OTC acne active ingredient.

Application of Omeza Acne-F to teenagers having blackheads and whiteheads and inflamed red spots has eliminated these blackheads (from blocked pores) and whiteheads (from bacterial infection) and has controlled healing of inflamed red spots. Reference is made to FIGS. 7 and 8. In FIGS. 7 and 8, the patient had a severe case of acne. However, application of Omeza Acne-F to the patients everyday by rubbing the composition on the acne daily resulted in a significant reduction in pimples within 4 days. Moreover, there was no scarring.

### Explanation of Example 14

Omeza Acne-F is different from all other gel formulations in that MCT >60% in a mixture with vegetable oil and cod liver oil plus salicylic acid. There are three simultaneous aspects needed for acne relief:

Removal of “dead cells” from plugged hair follicle pores (via salicylic acid).

Control of Propionibacterium acnes (P. acnes). Monolaurin is a well-known antimicrobial compound.

Healing of inflamed skin (MCT and omega3).

The high amount of MCT helps dissolve the lipid-rich dead cells and open clogged pores. The high monolaurin and salicylic acid help kill P. acnes bacteria. Omega3 fatty acids reduce inflammation and jump start the healing process. Further, there is no scarring.

Scarring is caused by excess collagen growth and inflammation. Omega3 and MCT reduce inflammation, reducing the cross-sectional area of the top of the inflamed pore and the distance between the sides of the sores. Reducing local inflammation reduces the need for collagen to “bridge-the-gap” of the sore. If the gap does not need bridging, scarring (i.e. excess collagen build-up) is not needed to close the space between one side of the sore pore and the other side.

### Example 15

The following formulations are prepared in accordance with the procedure described herein:

Omeza Homeopathic
Transdermal - RG4

| | |
|---|---|
| MCT Oil | 41.30% |
| Hemp Oil | 24.97% |
| Cod Liver Oil | 24.00% |
| Monolaurin | 0.60% |
| Cetyl esters NF | 1.00% |
| RBD palm oil | 4.00% |
| Crude coconut oil | 4.00% |
| Protein | 0.13% |
| Total | 100.00% |
| C8 + 10/sum unsat (Unsat Fat Permeate Factor) | 1.00 |
| sum wax (Retentate Factor) | 1.60 |
| (ALA + SDA)/(EPA + DHA) [PUFA Ratio] | 1.00 |
| ALA + SDA + EPA + DHA (sum omega3) | 10.17 |
| Sat'd Fat > C10:0 | 11.73 |
| (C8 + 10)/sum fat > C10:0 (Sat Fat Permeate Factor) | 3.56 |
| C18:0 | 1.53 |

Omeza Homeopathic
Intradermal Gel - RG5

| | |
|---|---|
| MCT Oil | 52.09% |
| Hemp Oil | 12.00% |
| Cod Liver Oil | 16.21% |
| Monolaurin | 9.50% |
| Cetyl esters NF | 1.00% |
| RBD palm oil | 8.25% |
| Crude coconut oil | 0.33% |
| Colloidal oatmeal | 0.50% |
| Protein | 0.12% |
| Total | 100.00% |
| C8 + 10/sum unsat (Unsat Fat Permeate Factor) | 1.96 |
| sum wax (Retentate Factor) | 10.50 |
| (ALA + SDA)/(EPA + DHA) [PUFA Ratio] | 0.75 |
| ALA + SDA + EPA + DHA (sum omega3) | 6.02 |
| Sat'd Fat > C10:0 | 8.35 |
| (C8 + 10)/sum fat > C10:0 (Sat Fat Permeate Factor) | 6.23 |
| C18:0 | 1.10 |

The proteins used in these formulations in this example (RG5) are water soluble, lipid-soluble or something in between in RG4. These formulations hereinabove are homeopathic compounds, i.e., a group of ingredients that are nutritional supplements. These products are typically designed to deliver APIs or cosmetics or lipid-soluble vitamins like Vitamins A, D, E & K. Many of these APIs can be proteins.

In Experiment 15, an oil (RG4) was designed to transport lipid soluble homeopathic proteins (or vitamins) through the dermis into the bloodstream (transdermal). A gel (RG5) was designed to deliver partially lipophilic and lipophobic proteins intradermally between the epidermal basement membrane and the distal side of the upper dermis.

### Explanation of Example 15

RG4 oil is a high Sat Fat Permeate Factor (3.56; (≥3), low Retentate Factor (1.6; (<8.0)), while RG5 is a higher Sat Fat Permeate Factor (5.23; ≥1.0), high Retentate Factor (10.5; ≥8.0). RG4 will drag lipid soluble proteins through the stratum corneum (intradermal). RG5 will form a gel that will support lipid-insoluble proteins during storage and distribution that are not completely lipid soluble. The high RG5 Retentate Factor will meter proteins into the blood stream slowly.

### Example 16

This example illustrates how the formulations of this application can be used in series with other fish oil formulations in treating chronic wounds. These three formulations comprise what will be referenced herein as the “Omeza Bundle”. It consists of Omeza Lidocaine Lavage-AA which is described in the application entitled “A GEL TOPICAL COMPOSITION COMPRISED OF COD LIVER OIL FOR TREATING BURNS AND SKIN DISORDERS” having Serial Number U.S. Ser. No. 63/013,167 (SSMP DOCKET 38029P), the contents of which are incorporated by reference, Collagen Matrix CR, described in International Publication WO 2019/200364 entitled “TOPICAL COMPOSITION OF COD LIVER OIL FOR TREATING WOUNDS AND SKIN DISORDERS, the contents of which are incorporated and Omeza Gel CS1c described hereinabove. The composition of these formulations are provided hereinbelow:

Omeza Lidocaine Lavage - AA

| | Weight % |
|---|---|
| MCT Oil | 32.0% |
| C8 FFA | 0.3% |
| Monolaurin | 0.6% |
| Cetyl Esters NF | 1.0% |
| Lidocaine | 0.8% |
| RBD Palm Oil - CP6 | 14.64% |
| Red Palm concentrate | 0.26% |
| Hemp Oil | 25.2% |
| Cod Liver Oil | 25.2% |
| Total | 100.00% |
| C8 + 10/sum unsat (Unsat Fat Permeate Factor) | 0.66 |
| sum wax (Retentate Factor) | 1.6 |
| (ALA + SDA)/(EPA + DHA) [PUFA Ratio] | 0.97 |
| ALA + SDA + EPA + DHA (sum omega3) | 10.54 |
| Sat'd Fat > C10:0 | 14.20 |
| (C8 + 10)/sum fat > C10:0 (Sat Fat Permeate Factor) | 2.25 |
| C18:0 | 1.95 |

Omeza Gel CS1c
Skin Protectant

| | |
|---|---|
| MCT Oil | 52.09% |
| Hemp Oil | 12.00% |
| Cod Liver Oil | 16.21% |
| Monolaurin | 9.50% |
| Cetyl esters NF | 2.00% |
| virgin coconut oil | 0.33% |
| Colloidal oatmeal | 0.50% |
| RBD palm oil | 7.12% |
| ascorbyl palmitate | 0.25% |
| Total | 100.00% |
| C8 + 10/sum unsat (Unsat Fat Permeate Factor) | 2.00 |
| sum wax (Retentate Factor) | 11.75 |
| (ALA + SDA)/(EPA + DHA) [PUFA Ratio] | 0.75 |
| ALA + SDA + EPA + DHA (sum omega3) | 6.01 |
| Sat'd Fat > C10:0 | 7.78 |
| (C8 + 10)/sum fat > C10:0 (Sat Fat Permeate Factor) | 6.69 |
| C18:0 | 1.05 |

Collagen Matrix
Collagen Matrix CR

| | Weight % |
|---|---|
| MCT Oil | 8.00% |
| C8 FFA | 1.30% |
| Monolaurin | 4.30% |
| Cetyl Esters | 2.00% |
| Yellow Beeswax | 4.00% |
| Triethyl citrate | 2.00% |
| Ethyl linoleate | 2.00% |
| Virgin Coconut Oil | 10.00% |
| Ascorbyl Palmitate | 0.50% |
| Red palm concentrate | 0.20% |
| Hemp oil | 12.00% |
| Cod Liver Oil | 11.40% |
| Fish Collagen | 41.40% |
| Ground Sea Salt | 0.90% |
| Total | 100.00% |
| C8 + 10/sum unsat (Unsat Fat Permeate Factor) | 0.49 |
| sum wax (Retentate Factor) | 10.8 |
| (ALA + SDA)/(EPA + DHA) [PUFA Ratio] | 1.01 |
| ALA + SDA + DHA + EPA (sum omega3) | 4.68 |
| Sat'd Fat > C10:0 | 11.17 |
| (C8 + 10)/sum fat > C10:0 (Sat Fat Permeate Factor) | 0.83 |
| C18:0 | 0.86 |

Lidocaine Lavage-AA is an oil that is applied to non-healing wounds to temporarily numb the site and to encourage a robust oxygen burst. After 5-8 minutes, excess oil is wiped out along with loosened slough and dead microbes. The residual oil from the low Retentate Factor (<8), excess oil mixture leaves a greasy film over the wound that inhibits biofilm attachment.

Collagen Matrix CR is applied to the open wound bed in parallel stripes. Collagen Matrix CR softens at wound temperature and 1.6 g of CR migrates across 18 cm² of wounded surface. Collagen Matrix CR delivers slow-release collagen and low-dose oil over several days.

Omeza Gel-CS1c is rubbed into the periwound from the knee to the toes. High MCT creates a high Permeate Factor (6.23; (≥3)) that tends to drive oil deep into the dermis, but monolaurin >6% (Retentate Factor=10.5 (high); ≥8.0) tends to inhibit the rate of oil transfer. The net effect is to dribble unsaturated lipids into the local dermis and encourage angiogenesis.

### Explanation of Example 16

The Omeza Bundle represents a practical solution to a changing situation.

The chronic wound, as presented, is stuck in the inflammatory stage.

There is little blood flow to the wound bed

There is often a large bioburden in the wound bed.

Necrotic tissue is common.

There is no epidermis in an open wound; the dermis is exposed.

Omeza Lidocaine Lavage-AA (Step 1) numbs the wound to allow sharp debridement without patient pain

During the 5-8 minutes of numbing, low surface tension oil (AA: 28.43 mN/m @ 37° C.) undermines attached necrotic tissue, loosening it.

Sharp debridement and 4×4 slough removal clean the wound bed surface and remove excess oil.

The exposed dermal tissue is saturated with oil.

Low Permeate Factor oil (2.25) combined with low Retentate Factor (1.6; (≥8.0)) creates a sharp surface oxidative burst that uses ROS to “sanitize” the wound surface

Antioxidants (red palm concentrate) quench excess ROS to prevent tissue damage.

There is residual oil retentate on the wound bed that inhibits future biofilm attachment.

Collagen Matrix CR (Step 2) brings hydrolyzed collagen to the wound bed.

Collagen Matrix CR's soluble collagen is encapsulated with wax, inhibiting dissolution.

The low CR Permeate Factor (0.83), combined with Step 1 tissue oil saturation, means Step 2 oil is dribbled into the wound over one week's time.

Omeza Gel-CS1c is a skin protectant rubbed into the entire lower leg.

Omeza Gel-CS1c has a high Permeate Factor (6.23), well above the ≥3 threshold.

High MCT tends to drive unsaturated fats into the periwound dermis (yin).

Monolaurin >6% (Retentate Factor=10.5; ≥8.0) inhibits unsaturated fat transfer into the dermis (yang).

The two forces offset each other.

There is a slight, constant skin temperature rise (Experiment 1).

There is no oxidative burst in the 0-1 mm dermis.

There is a slight perfusion decrease in the 1-3 mm dermis.

Anti-inflammatory oils penetrate deep into the dermis reducing resistance to blood flow.
The body's previously-inhibited natural blood flow control mechanisms can now operate normally.
Use of the three-part Omeza Bundle also reduces scarring.
Other oils (e.g. Wash—C or Wash C with 0.8% added lidocaine) can be used in place of Lidocaine Lavage—AA. Other skin protectant oils (e.g. CB) can be used in place of CS1c.

### Example 17

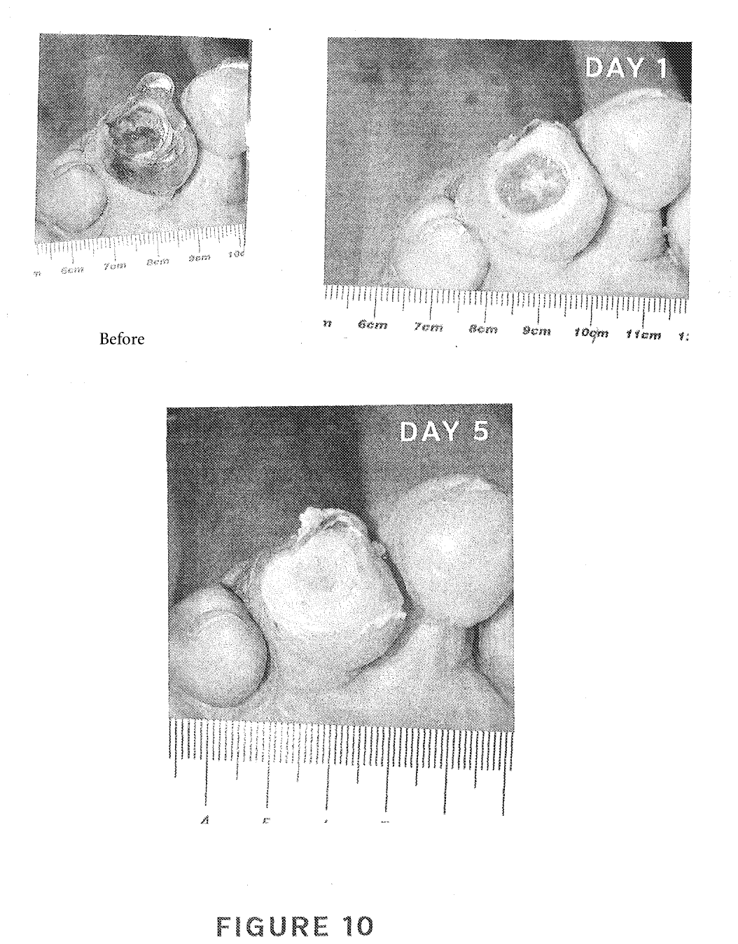

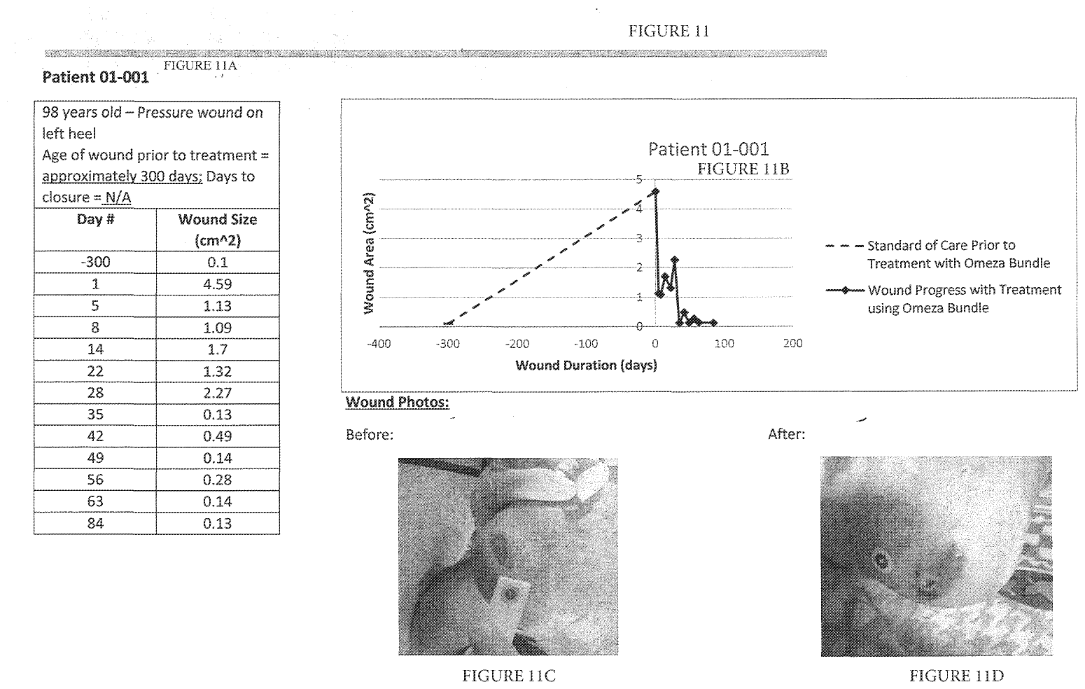

The Omeza Bundle described in Example 16 was utilized on a patient in accordance with the procedure described therein. A male patient developed a diabetic ulcer on his toe. Reference is made to FIG. 10, wherein the injury to the toe is shown. The patient was treated with the Omeza Bundle of Example 16. The rate of healing was unexpectedly rapid. Within 5 days, the toe exhibited significant healing.

### Example 18

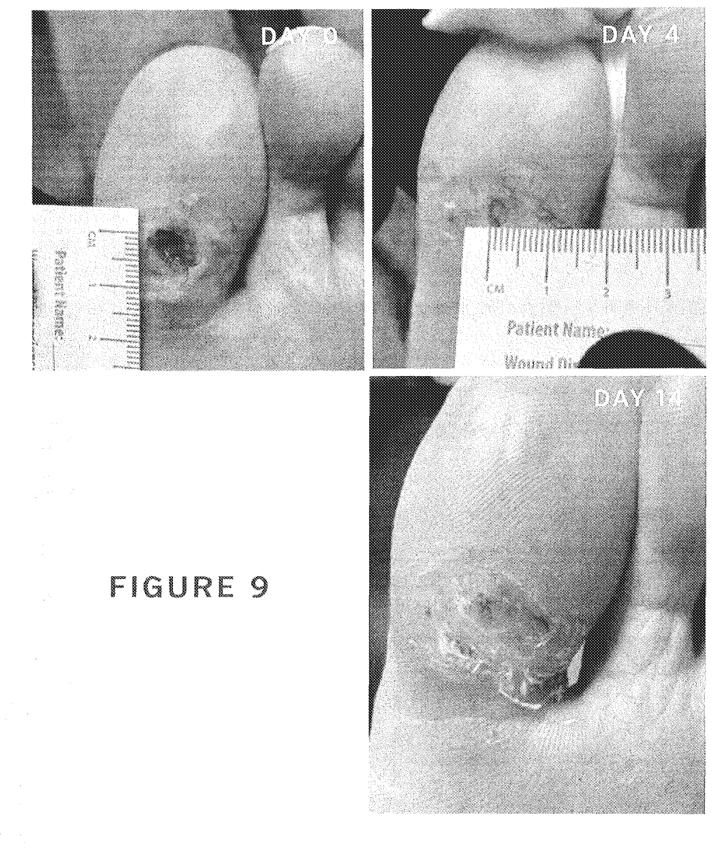

The Omeza Bundle described in Example 16 was utilized on a patient in accordance with the procedure described therein. Reference is made to FIG. 9 showing another patient with diabetic toe ulcer. This diabetic toe ulcer was treated with the Omeza Bundle. The rate of healing is very rapid, as shown in FIG. 9, closing in just 14 days.

### Example 19

Examples 1-18 are all anhydrous mixtures of oils and esters. However, these compositions described herein, including those in examples 1-18 can be mixed with incompatible alcohol/water mixtures to make a hand sanitizer that heals cracked hands (rather than cause them).

The following formulation is prepared in accordance with the procedure described herein and is exemplary of preparing the formulations described herein with alcohol/water mixtures:

Omeza - GOJO E

| | |
|---|---|
| MCT Oil | 45.10% |
| virgin coconut oil | 4.90% |
| Caprylic acid | 1.61% |
| Hemp Oil | 11.92% |
| Cod Liver Oil | 16.17% |
| RBD palm oil | 9.80% |
| Monolaurin | 9.50% |
| Cetyl esters NF | 1.00% |
| Total | 100.00% |
| C8 + 10/sum unsat (Unsat Fat Permeate Factor) | 1.68 |
| sum wax (Retentate Factor) | 10.5 |
| (ALA + SDA)/(EPA + DHA) [PUFA Ratio] | 0.75 |
| ALA + SDA + EPA + DHA (sum omega3) | 6.00 |
| Sat'd Fat > C10:0 | 12.75 |
| (C8 + 10)/sum fat > C10:0 (Sat Fat Permeate Factor) | 3.58 |
| C18:0 | 1.29 |

A standard 70% ethanol/IPA hand sanitizer (99.7%) was mixed with 0.3 wt % GOJO Gel E. Gel E has a high Sat Fat Permeation Factor (3.58 (≥3)) and a high Retentate Factor (10.5; ≥8.0). The Gel is melted and dribbled into the ambient alcohol mixture under high shear (homogenizing) conditions. The gel freezes during shearing and forms a milky suspension in the alcohol.

When rubbed into the hands, the hands are sanitized and have a silky-smooth finish, particularly after multiple uses.

### Explanation of Example 19

Fats are not soluble in alcohol. They coalesce over time and settle to the bottom of the low specific gravity, alcohol bottle. If the fatty mixture has a melting point less than distribution temperatures, the mixture is inherently unstable. By using the gel technology with homogenizing shear, the fat globules become gel globules suspended in alcohol and do not coalesce. In use, the alcohol and water evaporate, leaving high Retentate Factor fat and esters on the skin and low dose, omega3 oil intradermally. The skin is protected; cracks heal. Users become more compliant because their hands do not hurt.

The low doses of GOJO E are necessary because, for example, hospital workers or bank tellers, sanitize their hands in excess of 100 times per 8 hour shift. The build-up on the skin would be excessive if the GOJO E dose were higher, for example 0.5 wt %.

### Example 20

The following compositions are prepared in accordance with the procedures described herein:

Omeza Periwound Protection - A8

| | |
|---|---|
| MCT Oil | 40.10% |
| Cod Liver Oil | 47.15% |
| Monolaurin | 10.00% |
| yellow beeswax | 1.00% |
| Red Palm concentrate | 0.13% |
| Colloidal oatmeal | 0.12% |
| caprylic acid | 1.50% |
| Total | 100.00% |
| (C8 + C10)/sum unsat (Unsat Fat Permeate Factor) | 1.18 |
| sum wax (Retentate Factor) | 11.00 |
| (ALA + SDA)/(EPA + DHA) [PUFA Ratio] | 0.11 |
| ALA + SDA + EPA + DHA (sum omega3) | 11.13 |
| Sat'd Fat > C10:0 | 7.92 |
| (C8 + 10)/sum fat > C10:0 (Sat Fat Permeate Factor) | 5.05 |
| C18:0 | 1.23 |

Formulation A8 is very different than any other formulation presented herein because it does not contain hemp oil nor cetyl esters. It is deliberately greasy. Yellow beeswax is added. Yellow beeswax is added to raise the melting point of the gel above body temperature. A8 is used in combination with sheets of, for example, cross-linked collagen as a way to “caulk” the sheet to the periwound skin. This is particularly efficacious on wounds >18 cm². A8 does not melt in use and so the edges of the sheet remain in close contact with the wound. Hemp oil is replaced by cod liver oil. A8 is used for reduced periwound inflammation. A8 is a thick grease that “caulks” the now-flexible sheet into place in the wound bed. Care is taken to coat the periwound “face” directly adjacent to the open wound with A8.

Omeza Wound Wash - C
Spray

| | |
|---|---|
| MCT Oil | 52.00% |
| Hemp Oil | 13.70% |
| Cod Liver Oil | 13.500% |
| Monolaurin | 0.60% |
| Cetyl esters NF | 1.00% |
| virgin coconut oil | 0.33% |
| RBD palm oil | 18.40% |
| caprylic acid | 0.47% |
| Total | 100.00% |
| C8 + 10/sum unsat (Unsat Fat Permeate Factor) | 1.67 |
| sum wax (Retentate Factor) | 1.60 |
| (ALA + SDA)/(EPA + DHA) [PUFA Ratio] | 1.00 |
| ALA + SDA + EPA + DHA (sum omega3) | 5.73 |
| Sat'd Fat > C10:0 | 13.17 |
| (C8 + 10)/sum fat > C10:0 (Sat Fat Permeate Factor) | 3.94 |
| C18:0 | 1.53 |

Omeza Wound Wash—C is a 510k medical device that is used to lubricate a dry collagen sheet before applying to the wound bed. Typically, the stiff dry sheet is cut-to-fit the wound and then lubricated with C. The now-flexible sheet is placed in the wound. But the fit is never perfect.

Wound Wash-C can be modified by adding 0.8 wt % lidocaine and removing 0.8 wt % palm oil (to balance the formula). In this guise, Wound Wash-D becomes an OTC alternate (topical analgesic) in the Omeza bundle for Lidocaine Lavage AA.

Omeza Topical Supplement - D
Spray

| | |
|---|---|
| MCT Oil | 41.30% |
| Hemp Oil | 24.97% |
| Cod Liver Oil | 24.000% |
| Monolaurin | 0.60% |
| Cetyl esters NF | 1.00% |
| virgin coconut oil | 4.00% |
| RBD palm oil | 4.00% |
| perfume-B | 0.13% |
| Total | 100.00% |
| C8 + 10/sum unsat (Unsat Fat Permeate Factor) | 1.00 |
| sum wax (Retentate Factor) | 1.6 |
| (ALA + SDA)/(EPA + DHA) [PUFA Ratio] | 1.00 |
| ALA + SDA + EPA + DHA (sum omega3) | 10.17 |
| Sat'd Fat > C10:0 | 11.73 |
| (C8 + 10)/sum fat > C10:0 (Sat Fat Permeate Factor) | 3.56 |
| C18:0 | 1.53 |

### Explanation of Example 20

Matrix CR is best used on small and medium sized wounds (i.e. up to 18 cm²). For bigger wounds, it is often best to use a large sheet of collagen. But large sheets have their own defects, primarily their inability to conform to the wound irregularities. By lubricating them with Wound Wash C or Wound Wash D, the sheet becomes flexible enough to contour to the irregularities. But even the most carefully laid sheet does not 100% cover the wound, particularly at the edges of the sheet. Wound Wash C or Wound Wash D is used to “wet” the sheet and edges and bind the edges to the periwound. Both Wound Wash C (Sat Fat Permeate Factor=3.94) and Wound Wash D (Sat Fat Permeate Factor=4.07) have Permeate Factors >3, so they can be applied from spray cans without fear of nozzle clogging from cloudiness.

The periwound is prone to excess moisture, particularly when the collagen sheet is oil impregnated. Since oil and water are incompatible, exudate (mostly saline water) is shunted towards the periwound. The “A8” grease has a melting point just higher than body temperature, so the high retentate factor (11.0) does not melt, instead stays attached to the periwound and protects the periwound from excess moisture (and consequent maceration).

Large wounds are large exudate producers and need periwound protection to heal.

Prior art exudate control strategies use negative pressure vacuum devices to suck excess exudate away. But this strategy makes the wound anaerobic and slows healing.

Still other large wound strategies involve high pressure oxygen (barometric). This strategy selects for aerobic microbial growth, keeping the wound infected. If high strength antibiotics are used, commensal bacteria are inhibited, and the wound doesn't heal.

The negative pressure and barometric strategies often reduce the size of the wound, but rarely close the wound. As such, these very expensive modalities are used to boost Medicare reimbursements without healing the patient. The Example 20 embodiment heals patients with oversized wounds.

The inventive embodiment allows excess exudate to leak out and away from the wound and evaporate. The residual oil keeps the wound bed and periwound greasy, so biofilm cannot attach. Together, commensal bacteria outcompete pathogens; wounds heal without periwound maceration. When the wound closes to within 18 cm², the Omeza Bundle is used to finish the job.

Stem cells from adjacent hair follicles and the interfollicular skin migrate to the wound when signaled for. If the periwound is inflamed (e.g. macerated), then the periwound is swollen above normal height (via inflammation), and stem cell migration is physically inhibited (cannot “climb” over the inflamed periwound “hill”). When stem cells cannot physically enter the wound bed (for example because the macerated periwound acts like a wall around the wound bed), then the wound does not heal.

There is no hemp oil or any other vegetable oil having an omega3 fatty acid content greater than 9 wt % present in A8.

Eliminating hemp oil eliminates C18 omega3 oils that are inflammatory.

The C>18 omega3 oils are anti-inflammatory

Yellow beeswax replaces Cetyl Esters NF (both are waxes)

Yellow beeswax increases the gel melting point from 36.9° C. to 38.4° C.

This mathematically small change affects the melting properties of A8 when applied topically to the human body because the two melting points are on either side of normal body temperature (37.0° C.). Thus, other gels melt when applied to the body (36.9° C. <37.0° C.); A8 does not melt (38.4° C.>37° C.).

This keeps A8 applied to the periwound to protect it continuously between treatments.

Colloidal oatmeal is present in A8.

Colloidal oatmeal >0.07 wt % is an OTC skin protectant active ingredient.

A8 is useful as a “Skin Protectant”

A8 cannot be applied to large or open wounds, but it can be applied along the periwound edge of the wound on top of an FDA-approved ECM (Extra-Cellular Matrix) to help protect periwound skin from maceration.

Caprylic acid and monolaurin inhibit biofilm adherence to periwound skin. They keep the periwound greasy and inhibit physical bacterial attachment to greasy periwound skin.

Large chronic wounds (>18 cm²) are different than small chronic wounds because they are growing wounds, not static or declining wounds. They are often infected; they typically ooze exudate that can macerate the immediate periwound. A macerated periwound is a physical barrier to stem cell migration into the wound bed.

The sheer size of large wounds makes it difficult to apply a semi-solid collagen matrix evenly over the wound bed. With large wounds, therapy is often a negative pressure vacuum device designed to create an anaerobic environment and to vacuum excess exudate away. An alternate therapy is hyperbaric oxygen which creates an aerobic environment.

### Example 21

Another therapy involves placing saline-hydrated external ECM (Extra Cellular Matrix) over the wound bed as a scaffold to attract healing compounds or as a source of collagen. Examples of ECM include placental tissue, cross-linked animal collagen, fish skin collagen and other non-cross-linked collagens that are pressed or woven into a sheet. There are literally 100's of commercially available ECM examples.

The practitioner takes the sterile ECM and shapes the piece to the specific wound geometry, wets the sheet with sterile saline and carefully places the cut sheet into the wound (like placing a final jigsaw puzzle piece in place). Invariably, the fit is inexact as the wound heals. Incorporated ECM stays in the wound; excess ECM is removed, sometimes traumatically. If the ECM extends onto the immediate periwound the ECM dries out eventually and has to be physically removed, traumatizing the periwound.

A better solution is to “caulk” the ECM into the “periwound frame” and to “wet” the ECM edge (thus holding the ECM edge in place). If the “caulk” is greasy, the periwound is simultaneously protected from excess moisture maceration between treatments.

In Example 21, an improved Lidocaine Lavage (AF2) is used to “wet” the ECM sheet with an anhydrous oil mixture. A greasy gel (A8) is the “caulk”.

Omeza Lidocaine Lavage - AF2

| | Weight % |
|---|---|
| MCT Oil | 32.00% |
| C8 FFA | 0.55% |
| Monolaurin | 0.60% |
| Cetyl Esters NF | 1.00% |
| Lidocaine | 0.80% |
| RBD Palm Oil | 14.80% |
| Hemp Oil | 25.50% |
| Cod Liver Oil | 24.75% |
| Total | 100.00% |
| C8 + 10/sum unsat (Unsat Fat Permeate Factor) | 0.66 |
| sum wax (Retentate Factor) | 1.60 |
| (ALA + SDA)/(EPA + DHA) [PUFA Ratio] | 1.00 |
| ALA + SDA + EPA + DHA (sum omega3) | 10.49 |
| Sat'd Fat > C10:0 | 14.13 |
| (C8 + 10)/sum fat > C10:0 (Sat Fat Permeate Factor) | 2.26 |
| C18:0 | 1.95 |

The practitioner cuts the ECM to fit the wound. Instead of saline, ECM is “wet” with anhydrous oil (AF2) and placed in the wound bed. Then Gel A8 is added to the immediate periwound on top of wet ECM, caulking ECM into the wound. The periwound face is covered with A8 grease to protect the periwound face from maceration. The wound is covered and wrapped using prior art practice.

It is well known that hair follicles are intimately involved with wound healing. In a large wound, there are no hair follicles in the wound bed. Hair follicles are a reservoir for several types of epithelial stem cells. The skin between adjacent hair follicles also contain epithelial stem cells. The stem cells are not the same. When called by signals from the wound, stem cells (particularly their progeny) migrate across the interfollicular basement membrane towards the wound. Stem cells from hair follicles mix together with skin (basal cell) stem cells during this transition and flow together (“like lava”) across the periwound and onto the wound bed.

If the periwound is macerated and swollen, this migration is physically inhibited. Conversely, if the periwound is protected by A8 from maceration, the periwound is not swollen and stem cell flows migrate without physical inhibition into the wound. Chronic wounds treated with A8 heal as acute wounds. The change is subtle, but surprisingly, converts chronic wounds that cannot heal into acute wounds that heal in an orderly progression.

### Example 22

This example shows the results of the effect of the Omeza Bundle, described in Example 16 in healing eight elderly patients having different wounds, using the procedure described therein. One elderly patient, age 98, had a stage 3 ulcer pressure wound on the left heel with a surface area of 4.6 cm²; another, age 93, had a stage 2 ulcer pressure wound on the left gluteus having a surface area of about 1.18 cm²; another elderly patient, 86 years old, had a stage 2 ulcer pressure wound on the left ischial having a surface area of about 2.9 cm²; fourth elderly patient, age 86, had pressure wound on left coccyx; a fifth elderly patient, age 97, had two wounds, one being a left elbow laceration having a surface area of about 4.4 cm², and the other being a scalp laceration, having a surface area of about 1.3 cm²; a sixth elderly patient, age 87, had a wound on the rear right trochanter; a seventh elderly patient, age 86, had a right lateral lower leg laceration and an eighth elderly patient, age, 94, had a stage 3 pressure wound on the right gluteal fold. Each were given the same treatment regimen. Lidocaine Lavage-AA was applied to cover each of the wounds. After 5 minutes, loose necrotic tissue and any unabsorbed composition are wiped out of the wound bed loci, Additional necrotic tissue may be sharply debrided from the bed of the wound with a scalpel. Additional Lidocaine Lavage-AA was applied to the wound loci, and after 5 minutes, any excess composition and any additional necrotic tissue were wiped away. Next Collagen Matrix CR is applied to the wound area to cover the wound as the primary dressing. If the wound is on the leg or foot, Omeza Gel CS1 is applied on the periwound and surrounding intact skin from the knees to the toes. For wounds located in other areas, Omeza Gel CS1 is applied in the periwound all around the wound, which commences on the wound edge and extends to a five cm diameter from the edge of the wound. A foam or other specialty absorptive dressing is used as a secondary dressing. The dressing applications, i.e., Collagen Matrix CR and the Omeza Gel CS1, were changed twice a week for up to 12 weeks or until the wound closes prior to 12 weeks. If the wound closes prior to 12 weeks, the application of the Collagen Matrix CR dressing is discontinued, but the application of the Omeza Gel CS1 is continued and applied twice weekly until the end of the 12 week period to keep the skin supple and reduce the risk of wound reoccurrence.

The results of eight patients are shown in FIGS. 11-19. The x-axis in each of the graphs represents the time (days) that it took for the wound to close, while the y-axis is the calculated surface area of the wound (cm²), as measured at the time of treatment. As shown by FIG. 11, the pressure wound on the 98 year old patient having an area of 4.59 cm² was significantly reduced within 84 days. Further, the pressure wound on the 93 year old patient having a surface area of 1.14 cm² closed within 80 days. The pressure wound having a surface area of 2.81 cm² on the 86 year old patient closed within 30 days, as shown in FIG. 13. The wound having a surface area of 1.16 cm² on a different 86 year patient had closed within 90 days, as shown in FIG. 14. Further, the scalp laceration of 0.1 cm² on the 97 year old patient healed in 23 days (FIG. 15), while the left elbow laceration having a surface area of 4.28 cm² on the same patient closed within 43 days (FIG. 16). The wound having a surface area of 1.86 cm² on the 87 year patient closed within 43 days (FIG. 17). The wound of 10.81 cm² on the 86 year old closed within 71 days, as shown in FIG. 18. Finally, the wound on the 94 year old patient closed within 64 days.

In FIGS. 11, 14, 18, 19 the wound area was enlarged before it got smaller. Chronic wounds are not like acute wounds, particularly pressure wounds. The immediate periwound is often damaged; epithelial separation from the underlying dermis may not yet have occurred. Treatment with low surface tension Lidocaine Lavage AA undermines the terminally-damaged tissue, releasing the damaged epithelial tissue from the underlying dermis. The wound enlarges and then heals. Pressure wounds are historically hard to heal for this reason. The Omeza Bundle removes terminally damaged skin by non-enzymatic autolytic debridement and then heals the wound. Other autolytic debridement protocols use added enzymes. Sharp debridement uses a scalpel, but may miss some terminally damaged epithelial tissue. On the other hand, the Omeza Bundle uses low surface tension oil (Lidocaine Lavage-AA) to release the damaged epithelium from the underlying dermis and then oils and collagen (CR) to heal the wound. This worse-before-it gets-better is characteristic of the Omeza Bundle and is a major reason why the repairs not only close the wound, but keep them closed. Other protocols may close the wound, but the repair is on terminally-damaged periwound skin. To get a permanent repair, the wound+terminally damaged skin have to be replaced by new epithelium. The Skin Protectant (CS1) helps stem cell progeny migrate from the undamaged periwound to the now-enlarged wound site. After the migration, healing is rapid, even though the wound area apparently increased. [The wound area that heals=initial visible wound area+terminally damaged periwound.]

### Example 23

There are many formulations presented with different permeation and retentate parameters.

Table 2 summarizes the presented formulations in Examples 1-22 in tabular form for easier comparison.

TABLE 2

| | | Sat Fat | |
|---|---|---|---|
| | | Permeate | Retentate |
| | | Factor | Factor |
| OIL | | | |
| | Oil BV | 2.1 | 1.60 |
| | Lidocaine Lavage-AA | 2.25 | 1.60 |
| | Spray D | 3.56 | 1.60 |
| | Transdermal RG4 | 3.56 | 1.60 |
| | Oil CB | 1.59 | 1.60 |
| | Wash- C | 3.94 | 1.60 |
| GEL | | | |
| | Sleep Aid PM3 | 4.26 | 10.50 |
| | Protection A8 | 5.05 | 11.00 |
| | GOJO E | 3.58 | 10.50 |
| | Gel AF | 1.32 | 11.50 |
| | CS1c gel | 6.69 | 11.75 |
| | Intradermal RG5 | 6.23 | 10.50 |
| | Gel CS4 | 6.42 | 10.50 |
| | Gel CS3a | 7.32 | 10.50 |
| | Pain Relief CN | 8.12 | 10.50 |
| | Acne F | 15.02 | 10.50 |
| RINSE-OFF | | | |
| | Collagen Matrix DD | 2.53 | 9.80 |
| | Collagen Matrix DE | 2.63 | 9.86 |
| LEAVE-ON | | | |
| | Collagen Matrix CR | 0.83 | 10.80 |
| | First Aid Antiseptic | | |
| | DF | 1.45 | 10.25 |

The various combinations of permeate and retentate are shown in the following embedded tables:

Oils and Gels
Retentate Factor

| | | |
|---|---|---|
| high | oil; non-greasy residual | gel; non-greasy residual |
| Permeate Factor | fast penetration | slow penetration |
| low | oil; greasy residual | gel; greasy residual |
| | low | high |

Collagen Gels
Retentate Factor

| | | |
|---|---|---|
| high | sustained oil release | rapid oil penetration |
| Permeate Factor | unstable | unstable |
| low | | |
| | low | high |

Low:low, high:high, low:high, high:low combinations are shown for oil and gel embodiments and also for collagen embodiments. The primary differences are described in the various quadrants. The Example Key Ratios table shown just before the examples shows the clear breaks between low and high Retentate Factors and Permeate Factors for oils/gels and collagen. What is significant is that formulas with distinct characteristics can be grouped in each quadrant. Thus the quadrant technique is a vehicle for communicating the differences to others. The specific characteristics of each formulation are described in the examples.

It is clear that collagen products differ from oil&gel products, so the data are presented as different quadrant groups in a collagen and separately in quadrant groups for oils & gels. Oils & gels differ in retentate factor; leave-behind collagens and rinse-off collagens differ in permeate factor.

### Example 24

In FIG. 6 (Example 4), a chemical burn on a human scalp is shown. This wound is treated with Lidocaine Lavage-AA and First Aid Antiseptic DF. Initial healing is rapid; later healing is slow but steady.

The patient was non-compliant for 30 days after the Day 34 picture as she decided whether to continue the Omeza treatment or begin a 12-month regimen of skin grafting and surgical hair implants. The Omeza treatment restarted around Day 60.

Human head hair is in anagen growth for almost 30 years, so there is no telogen-to-anagen activation that generates a pulse of stem cell progeny. Instead, once the wound skin has healed, hair infiltration is slow. In the 92 day photo, the wound skin has healed and there is slow hair migration into the wounded area.

### Comparative Example 2

A man's thumb was crushed between a toe hitch and the trailer. Doctors recommended amputation. The patient refused and used Lidocaine Lavage AA (oil) as a daily treatment. The thumb healed as shown in FIG. 22, without extensive scarring beyond that caused by the actual sutures.

### Explanation of Comparative Example 2

The man's thumb healed because the saturated fats on the wound surface prevented biofilm attachment. There is suture scarring in Day 26. There was no indication of hair follicle telogen-to-anagen transition. Hair growth was not observed. Scar-free healing requires inducing follicles to transition from resting to active growth using novel gels.

In the example below, the weight ratio of the sum of the weight of the medium chain triglycerides to long chain triglycerides (C8+C10 saturated triglycerides)/sum of the weight of the saturated triglycerides larger than 10 carbon atoms is the MCT/LCT ratio.

### Example 25 and Comparative Example 3

In Example 25, the various compounds, except the perfumes, are heated to 145° F. until the mixture is homogenous and clear. The mixture is allowed to cool (with gentle stirring) to 110° F. Perfume is next added. Mixing is stopped after 2 additional minutes and the clear oil is transferred to a 5 quart mixing bowl. With a dough hook mixer, the bowl is stirred (1.5 rpm) until the mixture first becomes opaque (˜102.5° F.). Mixing stops and the bowl is left to cool and congeal overnight.

Omeza Gel AF
Skin Protectant
Omeza Skin Protectant

| | |
|---|---|
| MCT Oil | 32.14% |
| C8 FFA | 0.79% |
| Monolaurin | 9.50% |
| Cetyl esters NF | 2.00% |
| Coconut oil | 29.70% |
| Hemp Oil | 13.20% |
| Cod Liver Oil | 12.64% |
| perfume | 0.03% |
| Total | 100.00% |
| C8 + 10/sum unsat (Unsat Fat Permeate Factor) | 1.70 |
| sum wax (Retentate Factor) | 11.5 |
| (ALA + SDA)/(EPA + DHA) [PUFA Ratio] | 1.000 |
| ALA + SDA + EPA + DHA (sum omega3) | 5.36 |
| (C8 + 10)/sum fat > C10:0 (Sat Fat Permeate Factor) | 1.32 |
| C18:0 | 1.43 |

SP-AF was a firm gel, easily spread and melted @ 97.1° F., just below body temperature. SP-AF had no fish odor.

The rate of absorption was compared with that of the following composition:

Omeza Lidocaine Lavage - AA

| | Weight % |
|---|---|
| MCT Oil | 32.0% |
| C8 FFA | 0.3% |
| Monolaurin | 0.6% |
| Cetyl Esters NF | 1.0% |
| Lidocaine | 0.8% |
| RBD Palm Oil - CP6 | 14.64% |
| Red Palm concentrate | 0.26% |
| Hemp Oil | 25.2% |
| Cod Liver Oil | 25.2% |
| Total | 100.00% |
| C8 + 10/sum unsat (Unsat Fat Permeate Factor) | 0.66 |
| sum wax (Retentate Factor) | 1.6 |
| (ALA + SDA)/(EPA + DHA) [PUFA Ratio] | 0.97 |
| ALA + SDA + EPA + DHA (sum omega3) | 10.54 |
| Sat'd Fat > C10:0 | 14.20 |
| (C8 + 10)/sum fat > C10:0 (Sat Fat Permeate Factor) | 2.25 |
| C18:0 | 1.95 |

This product is Example 28 in WO/2019/200361.

For purposes of comparison, 1.0 microliter drop of Omeza Lidocaine Lavage AA and Omeza Gel AF were each placed topically on the forearm skin of three females and one male. The contact angle of each drop was measured in real time and the drop volume calculated. The amount absorbed is the difference in volume of the initial drop and the remaining drop volume at each measuring point. On the average, the data (shown in the Example 3 table) indicated that Omeza Lidocaine Lavage AA absorbs into skin approximately 1.75 times faster than Omeza Gel AF.

The omega3 oils in the gelled formulation penetrated into the skin slower than the non-gelled oil.

### Example 26

The following example is prepared in accordance with the procedure of Example 25:

Oily Skin - H

| | |
|---|---|
| MCT Oil | 29.05% |
| Hemp Oil | 13.15% |
| Cod Liver Oil | 13.00% |
| Monolaurin | 9.50% |
| Cetyl esters NF | 2.00% |
| virgin coconut oil | 13.00% |
| RBD palm oil | 19.00% |
| caprylic acid | 1.20% |
| perfume B | 0.10% |
| Total | 100.00% |
| C8 + 10/sum unsat (Unsat Fat Permeate Factor) | 1.00 |
| sum wax (Retentate Factor) | 11.50 |
| (ALA + SDA)/(EPA + DHA) [PUFA Ratio] | 1.00 |
| ALA + SDA + EPA + DHA (sum omega3) | 5.51 |
| Sat'd Fat > C10:0 | 23.47 |
| (C8 + 10)/sum fat > C10:0 (Sat Fat Permeate Factor) | 1.31 |
| C18:0 | 1.86 |

The product of Example 26 is a gel that is a clear liquid at 40° C. It turns to a uniform gel at ˜35° C. without external shear. When rubbed into the legs, body temperature melts the gel and it is absorbed. The absorption rate is about half the absorption rate of an equivalent oil (with 0.6% monolaurin).

There is an unexpected benefit: better odor control. With the product of Example 24 oil, the absorption rate is about half that of Comparative Example 3. A trained panel rubbed the oil into the underside of the forearm; gel was rubbed into the other forearm. Odor perceptions were recorded on a 1-5 scale and results compared. The semi-occlusive layer reduced odor escape. This is executed by keeping the (C16:0+C18:1) triglyceride/monolaurin ratio in a range of 1 to 3. C16:0 and C18:1 chain-lengths are ubiquitous in most of the natural oils (no C18:1 in virgin coconut oil). The time to rub-in 0.25 g of product into the left and right forearm was compared. Trained testers compared and contrasted the odor and skin feel of Omeza Oily Skin—H to Lidocaine Lavage AA in Comparative Example 3. The gel has higher monolaurin and meters the oil into the skin slowly. The rub-in time is extended 75%, but the residual odor is reduced significantly (from very little to non-detect).

### Example 26a

The following example is prepared as in Example 26:

Skin Protectant Gel - G

| | |
|---|---|
| MCT Oil | 43.20% |
| Hemp Oil | 11.49% |
| Cod Liver Oil | 11.00% |
| Monolaurin | 9.50% |
| Cetyl esters NF | 2.00% |
| virgin coconut oil | 21.50% |
| caprylic acid | 1.21% |
| perfume B | 0.10% |
| Total | 100.00% |
| C8 + 10/sum unsat (Unsat Fat Permeate Factor) | 2.49 |
| sum wax (Retentate Factor) | 11.50 |
| (ALA + SDA)/(EPA + DHA) [PUFA Ratio] | 1.00 |
| ALA + SDA + EPA + DHA (sum omega3) | 4.66 |
| Sat'd Fat > C10:0 | 20.23 |
| (C8 + 10)/sum fat > C10:0 (Sat Fat Permeate Factor) | 2.27 |
| C18:0 | 1.14 |

Gel-G, was an attempt to increase the absorption rate to reduce odor. Using the forearm procedure described in Example 25 above, the opposite occurred; it slowed the absorption rate (data not shown) and had a strong fish odor. 5 trained panelists rubbed each product into the forearm until the skin was silky smooth, then rated the skin for odor on a 1 (no odor)-5 (malodor) scale. Gel-G was rated 4 (on average). There was almost 1.5× more MCT to solubilize the fat and drive it into the dermis. The omega3 oils were reduced to reduce the odor problem. Palm oil was removed to reduce the C16:0 and C18:1. But it didn't work.

The palm oil used in Example 26 is RBD palm oil CP6 (often referred to as Super Olein, or double winterized). In another formulation, Red palm oil (not winterized) was substituted. A white fatty layer precipitated @ 25° C. after 2 days. This product was also greasy and not satisfactory.

Without wishing to be bound, it is believed that layering is taking place with the Cetyl Esters predominant in the outside layer (the silky-smooth layer). C12:0 triglyceride forms an amorphous layer beneath the Cetyl Ester layer that is absorbed @ 37° C., allowing odor to escape. C16:0 is the major fatty acid in the epidermis. C16:0+C18:1 is a structured-but-flexible layer, that stops in the epidermis and moves with the skin, so it maintains its odor-barrier integrity.

This example shows that excess monolaurin (9.5 wt % versus 0.6 wt %) materially slows down absorption, changes perfusion deep in the dermis, but does create a gel that eliminates the slip and slide of the oily composition of WO2019/200364.

### Example 27

The following example is prepared as in Example 25:

Oily Skin - I Eye Gel

| | |
|---|---|
| MCT Oil | 47.97% |
| Hemp Oil | 10.09% |
| Cod Liver Oil | 10.00% |
| Monolaurin | 9.50% |
| Cetyl esters NF | 1.00% |
| virgin coconut oil | 5.24% |
| RBD palm oil | 16.10% |
| perfume B | 0.10% |
| Total | 100.00% |
| C8 + 10/sum unsat (Unsat Fat Permeate Factor) | 2.00 |
| sum wax (Retentate Factor) | 10.50 |
| (ALA + SDA)/(EPA + DHA) [PUFA Ratio] | 1.00 |
| ALA + SDA + EPA + DHA (sum omega3) | 4.24 |
| Sat'd Fat > C10:0 | 15.04 |
| (C8 + 10)/sum fat > C10:0 (Sat Fat Permeate Factor) | 3.23 |
| C18:0 | 1.38 |

Omeza Skin-I composition has a 3-to-1 palm-to-coconut weight ratio. This formula is absorbed quickly, and there is no odor; circulation is stimulated; there is no slip/fall issue. What was determined is there is a need for both palm and coconut oil.

### Example 28

The following compositions are prepared as in Example 25:

Oily Skin - I Eye Gel

| | |
|---|---|
| MCT oil | 47.97% |
| Hemp Oil | 10.09% |
| Cod Liver Oil | 10.00% |
| Monolaurin | 9.50% |
| Cetyl esters NF | 1.00% |
| virgin coconut oil | 5.24% |
| RBD palm oil | 16.10% |
| perfume B | 0.10% |
| Total | 100.00% |
| C8 + 10/sum unsat (Unsat Fat Permeate Factor) | 2.00 |
| sum wax (Retentate Factor) | 10.50 |
| (ALA + SDA)/(EPA + DHA) [PUFA Ratio] | 1.00 |
| ALA + SDA + EPA + DHA (sum omega3) | 4.24 |
| Sat'd Fat > C10:0 | 15.04 |
| (C8 + 10)/sum fat > C10:0 (Sat Fat Permeate Factor) | 3.23 |
| C18:0 | 1.38 |

Oily Skin - E Leg Gel

| | |
|---|---|
| MCT Oil | 39.34% |
| Hemp Oil | 13.38% |
| Cod Liver Oil | 13.00% |
| Monolaurin | 9.50% |
| Cetyl esters NF | 2.00% |
| virgin coconut oil | 13.00% |
| RBD palm oil | 9.00% |
| caprylic acid | 0.68% |
| perfume B | 0.10% |
| Total | 100.00% |
| C8 + 10/sum unsat (Unsat Fat Permeate Factor) | 1.58 |
| sum wax (Retentate Factor) | 11.50 |
| (ALA + SDA)/(EPA + DHA) [PUFA Ratio] | 1.00 |
| ALA + SDA + EPA + DHA (sum omega3) | 5.51 |
| Sat'd Fat > C10:0 | 18.48 |
| (C8 + 10)/sum fat > C10:0 (Sat Fat Permeate Factor) | 2.22 |
| C18:0 | 1.42 |

This example shows two different formulations for different parts of the body. The face formulations have very low surface tensions (˜28 mN/m). This is lower than the surface tensions of known oils (≥30 mn/m). This small difference is important; it allows cosmetic make-up to be smoothly applied.

The face formulation has weight ratio of (C8+C10) triglyceride/sum unsat ratios ≥2. The leg gel has a ratio <2.

MCT is used to drive the other fats through the stratum corneum. Once in the dermis, MCT feeds cells to produce anti-inflammatory M2 macrophages. The omega3 fats are natural anti-inflammatory compounds.

When the face gel is applied directly after a shower, the low surface tension moves produced sebum uniformly across the face. In combination with the monolaurin and cetyl ester, a smooth low surface tension surface is presented. Cosmetics are applied smoothly, and the beauty effect is obvious to users.

The leg gel is applied as a gel to eliminate the slip and fall concerns of prior art formulations. When the skin surface is rinsed with tap water, a water-soluble film (sebum+) is removed, leaving behind a silky smooth contiguous, cosmetically elegant surface.

### Explanation of Example 28

In recent years, sebum oil has been found to play a key role in the regulation of the hydration of the outermost layer of the skin, the stratum corneum. Sebum contains triglycerides, cholesterol, ceramides and various waxes. The triglycerides and their relative abundance are listed in the Table below. These triglycerides are present in Example 28 gels (crude coconut (sometimes known as virgin), palm oil, hemp oil and cod liver oil). Cetyl Esters NF is a mixture of chain lengths, closest to #7 Myristyl palmitate and #8 (palmityl palmitate). Example 27 gels contain many similarities to sebum.

| | Sebum Triglycerides |
|---|---|
| | (recovered from hair) |
| | Index |
| Triglyceride | |
| 1. Myristic acid (C14) | 100 |
| 2. Palmitic acid (C16) | 215 |
| 3. Stearic acid (C18) | 99 |
| 4. Oleic acid (C18.1) | 163 |
| 5. Cholesterol | |
| Waxes | |
| 6. Squalene | |
| 7. Myristyl palmitate (C14-C16) | |
| 8. Palmityl palmitate (C16-C16) | |
| 9. Stearyl palmitate (C16-C18) | |

The ability of the skin to be wetted (low surface tension) by water is an important parameter for the application of cosmetic products and is also involved in the cutaneous ecosystem. The hair follicle is a vector for transport of fluids from the dermis to the skin surface. The absence of a hair follicle is a vector for transporting excess fluids away from the skin surface and deeper into the dermis.

By analogy, the hair follicle/sebaceous gland/sweat gland is a bioreactor with an upward relief valve. When fluids (like MCT and omega3) feed the sebaceous gland, enzymes convert the triglycerides into free fatty acids, ceramides and wax esters. The produced fluid fills the annular space around the follicle and is extruded up and out to the surface, producing the acid mantle. However, the topical application of the gel composition to the skin of the subject results in feeding the sebaceous gland and hair follicle simultaneous high levels of MCT, palm oil, coconut oil, and omega3 fats, changing the chemistry of the produced sebum.

The increased sebum production brings fish oil to the surface where it can be smelled 20 minutes after application. The solution was to increase the palm oil/coconut oil ratio to >1. The leg formula palm/coconut ratio=0.7. The leg is far enough away from the nose that the slight late-blooming odor is not perceived.

The gels that are applied to the hairy regions leave behind a wax layer as the oils are absorbed. Produced sebum coats the waxy layer with triglycerides, FFA, ceramides (etc.) and water. When the water is rinsed away, soluble FFA is rinsed away and the wax/insoluble-sebum layer is silky smooth and ready for cosmetic application (on the face).

### Example 29

The following formulation is prepared as in Example 25:

Omeza Gel AF
Skin Protectant
Omeza Skin Protectant

| | |
|---|---|
| MCT Oil | 32.14% |
| C8 FFA | 0.79% |
| Monolaurin | 9.50% |
| Cetyl esters NF | 2.00% |
| Coconut oil | 29.70% |
| Hemp Oil | 13.20% |
| Cod Liver Oil | 12.64% |
| perfume | 0.03% |
| Total | 100.00% |
| C8 + 10/sum unsat (Unsat Fat Permeate Factor) | 1.70 |
| sum wax (Retentate Factor) | 11.5 |
| (ALA + SDA)/(EPA + DHA) [PUFA Ratio] | 1.000 |
| ALA + SDA + EPA + DHA (sum omega3) | 5.36 |
| (C8 + 10)/sum fat > C10:0 (Sat Fat Permeate Factor) | 1.32 |
| C18:0 | 1.43 |

### Example 30

The following Omeza Skin-J composition is prepared in accordance with the procedure described in Example 25:

Oily Skin - J Leg Gel

| | |
|---|---|
| MCT Oil | 47.00% |
| Hemp Oil | 10.09% |
| Cod Liver Oil | 10.00% |
| Monolaurin | 9.50% |
| Cetyl esters NF | 2.00% |
| virgin coconut oil | 5.24% |
| RBD palm oil | 16.10% |
| perfume B | 0.07% |
| Total | 100.00% |
| C8 + 10/sum unsat (Unsat Fat Permeate Factor) | 1.96 |
| sum wax (Retentate Factor) | 11.50 |
| (ALA + SDA)/(EPA + DHA) [PUFA Ratio] | 1.00 |
| ALA + SDA + EPA + DHA (sum omega3) | 4.24 |
| Sat'd Fat > C10:0 | 15.02 |
| (C8 + 10)/sum fat > C10:0 (Sat Fat Permeate Factor) | 3.17 |
| C18:0 | 1.38 |

This composition is compared to Omeza Lidocaine Lavage AE composition, another composition of the present disclosure. This composition is prepared in accordance with the procedure described herein:

Omeza Lidocaine Lavage - AE

| | Weight % |
|---|---|
| MCT Oil | 47.10% |
| Monolaurin | 0.60% |
| Cetyl Esters NF | 1.00% |
| Lidocaine | 0.80% |
| crude coconut oil | 6.00% |
| RBD Palm Oil - CP6 | 17.91% |
| Hemp Oil | 13.39% |
| Cod Liver Oil | 13.20% |
| Total | 100.00% |
| C8 + 10/sum unsat (Unsat Fat Permeate Factor) | 1.57 |
| sum wax (Retentate Factor) | 1.60 |
| (ALA + SDA)/(EPA + DHA) [PUFA Ratio] | 1.00 |
| ALA + SDA + EPA + DHA (sum omega3) | 11.29 |
| Sat'd Fat > C10:0 | 14.25 |
| (C8 + 10)/sum fat > C10:0 (Sat Fat Permeate Factor) | 2.10 |
| C18:0 | 2.01 |

This example highlights the difference between oil and gel.

2 g of oil and gel were rubbed into the left hand of a senior male for 15 seconds. Temperature of the left-hand palm's heel was collected before, immediately after rub-in and every 15 seconds for 5 minutes and then every 30 seconds until the experiment terminated. The comparative data is depicted in FIG. 23.

As shown in the figure, the oil peaked @˜+3° F.; gel was unchanged for 8 minutes, but then cooled ˜−1.5° F.

### Explanation of Example 30

2 g of oil or gel is more product than one hand can absorb in a 15-second rub-in, so there is a gross excess of product.

Without wishing to be bound it is believed that Torso blood flow (98.7° F.) flows through the dermis and warms the cold hand (91.7° F.) that used the oil composition. The blood flow increases because anti-inflammatory omega3 oils passed through the epidermis, through the basement membrane and into the dermis. Palm temperature rises as blood flow temporarily increases in response to the anti-inflammatory omega3's migrating into the dermis.

It is further believed that the same warm blood flowed through the dermis but did not warm the cold hand (91.5° F.) that used the gelled composition because no anti-inflammatory omega3's migrated into the dermis from the gel. There is no blood flow in the epidermis. Therefore, the gel must have stayed in the epidermis, as no anti-inflammatory activity was observed in the dermis. Consistent with this, the gel causes increased epidermal hydration because of non-migrating fat build-up between keratinocytes in the epidermis, helping prevent moisture loss. The gel is more effective in moisturizing the skin. It moisturizes the skin for eight hours.

### Example 31

A study was done with Gel-J, prepared in Example 30 with 15-seconds and 60-seconds rub-in to the heel of the palm. The results are depicted in FIG. 24. The longer rub-in caused a higher initial temperature rise, but the slopes of the temperature decay lines are parallel, suggesting that rub-in time effects initial temperature rise, but not the decay back to normal.

The application of the gel provides softer and smoother skin and smaller pores than application of the oil in WO 2019/200364. Without wishing to be bound, it is believed that MCT will preferentially feed basal cells if confined in the epidermis. Glucose-fed basal cells produce lactate, an inflammatory compound; MCT-fed cells do not produce lactate. MCT is in all the oil and gel compositions. The gel delays absorption; MCT accelerates absorption. The amount of MCT can be adjusted to drive through the gelation to achieve the absorption rate optimum. For example, when monolaurin is increased to 9.5%, absorption is slowed. Additional MCT is added to partially offset the loss in absorption rate.

Since the MCT in the gel remained in the epidermis, as opposed to the oil compositions of WO 2019/200364, the effect from the gel is more dramatic than the oil.

Further, when applied to the face, the oil composition of WO 2019/200364 causes sweating, while the gelled composition has a cooling effect. This temperature rise is troubling to post-menopausal women. The gel of the present disclosure would maximize the absorption rate without inducing a perceived temperature rise.

The oil composition of WO 2019/200364 and gel composition of this disclosure are very different products because the former is transdermal, and this gel is intradermal.

What is concluded from all of the experiments is that the gel system herein is a mixture of formulation components that can engineer the amount of omega3 and MCT that stops in the epidermis, stops in the dermis or goes transdermal.

The gelled composition of the present disclosure is a wax-gelled, pumpable product that resists running off the patient during application. MCT [Caprylic triglyceride (C8:0)+capric triglyceride (C10:0)] is present in greater than 30 wt % by weight. Without wishing to be bound, it is believed that MCT is directly consumed by viable cells as an alternative to glucose. Glucose-fed cells produce lactate, an inflammatory compound; MCT-fed cells do not produce lactate. MCT-fed cells, with sufficient available blood oxygen, also produce anti-inflammatory macrophage M2. The substitution of MCT for glucose is a net anti-inflammatory step.

Further, without wishing to be bound, it is believed that MCT in the amount provided in the formulations described herein has three distinct roles:

(1) As a solvent, it “drags” long chain fats through the stratum corneum and through the basement membrane into the dermis.

(2) There is a monolayer of viable basal cells (stratum basale) on the distal side of the basement membrane separating the epidermis from the dermis. Over a ˜40 day life span, they become keratinocytes as they migrate to the surface and slough off. These basal keratinocyte cells normally consume glucose that migrates through the basement membrane up to the basal cells. When distal-side MCT arrives topically, basal cells preferentially consume MCT. Their respiratory byproducts help make the dermis anti-inflammatory.

(3) Excess MCT (i.e. not yet consumed) passes into the dermis where it indirectly contributes to reducing inflammation.

In addition, without wishing to be bound, it is believed that topical coconut oil tends to migrate through the basement membrane and enter the dermis as a topical nutrient. Palm oil is primarily a mixture of oleic (C18:1) and palmitic (C16:0) triglycerides. Long chain saturated fats do not easily transfer out of the epidermis. They tend to stay in the epidermis to lubricate the keratinocytes, adding suppleness and softness to the skin and increasing the moisture barrier properties of the epidermis.

Further, the gelled composition described herein provides moisture protection for at least 8 hours. As described herein, the weight of C18 omega3 fats approximately equals the weight of C>18 omega3 fats. That is (ALA+SDA)/(EPA+DHA) approximately equals 1, providing a balance between inflammatory and anti-inflammatory byproducts.

PUFA fats are also antioxidants that help control excess ROS. Part of the skin protection is believed to come from inducing ROS; part comes from quenching ROS. It is a situation where balance is key. The net anti-inflammatory response induces more blood flow to intact skin which induces reactive Oxygen Species (ROS) to help stop infections that could migrate down the skin to the open wound.

Further, without wishing to be bound, it is believed that when the gelled compositions are applied to the skin topically, there are three distinct response zones:

Oxidative Burst

The temperature rise rate (A° F./minute) is independent of the dose.

The rate of temperature rise is controlled by diffusion through the epidermis into the dermis.

Vascular Response begins.

Temperature rise reaches a peak and then holds steady, depending on dose.

Sphincters in the vascular system reduce flow in response to “too much” oxygen from the oxidative burst.

Vascular Control achieved

Temperature declines linearly until a new equilibrium is established

The skin cools as the vascular system makes its adjustment

A new temperature equilibrium is achieved.

### Example 32

Example 32 illustrates how temporary and long-lasting pain relief can coexist in a topical gel. The following gel was prepared in accordance with the procedure of Example 25:

Omeza Deep Pain Relief - CN2
4% + camphor

| | |
|---|---|
| MCT Oil | 56.42% |
| Hemp Oil | 12.20% |
| Cod Liver Oil | 16.25% |
| Monolaurin | 9.50% |
| Cetyl esters NF | 1.00% |
| virgin coconut oil | 0.33% |
| Lidocaine | 4.00% |
| Camphor | 0.30% |
| Total | 100.00% |
| C8 + 10/sum unsat (Unsat Fat Permeate Factor) | 2.49 |
| sum wax (Retentate Factor) | 10.50 |
| (ALA + SDA)/(EPA + DHA) [PUFA Ratio] | 0.75 |
| ALA + SDA + EPA + DHA (sum omega3) | 6.03 |
| Sat'd Fat > C10:0 | 4.24 |
| (C8 + 10)/sum fat > C10:0 (Sat Fat Permeate Factor) | 13.29 |
| C18:0 | 0.74 |

### Explanation of Example 32

Pain relief is complex. There are at least 3 aspects: itching relief, skin relief and inflammation relief. Camphor provides immediate itching relief by evaporation and consequent cooling. Lidocaine provides shallow relief after 5-10 minutes. These effects are well-known. MCT provides deep long-lasting, but slow-to-materialize pain relief by reducing inflammation. MCT slowly reduces inflammation when cells convert from glucose (inflammatory lactic acid producing) to MCT (no inflammatory lactate production).

But consumers expect fast relief, not slow relief. In Example 31, CN2 delivers immediate, intermediate and long-term pain relief. Camphor cools the hot surface during rub-in (immediate relief); the lidocaine provides temporary (intermediate relief) pain relief that allows sufficient time for the MCT to begin to reduce inflammation, the true source of deep pain (long lasting relief).

### Example 33

Example 33 illustrates how a topical product can improve sleep in the short term. It was prepared in accordance with the procedure of Example 25.

Omeza Sleep Aid - PM5

| | |
|---|---|
| MCT Oil | 61.00% |
| Hemp Oil | 11.40% |
| Cod Liver Oil | 15.10% |
| Monolaurin | 9.50% |
| Cetyl esters NF | 1.00% |
| C8 FFA | 2.00% |
| Total | 100.00% |
| C8 + 10/sum unsat (Unsat Fat Permeate Factor) | 2.89 |
| sum wax (Retentate Factor) | 10.5 |
| (ALA + SDA)/(EPA + DHA) [ PUFA Ratio] | 0.75 |
| ALA + SDA + EPA + DHA (sum omega3) | 5.62 |
| Sat'd Fat > C10:0 | 3.71 |
| (C8 + 10)/sum fat > C10:0 (Sat Fat Permeate Factor) | 16.41 |
| C18:0 | 0.68 |

### Explanation of Example 33

Caprylic acid (protonated) is volatile at body temperature. When PM5 is rubbed onto the chest at bedtime, caprylic acid fumes are inhaled, clearing the sinuses and helping control bacterial growth in the lungs. The net effect, overnight, is less snoring, easier breathing and better sleep. This same effect is seen with athletes who apply PM5 on the chest before a workout. Breathing is better during the exercise period. There is no coconut or palm oil to impede caprylic acid fumes.

MCT is increased to >60%. MCT is an indirect anti-inflammatory compound in two ways: 1. When MCT is ingested by cells, the glucose-lactic-acid pathway is avoided and inflammatory lactate is not produced, 2. When MCT is ingested by cells, one of the by-products is M2 macrophage, a well-known anti-inflammatory macrophage. When MCT >60%, an unexpected response occurs. Sore muscles are relieved. The consequence of arterial plaque is reduced. Plaque is a fatty foam containing cholesterol. Drugs, like statins, will reduce cholesterol and if cholesterol <80 mg/dl using, for example, statins, cholesterol can be removed from immature plaque buildups. But the release of cholesterol causes inflammation elsewhere. This is where PM5 may have a benefit, in reducing consequent inflammation (via omega3 and reduced lactate). When PM5 is rubbed into the chest, inflammation is reduced in and around the heart and lungs. These observations are preliminary.

### Example 34

Example 34 is an improved gel, designed to reduce arterial plaque build-up in conjunction with statin treatment. It is prepared as described in Example 25.

Omeza Keto Supplement Gel - K3

| | |
|---|---|
| MCT Oil | 46.73% |
| Hemp Oil | 10.67% |
| Cod Liver Oil | 30.000% |
| Monolaurin | 9.50% |
| Cetyl esters NF | 1.00% |
| virgin coconut oil | |
| colloidal oatmeal | 0.08% |
| perfume-B | 0.03% |
| caprylic acid | 1.99% |
| Total | 100.00% |
| C8 + 10/sum unsat (Unsat Fat Permeate Factor) | 1.50 |
| sum wax (Retentate Factor) | 10.50 |
| (ALA + SDA)/(EPA + DHA) [PUFA Ratio] | 0.42 |
| ALA + SDA + EPA + DHA (sum omega3) | 9.00 |
| Sat'd Fat > C10:0 | 6.11 |
| (C8 + 10)/sum fat > C10:0 (Sat Fat Permeate Factor) | 7.63 |
| C18:0 | 1.05 |

### Explanation of Example 34

PM5 is similar to K3. K3 is dispensed from a unit dose, bag-on-valve dispenser that passes through a shear inducing tip. The shear tends to break up 9.5% monolaurin gels. Practically, this means that gel applied to a vertical chest can drip off before rub-in is complete. Increasing cod liver oil to 30% (and correspondingly reducing hemp oil) resolves this issue.

K3 is a Topical Supplement product because it claims to provide a high level of keto fatty acids. It may also interact with existing statins, of various sorts to reduce body-produced cholesterol; heart-healthy diets reduce triglycerides and food-supplied cholesterol. Both are important for K3 to be effective.

What the medical community needs is a way to reverse cholesterol build-up concurrent with removal of early cholesterol deposits. This is complex. Plaque builds up “unknown” when not taking statins/aspirin and not following a heart-healthy diet. Eventually high blood pressure or atrial fibrillation or combination thereof triggers effective remedial action. These actions inhibit further deterioration, or require blockage removal surgically. Even after bypass surgery or arterial intervention, there is still plaque build-up that represents a risk. The mechanism of action for K3 is incompletely understood. Venous blood enters the heart and is pumped to the lungs where it is oxygenated and distributed throughout the body. When K3 is rubbed into the chest once per day, anti-inflammatory omega3 and anti-inflammatory MCT enter the blood stream. Without wishing to be bound, it is believed that omega3 fatty acids reduce the tendency of lipoproteins to stick together. Moreover, it is also believed, without wishing to be bound, that MCT in combination with the fish oils, as described herein, decreases the host response to inflammatory challenge

Cardiovascular disease is an inflammatory disease. The unique combination of omega3 and MCT at K3 ratios is a safe and effective anti-inflammatory countermeasure when applied topically to the chest twice per day. The total amount of omega3 (9%) sets K3 from other embodiments. Adding K3 to the chest twice daily increases the keto fats entering the body. The cardiovascular effect is complicated and needs to be proven clinically.

### Example 35

RG4 gel was used in human trials. Approximately 1 gram of RG4 gel was rubbed into the skin of three volunteers. Arterial blood was drawn and analyzed for triglycerides over 1500 minutes. The individual chain lengths varied with time as triglycerides were metabolized into other triglycerides. The mean Total Triglycerides over time is plotted in FIG. 25.

Triglycerides increased for 4.78 hours (peaked @ 57.2 mg/DL) and then declined below baseline

### Explanation of Example 35

RG4 is transdermal. It takes between 4 and 5 hours to be seen as increased triglycerides in blood sampled from the brachial artery in the arm. Overnight the pulse increase in triglycerides is metabolized and overnight triglyceride was reduced below the starting point.

Polyunsaturated triglycerides are enzymatically converted into other compounds and eventually the residual is hydrolyzed into free fatty acids. Polyunsaturated free fatty acids are often incorporated into the cell wall lipid bilayer.

### Example 36

The following example is prepared in accordance with the procedure described herein in Example 13:

Collagen Matrix CZ
Next Gen

| | Weight % |
|---|---|
| MCT Oil | 17.20% |
| C8 FFA | 1.20% |
| Monolaurin | 11.00% |
| Cetyl Esters | 1.00% |
| Crude Coconut Oil | 10.00% |
| Ascorbyl Palmitate | 0.50% |
| Red palm concentrate | 0.20% |
| Hemp oil | 5.20% |
| Cod Liver Oil | 11.40% |
| Fish Collagen | 41.40% |
| Ground Sea Salt | 0.90% |
| Total | 100.00% |
| C8 + 10/sum unsat (Unsat Fat Permeate Factor) | 1.42 |
| sum wax (Retentate Factor) | 12.50 |
| (ALA + SDA)/(EPA + DHA) [PUFA Ratio] | 0.50 |
| ALA + SDA + EPA + DHA (sum omega3) | 3.63 |
| Sat'd Fat > C10:0 | 10.50 |
| (C8 + 10)/sum fat > C10:0 (Sat Fat Permeate Factor) | 1.76 |
| C18:0 | 0.69 |

Collagen Matrix
Collagen Matrix CR

| | Weight % |
|---|---|
| MCT Oil | 8.00% |
| C8 FFA | 1.30% |
| Monolaurin | 4.30% |
| Cetyl Esters | 2.00% |
| Yellow Beeswax | 4.00% |
| Triethyl citrate | 2.00% |
| Ethyl linoleate | 2.00% |
| Virgin Coconut Oil | 10.00% |
| Ascorbyl Palmitate | 0.50% |
| Red palm concentrate | 0.20% |
| Hemp oil | 12.00% |
| Cod Liver Oil | 11.40% |
| Fish Collagen | 41.40% |
| Ground Sea Salt | 0.90% |
| Total | 100.00% |
| C8 + 10/sum unsat (Unsat Fat Permeate Factor) | 0.49 |
| sum wax (Retentate Factor) | 10.8 |
| (ALA + SDA)/(EPA + DHA) [PUFA Ratio] | 1.01 |
| ALA + SDA + DHA + EPA (sum omega3) | 4.68 |
| Sat'd Fat > C10:0 | 11.17 |
| (C8 + 10)/sum fat > C10:0 (Sat Fat Permeate Factor) | 0.83 |
| C18:0 | 0.86 |

In Example 36, beeswax, triethyl citrate and ethyl linoleate were removed from the prior art Matrix CR formula. Monolaurin was increased from 4.3% to 11% to compensate for the loss of beeswax. MCT oil was increased to balance the formula. The Sat Fat Permeate Factor increased 112% (0.83 to 1.76) and the (ALA+SDA)/(EPA+DHA) ratio was reduced to 0.5.

The specific gravity, after whipping, dropped from 0.78 to 0.57, a 22% reduction. Surprisingly, the stability of the CZ Matrix was superior. After 15 hours at 50° C., there was no separation; there was no loss in gas (specific gravity unchanged) and no change in viscosity. This means that Matrix CZ better tolerates normal distribution. Matrix CR has to be shipped refrigerated to prevent gas loss during distribution.

### Explanation of Example 36

Triethyl citrate and ethyl linoleate are process aids that reduce viscosity caused by adding beeswax. Increasing monolaurin by 156% (4.3 to 11%) increased the gas-holding capacity of Matrix CZ with no change in active ingredients (cod liver oil, fish collagen, sea salt). By whipping more nitrogen gas into the matrix, specific gravity and viscosity are reduced. Matrix CZ spreads across the wound more easily.

### Example 37

In Example 37, the learning from Example 35 was used to make a formula without sea salt (Matrix CZ4) and one with benzethonium chloride (CZ3).

CZ3 is an OTC first aid antiseptic that is pre-approved by FDA for minor wounds. CZ4 requires FDA approval (510(k)) but is a lower cost-to-manufacture product designed for Home Health users.

Both CZ3 and CZ4 are prepared in accordance with the procedure described herein:

Collagen Matrix CZ3
First Aid (white)

| | Weight % |
|---|---|
| MCT Oil | 18.13% |
| C8 FFA | 1.20% |
| Monolaurin | 11.00% |
| Cetyl Esters | 1.00% |
| Crude Coconut Oil | 10.00% |
| Ascorbyl Palmitate | 0.50% |
| Hemp oil | 5.20% |
| Cod Liver Oil | 11.40% |
| Fish Collagen | 41.40% |
| Benzethonium chloride | 0.17% |
| Total | 100.00% |
| C8 + 10/sum unsat (Unsat Fat Permeate Factor) | 1.50 |
| sum wax (Retentate Factor) | 12.50 |
| (ALA + SDA)/(EPA + DHA) [PUFA Ratio] | 0.50 |
| ALA + SDA + EPA + DHA (sum omega3) | 3.63 |
| Sat'd Fat > C10:0 | 10.41 |
| (C8 + 10)/sum fat > C10:0 (Sat Fat Permeate Factor) | 1.86 |
| C18:0 | 0.69 |

Collagen Matrix CZ4
A6010 (light yellow)

| | Weight % |
|---|---|
| MCT Oil | 18.25% |
| C8 FFA | 1.20% |
| Monolaurin | 11.00% |
| Cetyl Esters | 1.00% |
| Crude Coconut Oil | 10.00% |
| Ascorbyl Palmitate | 0.50% |
| Hemp oil | 5.20% |
| Cod Liver Oil | 11.40% |
| Fish Collagen | 41.40% |
| red palm concentrate | 0.05% |
| Total | 100.00% |
| C8 + 10/sum unsat (Unsat Fat Permeate Factor) | 1.51 |
| sum wax (Retentate Factor) | 12.50 |
| (ALA + SDA)/(EPA + DHA) [PUFA Ratio] | 0.50 |
| ALA + SDA + EPA + DHA (sum omega3) | 3.63 |
| Sat'd Fat > C10:0 | 10.43 |
| (C8 + 10)/sum fat > C10:0 (Sat Fat Permeate Factor) | 1.87 |
| C18:0 | 0.69 |

CZ4 (light yellow) and CZ3 (white) are differentiated by color from CR (prior art; canary yellow). CZ4 and CZ3 are more thermally stable because monolaurin was increased (11% versus 4.3%) and beeswax, triethyl citrate, ethyl linoleate and sea salt were eliminated. MCT was increased to balance mass. CZ3 is a First Aid Antiseptic (benzethonium chloride at 0.17%). The collagen and cod liver oil concentrations are identical in CR, CZ3 and CZ4.

The permeate ratio increases (CR at 0.83; CZ3 at 1.76; CZ4 at 1.87). The PUFA ratio drops from CR 1.01 to 0.50 (CZ3 & CZ4).

Color is adjusted by reducing (CZ4) or eliminating (CZ3) red palm concentrate.

What was unexpected was the increase in physical phase stability by eliminating beeswax, triethyl citrate, ethyl linoleate and sea salt. Higher melting point beeswax should have made the product more stable.

What was discovered is that higher monolaurin allowed the cooled CZ3/CZ4 product to be whipped into a stable crème. Monoglycerides, like monolaurin, are well-known emulsifiers for stabilizing fatty formulas in ice cream or whipped crème, but they require cold temperatures to retain foam stability. In a 15-hour 120° F. (48.9° C.) stress test, there was no visible oil separation for CZ3 or CZ4 and the foam stayed flexible with no change in specific gravity. In the same test, CR always has a slight amount of weeping and always lost all its gas (becoming too hard).

The net result is that FDA-approved CR has sea salt, a canary yellow color and is sterile to justify premium pricing. FDA-approved CZ4 has no salt, is sterile and has a light-yellow color. CZ4 has the same concentration of active ingredients and is sterile (so CR can be CZ4's FDA 510(k) “predicate”), but CZ4 is much lighter, easier to use and is cheaper to manufacture (eliminating sea salt eliminates a multi-day processing step). CZ4 is profitable in a low-price market. CZ3 is almost the same as CZ4, but without the colorant, is not sterile (cheaper) plus has a first aid antiseptic active ingredient which allows CZ3 to compete in the retail first aid aisle with non-sterile, Neosporin-type first aid antiseptics.

The three products, CZ3, CZ4 and CR, are also separated by differentiated packaging variants. CZ3 is packed in a low-priced tube. CZ4 is packed in a generic single-use vial. CR is packed in an oxygen-barrier, premium single-use vial.

### Explanation of Example 37

Sea salt adds considerable manufacturing complexity because it is gritty and corrosive. Product has to be aged 24 hours to form a strong enough gel to prevent high density salt from precipitating. Then the product is whipped once to incorporate the sea salt. Filling is difficult because the abrasive nature of ground salt in a high viscosity matrix erodes moving parts in pumps, causing variation in filling and frequent pump replacement.

CZ3 is similar to CZ4 in that both eliminate sea salt, but different in that benzethonium chloride replaces red palm concentrate. This provides expected color differentiation but adding benzethonium chloride materially changes the regulatory complexity of marketing these collagen-rich matrices.

### Example 38

In Example 38, the learning from Example 35 was reapplied to a gel (HM2). The (ALA+SDA)/(EPA+DHA) ratio is 0.5. This example is prepared in accordance with the procedure described for Example 35.

The base of the right-hand palm was measured per Example 2. Temperature rose for 10 minutes and then was constant for 25 minutes (FIG. 26). Gel J was used as a control. Gel J temperature dropped for 10 minutes and was then constant. The palm temperature response of Gel HM2 and Gel J were mirror images of each other.

Gel HM2 has much higher permeate factors (14.66 versus 3.17). Retentate factors are similar, but J has slightly higher saturated fats >C10:0 (15.02 versus 14.66).

### Explanation of Example 38

HM2 has a higher permeation factor that leads to immediate blood flow stimulation and ensuing palm temperature rise. Gel J inhibits permeation with a high level of long chain saturated fats. After the 10 minute stimulation, both palm temperatures are unchanged.

The higher permeate factor increases the rate of oil penetration into the dermis.

Omeza Skin Protectant Gel - HM2

| | |
|---|---|
| MCT Oil | 61.24% |
| Hemp Oil | 8.30% |
| Cod Liver Oil | 18.00% |
| Monolaurin | 11.00% |
| Cetyl esters NF | 1.00% |
| coconut oil | 0.33% |
| colloidal oatmeal | 0.08% |
| perfume B | 0.05% |
| Total | 100.00% |
| C8 + 10/sum unsat (Unsat Fat Permeate Factor) | 3.00 |
| sum wax (Retentate Factor) | 12.00 |
| (ALA + SDA)/(EPA + DHA) [PUFA Ratio] | 0.50 |
| ALA + SDA + EPA + DHA (sum omega3) | 5.74 |
| Sat'd Fat > C10:0 | 4.17 |
| (C8 + 10)/sum fat > C10:0 (Sat Fat Permeate Factor) | 14.66 |
| C18:0 | 0.69 |

Oily Skin - J Leg Gel

| | |
|---|---|
| MCT Oil | 47.00% |
| Hemp Oil | 10.09% |
| Cod Liver Oil | 10.00% |
| Monolaurin | 9.50% |
| Cetyl esters NF | 2.00% |
| virgin coconut oil | 5.24% |
| RBD palm oil | 16.10% |
| perfume B | 0.07% |
| Total | 100.00% |
| C8 + 10/sum unsat (Unsat Fat Permeate Factor) | 1.96 |
| sum wax (Retentate Factor) | 11.50 |
| (ALA + SDA)/(EPA + DHA) [PUFA Ratio] | 1.00 |
| ALA + SDA + EPA + DHA (sum omega3) | 4.24 |
| Sat'd Fat > C10:0 | 15.02 |
| (C8 + 10)/sum fat > C10:0 (Sat Fat Permeate Factor) | 3.17 |
| C18:0 | 1.38 |

### Example 39

The following example is prepared in accordance with the procedure described herein:

Omeza Skin Protectant Gel - HM3

| | |
|---|---|
| MCT Oil | 60.24% |
| Hemp Oil | 8.30% |
| Cod Liver Oil | 18.00% |
| Monolaurin | 12.00% |
| Cetyl esters NF | 1.00% |
| coconut oil | 0.33% |
| colloidal oatmeal | 0.08% |
| perfume B | 0.05% |
| Total | 100.00% |
| C8 + 10/sum unsat (Unsat Fat Permeate Factor) | 2.95 |
| sum wax (Retentate Factor) | 13.00 |
| (ALA + SDA)/(EPA + DHA) [PUFA Ratio] | 0.50 |
| ALA + SDA + EPA + DHA (sum omega3) | 5.74 |
| Sat'd Fat > C10:0 | 4.17 |
| (C8 + 10)/sum fat > C10:0 (Sat Fat Permeate Factor) | 14.43 |
| C18:0 | 0.69 |

HM3 is similar to HM2 in Example 37, except the monolaurin is increased to 12% and the product is whipped after cooling to 95° F. Gels are stable, but hard to fill on normal filling machines because colloidal oatmeal is not soluble in anhydrous systems and precipitate, making the active ingredient concentration inconsistent.

To alleviate this deficiency, monolaurin was increased, and the cooled product whipped with nitrogen gas. The gel was transformed into a stable “whipped crème”. Colloidal oatmeal was consistent, the product applied smoothly, and the whipped gel was absorbed readily. Unfortunately, this formulation was phase stable only in small containers. The foam collapsed and oil leaked to the bottom of a 1,000 liter beaker.

### Example 40

The following example is prepared in accordance with the procedure described herein:

Omeza Keto Supplement - K1a
Spray

| | |
|---|---|
| MCT Oil | 43.77% |
| Hemp Oil | 27.00% |
| Cod Liver Oil | 27.000% |
| Monolaurin | 0.60% |
| Cetyl esters NF | 1.00% |
| virgin coconut oil | 0.33% |
| RBD palm oil | |
| perfume-B | |
| caprylic acid | 0.30% |
| Total | 100.00% |
| C8 + 10/sum unsat (Unsat Fat Permeate Factor) | 1.00 |
| sum wax (Retentate Factor) | 1.60 |
| (ALA + SDA)/(EPA + DHA) [PUFA Ratio] | 0.96 |
| ALA + SDA + EPA + DHA (sum omega3) | 11.23 |
| Sat'd Fat > C10:0 | 7.49 |
| (C8 + 10)/sum fat > C10:0 (Sat Fat Permeate Factor) | 5.83 |
| C18:0 | 1.39 |

### Explanation of Example 40

Two swine trials were conducted with Collagen Matrix CR by itself and with Lidocaine Lavage-AA plus Collagen Matrix CR. 10 wounds were inflicted on the swine in a controlled, validated biofilm kill protocol. Pseudomonas aeruginosa and Staphylococcus epidermis were added at 10⁸ to each wound and then the surviving bacteria were measured at various time points.

Lidocaine Lavage-AA does not have direct bactericidal activity, but yet staph and pseudomonas were reduced significantly.

LL-AA has a high level (>8.0) of saturated fats >C10:0 (14.20) and a low permeate (<3), (@2.25) and thus leaves a greasy residue on the skin surface. The greasy surface inhibits biofilm attachment to the open wound. When bacteria cannot attach to a surface, biofilm cannot form. When biofilms cannot form, natural commensal bacterial activity gradually reduces pathogens on the wound (as shown in FIG. 25). In an open wound, the residual greasy surface is a major benefit because pathogen kill is natural, and resistance-to-antibiotics cannot occur.

Conversely, on intact skin, greasy residue is not cosmetically acceptable. Many formulations with sum saturated fats <8.0 are presented herein in other examples that eliminate the greasy feeling after rub-in.

But there remains a need for a product that is at once greasy (to inhibit biofilm development) but does not feel greasy to the touch. An example is Omeza Keto Supplement K1a. The keto diet is a fat-rich, carbohydrate-lean diet that purports many benefits, including weight loss. The problem is ingesting fats, particularly MCT, upsets the digestive system.

K1a overcomes this digestive disturbance by introducing keto fats topically. The problem has always been how to do this in sufficient quantity and in a cosmetically acceptable way.

What was determined is that if the omega3 Permeate Factor is >3, then a physiologically important keto fat dose can be delivered topically without offending the users.

K1a (@ 2.1 ml/app) is rubbed onto the chest once per day. The oil penetrates the skin; the caprylic acid is volatile at body temperature and is breathed through the sinuses and into the lungs. Caprylic acid is a mild antibacterial/antiviral ingredient and helps control bacterial and viral infections in the breathing pathways. Users who wheeze, stop wheezing; users with blocked sinuses can breathe through their nose again.

The keto effect is modest (per app) and requires continuous daily use. Compliance can fall off if consumers do not perceive a benefit or if they perceive a greasy defect. Caprylic acid provides an immediate breathing benefit that consumers visualize as a positive effect; the high permeate, high fat, high omega3 combination reduces the greasy negative just enough to encourage continual use until the keto-effect, over time, delivers the weight loss benefit of the keto diet topically.

In other words, an immediate benefit is used to help sustain continuous use until the long-term benefit is apparent and early adopters become long-term advocates.

The following table summarizes the permeate factors, retentate factors and the sum of MCT and monolaurin and cetyl esters and wax of examples described hereinabove:

Example Ratios

| | | | | |
|---|---|---|---|---|
| | | Retentate | Permeate | Sum (MCT + monolaurin + |
| Formula | Example # | Factor | Factor | cetyl esters + wax) |

OILS

| | | | | | | | | |
|---|---|---|---|---|---|---|---|---|
| | | Example 29 | 1.60 | low | 1.54 | low | <42 | 41.6 |
| | | (Counter | | | | | | |
| | | Example #1) | | | | | | |
| | | Example 30 | 1.60 | | 1.54 | | | 41.6 |
| | | (Counter | | | | | | |
| | | Example #1) | | | | | | |
| | CB | 3 | 1.60 | | 1.59 | | | 40.3 |
| | BV | 1 | 1.60 | | 2.10 | | | 31.6 |
| | AA | 20 | 1.60 | | 2.25 | | | 33.6 |
| | AF2 | 21 | 1.60 | | 2.30 | | | 33.6 |
| | LL AE | 30 | 1.60 | | 2.10 | | | 48.7 |
| Permeate | RG4 | 15 | 1.60 | low | 3.56 | high | | 42.9 |
| Factor ≥3 | D | 20 | 1.60 | | 3.56 | | | 42.9 |
| | K1a | 20 | 1.60 | | 5.83 | | | 45.4 |
| | Wound Wash C | 20 | 1.60 | | 3.94 | | | 53.6 |
| | Wound Wash D | 20 | 1.60 | | 4.07 | | | 53.6 |

GELS

| | | | | | | | | |
|---|---|---|---|---|---|---|---|---|
| Retentate | Gel AF | 2 | 11.50 | high | 1.32 | low | | 43.6 |
| Factor >8 | Gel E | 26a | 11.50 | | 2.22 | | | 50.8 |
| | Skin H | 26 | 11.50 | | 2.27 | | | 40.6 |
| | Skin G | 25 | 11.50 | | 2.27 | | | 54.7 |
| Permeate | Gel J | 30 | 11.50 | high | 3.17 | high | ≥42 | 58.5 |
| Factor ≥3 | Eye I | 28 | 10.50 | | 3.23 | | | 58.5 |
| | GOJO E | 19 | 10.50 | | 3.58 | | | 55.6 |
| | PM3 | 10 | 10.50 | | 4.26 | | | 40.5 |
| | A8 | 20 | 11.00 | | 5.05 | | | 50.1 |
| | CS1 | 1 | 10.50 | | 6.23 | | | 62.59 |
| | CS1c | 6 | 11.75 | | 6.69 | | | 62.6 |
| | RG5 | 15 | 10.50 | | 6.23 | | | 62.6 |
| | CS4 | 8 | 10.50 | | 6.42 | | | 62.6 |
| | CS3a | 7 | 10.50 | | 7.32 | | | 62.6 |
| | K3 | 34 | 10.50 | | 7.63 | | | 57.2 |
| | CN | 9 | 10.50 | | 8.12 | | | 62.6 |
| | CN2 | 32 | 10.50 | | 13.29 | | | 66.9 |
| | HM3 | 39 | 13.00 | | 14.43 | | | 73.2 |
| | HM2 | 38 | 12.00 | | 14.66 | | | 73.2 |
| | Acne F | 14 | 10.50 | | 15.02 | | | 77.9 |
| | PM5 | 33 | 10.50 | | 16.41 | | | 71.5 |

COLLAGEN

| | | | | | | | | |
|---|---|---|---|---|---|---|---|---|
| | CR | 5 | 10.80 | high | 0.83 | low | <20 | 18.3 |
| Permeate | DF | 4 | 10.25 | high | 1.45 | high | ≥20 | 25.1 |
| Factor ≥1 | CZ | 36 | 12.50 | | 1.76 | | | 29.2 |
| | CZ3 | 37 | 12.50 | | 1.86 | | | 30.3 |
| | CZ4 | 37 | 12.50 | | 1.87 | | | 28.9 |
| | DD | 12 | 9.80 | | 2.53 | | | 32.9 |

The above preferred embodiments and examples were given to illustrate the scope and spirit of the present invention. These embodiments and examples will make apparent to those skilled in the art other embodiments and examples. The other embodiments and examples are within the contemplation of the present invention. Therefore, the present invention should be limited only by the amended claims.

1-111. (canceled)

112. An anhydrous topical composition comprised of marine oil comprised of fish oil and comprised of ALA, SDA, EPA and DHA; vegetable oil having an omega3 fatty acid content greater than 9 wt %; linear fatty acid ester having 12-50 carbon atoms; monolaurin; and medium chain triglycerides (“MCT”), and optionally collagen; said vegetable oil having an omega3 fatty acid content greater than 9 wt %, wherein the weight ratio of the sum of the weights of (ALA+SDA)/sum of the weights of (EPA and DHA) present in the composition ranges from about 0.5 to about 1.5, and in addition, in the absence of collagen, when the composition is an oil, the sum of the wt % of MCT, monolaurin and linear fatty acid ester in said pharmaceutical composition greater than or equal to 42 wt %, or in the presence of collagen, the sum of the wt % of MCT, monolaurin and linear fatty acid ester in said pharmaceutical composition is greater than equal to 20 wt %.

113. The anhydrous topical composition according to claim 112 wherein the fish oil is cod liver oil, algal oil or salmon oil, and the vegetable oil having an omega3 fat content greater than 9 wt % is hempseed oil, canola oil, or flax seed oil.

114. The anhydrous topical composition according to claim 113 wherein the fish oil is cod liver oil, and the vegetable oil having an omega3 fat content greater than 9 wt % is hempseed oil, and the linear fatty acid ester is cetyl esters or wax or combination thereof.

115. The anhydrous topical composition according to claim 112 wherein the composition is collagen-free and has a permeate factor greater than or equal to 3.0 and a retentate factor greater than 8.0.

116. The anhydrous topical composition according to claim 115 wherein the marine oil content ranges from about 7 wt % to about 30 wt %, the vegetable oil having an omega3 fatty acid content greater than 9 wt % ranges from about 5 wt % to about 20 wt %, monolaurin is present in amounts ranging from about 4 wt % to about 15 wt %, linear fatty acid ester is present in amounts ranging from about 0.5 wt % to about 3 wt %, and MCT is present in amounts ranging from 25 wt % to 75 wt %, and wherein MCT is present in amounts from about 1 times to about five times the sum of the weight percents of the marine oil and the vegetable oil having an omega3 fatty acid content greater than 9 wt %.

117. The anhydrous topical composition according to claim 116 wherein marine oil ranges from about 10 wt % to about 19 wt %, the vegetable oil having an omega3 fatty acid content greater than 9 wt % ranges from about 7 wt % to about 14 wt %, monolaurin is present in amounts ranging from about 8 wt % to about 13 wt %, MCT is present in amounts ranging from about 31 wt % to about 68 wt %, and linear fatty acid ester is present in amounts ranging from about 1 wt % to about to about 2 wt %.

118. The anhydrous topical composition according to claim 112 wherein the composition is collagen-free and has a permeate factor greater than or equal to 3.0 and a retentate factor less than or equal to 8.0.

119. The anhydrous topical composition according to claim 118 wherein marine oil ranges from about 8 wt % to about 35 wt %, the vegetable oil having an omega3 fatty acid content greater than 9 wt % ranges from about 10 wt % to about 33 wt %, monolaurin is present in amounts ranging from about 0.1 wt % to about to about 1 wt %, MCT is present in amounts ranging from about 30 wt % to about to about 60 wt %, and linear fatty acid ester is present in amounts ranging from about 0.5 wt % to about to about 2 wt %, and wherein the sum of the wt % of marine oil and vegetable oil having an omega3 fatty acid content greater than 9 wt % is about 1 times to about two times the % weight of MCT.

120. The anhydrous topical composition according to claim 119 wherein the marine oil is present from about 23 wt % to about 27 wt %, the vegetable oil having an omega3 fatty acid content greater than 9 wt % ranges from about 23 wt % to about 27 wt %, monolaurin is present in amounts ranging from about 0.5 wt % to about 0.7 wt %, MCT is present in amounts ranging from about 30 wt % to about 42 wt %, and linear fatty acid ester is present in amounts ranging from about 0.8 wt % to about 1.2 wt %.

121. The anhydrous topical composition according to claim 112 wherein the composition is collagen-free and has a permeate factor less than 3.0 and a retentate factor less than or equal to 8.0.

122. The anhydrous topical composition according to claim 121 wherein the marine oil is present from about 20 wt % to about 30 wt %, the vegetable oil having an omega3 fatty acid content greater than 9 wt % ranges from about 20 wt % to about 30 wt %, monolaurin is present in amounts ranging from about 0.1 wt % to about 2 wt %, MCT is present in amounts ranging from about 25 wt % to about 50 wt %, and the linear fatty acid ester ranges from about 0.5 wt % to about 2 wt %, the sum of the wt % of marine oil and vegetable oil having an omega3 fatty acid content greater than 9 wt % is about 1 times to about two times the % weight of MCT, and the weight ratio of marine oil to vegetable oil having an omega3 fatty acid content greater than 9 wt %, ranges from about 1:1.3 to about 1.3:1.

123. The anhydrous topical composition according to claim 122 wherein the marine oil is present from about 23 wt % to about 27 wt %, the vegetable oil having an omega3 fatty acid content greater than 9 wt % ranges from about 23 wt % to about 27 wt %, monolaurin is present in amounts ranging from about 0.5 wt % to about 0.7 wt %, MCT is present in amounts ranging from about 30 wt % to about 42 wt %, and linear fatty acid ester is present in amounts ranging from about 0.8 wt % to about 1.2 wt %.

124. The anhydrous topical composition according to claim 112 wherein the composition is collagen-free and has a permeate factor less than 3 and a retentate factor greater than 8.

125. The anhydrous topical composition according to claim 124 wherein the marine oil is present from about 20 wt % to about 30 wt %, the vegetable oil having an omega3 fatty acid content greater than 9 wt % ranges from about 20 wt % to about 30 wt %, monolaurin is present in amounts ranging from about 7 wt % to about 14 wt %, MCT is present in amounts ranging from about 20 wt % to about 40 wt %, and the linear fatty acid ester present is present in amounts ranging from about 0.8 wt % to about to about 1.7 wt %, and the weight ratio of marine oil to vegetable oil having an omega 3 fatty acid content greater than 9 wt % ranges from about 1:1.3 to 1.3:1.

126. The anhydrous topical composition according to claim 125 wherein the marine oil is present from about 24 wt % to about 27 wt %, the vegetable oil having an omega3 fat content greater than 9 wt % ranges from about 24 wt % to about 27 wt %, monolaurin is present in amounts ranging from about 9 wt % to about 10 wt %, MCT is present in amounts ranging from about 28 wt % to about 33 wt %, and the linear fatty acid ester is present in amounts ranging from about 10 wt % to about to about 12 wt %.

127. The anhydrous topical composition according to claim 112 wherein collagen is present and the permeate factor is greater than or equal to 1.0 and the retentate factor is greater than or equal to 8.0.

128. The anhydrous topical composition according to claim 127 wherein marine oil is present from about 3 wt % to about 15 wt %, the vegetable oil having an omega3 fatty acid content greater than 9 wt % ranges from about 3 wt % to about 15 wt %, monolaurin is present in amounts ranging from about 2 wt % to about 15 wt %, MCT is present in amounts ranging from about 10 wt % to about 30 wt %, and the linear fatty acid ester is present in amounts ranging from about 0.3 wt % to about 10 wt %, and collagen is present in an amount ranging from about 35 wt % to about 45 wt %.

129. The anhydrous topical composition according to claim 128 wherein marine oil is present from about 5 wt % to about 12 wt %, the vegetable oil having an omega3 fatty acid content greater than 9 wt % ranges from about 5 wt % to about 12 wt %, monoglyceride is present in amounts ranging from about 4 wt % to about 12 wt %, MCT is present in amounts ranging from about 15 wt % to about 25 wt %, the linear fatty acid ester is present in amounts ranging from about 0.8 wt % to about 6 wt %, and collagen is present in an amount ranging from about 40 wt % to about 43 wt %.

130. The anhydrous topical composition according to claim 112 wherein collagen is present, the permeate factor is greater than equal to 1.0, and the retentate factor is less than 8.0.

131. The anhydrous topical composition according to claim 127 wherein the marine oil ranges from about 8 wt % to about 16 wt %, the vegetable oil having an omega3 fatty acid content greater than 9 wt % ranges from about 2 to about 16 wt %, monolaurin is present in amounts ranging from about 2 wt % to 18 wt %, MCT is present in an amount ranging from about 10 to about 25 wt %; linear fatty acid esters are present in amount ranging from about 0.5 to about 9 wt % and collagen is present in an amount ranging from about 30 to about 50 wt %.

132. The anhydrous topical composition according to claim 127 wherein marine oil ranges from about 10 wt % to about 13 wt %, vegetable oil having an omega3 fatty acid content greater than 9 wt % ranges from about 4.5 to 13 wt % of the formulation, monolaurin ranges from about 4 to about 13 wt %, MCT is present in an amount ranging from about 14 to about 20 wt %; linear fatty acid esters are present in amount ranging from about 0.9 to about 6 wt % and collagen is present in an amount ranging from about 38 to about 43 wt %.

133. The anhydrous topical composition according to claim 127 wherein salt or ascorbic palmitate or both are additionally present or saturated fatty acid having more than 10 carbon atoms in an amount greater than 10 wt % is additionally present or wherein the sum of the omega3 fatty acids present is greater than 10 wt %.

134. The anhydrous topical pharmaceutical composition according to claim 112 wherein an analgesic is additionally present.

135. A gelled collagen-free anhydrous topical composition, which comprises cod liver oil, hempseed oil, monolaurin, cetyl esters, medium chain triglycerides (“MCT”), coconut oil and palm oil, wherein said mixture has a C18:0 triglyceride concentration of 3 wt % or less, said composition being anhydrous and homogenous, and having a weight ratio of the sum of the weights of saturated (C8+C10) triglycerides/total sum of the weights of unsaturated triglycerides present in the composition ranging from 1 to 3, the ratio of the sum of the weights of (ALA+SDA)/(sum of the weights of EPA and DHA) present in the composition ranging from about 0.5 to about 1.5; and the amount of monolaurin being greater than 6 wt % of the composition, and the amount of MCT present being greater than 30 wt %.

136. The gelled anhydrous topical composition according to claim 135 wherein the cod liver oil is present in an amount ranging from about 5 wt % to about 30 wt %, hemp oil is present in an amount ranging from about 5 wt % to about 30 wt %, palm oil is present in an amount ranging from about 5 to about 20 wt %, coconut oil is present in an amount ranging from about 3 to 20 wt % MCT is present in an amount ranging from about 30 wt % to about 60 wt %, cetyl esters are present in an amount ranging from about 0.5 to about 3.0 wt %, monolaurin is present in an amount ranging from about 7 wt % to about 11 wt %, wherein the sum of the weights of cod liver oil, hempseed oil, monolaurin, cetyl esters, MCT, coconut oil and palm oil ranges from about 80 wt % to 100 wt % of the composition.

137. The gelled anhydrous topical composition according to claim 136 wherein the sum of the weights of cod liver oil, hempseed oil, monolaurin, cetyl esters, medium chain triglycerides (“MCT”), coconut oil and palm oil ranges from about 90 wt % of the composition to 100 wt %.

138. The gelled anhydrous topical composition according to claim 136 wherein cod liver oil is present from about 10 to about 15 wt %, hemp oil is present in about 10 to 15 wt %, MCT is present from about 40 to about 50 wt %, cetyl esters are present in an amount ranging from about 1.0 to about 2 wt %, monolaurin is present in an amount ranging from about 8.5 to about 10.5 wt %, palm oil is present in an amount ranging from about 10 to about 15 wt %, coconut oil is present from about 10 to about 13 wt % and the weight ratio of palm oil to coconut oil ranges from about 2:1 to about 1:1.

139. An anhydrous topical composition wherein the composition is Oil D, Omeza Gel CS1, Omeza Gel CS1c, OMEZA GEL AF, COLLAGEN MATRIX DF, OMEZA PSORIASIS RELIEF GEL-CS3a, OMEZA TOPICAL ANALGESIC GEL-CS4, OMEZA DEEP PAIN RELIEF-CN, OMEZA SLEEP AID-PM, OMEZA TOPICAL SUPPLEMENT-D SPRAY, COLLAGEN MATRIX DD, COLLAGEN ACNE MATRIX DE SKIN POLISH, OMEZA ACNE-F, OMEZA HOMEOPATHIC TRANSDERMAL-RG4, OMEZA HOMEOPATHIC INTRADERMAL-RG5, OMEZA GOJO E, Omeza Periwound Protection A8, Omeza Wound Wash-C Spray, Omeza Wound Wash-D Lidocaine Spray, Omeza Bundle, Collagen Matrix CZ, Collagen Matrix CZ3, Collagen Matrix CZ4, HM2, HM3, K1a, or an anhydrous composition comprising by weight

| | |
|---|---|
| MCT Oil | 32.14% |
| C8 FFA | 0.79% |
| Monolaurin | 9.50% |
| Cetyl esters NF | 2.00% |
| Coconut oil | 29.70% |
| Hemp Oil | 13.20% |
| Cod Liver Oil | 12.64% |
| perfume | 0.03%, |

or an anhydrous topical composition comprising by weight:

| | |
|---|---|
| MCT Oil | 29.05% |
| C8 FFA | 1.20% |
| Monolaurin | 9.50% |
| Cetyl Esters NF | 2.00% |
| RBD Palm Oil - CP6 | 19.00% |
| coconut oil | 13.00% |
| Hemp Oil | 13.15% |
| Cod Liver Oil | 13.00% |
| perfume-B | 0.10%, or |

an anhydrous topical composition comprising by weight:

| | |
|---|---|
| MCT Oil | 47.97% |
| Monolaurin | 9.50% |
| Cetyl Esters NF | 1.00% |
| RBD Palm Oil - CP6 | 16.10% |
| coconut oil | 5.24% |
| Hemp Oil | 10.09% |
| Cod Liver Oil | 10.00% |
| perfume-B | 0.10%, or |

an anhydrous topical composition wherein the comprising by weight:

| | |
|---|---|
| MCT Oil | 39.34% |
| C8 FFA | 0.68% |
| Monolaurin | 9.50% |
| Cetyl Esters NF | 2.00% |
| RBD Palm Oil - CP6 | 9.00% |
| coconut oil | 13.00% |
| Hemp Oil | 13.38% |
| Cod Liver Oil | 13.00% |
| perfume-B | 0.10%, or |

an anhydrous topical composition comprising by weight:

| | |
|---|---|
| MCT Oil | 32.14% |
| C8 FFA | 0.79% |
| Monolaurin | 9.50% |
| Cetyl esters NF | 2.00% |
| Coconut oil | 29.70% |
| Hemp Oil | 13.20% |
| Cod Liver Oil | 12.64% |
| perfume | 0.03%, or |

an anhydrous topical composition comprising by weight:

| | |
|---|---|
| MCT Oil | 47.00% |
| Monolaurin | 9.50% |
| Cetyl Esters NF | 2.00% |
| crude coconut oil | 5.24% |
| RBD Palm Oil - CP6 | 16.10% |
| Hemp Oil | 10.09% |
| Cod Liver Oil | 10.00% |
| perfume-B | 0.07%. |

140. A method of treating wounds, burns or skin conditions of a subject comprising applying topically the anhydrous topical composition of claim 112 to the area of the skin having the burns or skin conditions or to the periwound thereof.

141. A method of treating burns or skin conditions of a subject comprising applying topically the anhydrous topical composition of claim 134 to the area of the skin having the burns or skin conditions or to the periwound thereof.

142. A method of treating burns or skin conditions of a subject comprising applying topically the anhydrous topical composition of claim 139 to the area of the skin having the burns or skin conditions or to the periwound thereof.

143. A method of moisturizing the skin of a subject by applying daily to the skin the anhydrous topical composition of claim 112.

144. A method of moisturizing the skin of a subject by applying daily to the skin the anhydrous topical composition of claim 134.

145. A method of moisturizing the skin of a subject by applying daily to the skin the anhydrous topical composition of claim 139.

146. A method of treating dry eye syndrome comprising applying to the base of the dry eye the anhydrous topical composition according to claim 112.

147. A method of treating dry eye syndrome comprising applying to the base of the dry eye the anhydrous topical composition according to claim 134.

148. A method of treating dry eye syndrome comprising applying to the base of the dry eye the anhydrous topical composition according to claim 139.

149. A kit comprising Lidocaine Lavage-AA, Gel CS1, Collagen Matrix CR.
